SEQUENCE LISTING (6 sequences)

SEQ ID NO: 1 - DNA, 20 bp, Artificial Sequence
  Synthetic Construct
tgtcatgcagaagtacctgg

SEQ ID NO: 2 - DNA, 20 bp, Artificial Sequence
  Synthetic Construct
gatttgtcaaaacctccgag

SEQ ID NO: 3 - DNA, 20 bp, Artificial Sequence
  Synthetic Construct
ggtcccggtagaccacgaag

SEQ ID NO: 4 - DNA, 20 bp, Artificial Sequence
  Synthetic Construct
gcaccccagagagaaccagt

SEQ ID NO: 5 - DNA, 20 bp, Artificial Sequence
  Synthetic Construct
ctttcttgcgagcctccagc

SEQ ID NO: 6 - DNA, 20 bp, Artificial Sequence
  Synthetic Construct
gctgcgcgacgcctaccgcg

(19) United States
(12) Patent Application Publication
(10) Pub. No.: US 2023/0035892 A1
(43) Pub. Date: Feb. 2, 2023

(54) METHODS AND COMPOSITIONS FOR TREATING CANCER

(71) Applicant: Flagship Pioneering Innovations V, Inc., Cambridge, MA (US)

(72) Inventors: Jordi MATA-FINK, Happy Valley, CA (US); Avak KAHVEJIAN, Lexington, MA (US); Jonathan Barry HUROV, Bedford, MA (US); Alexandra LANTERMANN, Boston, MA (US)

(21) Appl. No.: 17/639,461

(22) PCT Filed: Sep. 3, 2020
(86) PCT No.: PCT/US2020/049211

§ 371 Date: Mar. 1, 2022

Related U.S. Application Data
(60) Provisional application No. 62/895,011, filed on Sep. 3, 2019.

(51) Int. Cl. C12N 9/12; C12Q 1/6886

(52) U.S. Cl. CPC C12N 9/12; C12Q 1/6886; C12Y 207/11015; C12Y 207/11016; C12Q 2600/158

(57) ABSTRACT

The present invention provides methods for treating cancer using GRK2 inhibitors such as small molecule inhibitors of GRK2 among others. The invention also features compositions containing GRK2 inhibitors, methods of diagnosing patients with GRK2-associated cancer and methods of predicting the response of cancer in a subject to treatment with GRK2 inhibitors.

### SEQUENCE LISTING

The instant application contains a Sequence Listing which has been submitted electronically in ASCII format and is hereby incorporated by reference in its entirety. The ASCII copy, created on Sep. 3, 2020, is named 51247-071WO2_Sequence_Listing_9_3_20_ST25 and is 1,405 bytes in size.

### BACKGROUND

Cancer is still one of the deadliest threats to human health. In 2012, there were 14 million new cases of cancer worldwide and 8.2 million cancer-related deaths. The number of new cancer cases is expected to rise to 22 million by 2030, and worldwide cancer deaths are projected to increase by 60%. Thus, there remains a need in the field for treatments for cancer.

### SUMMARY OF THE INVENTION

The present invention provides methods for treating cancer using G-protein-coupled receptor kinase 2 (GRK2) inhibitors, such as small molecule antagonists of GRK2, among others. The invention also features compositions containing GRK2 inhibitors, methods of diagnosing patients with GRK2-associated cancer, and methods of predicting the response of cancer in a subject to treatment with a GRK2 inhibitor.

In a first aspect, the invention provides a method of treating a subject with cancer, by administering to the subject an effective amount of a GRK2 inhibitor.

In another aspect, the invention provides a method of treating a subject with cancer by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject identified as having cancer by administering to the subject an effective amount of a GRK2 inhibitor.

In another aspect, the invention provides a method of treating a subject identified as having cancer by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject with cancer by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject with cancer by contacting a tumor, tumor microenvironment, site of metastasis, cancer cell, metastatic cancer cell, or stromal cell in a tumor microenvironment with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject identified as having cancer by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject identified as having cancer by contacting a tumor, tumor microenvironment, site of metastasis, cancer cell, metastatic cancer cell, or stromal cell in a tumor microenvironment with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject with pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma by administering to the subject an effective amount of a GRK2 inhibitor.

In another aspect, the invention provides a method of treating a subject identified as having pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma by administering to the subject an effective amount of a GRK2 inhibitor.

In another aspect, the invention provides a method of treating a subject with pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject identified as having pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of decreasing or inhibiting cancer cell proliferation in a subject in need thereof by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of decreasing or inhibiting cancer cell proliferation in a subject in need thereof by contacting a cancer cell with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of decreasing or inhibiting cancer or tumor growth in a subject in need thereof by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of decreasing or inhibiting cancer or tumor growth in a subject in need thereof by contacting a tumor, tumor microenvironment, or cancer cell with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of decreasing or inhibiting cancer or tumor metastasis in a subject in need thereof by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of increasing or inducing cancer cell death (e.g., apoptosis) in a subject in need thereof by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In some embodiments of any of the above aspects, the method includes identifying the cancer as GRK2-associated cancer prior to administration of the GRK2 inhibitor (e.g., the GRK2 small molecule antagonist).

In another aspect, the invention provides a method of treating a subject with cancer by: (a) identifying a subject with GRK2-associated cancer; and (b) administering to the subject an effective amount of a GRK2 inhibitor.

In another aspect, the invention provides a method of treating a subject with cancer by: (a) identifying a subject with GRK2-associated cancer; and (b) contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject with GRK2-associated cancer by administering to the subject an effective amount of a GRK2 inhibitor.

In another aspect, the invention provides a method of treating a subject with GRK2-associated cancer by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject identified as having GRK2-associated cancer by administering to the subject an effective amount of a GRK2 inhibitor.

In another aspect, the invention provides a method of treating a subject identified as having GRK2-associated cancer by contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of decreasing the number or activity of nerve fibers in a tumor, tumor microenvironment or site of metastasis by contacting a tumor, tumor microenvironment or site of metastasis with a GRK2 inhibitor.

In another aspect, the invention provides a method of treating a subject with cancer, the method including the steps of: (a) identifying a subject with GRK2-associated cancer; and (b) administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject with pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma, the method including the steps of: (a) identifying a subject with GRK2-associated cancer; and (b) administering to the subject an effective amount of a GRK2 inhibitor.

In another aspect, the invention provides a method of treating a subject with cancer, the method including the steps of: (a) identifying a subject with GRK2-associated cancer; and (b) contacting a tumor, tumor microenvironment, site of metastasis, cancer cell, metastatic cancer cell, or stromal cell in a tumor microenvironment with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject with pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma, the method including the steps of: (a) identifying a subject with GRK2-associated cancer; and (b) contacting a tumor, tumor microenvironment, site of metastasis, cancer cell, metastatic cancer cell, or stromal cell in a tumor microenvironment with an effective amount of a GRK2 inhibitor.

In another aspect, the invention provides a method of treating a subject with GRK2-associated cancer by administering to the subject an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of treating a subject with GRK2-associated cancer by contacting a tumor, tumor microenvironment, site of metastasis, cancer cell, metastatic cancer cell, or stromal cell in a tumor microenvironment with an effective amount of a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of decreasing the number or activity of nerve fibers in a tumor, tumor microenvironment or site of metastasis in a GRK2-associated cancer by contacting a tumor, tumor microenvironment or site of metastasis with a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In some embodiments of any of the above aspects, the method includes contacting a tumor with an effective amount of a GRK2 inhibitor. In some embodiments of any of the above aspects, the method includes contacting a tumor microenvironment with an effective amount of a GRK2 inhibitor. In some embodiments of any of the above aspects, the method includes contacting a site of metastasis with an effective amount of a GRK2 inhibitor. In some embodiments of any of the above aspects, the method includes contacting a cancer cell with an effective amount of a GRK2 inhibitor. In some embodiments of any of the above aspects, the method includes contacting a metastatic cancer cell with an effective amount of a GRK2 inhibitor. In some embodiments of any of the above aspects, the method includes contacting a stromal cell in a tumor microenvironment with an effective amount of a GRK2 inhibitor.

In another aspect, the invention provides a method of predicting the response of a cancer in a subject to treatment with a GRK2 inhibitor by contacting a cancer cell isolated from the subject with a GRK2 inhibitor and evaluating the response of the cancer cell.

In another aspect, the invention provides a method of predicting the response of a cancer in a subject to treatment with a GRK2 inhibitor by contacting a cancer cell isolated from the subject with a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3 and evaluating the response of the cancer cell.

In another aspect, the invention provides a method of predicting the response of a pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma in a subject to treatment with a GRK2 inhibitor by contacting a cancer cell isolated from the subject with a GRK2 inhibitor and evaluating the response of the cancer cell.

In some embodiments of any of the above aspects, the evaluating includes assessing cancer cell growth, cancer cell proliferation, cancer cell metastasis, cancer cell invasion, cancer cell migration, cancer cell death, cancer cell autophagy, cancer cell GRK2 expression, or cancer cell innervation. In some embodiments of any of the above aspects, the evaluating includes assessing cancer cell proliferation. In some embodiments of any of the above aspects, the evaluating includes assessing cancer cell growth.

In another aspect, the invention provides a method of predicting the response of a cancer in a subject to treatment with a GRK2 inhibitor by: (a) isolating a cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject will respond to treatment with a GRK2 inhibitor.

In another aspect, the invention provides a method of predicting the response of a cancer in a subject to treatment with a GRK2 inhibitor, the method including the steps of: (a) isolating a cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject will respond to treatment with a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3.

In another aspect, the invention provides a method of predicting the response of a pancreatic cancer, small cell lung cancer, non-small cell lung cancer, gastric cancer, colorectal cancer, head and neck cancer, testicular cancer, thymoma, uterine cancer, kidney cancer, acute myeloid leukemia, diffuse large B-cell lymphoma, or hepatocellular carcinoma in a subject to treatment with a GRK2 inhibitor, the method including the steps of: (a) isolating a cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject will respond to treatment with a GRK2 inhibitor.

In some embodiments of any of the above aspects, the method further includes contacting the cancer cell with a GRK2 inhibitor.

In another aspect, the invention provides a method of characterizing a cancer in a subject by: (a) isolating a cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject has GRK2-associated cancer.

In another aspect, the invention provides a method of identifying a subject as having GRK2-associated cancer by: (a) isolating cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject has GRK2-associated cancer.

In some embodiments of any of the above aspects, the method further includes providing a GRK2 inhibitor suitable for administration to the subject. In some embodiments of any of the above aspects, the method further includes administering to the subject an effective amount of a GRK2 inhibitor. In some embodiments of any of the above aspects, the GRK2 inhibitor is a GRK2-specific inhibitor.

In some embodiments of any of the above aspects, the cancer is GRK2-associated cancer.

In some embodiments of any of the above aspects, the GRK2-associated cancer expresses GRK2. In some embodiments of any of the above aspects, the GRK2-associated cancer overexpresses GRK2.

In another aspect, the invention provides an anti-cancer therapy containing a GRK2 inhibitor and a second agent selected from the group including checkpoint inhibitors, chemotherapeutic agents, biologic cancer agents (e.g., an antibody listed in Table 4), cancer-specific agents (e.g., an agent listed in Table 5), CAR-T therapy, cytokine therapy (e.g., an interferon or interleukin, such as IL-2 or IL-12), oncolytic viruses (e.g., tamilogene laherparepvec (T-VEC), canerpaturev, enadenotucirev, pelareorep, pexastimogene devacirepvec (JX-594), or tasadenoturev), anti-angiogenic drugs, drugs that target cancer metabolism, antibodies that mark a cancer cell surface for destruction, antibody-drug conjugates, cell therapies, commonly used anti-neoplastic agents, non-drug therapies, neurotransmission blockers, and neuronal growth factor blockers.

In another aspect, the invention provides an anti-cancer therapy containing a GRK2 inhibitor selected from the group consisting of a GRK2 small molecule antagonist, a nuclease directed to GRK2, an inhibitory RNA (e.g., shRNA, siRNA, or miRNA) directed to a GRK2 binding partner listed in Table 2, a nuclease directed to a GRK2 binding partner listed in Table 2, and a GRK2 signaling inhibitor listed in Table 3 and a second agent selected from the group consisting of chemotherapeutic agents, checkpoint inhibitors, biologic cancer agents (e.g., an antibody listed in Table 4), cancer-specific agents (e.g., an agent listed in Table 5), cytokine therapies (e.g., an interferon or interleukin, such as IL-2 or IL-12), anti-angiogenic drugs, drugs that target cancer metabolism, antibodies that mark a cancer cell surface for destruction, antibody-drug conjugates, cell therapies, commonly used anti-neoplastic agents, CAR-T therapies, oncolytic viruses (e.g., tamilogene laherparepvec (T-VEC), canerpaturev, enadenotucirev, pelareorep, pexastimogene devacirepvec (JX-594), or tasadenoturev), non-drug therapies, neurotransmission blockers, and neuronal growth factor blockers.

In another aspect, the invention provides an anti-cancer therapy containing a GRK2 small molecule antagonist and a second agent selected from the group consisting of chemotherapeutic agents, checkpoint inhibitors, biologic cancer agents, cancer-specific agents, cytokine therapies, anti-angiogenic drugs, drugs that target cancer metabolism, antibodies that mark a cancer cell surface for destruction, antibody-drug conjugates, cell therapies, commonly used anti-neoplastic agents, CAR-T therapies, oncolytic viruses, non-drug therapies, neurotransmission blockers, and neuronal growth factor blockers.

In some embodiments of any of the above aspects, the second agent is a chemotherapeutic agent. In some embodiments of any of the above aspects, the second agent is a checkpoint inhibitor.

In another aspect, the invention provides a pharmaceutical composition containing a GRK2 small molecule antagonist and an anti-cancer therapeutic.

In some embodiments of any of the above aspects, the GRK2 inhibitor is a GRK2-specific inhibitor.

In some embodiments of any of the above aspects, the GRK2 inhibitor is a GRK2 small molecule antagonist.

In some embodiments of any of the foregoing aspects, the GRK2 small molecule antagonist is any compound described herein (e.g., a compound having the structure of any one of Formula I (e.g., any one of compounds of Formula I-1 to I-39), Formula II (e.g., any one of compounds of Formula II-1 to II-35), Formula III (e.g., any one of compounds of Formula III-1 to III-56), Formula IV (e.g., any one of compounds of Formula IV-1 to IV-80), or Compounds 1-70). In some embodiments of any of the above aspects, the GRK2 small molecule antagonist is Compound 1. In some embodiments of any of the above aspects, the GRK2 small molecule antagonist is Compound 2. In some embodiments of any of the above aspects, the GRK2 small molecule antagonist is Compound 3. In some embodiments of any of the above aspects, the GRK2 small molecule antagonist is Compound 4. In some embodiments of any of the above aspects, the GRK2 small molecule antagonist is Compound 5. In some embodiments of any of the above aspects, the GRK2 small molecule antagonist is Compound 6. In some embodiments of any of the above aspects, the GRK2 small molecule antagonist is Compound 7. In some embodiments of any of the above aspects, the GRK2 small molecule antagonist is Compound 8. In some embodiments of any of the above aspects, the GRK2 small molecule antagonist is Compound 9. In some embodiments of any of the above aspects, the GRK2 small molecule antagonist is Compound 10. In some embodiments of any of the above aspects, the GRK2 small molecule antagonist is Compound 11. In some embodiments of any of the above aspects, the GRK2 small molecule antagonist is Compound 12. In some embodiments of any of the above aspects, the GRK2 small molecule antagonist is Compound 13. In some embodiments of any of the above aspects, the GRK2 small molecule antagonist is Compound 14. In some embodiments of any of the above aspects, the GRK2 small molecule antagonist is Compound 15. In some embodiments of any of the above aspects, the GRK2 small molecule antagonist is Compound 16. In some embodiments of any of the above aspects, the GRK2 small molecule antagonist is Compound 17. In some embodiments of any of the above aspects, the GRK2 small molecule antagonist is Compound 18. In some embodiments of any of the above aspects, the GRK2 small molecule antagonist is Compound 19. In some embodiments of any of the above aspects, the GRK2 small molecule antagonist is Compound 20. In some embodiments of any of the above aspects, the GRK2 small molecule antagonist is Compound 21. In some embodiments of any of the above aspects, the GRK2 small molecule antagonist is Compound 22. In some embodiments of any of the above aspects, the GRK2 small molecule antagonist is Compound 23. In some embodiments of any of the above aspects, the GRK2 small molecule antagonist is Compound 24. In some embodiments of any of the above aspects, the GRK2 small molecule antagonist is Compound 25. In some embodiments of any of the above aspects, the GRK2 small molecule antagonist is Compound 26. In some embodiments of any of the above aspects, the GRK2 small molecule antagonist is Compound 27. In some embodiments of any of the above aspects, the GRK2 small molecule antagonist is Compound 28. In some embodiments of any of the above aspects, the GRK2 small molecule antagonist is Compound 29. In some embodiments of any of the above aspects, the GRK2 small molecule antagonist is Compound 30. In some embodiments of any of the above aspects, the GRK2 small molecule antagonist is Compound 31. In some embodiments of any of the above aspects, the GRK2 small molecule antagonist is Compound 32. In some embodiments of any of the above aspects, the GRK2 small molecule antagonist is Compound 33. In some embodiments of any of the above aspects, the GRK2 small molecule antagonist is Compound 34. In some embodiments of any of the above aspects, the GRK2 small molecule antagonist is Compound 35. In some embodiments of any of the above aspects, the GRK2 small molecule antagonist is Compound 36. In some embodiments of any of the above aspects, the GRK2 small molecule antagonist is Compound 37. In some embodiments of any of the above aspects, the GRK2 small molecule antagonist is Compound 38. In some embodiments of any of the above aspects, the GRK2 small molecule antagonist is Compound 39. In some embodiments of any of the above aspects, the GRK2 small molecule antagonist is Compound 40. In some embodiments of any of the above aspects, the GRK2 small molecule antagonist is Compound 41. In some embodiments of any of the above aspects, the GRK2 small molecule antagonist is Compound 42. In some embodiments of any of the above aspects, the GRK2 small molecule antagonist is Compound 43. In some embodiments of any of the above aspects, the GRK2 small molecule antagonist is Compound 44. In some embodiments of any of the above aspects, the GRK2 small molecule antagonist is Compound 45. In some embodiments of any of the above aspects, the GRK2 small molecule antagonist is Compound 46. In some embodiments of any of the above aspects, the GRK2 small molecule antagonist is Compound 47. In some embodiments of any of the above aspects, the GRK2 small molecule antagonist is Compound 48. In some embodiments of any of the above aspects, the GRK2 small molecule antagonist is Compound 49. In some embodiments of any of the above aspects, the GRK2 small molecule antagonist is Compound 50. In some embodiments of any of the above aspects, the GRK2 small molecule antagonist is Compound 51. In some embodiments of any of the above aspects, the GRK2 small molecule antagonist is Compound 52. In some embodiments of any of the above aspects, the GRK2 small molecule antagonist is Compound 53. In some embodiments of any of the above aspects, the GRK2 small molecule antagonist is Compound 54. In some embodiments of any of the above aspects, the GRK2 small molecule antagonist is Compound 55. In some embodiments of any of the above aspects, the GRK2 small molecule antagonist is Compound 56. In some embodiments of any of the above aspects, the GRK2 small molecule antagonist is Compound 57. In some embodiments of any of the above aspects, the GRK2 small molecule antagonist is Compound 58. In some embodiments of any of the above aspects, the GRK2 small molecule antagonist is Compound 59. In some embodiments of any of the above aspects, the GRK2 small molecule antagonist is Compound 60. In some embodiments of any of the above aspects, the GRK2 small molecule antagonist is Compound 61. In some embodiments of any of the above aspects, the GRK2 small molecule antagonist is Compound 62. In some embodiments of any of the above aspects, the GRK2 small molecule antagonist is Compound 63. In some embodiments of any of the above aspects, the GRK2 small molecule antagonist is Compound 64. In some embodiments of any of the above aspects, the GRK2 small molecule antagonist is Compound 65. In some embodiments of any of the above aspects, the GRK2 small molecule antagonist is Compound 66. In some embodiments of any of the above aspects, the GRK2 small molecule antagonist is Compound 67. In some embodiments of any of the above aspects, the GRK2 small molecule antagonist is Compound 68. In some embodiments of any of the above aspects, the GRK2 small molecule antagonist is Compound 69. In some embodiments of any of the above aspects, the GRK2 small molecule antagonist is Compound 70. In some embodiments of any of the above aspects, the GRK2 small molecule antagonist is a Compound of Formula I. In some embodiments of any of the above aspects, the GRK2 small molecule antagonist is a Compound of Formula I-1. In some embodiments of any of the above aspects, the GRK2 small molecule antagonist is a Compound of Formula I-2. In some embodiments of any of the above aspects, the GRK2 small molecule antagonist is a Compound of Formula I-3. In some embodiments of any of the above aspects, the GRK2 small molecule antagonist is a Compound of Formula I-4. In some embodiments of any of the above aspects, the GRK2 small molecule antagonist is a Compound of Formula I-5. In some embodiments of any of the above aspects, the GRK2 small molecule antagonist is a Compound of Formula I-6. In some embodiments of any of the above aspects, the GRK2 small molecule antagonist is a Compound of Formula I-7. In some embodiments of any of the above aspects, the GRK2 small molecule antagonist is a Compound of Formula I-8. In some embodiments of any of the above aspects, the GRK2 small molecule antagonist is a Compound of Formula I-9. In some embodiments of any of the above aspects, the GRK2 small molecule antagonist is a Compound of Formula I-10. In some embodiments of any of the above aspects, the GRK2 small molecule antagonist is a Compound of Formula I-11. In some embodiments of any of the above aspects, the GRK2 small molecule antagonist is a Compound of Formula I-12. In some embodiments of any of the above aspects, the GRK2 small molecule antagonist is a Compound of Formula I-13. In some embodiments of any of the above aspects, the GRK2 small molecule antagonist is a Compound of Formula I-14. In some embodiments of any of the above aspects, the GRK2 small molecule antagonist is a Compound of Formula I-15. In some embodiments of any of the above aspects, the GRK2 small molecule antagonist is a Compound of Formula I-16. In some embodiments of any of the above aspects, the GRK2 small molecule antagonist is a Compound of Formula I-17. In some embodiments of any of the above aspects, the GRK2 small molecule antagonist is a Compound of Formula I-18. In some embodiments of any of the above aspects, the GRK2 small molecule antagonist is a Compound of Formula I-19. In some embodiments of any of the above aspects, the GRK2 small molecule antagonist is a Compound of Formula I-20. In some embodiments of any of the above aspects, the GRK2 small molecule antagonist is a Compound of Formula I-21. In some embodiments of any of the above aspects, the GRK2 small molecule antagonist is a Compound of Formula I-22. In some embodiments of any of the above aspects, the GRK2 small molecule antagonist is a Compound of Formula I-23. In some embodiments of any of the above aspects, the GRK2 small molecule antagonist is a Compound of Formula I-24. In some embodiments of any of the above aspects, the GRK2 small molecule antagonist is a Compound of Formula I-25. In some embodiments of any of the above aspects, the GRK2 small molecule antagonist is a compound of Formula I-26. In some embodiments of any of the above aspects, the GRK2 small molecule antagonist is a Compound of Formula I-27. In some embodiments of any of the above aspects, the GRK2 small molecule antagonist is a Compound of Formula I-28. In some embodiments of any of the above aspects, the GRK2 small molecule antagonist is a Compound of Formula I-29. In some embodiments of any of the above aspects, the GRK2 small molecule antagonist is a Compound of Formula I-30. In some embodiments of any of the above aspects, the GRK2 small molecule antagonist is a Compound of Formula I-31. In some embodiments of any of the above aspects, the GRK2 small molecule antagonist is a Compound of Formula I-32. In some embodiments of any of the above aspects, the GRK2 small molecule antagonist is a Compound of Formula I-33. In some embodiments of any of the above aspects, the GRK2 small molecule antagonist is a Compound of Formula I-34. In some embodiments of any of the above aspects, the GRK2 small molecule antagonist is a Compound of Formula I-35. In some embodiments of any of the above aspects, the GRK2 small molecule antagonist is a Compound of Formula I-36. In some embodiments of any of the above aspects, the GRK2 small molecule antagonist is a Compound of Formula I-37. In some embodiments of any of the above aspects, the GRK2 small molecule antagonist is a Compound of Formula I-38. In some embodiments of any of the above aspects, the GRK2 small molecule antagonist is a Compound of Formula I-39. In some embodiments of any of the above aspects, the GRK2 small molecule antagonist is a Compound of Formula II. In some embodiments of any of the above aspects, the GRK2 small molecule antagonist is a Compound of Formula II-1. In some embodiments of any of the above aspects, the GRK2 small molecule antagonist is a Compound of Formula II-2. In some embodiments of any of the above aspects, the GRK2 small molecule antagonist is a Compound of Formula II-3. In some embodiments of any of the above aspects, the GRK2 small molecule antagonist is a Compound of Formula II-4. In some embodiments of any of the above aspects, the GRK2 small molecule antagonist is a Compound of Formula II-5. In some embodiments of any of the above aspects, the GRK2 small molecule antagonist is a Compound of Formula II-6. In some embodiments of any of the above aspects, the GRK2 small molecule antagonist is a Compound of Formula II-7. In some embodiments of any of the above aspects, the GRK2 small molecule antagonist is a Compound of Formula II-8. In some embodiments of any of the above aspects, the GRK2 small molecule antagonist is a Compound of Formula II-9. In some embodiments of any of the above aspects, the GRK2 small molecule antagonist is a Compound of Formula II-10. In some embodiments of any of the above aspects, the GRK2 small molecule antagonist is a Compound of Formula II-11. In some embodiments of any of the above aspects, the GRK2 small molecule antagonist is a Compound of Formula II-12. In some embodiments of any of the above aspects, the GRK2 small molecule antagonist is a Compound of Formula II-13. In some embodiments of any of the above aspects, the GRK2 small molecule antagonist is a Compound of Formula II-14. In some embodiments of any of the above aspects, the GRK2 small molecule antagonist is a Compound of Formula II-15. In some embodiments of any of the above aspects, the GRK2 small molecule antagonist is a Compound of Formula II-16. In some embodiments of any of the above aspects, the GRK2 small molecule antagonist is a Compound of Formula II-17. In some embodiments of any of the above aspects, the GRK2 small molecule antagonist is a Compound of Formula II-18. In some embodiments of any of the above aspects, the GRK2 small molecule antagonist is a Compound of Formula II-19. In some embodiments of any of the above aspects, the GRK2 small molecule antagonist is a Compound of Formula II-20. In some embodiments of any of the above aspects, the GRK2 small molecule antagonist is a Compound of Formula II-21. In some embodiments of any of the above aspects, the GRK2 small molecule antagonist is a Compound of Formula II-22. In some embodiments of any of the above aspects, the GRK2 small molecule antagonist is a Compound of Formula II-23. In some embodiments of any of the above aspects, the GRK2 small molecule antagonist is a Compound of Formula II-24. In some embodiments of any of the above aspects, the GRK2 small molecule antagonist is a Compound of Formula II-25. In some embodiments of any of the above aspects, the GRK2 small molecule antagonist is a Compound of Formula II-26. In some embodiments of any of the above aspects, the GRK2 small molecule antagonist is a Compound of Formula II-27. In some embodiments of any of the above aspects, the GRK2 small molecule antagonist is a Compound of Formula II-28. In some embodiments of any of the above aspects, the GRK2 small molecule antagonist is a Compound of Formula II-29. In some embodiments of any of the above aspects, the GRK2 small molecule antagonist is a Compound of Formula II-30. In some embodiments of any of the above aspects, the GRK2 small molecule antagonist is a Compound of Formula II-31. In some embodiments of any of the above aspects, the GRK2 small molecule antagonist is a Compound of Formula II-32. In some embodiments of any of the above aspects, the GRK2 small molecule antagonist is a Compound of Formula II-33. In some embodiments of any of the above aspects, the GRK2 small molecule antagonist is a Compound of Formula II-34. In some embodiments of any of the above aspects, the GRK2 small molecule antagonist is a Compound of Formula II-35. In some embodiments of any of the above aspects, the GRK2 small molecule antagonist is a Compound of Formula III. In some embodiments of any of the above aspects, the GRK2 small molecule antagonist is a Compound of Formula III-1. In some embodiments of any of the above aspects, the GRK2 small molecule antagonist is a Compound of Formula III-2. In some embodiments of any of the above aspects, the GRK2 small molecule antagonist is a Compound of Formula III-3. In some embodiments of any of the above aspects, the GRK2 small molecule antagonist is a Compound of Formula III-4. In some embodiments of any of the above aspects, the GRK2 small molecule antagonist is a Compound of Formula III-5. In some embodiments of any of the above aspects, the GRK2 small molecule antagonist is a Compound of Formula III-6. In some embodiments of any of the above aspects, the GRK2 small molecule antagonist is a Compound of Formula III-7. In some embodiments of any of the above aspects, the GRK2 small molecule antagonist is a Compound of Formula III-8. In some embodiments of any of the above aspects, the GRK2 small molecule antagonist is a Compound of Formula III-9. In some embodiments of any of the above aspects, the GRK2 small molecule antagonist is a Compound of Formula III-10. In some embodiments of any of the above aspects, the GRK2 small molecule antagonist is a Compound of Formula III-11. In some embodiments of any of the above aspects, the GRK2 small molecule antagonist is a Compound of Formula III-12. In some embodiments of any of the above aspects, the GRK2 small molecule antagonist is a Compound of Formula III-13. In some embodiments of any of the above aspects, the GRK2 small molecule antagonist is a Compound of Formula III-14. In some embodiments of any of the above aspects, the GRK2 small molecule antagonist is a Compound of Formula III-15. In some embodiments of any of the above aspects, the GRK2 small molecule antagonist is a Compound of Formula III-16. In some embodiments of any of the above aspects, the GRK2 small molecule antagonist is a Compound of Formula III-17. In some embodiments of any of the above aspects, the GRK2 small molecule antagonist is a Compound of Formula III-18. In some embodiments of any of the above aspects, the GRK2 small molecule antagonist is a Compound of Formula III-19. In some embodiments of any of the above aspects, the GRK2 small molecule antagonist is a Compound of Formula III-20. In some embodiments of any of the above aspects, the GRK2 small molecule antagonist is a Compound of Formula III-21. In some embodiments of any of the above aspects, the GRK2 small molecule antagonist is a Compound of Formula III-22. In some embodiments of any of the above aspects, the GRK2 small molecule antagonist is a Compound of Formula III-23. In some embodiments of any of the above aspects, the GRK2 small molecule antagonist is a Compound of Formula III-24. In some embodiments of any of the above aspects, the GRK2 small molecule antagonist is a Compound of Formula III-25. In some embodiments of any of the above aspects, the GRK2 small molecule antagonist is a Compound of Formula III-26. In some embodiments of any of the above aspects, the GRK2 small molecule antagonist is a Compound of Formula III-27. In some embodiments of any of the above aspects, the GRK2 small molecule antagonist is a Compound of Formula III-28. In some embodiments of any of the above aspects, the GRK2 small molecule antagonist is a Compound of Formula III-29. In some embodiments of any of the above aspects, the GRK2 small molecule antagonist is a Compound of Formula III-30. In some embodiments of any of the above aspects, the GRK2 small molecule antagonist is a Compound of Formula III-31. In some embodiments of any of the above aspects, the GRK2 small molecule antagonist is a Compound of Formula III-32. In some embodiments of any of the above aspects, the GRK2 small molecule antagonist is a Compound of Formula III-33. In some embodiments of any of the above aspects, the GRK2 small molecule antagonist is a Compound of Formula III-34. In some embodiments of any of the above aspects, the GRK2 small molecule antagonist is a Compound of Formula III-35. In some embodiments of any of the above aspects, the GRK2 small molecule antagonist is a Compound of Formula III-36. In some embodiments of any of the above aspects, the GRK2 small molecule antagonist is a Compound of Formula III-37. In some embodiments of any of the above aspects, the GRK2 small molecule antagonist is a Compound of Formula III-38. In some embodiments of any of the above aspects, the GRK2 small molecule antagonist is a Compound of Formula III-39. In some embodiments of any of the above aspects, the GRK2 small molecule antagonist is a Compound of Formula III-40. In some embodiments of any of the above aspects, the GRK2 small molecule antagonist is a Compound of Formula III-41. In some embodiments of any of the above aspects, the GRK2 small molecule antagonist is a Compound of Formula III-42. In some embodiments of any of the above aspects, the GRK2 small molecule antagonist is a Compound of Formula III-43. In some embodiments of any of the above aspects, the GRK2 small molecule antagonist is a Compound of Formula III-44. In some embodiments of any of the above aspects, the GRK2 small molecule antagonist is a Compound of Formula III-45. In some embodiments of any of the above aspects, the GRK2 small molecule antagonist is a Compound of Formula III-46. In some embodiments of any of the above aspects, the GRK2 small molecule antagonist is a Compound of Formula III-47. In some embodiments of any of the above aspects, the GRK2 small molecule antagonist is a Compound of Formula III-48. In some embodiments of any of the above aspects, the GRK2 small molecule antagonist is a Compound of Formula III-49. In some embodiments of any of the above aspects, the GRK2 small molecule antagonist is a Compound of Formula III-50. In some embodiments of any of the above aspects, the GRK2 small molecule antagonist is a Compound of Formula III-51. In some embodiments of any of the above aspects, the GRK2 small molecule antagonist is a Compound of Formula III-52. In some embodiments of any of the above aspects, the GRK2 small molecule antagonist is a Compound of Formula III-53. In some embodiments of any of the above aspects, the GRK2 small molecule antagonist is a Compound of Formula III-54. In some embodiments of any of the above aspects, the GRK2 small molecule antagonist is a Compound of Formula III-55. In some embodiments of any of the above aspects, the GRK2 small molecule antagonist is a Compound of Formula III-56. In some embodiments of any of the above aspects, the GRK2 small molecule antagonist is a Compound of Formula IV. In some embodiments of any of the above aspects, the GRK2 small molecule antagonist is a Compound of Formula IV-1. In some embodiments of any of the above aspects, the GRK2 small molecule antagonist is a Compound of Formula IV-2. In some embodiments of any of the above aspects, the GRK2 small molecule antagonist is a Compound of Formula IV-3. In some embodiments of any of the above aspects, the GRK2 small molecule antagonist is a Compound of Formula IV-4. In some embodiments of any of the above aspects, the GRK2 small molecule antagonist is a Compound of Formula IV-5. In some embodiments of any of the above aspects, the GRK2 small molecule antagonist is a Compound of Formula IV-6. In some embodiments of any of the above aspects, the GRK2 small molecule antagonist is a Compound of Formula IV-7. In some embodiments of any of the above aspects, the GRK2 small molecule antagonist is a Compound of Formula IV-8. In some embodiments of any of the above aspects, the GRK2 small molecule antagonist is a Compound of Formula IV-9. In some embodiments of any of the above aspects, the GRK2 small molecule antagonist is a Compound of Formula IV-10. In some embodiments of any of the above aspects, the GRK2 small molecule antagonist is a Compound of Formula IV-11. In some embodiments of any of the above aspects, the GRK2 small molecule antagonist is a Compound of Formula IV-12. In some embodiments of any of the above aspects, the GRK2 small molecule antagonist is a Compound of Formula IV-13. In some embodiments of any of the above aspects, the GRK2 small molecule antagonist is a Compound of Formula IV-14. In some embodiments of any of the above aspects, the GRK2 small molecule antagonist is a Compound of Formula IV-15. In some embodiments of any of the above aspects, the GRK2 small molecule antagonist is a Compound of Formula IV-16. In some embodiments of any of the above aspects, the GRK2 small molecule antagonist is a Compound of Formula IV-17. In some embodiments of any of the above aspects, the GRK2 small molecule antagonist is a Compound of Formula IV-18. In some embodiments of any of the above aspects, the GRK2 small molecule antagonist is a Compound of Formula IV-19. In some embodiments of any of the above aspects, the GRK2 small molecule antagonist is a Compound of Formula IV-20. In some embodiments of any of the above aspects, the GRK2 small molecule antagonist is a Compound of Formula IV-21. In some embodiments of any of the above aspects, the GRK2 small molecule antagonist is a Compound of Formula IV-22. In some embodiments of any of the above aspects, the GRK2 small molecule antagonist is a Compound of Formula IV-23. In some embodiments of any of the above aspects, the GRK2 small molecule antagonist is a Compound of Formula IV-24. In some embodiments of any of the above aspects, the GRK2 small molecule antagonist is a Compound of Formula IV-25. In some embodiments of any of the above aspects, the GRK2 small molecule antagonist is a Compound of Formula IV-26. In some embodiments of any of the above aspects, the GRK2 small molecule antagonist is a Compound of Formula IV-27. In some embodiments of any of the above aspects, the GRK2 small molecule antagonist is a Compound of Formula IV-28. In some embodiments of any of the above aspects, the GRK2 small molecule antagonist is a Compound of Formula IV-29. In some embodiments of any of the above aspects, the GRK2 small molecule antagonist is a Compound of Formula IV-30. In some embodiments of any of the above aspects, the GRK2 small molecule antagonist is a Compound of Formula IV-31. In some embodiments of any of the above aspects, the GRK2 small molecule antagonist is a Compound of Formula IV-32. In some embodiments of any of the above aspects, the GRK2 small molecule antagonist is a Compound of Formula IV-33. In some embodiments of any of the above aspects, the GRK2 small molecule antagonist is a Compound of Formula IV-34. In some embodiments of any of the above aspects, the GRK2 small molecule antagonist is a Compound of Formula IV-35. In some embodiments of any of the above aspects, the GRK2 small molecule antagonist is a Compound of Formula IV-36. In some embodiments of any of the above aspects, the GRK2 small molecule antagonist is a Compound of Formula IV-37. In some embodiments of any of the above aspects, the GRK2 small molecule antagonist is a Compound of Formula IV-38. In some embodiments of any of the above aspects, the GRK2 small molecule antagonist is a Compound of Formula IV-39. In some embodiments of any of the above aspects, the GRK2 small molecule antagonist is a Compound of Formula IV-40. In some embodiments of any of the above aspects, the GRK2 small molecule antagonist is a Compound of Formula IV-41. In some embodiments of any of the above aspects, the GRK2 small molecule antagonist is a Compound of Formula IV-42. In some embodiments of any of the above aspects, the GRK2 small molecule antagonist is a Compound of Formula IV-43. In some embodiments of any of the above aspects, the GRK2 small molecule antagonist is a Compound of Formula IV-44. In some embodiments of any of the above aspects, the GRK2 small molecule antagonist is a Compound of Formula IV-45. In some embodiments of any of the above aspects, the GRK2 small molecule antagonist is a Compound of Formula IV-46. In some embodiments of any of the above aspects, the GRK2 small molecule antagonist is a Compound of Formula IV-47. In some embodiments of any of the above aspects, the GRK2 small molecule antagonist is a Compound of Formula IV-48. In some embodiments of any of the above aspects, the GRK2 small molecule antagonist is a Compound of Formula IV-49. In some embodiments of any of the above aspects, the GRK2 small molecule antagonist is a Compound of Formula IV-50. In some embodiments of any of the above aspects, the GRK2 small molecule antagonist is a Compound of Formula IV-51. In some embodiments of any of the above aspects, the GRK2 small molecule antagonist is a Compound of Formula IV-52. In some embodiments of any of the above aspects, the GRK2 small molecule antagonist is a Compound of Formula IV-53. In some embodiments of any of the above aspects, the GRK2 small molecule antagonist is a Compound of Formula IV-54. In some embodiments of any of the above aspects, the GRK2 small molecule antagonist is a Compound of Formula IV-55. In some embodiments of any of the above aspects, the GRK2 small molecule antagonist is a Compound of Formula IV-56. In some embodiments of any of the above aspects, the GRK2 small molecule antagonist is a Compound of Formula IV-57. In some embodiments of any of the above aspects, the GRK2 small molecule antagonist is a Compound of Formula IV-58. In some embodiments of any of the above aspects, the GRK2 small molecule antagonist is a Compound of Formula IV-59. In some embodiments of any of the above aspects, the GRK2 small molecule antagonist is a Compound of Formula IV-60. In some embodiments of any of the above aspects, the GRK2 small molecule antagonist is a Compound of Formula IV-61. In some embodiments of any of the above aspects, the GRK2 small molecule antagonist is a Compound of Formula IV-62. In some embodiments of any of the above aspects, the GRK2 small molecule antagonist is a Compound of Formula IV-63. In some embodiments of any of the above aspects, the GRK2 small molecule antagonist is a Compound of Formula IV-64. In some embodiments of any of the above aspects, the GRK2 small molecule antagonist is a Compound of Formula IV-65. In some embodiments of any of the above aspects, the GRK2 small molecule antagonist is a Compound of Formula IV-66. In some embodiments of any of the above aspects, the GRK2 small molecule antagonist is a Compound of Formula IV-67. In some embodiments of any of the above aspects, the GRK2 small molecule antagonist is a Compound of Formula IV-68. In some embodiments of any of the above aspects, the GRK2 small molecule antagonist is a Compound of Formula IV-69. In some embodiments of any of the above aspects, the GRK2 small molecule antagonist is a Compound of Formula IV-70. In some embodiments of any of the above aspects, the GRK2 small molecule antagonist is a Compound of Formula IV-71. In some embodiments of any of the above aspects, the GRK2 small molecule antagonist is a Compound of Formula IV-72. In some embodiments of any of the above aspects, the GRK2 small molecule antagonist is a Compound of Formula IV-73. In some embodiments of any of the above aspects, the GRK2 small molecule antagonist is a Compound of Formula IV-74. In some embodiments of any of the above aspects, the GRK2 small molecule antagonist is a Compound of Formula IV-75. In some embodiments of any of the above aspects, the GRK2 small molecule antagonist is a Compound of Formula IV-76. In some embodiments of any of the above aspects, the GRK2 small molecule antagonist is a Compound of Formula IV-77. In some embodiments of any of the above aspects, the GRK2 small molecule antagonist is a Compound of Formula IV-78. In some embodiments of any of the above aspects, the GRK2 small molecule antagonist is a Compound of Formula IV-79. In some embodiments of any of the above aspects, the GRK2 small molecule antagonist is a Compound of Formula IV-80.

In some embodiments of any of the above aspects, the GRK2 small molecule antagonist is a compound of Formula I:

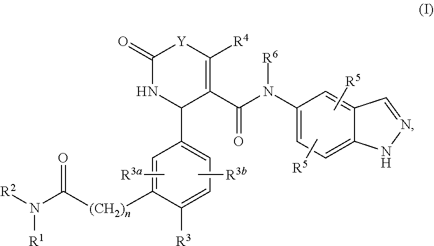

or a pharmaceutically acceptable salt thereof, where:

n is 0, 1, or 2;

X and Y are each N(R⁷), or one of X and Y is N(R⁷) and the other is CH₂;

R¹ is H, C₁-C₈ alkyl, C₂-C₈ alkenyl, C₂-C₈ alkynyl, C₃-C₈ cycloalkyl, C₃-C₈ cycloalkenyl, C₁-C₄ alkylene-aryl, C₁-C₄ alkylene-heteroaryl, C₃-C₈ cycloalkylene-aryl, or C₃-C₈ cycloalkylene-heteroaryl;

R² is C₁-C₈ alkyl, C₂-C₈ alkenyl, C₃-C₈ cycloalkyl, C₃-C₈ cycloalkenyl, C₁-C₄alkylene-cycloalkyl, C₀-C₄ alkylene-heterocycloalkyl, C₁-C₄ alkylene-aryl, C₁-C₄ alkylene-heteroaryl, C₃-C₈ cycloalkylene-aryl, or C₃-C₈ cycloalkylene-heteroaryl;

or R¹ and R², together with the nitrogen atom to which they are attached, form a 3-8-membered heterocycloalkyl group;

R³ is H, F, Cl, or CH₃;

R^{3a} and R^{3b} are each independently H, F, Cl, or CH₃;

R⁴ is H, CH₃, CF₃, CH₂CH₃, or CH₂CH₂CH₃;

each R⁵ independently is H or F;

R⁶ is H or C₁₋₆ alkyl; and

each R⁷ independently is H or C₁-C₆ alkyl.

In some embodiments, the compound of Formula I has a structure of Formula I-1:

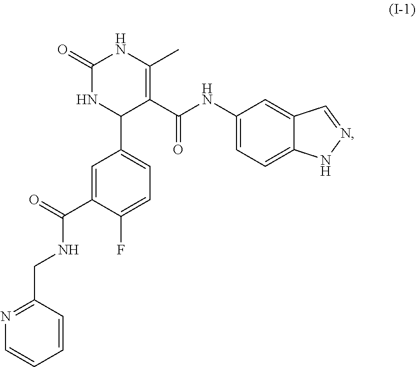 [SMILES: CC1=C(C(=O)Nc2ccc3[nH]ncc3c2)C(c2ccc(F)c(C(=O)NCc3ccccn3)c2)NC(=O)N1]

or a pharmaceutically acceptable salt thereof.

In some embodiments, the compound of Formula I has a structure of Formula I-2:

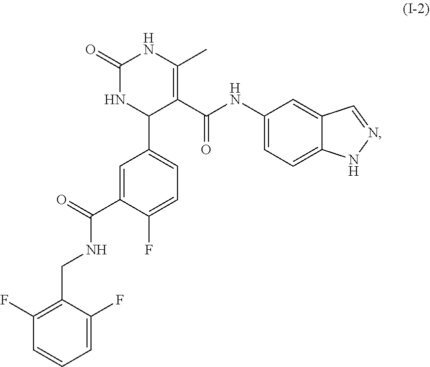 [SMILES: CC1=C(C(=O)Nc2ccc3[nH]ncc3c2)C(c2ccc(F)c(C(=O)NCc3c(F)cccc3F)c2)NC(=O)N1]

or a pharmaceutically acceptable salt thereof.

In some embodiments, the compound of Formula I has a structure of Formula I-3:

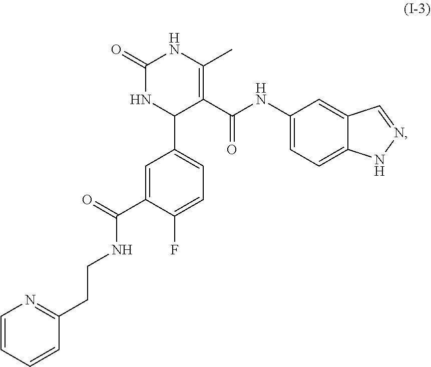 [SMILES: CC1=C(C(=O)Nc2ccc3[nH]ncc3c2)C(c2ccc(F)c(C(=O)NCCc3ccccn3)c2)NC(=O)N1]

or a pharmaceutically acceptable salt thereof.

In some embodiments, the compound of Formula I has a structure of Formula I-4:

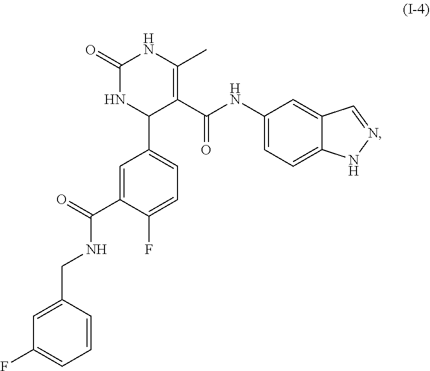 [SMILES: CC1=C(C(=O)Nc2ccc3[nH]ncc3c2)C(c2ccc(F)c(C(=O)NCc3cccc(F)c3)c2)NC(=O)N1]

or a pharmaceutically acceptable salt thereof.

In some embodiments, the compound of Formula I has a structure of Formula I-5:

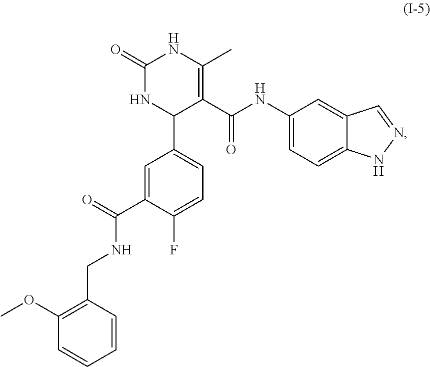 [SMILES: COc1ccccc1CNC(=O)c1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-6:

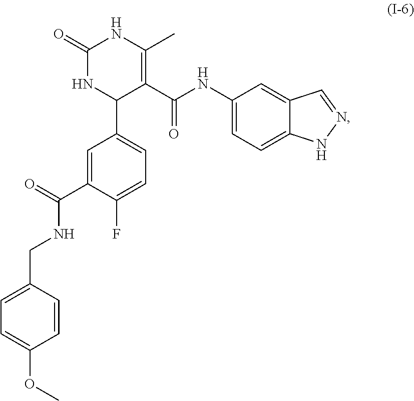 [SMILES: COc1ccc(CNC(=O)c2cc(C3NC(=O)NC(C)=C3C(=O)Nc3ccc4[nH]ncc4c3)ccc2F)cc1]

or a pharmaceutically acceptable salt thereof.

In some embodiments, the compound of Formula I has a structure of Formula I-7:

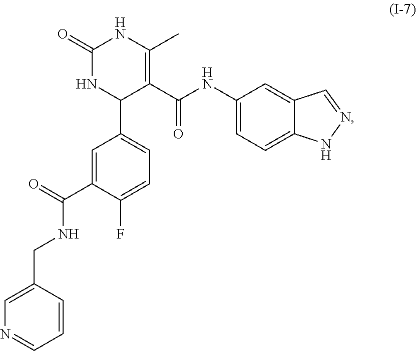 [SMILES: CC1=C(C(=O)Nc2ccc3[nH]ncc3c2)C(c2ccc(F)c(C(=O)NCc3cccnc3)c2)NC(=O)N1]

or a pharmaceutically acceptable salt thereof.

In some embodiments, the compound of Formula I has a structure of Formula I-8:

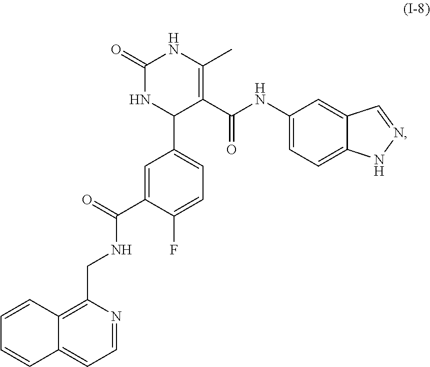 [SMILES: CC1=C(C(=O)Nc2ccc3[nH]ncc3c2)C(c2ccc(F)c(C(=O)NCc3nccc4ccccc34)c2)NC(=O)N1]

or a pharmaceutically acceptable salt thereof.

In some embodiments, the compound of Formula I has a structure of Formula I-9:

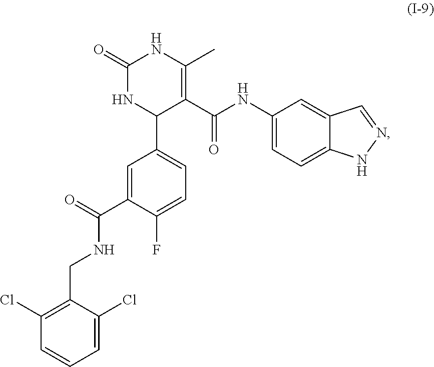 [SMILES: CC1=C(C(=O)Nc2ccc3[nH]ncc3c2)C(c2ccc(F)c(C(=O)NCc3c(Cl)cccc3Cl)c2)NC(=O)N1]

or a pharmaceutically acceptable salt thereof.

In some embodiments, the compound of Formula I has a structure of Formula I-10:

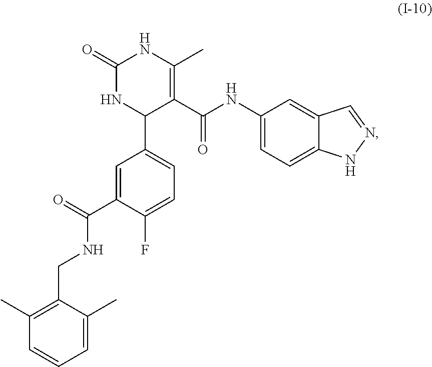 [SMILES: CC1=C(C(=O)Nc2ccc3[nH]ncc3c2)C(c2ccc(F)c(C(=O)NCc3c(C)cccc3C)c2)NC(=O)N1]

or a pharmaceutically acceptable salt thereof.

In some embodiments, the compound of Formula I has a structure of Formula I-11:

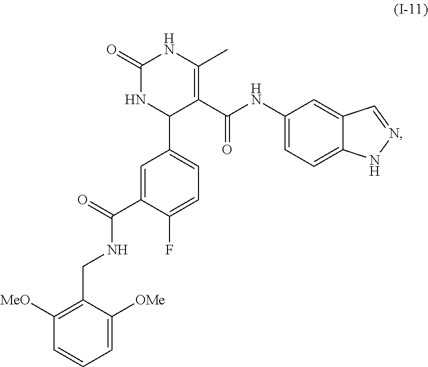 [SMILES: COc1cccc(OC)c1CNC(=O)c1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-12:

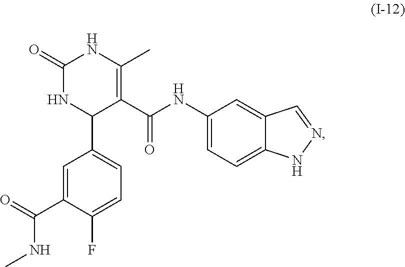 [SMILES: CNC(=O)c1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-13:

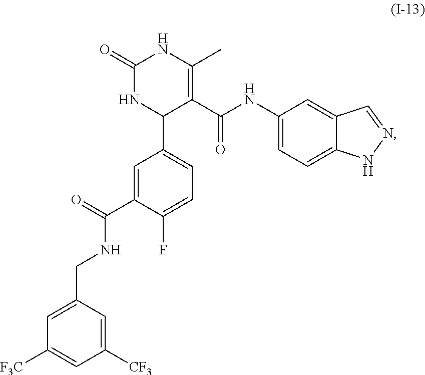 [SMILES: CC1=C(C(=O)Nc2ccc3[nH]ncc3c2)C(c2ccc(F)c(C(=O)NCc3cc(C(F)(F)F)cc(C(F)(F)F)c3)c2)NC(=O)N1]

or a pharmaceutically acceptable salt thereof.

In some embodiments, the compound of Formula I has a structure of Formula I-14:

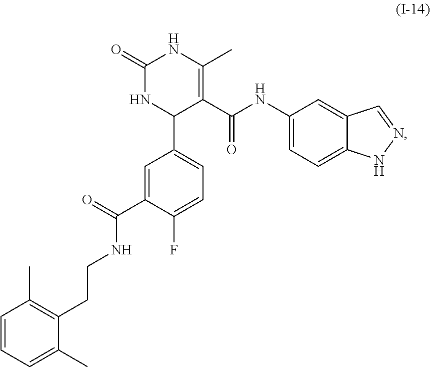 [SMILES: CC1=C(C(=O)Nc2ccc3[nH]ncc3c2)C(c2ccc(F)c(C(=O)NCCc3c(C)cccc3C)c2)NC(=O)N1]

or a pharmaceutically acceptable salt thereof.

In some embodiments, the compound of Formula I has a structure of Formula I-15:

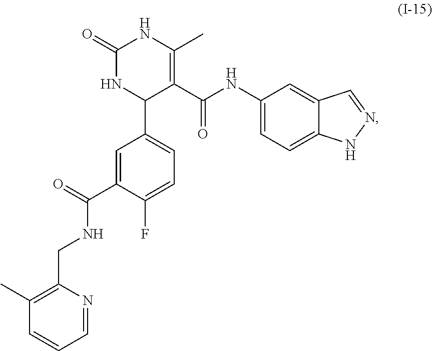 [SMILES: CC1=C(C(=O)Nc2ccc3[nH]ncc3c2)C(c2ccc(F)c(C(=O)NCc3ncccc3C)c2)NC(=O)N1]

or a pharmaceutically acceptable salt thereof.

In some embodiments, the compound of Formula I has a structure of Formula I-16:

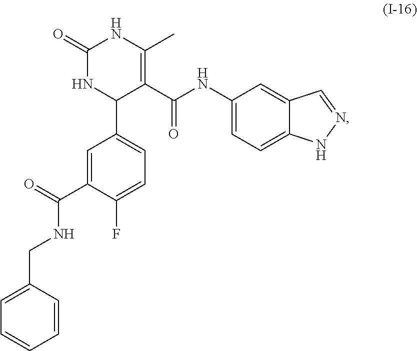 [SMILES: CC1=C(C(=O)Nc2ccc3[nH]ncc3c2)C(c2ccc(F)c(C(=O)NCc3ccccc3)c2)NC(=O)N1]

or a pharmaceutically acceptable salt thereof.

In some embodiments, the compound of Formula I has a structure of Formula I-17:

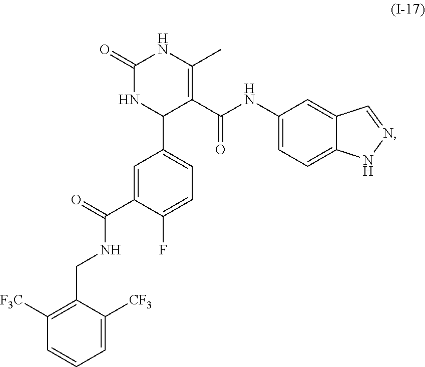 [SMILES: CC1=C(C(=O)Nc2ccc3[nH]ncc3c2)C(c2ccc(F)c(C(=O)NCc3c(C(F)(F)F)cccc3C(F)(F)F)c2)NC(=O)N1]

or a pharmaceutically acceptable salt thereof.

In some embodiments, the compound of Formula I has a structure of Formula I-18:

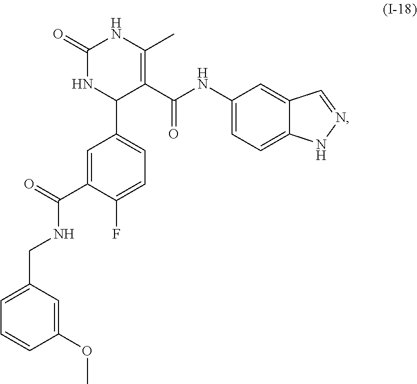 [SMILES: COc1cccc(CNC(=O)c2cc(C3NC(=O)NC(C)=C3C(=O)Nc3ccc4[nH]ncc4c3)ccc2F)c1]

or a pharmaceutically acceptable salt thereof.

In some embodiments, the compound of Formula I has a structure of Formula I-19:

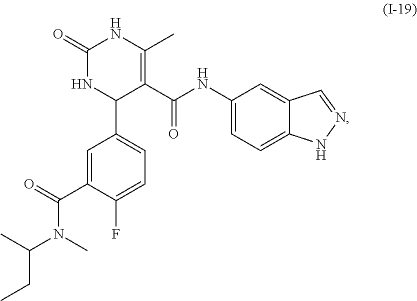 [SMILES: CCC(C)N(C)C(=O)c1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-20:

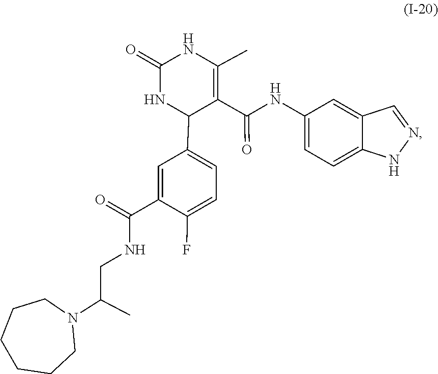 [SMILES: CC1=C(C(=O)Nc2ccc3[nH]ncc3c2)C(c2ccc(F)c(C(=O)NCC(C)N3CCCCCC3)c2)NC(=O)N1]

or a pharmaceutically acceptable salt thereof.

In some embodiments, the compound of Formula I has a structure of Formula I-21:

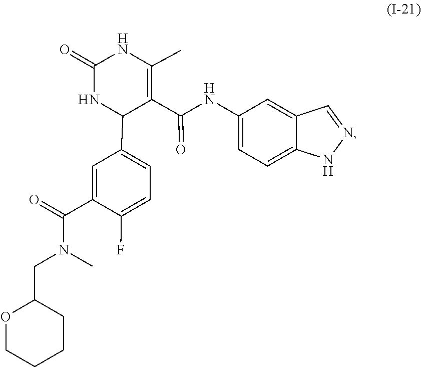 [SMILES: CC1=C(C(=O)Nc2ccc3[nH]ncc3c2)C(c2ccc(F)c(C(=O)N(C)CC3CCCCO3)c2)NC(=O)N1]

or a pharmaceutically acceptable salt thereof.

In some embodiments, the compound of Formula I has a structure of Formula I-22:

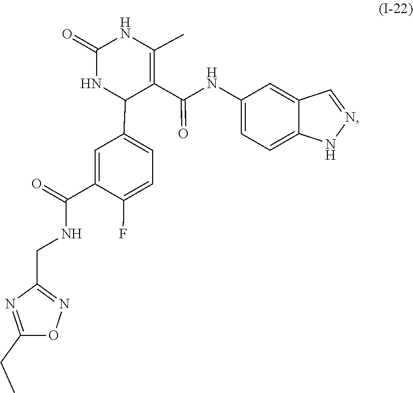 [SMILES: CCc1nc(CNC(=O)c2cc(C3NC(=O)NC(C)=C3C(=O)Nc3ccc4[nH]ncc4c3)ccc2F)no1]

or a pharmaceutically acceptable salt thereof.

In some embodiments, the compound of Formula I has a structure of Formula I-23:

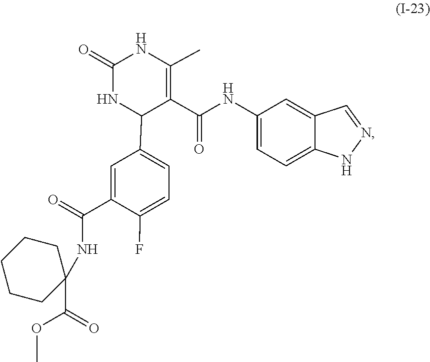 [SMILES: COC(=O)C1(NC(=O)c2cc(C3NC(=O)NC(C)=C3C(=O)Nc3ccc4[nH]ncc4c3)ccc2F)CCCCC1]

or a pharmaceutically acceptable salt thereof.

In some embodiments, the compound of Formula I has a structure of Formula I-24:

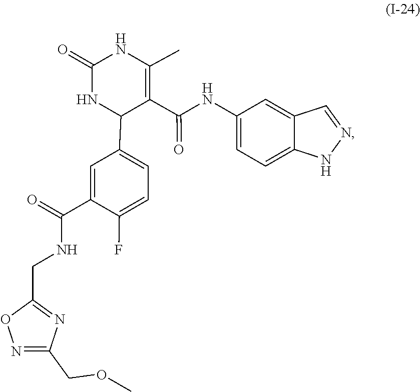 [SMILES: COCc1noc(CNC(=O)c2cc(C3NC(=O)NC(C)=C3C(=O)Nc3ccc4[nH]ncc4c3)ccc2F)n1]

or a pharmaceutically acceptable salt thereof.

In some embodiments, the compound of Formula I has a structure of Formula I-25:

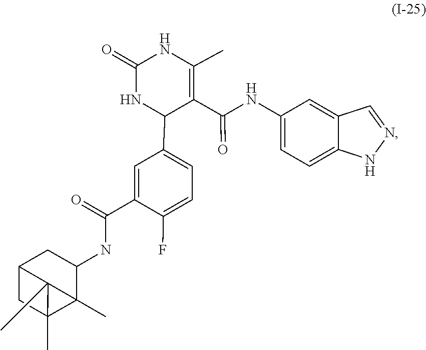 [SMILES: CCC12C3CC(NC(=O)c4cc(C5NC(=O)NC(C)=C5C(=O)Nc5ccc6[nH]ncc6c5)ccc4F)C1(C)C2(C)C3]

or a pharmaceutically acceptable salt thereof.

In some embodiments, the compound of Formula I has a structure of Formula I-26:

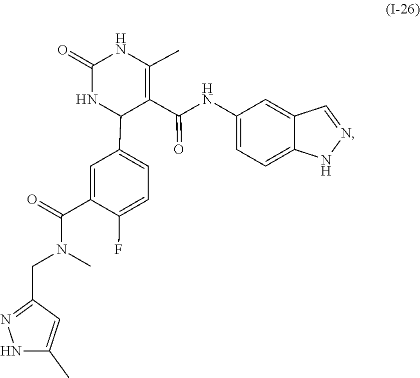 [SMILES: CC1=C(C(=O)Nc2ccc3[nH]ncc3c2)C(c2ccc(F)c(C(=O)N(C)Cc3cc(C)[nH]n3)c2)NC(=O)N1]

or a pharmaceutically acceptable salt thereof.

In some embodiments, the compound of Formula I has a structure of Formula I-27:

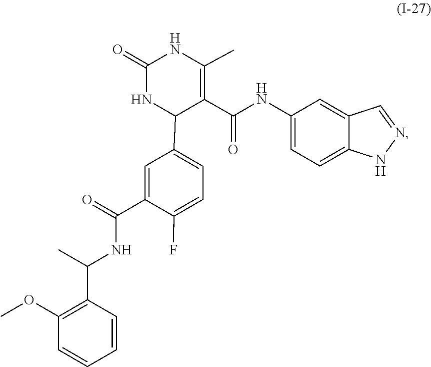 [SMILES: COc1ccccc1C(C)NC(=O)c1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-28:

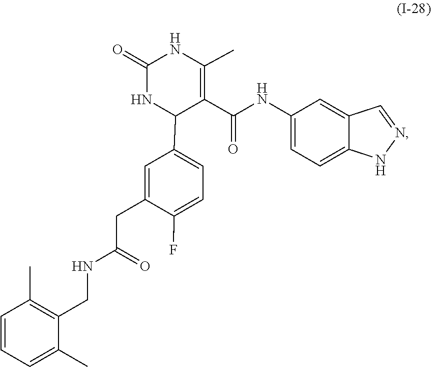 [SMILES: CC1=C(C(=O)Nc2ccc3[nH]ncc3c2)C(c2ccc(F)c(CC(=O)NCc3c(C)cccc3C)c2)NC(=O)N1]

or a pharmaceutically acceptable salt thereof.

In some embodiments, the compound of Formula I has a structure of Formula I-29:

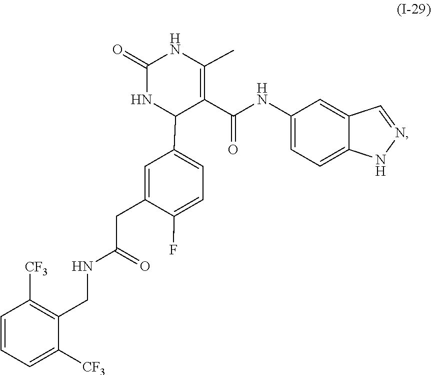 [SMILES: CC1=C(C(=O)Nc2ccc3[nH]ncc3c2)C(c2ccc(F)c(CC(=O)NCc3c(C(F)(F)F)cccc3C(F)(F)F)c2)NC(=O)N1]

or a pharmaceutically acceptable salt thereof.

In some embodiments, the compound of Formula I has a structure of Formula I-30:

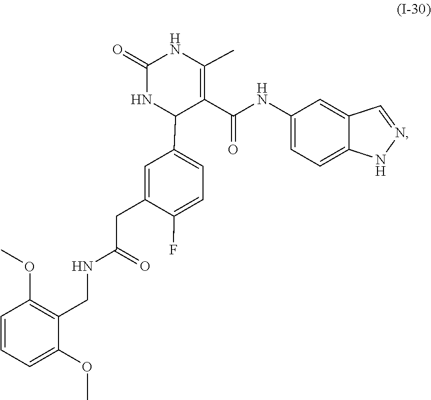 [SMILES: COc1cccc(OC)c1CNC(=O)Cc1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-31:

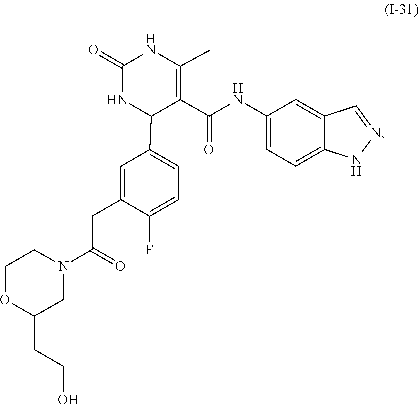 [SMILES: CC1=C(C(=O)Nc2ccc3[nH]ncc3c2)C(c2ccc(F)c(CC(=O)N3CCOC(CCO)C3)c2)NC(=O)N1]

or a pharmaceutically acceptable salt thereof.

In some embodiments, the compound of Formula I has a structure of I-32:

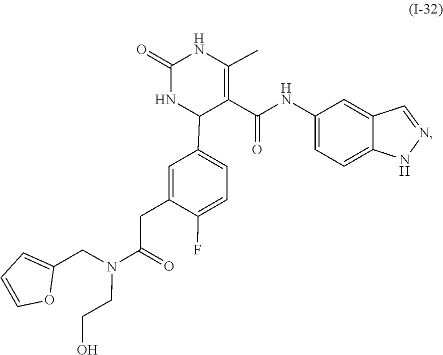 [SMILES: CC1=C(C(=O)Nc2ccc3[nH]ncc3c2)C(c2ccc(F)c(CC(=O)N(CCO)Cc3ccco3)c2)NC(=O)N1]

or a pharmaceutically acceptable salt thereof.

In some embodiments, the compound of Formula I has a structure of Formula I-33:

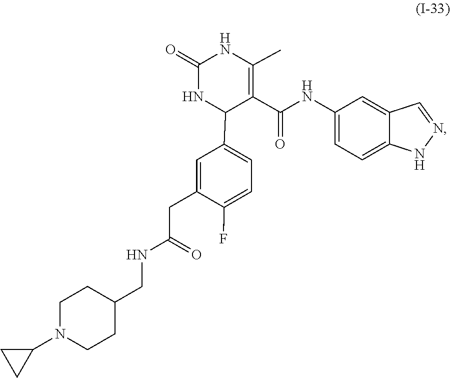 [SMILES: CC1=C(C(=O)Nc2ccc3[nH]ncc3c2)C(c2ccc(F)c(CC(=O)NCC3CCN(C4CC4)CC3)c2)NC(=O)N1]

or a pharmaceutically acceptable salt thereof.

In some embodiments, the compound of Formula I has a structure of Formula I-34:

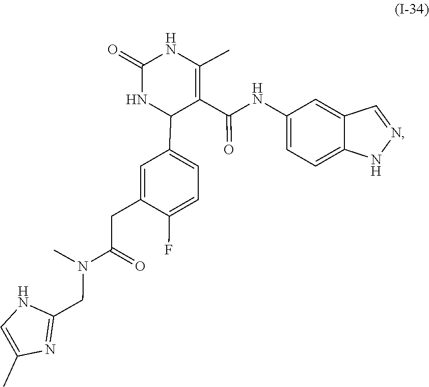 [SMILES: CC1=C(C(=O)Nc2ccc3[nH]ncc3c2)C(c2ccc(F)c(CC(=O)N(C)Cc3nc(C)c[nH]3)c2)NC(=O)N1]

or a pharmaceutically acceptable salt thereof.

In some embodiments, the compound of Formula I has a structure of Formula I-35:

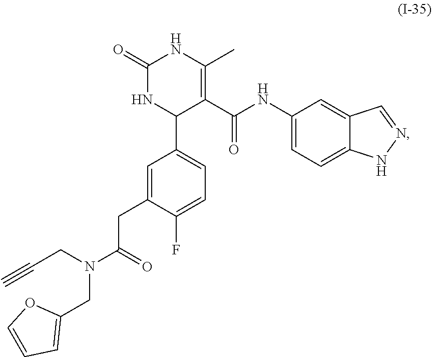 [SMILES: C#CCN(Cc1ccco1)C(=O)Cc1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-36:

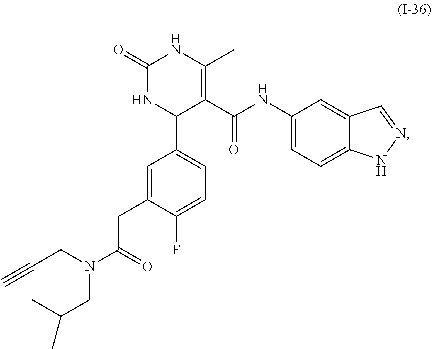 [SMILES: C#CCN(CC(C)C)C(=O)Cc1cc(C2NC(=O)NC(C)=C2C(=O)Nc2ccc3[nH]ncc3c2)ccc1F]

or a pharmaceutically acceptable salt thereof.

In some embodiments, the compound of Formula I has a structure of Formula I-37:

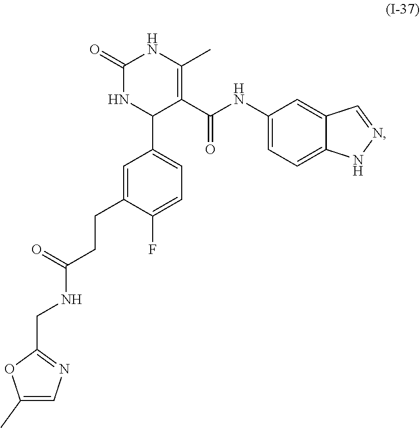 [SMILES: CC1=C(C(=O)Nc2ccc3[nH]ncc3c2)C(c2ccc(F)c(CCC(=O)NCc3ncc(C)o3)c2)NC(=O)N1]

or a pharmaceutically acceptable salt thereof.

In some embodiments, the compound of Formula I has a structure of Formula I-38:

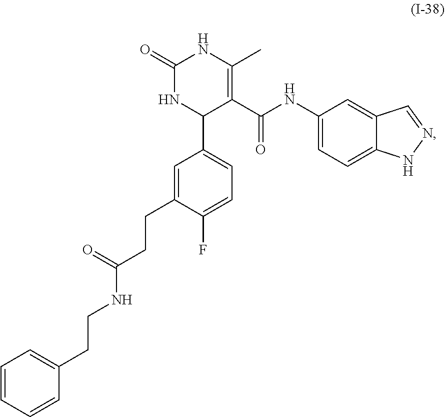 [SMILES: CC1=C(C(=O)Nc2ccc3[nH]ncc3c2)C(c2ccc(F)c(CCC(=O)NCCc3ccccc3)c2)NC(=O)N1]

or a pharmaceutically acceptable salt thereof.

In some embodiments, the compound of Formula I has a structure of Formula I-39:

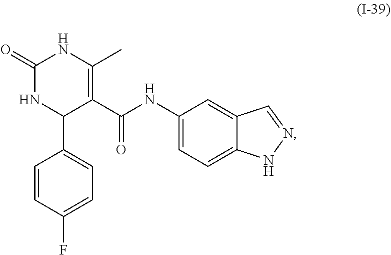 [SMILES: CC1=C(C(=O)Nc2ccc3[nH]ncc3c2)C(c2ccc(F)cc2)NC(=O)N1]

or a pharmaceutically acceptable salt thereof.

Compounds of Formula I may be synthesized by methods known in the art, e.g., those described in International application publication WO/2016/023028, which is incorporated herein by reference.

In some embodiments of any of the above aspects, the GRK2 small molecule antagonist is a compound of Formula II:

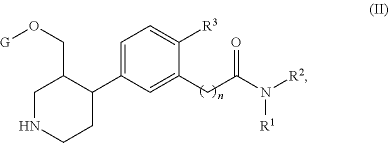

or a pharmaceutically acceptable salt thereof, where:

G is

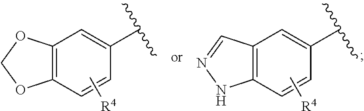

n is 0, 1, or 2;

R¹ is H, C₁-C₈ alkyl, C₂-C₈ alkenyl, C₃-C₈ cycloalkyl, C₃-C₈ cycloalkenyl, C₁-C₄alkylene-aryl, C₁-C₄ alkylene-heteroaryl, C₃-C₈ cycloalkylene-aryl, or C₃-C₈ cycloalkylene-heteroaryl;

R² is C₁-C₈ alkyl, C₂-C₈ alkenyl, C₀-C₄ alkylene-C₃-C₈ cycloalkyl, C₀-C₄alkylene-C₃-C₈ cycloalkenyl, C₁-C₄ alkylene-aryl, C₁-C₄ alkylene-heteroaryl, C₃-C₈ cycloalkylene-aryl, or C₃-C₈ cycloalkylene-heteroaryl;

R³ is H, F, Cl, CF₃, CHF₂, CH₂F, or CH₃; and

R⁴ is H, F, or Cl.

In some embodiments, the compound of Formula II has a structure of Formula II-1:

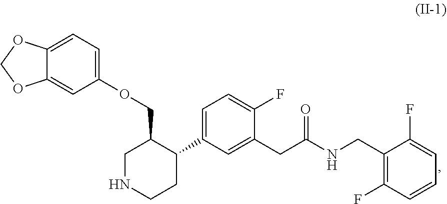 [SMILES: O=C(Cc1cc([C@@H]2CCNC[C@H]2COc2ccc3c(c2)OCO3)ccc1F)NCc1c(F)cccc1F]

or a pharmaceutically acceptable salt thereof.

In some embodiments, the compound of Formula II has a structure of Formula II-2:

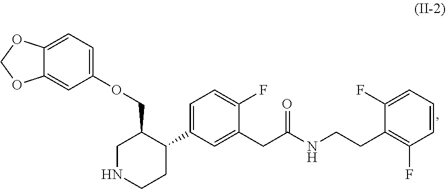 [SMILES: O=C(Cc1cc([C@@H]2CCNC[C@H]2COc2ccc3c(c2)OCO3)ccc1F)NCCc1c(F)cccc1F]

or a pharmaceutically acceptable salt thereof.

In some embodiments, the compound of Formula II has a structure of Formula II-3:

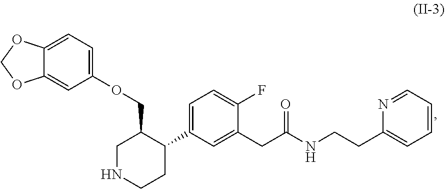 [SMILES: O=C(Cc1cc([C@@H]2CCNC[C@H]2COc2ccc3c(c2)OCO3)ccc1F)NCCc1ccccn1]

or a pharmaceutically acceptable salt thereof.

In some embodiments, the compound of Formula II has a structure of Formula II-4:

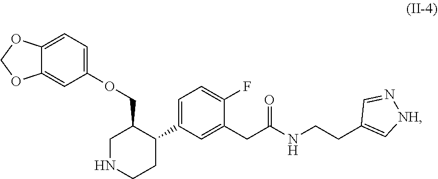 [SMILES: O=C(Cc1cc([C@@H]2CCNC[C@H]2COc2ccc3c(c2)OCO3)ccc1F)NCCc1cn[nH]c1]

or a pharmaceutically acceptable salt thereof.

In some embodiments, the compound of Formula II has a structure of Formula II-5:

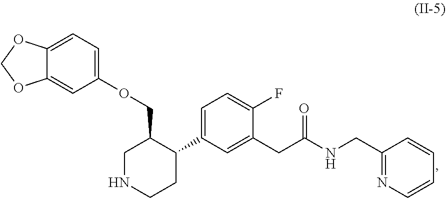 [SMILES: O=C(Cc1cc([C@@H]2CCNC[C@H]2COc2ccc3c(c2)OCO3)ccc1F)NCc1ccccn1]

or a pharmaceutically acceptable salt thereof.

In some embodiments, the compound of Formula II has a structure of Formula II-6:

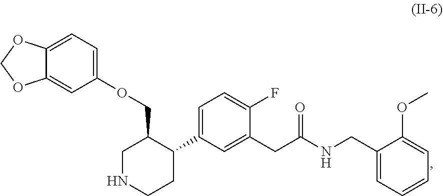 [SMILES: COc1ccccc1CNC(=O)Cc1cc([C@@H]2CCNC[C@H]2COc2ccc3c(c2)OCO3)ccc1F]

or a pharmaceutically acceptable salt thereof.

In some embodiments, the compound of Formula II has a structure of Formula II-7:

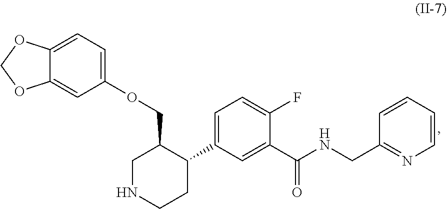 [SMILES: O=C(NCc1ccccn1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-8:

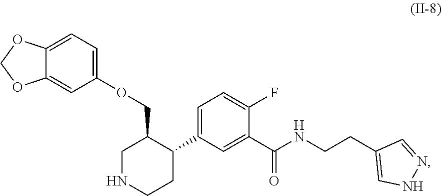 [SMILES: O=C(NCCc1cn[nH]c1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-9:

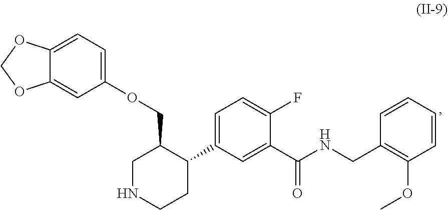 [SMILES: COc1ccccc1CNC(=O)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-10:

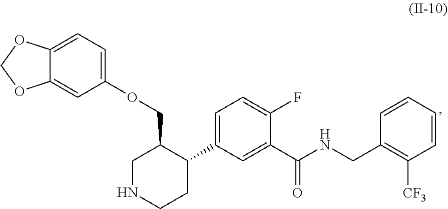 [SMILES: O=C(NCc1ccccc1C(F)(F)F)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-11:

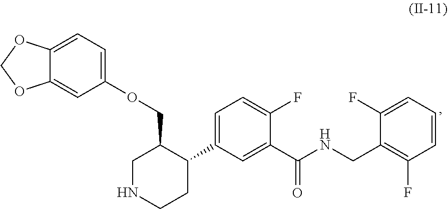 [SMILES: O=C(NCc1c(F)cccc1F)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-12:

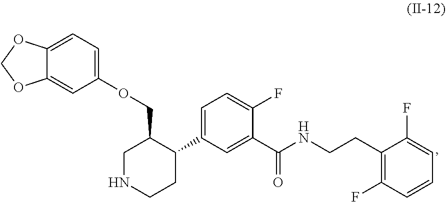 [SMILES: O=C(NCCc1c(F)cccc1F)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-13:

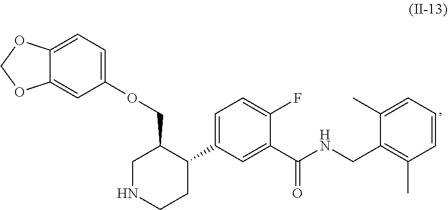 [SMILES: Cc1cccc(C)c1CNC(=O)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-14:

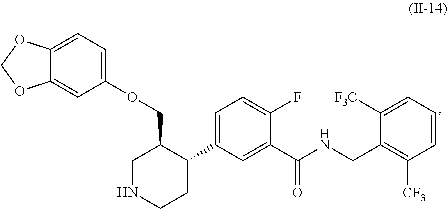 [SMILES: O=C(NCc1c(C(F)(F)F)cccc1C(F)(F)F)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-15:

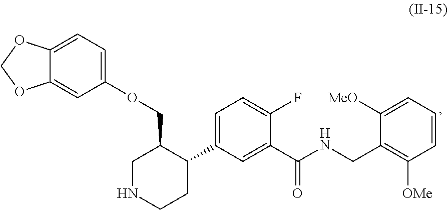 [SMILES: COc1cccc(OC)c1CNC(=O)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-16:

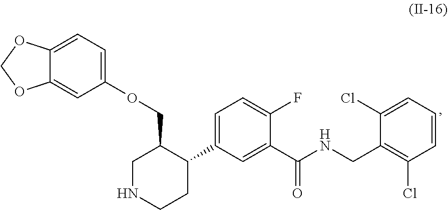 [SMILES: O=C(NCc1c(Cl)cccc1Cl)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-17:

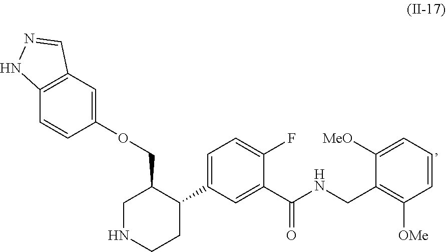 [SMILES: COc1cccc(OC)c1CNC(=O)c1cc([C@@H]2CCNC[C@H]2COc2ccc3[nH]ncc3c2)ccc1F]

or a pharmaceutically acceptable salt thereof.

In some embodiments, the compound of Formula II has a structure Formula of II-18:

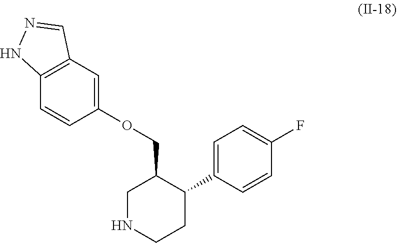 [SMILES: Fc1ccc([C@@H]2CCNC[C@H]2COc2ccc3[nH]ncc3c2)cc1]

or a pharmaceutically acceptable salt thereof.

In some embodiments, the compound of Formula II has a structure of Formula II-19:

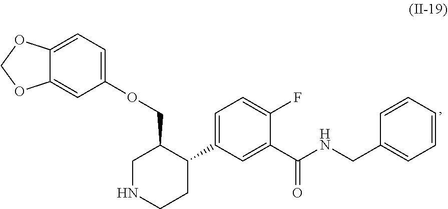 [SMILES: O=C(NCc1ccccc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-20:

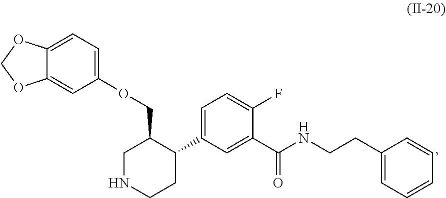 [SMILES: O=C(NCCc1ccccc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-21:

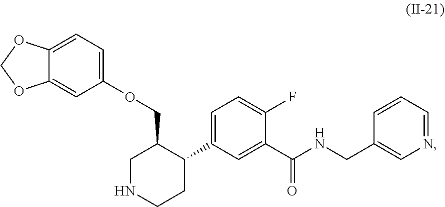 [SMILES: O=C(NCc1cccnc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-22:

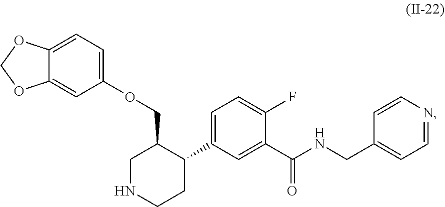 [SMILES: O=C(NCc1ccncc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-23:

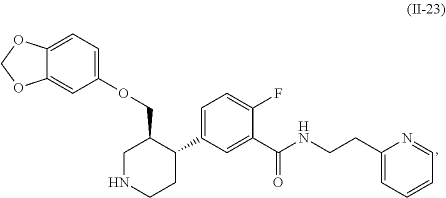 [SMILES: O=C(NCCc1ccccn1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-24:

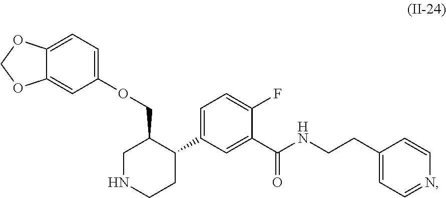 [SMILES: O=C(NCCc1ccncc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-25:

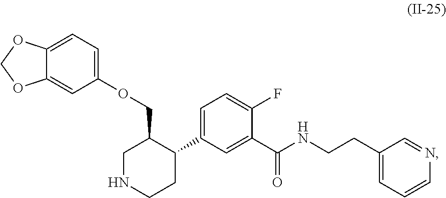 [SMILES: O=C(NCCc1cccnc1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-26:

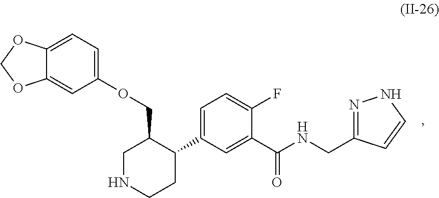 [SMILES: O=C(NCc1cc[nH]n1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-27:

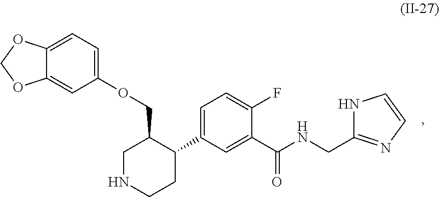 [SMILES: O=C(NCc1ncc[nH]1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-28:

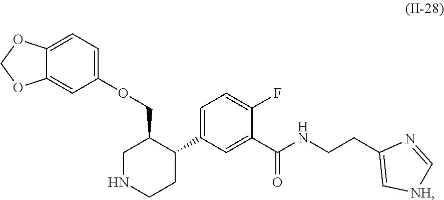 [SMILES: O=C(NCCc1c[nH]cn1)c1cc([C@@H]2CCNC[C@H]2COc2ccc3c(c2)OCO3)ccc1F]

or a pharmaceutically acceptable salt thereof.

In some embodiments, the compound of Formula II has a structure of Formula II-29:

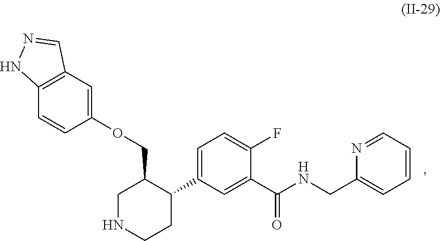 [SMILES: O=C(NCc1ccccn1)c1cc([C@@H]2CCNC[C@H]2COc2ccc3[nH]ncc3c2)ccc1F]

or a pharmaceutically acceptable salt thereof.

In some embodiments, the compound of Formula II has a structure of Formula II-30:

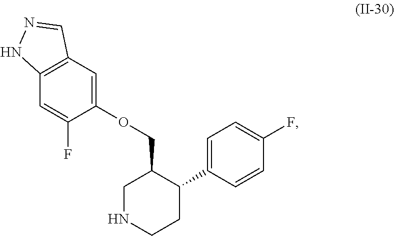 [SMILES: Fc1ccc([C@@H]2CCNC[C@H]2COc2cc3cn[nH]c3cc2F)cc1]

or a pharmaceutically acceptable salt thereof.

In some embodiments, the compound of Formula II has a structure of Formula II-31:

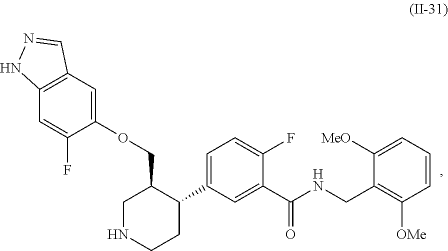 [SMILES: COc1cccc(OC)c1CNC(=O)c1cc([C@@H]2CCNC[C@H]2COc2cc3cn[nH]c3cc2F)ccc1F]

or a pharmaceutically acceptable salt thereof.

In some embodiments, the compound of Formula II has a structure of Formula II-32:

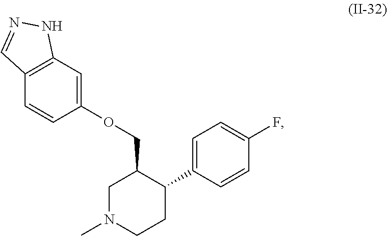 [SMILES: CN1CC[C@@H](c2ccc(F)cc2)[C@H](COc2ccc3cn[nH]c3c2)C1]

or a pharmaceutically acceptable salt thereof.

In some embodiments, the compound of Formula II has a structure of Formula II-33:

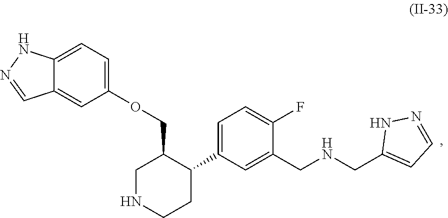 [SMILES: Fc1ccc([C@@H]2CCNC[C@H]2COc2ccc3[nH]ncc3c2)cc1CNCc1ccn[nH]1]

or a pharmaceutically acceptable salt thereof.

In some embodiments, the compound of Formula II has a structure of Formula II-34:

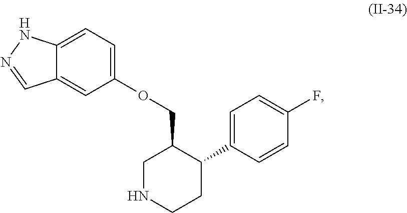 [SMILES: Fc1ccc([C@@H]2CCNC[C@H]2COc2ccc3[nH]ncc3c2)cc1]

or a pharmaceutically acceptable salt thereof.

In some embodiments, the compound of Formula II has a structure of Formula II-35:

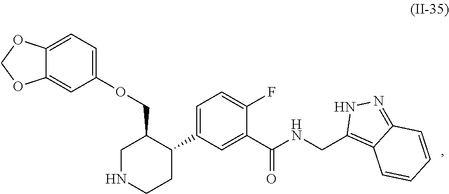 [SMILES: O=C(NCc1[nH]nc2ccccc12)c1cc([C@@H]2CCNC[C@H]2COc2ccc3c(c2)OCO3)ccc1F]

or a pharmaceutically acceptable salt thereof.

Compounds of Formula II may be synthesized by methods known in the art, e.g., those described in International application publication WO/2016/210403, which is incorporated herein by reference.

In some embodiments of any of the above aspects, the GRK2 small molecule antagonist is a compound of Formula III:

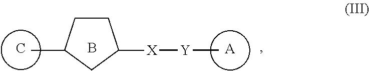

or a pharmaceutically acceptable salt thereof, where:

Ring A represents an optionally substituted aromatic ring; Ring B represents an optionally substituted 5-membered nitrogen-containing aromatic heterocycle;

Ring C represents an optionally substituted nitrogen-containing aromatic heterocycle,

X represents an optionally substituted C₁-C₄ alkylene group,

Y is an imino group which may be substituted, O— or —S(O)ₙ;

n is 0, 1, or 2.

In some embodiments, the compound of Formula III has a structure of Formula III-1:

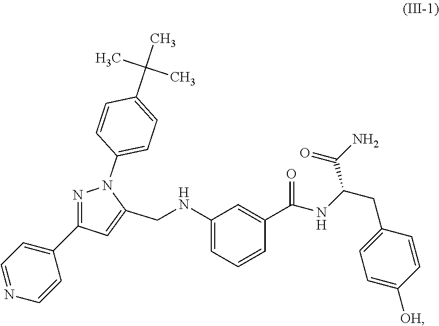 [SMILES: CC(C)(C)c1ccc(-n2nc(-c3ccncc3)cc2CNc2cccc(C(=O)N[C@@H](Cc3ccc(O)cc3)C(N)=O)c2)cc1]

or a pharmaceutically acceptable salt thereof.

In some embodiments, the compound of Formula III has a structure of Formula III-2:

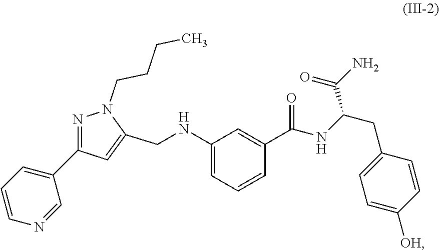 [SMILES: CCCCn1nc(-c2cccnc2)cc1CNc1cccc(C(=O)N[C@@H](Cc2ccc(O)cc2)C(N)=O)c1]

or a pharmaceutically acceptable salt thereof.

In some embodiments, the compound of Formula III has a structure of Formula III-3:

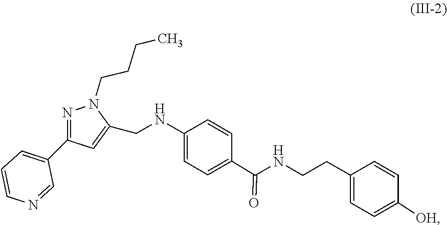 [SMILES: CCCCn1nc(-c2cccnc2)cc1CNc1ccc(C(=O)NCCc2ccc(O)cc2)cc1]

or a pharmaceutically acceptable salt thereof.

In some embodiments, the compound of Formula III has a structure of Formula III-4:

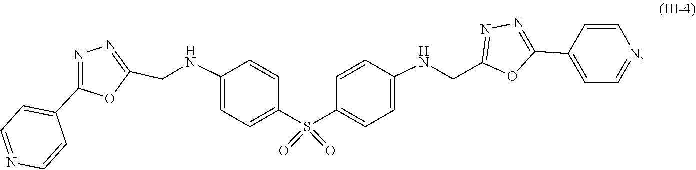 [SMILES: O=S(=O)(c1ccc(NCc2nnc(-c3ccncc3)o2)cc1)c1ccc(NCc2nnc(-c3ccncc3)o2)cc1]

or a pharmaceutically acceptable salt thereof.

In some embodiments, the compound of Formula III has a structure of Formula III-5:

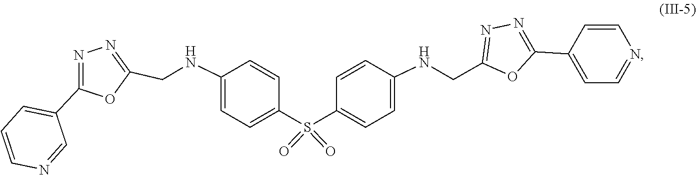 [SMILES: O=S(=O)(c1ccc(NCc2nnc(-c3ccncc3)o2)cc1)c1ccc(NCc2nnc(-c3cccnc3)o2)cc1]

or a pharmaceutically acceptable salt thereof.

In some embodiments, the compound of Formula III has a structure of III-6:

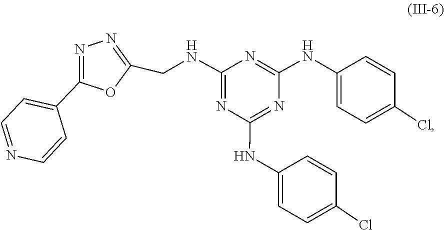 [SMILES: Clc1ccc(Nc2nc(NCc3nnc(-c4ccncc4)o3)nc(Nc3ccc(Cl)cc3)n2)cc1]

or a pharmaceutically acceptable salt thereof.

In some embodiments, the compound of Formula III has a structure of Formula III-7:

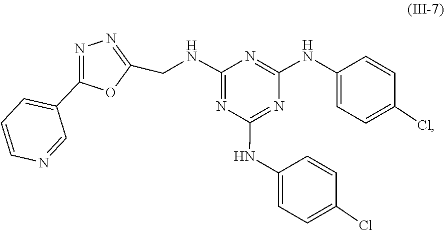 [SMILES: Clc1ccc(Nc2nc(NCc3nnc(-c4cccnc4)o3)nc(Nc3ccc(Cl)cc3)n2)cc1]

or a pharmaceutically acceptable salt thereof.

In some embodiments, the compound of Formula III has a structure of Formula III-8:

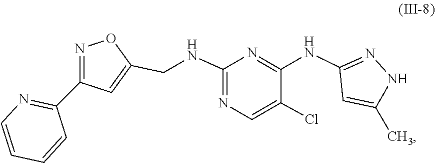 [SMILES: Cc1cc(Nc2nc(NCc3cc(-c4ccccn4)no3)ncc2Cl)n[nH]1]

or a pharmaceutically acceptable salt thereof.

In some embodiments, the compound of Formula III has a structure of Formula III-9:

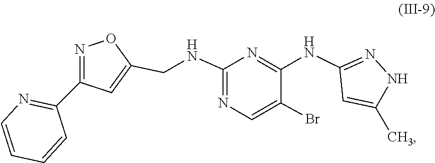 [SMILES: Cc1cc(Nc2nc(NCc3cc(-c4ccccn4)no3)ncc2Br)n[nH]1]

or a pharmaceutically acceptable salt thereof.

In some embodiments, the compound of Formula III has a structure of Formula III-10:

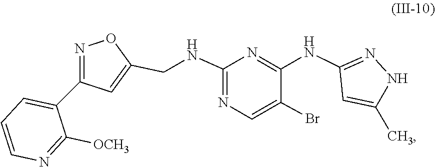 [SMILES: COc1ncccc1-c1cc(CNc2ncc(Br)c(Nc3cc(C)[nH]n3)n2)on1]

or a pharmaceutically acceptable salt thereof.

In some embodiments, the compound of Formula III has a structure of Formula III-11:

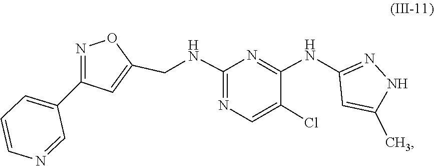 [SMILES: Cc1cc(Nc2nc(NCc3cc(-c4cccnc4)no3)ncc2Cl)n[nH]1]

or a pharmaceutically acceptable salt thereof.

In some embodiments, the compound of Formula III has a structure of Formula III-12:

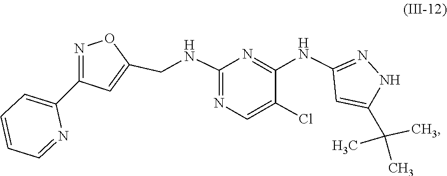 [SMILES: CC(C)(C)c1cc(Nc2nc(NCc3cc(-c4ccccn4)no3)ncc2Cl)n[nH]1]

or a pharmaceutically acceptable salt thereof.

In some embodiments, the compound of Formula III has a structure of Formula III-13:

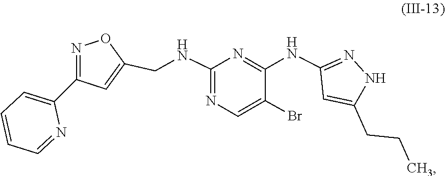 [SMILES: CCCc1cc(Nc2nc(NCc3cc(-c4ccccn4)no3)ncc2Br)n[nH]1]

or a pharmaceutically acceptable salt thereof.

In some embodiments, the compound of Formula III has a structure of Formula III-14:

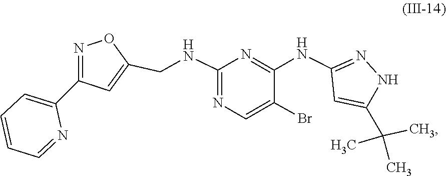 [SMILES: CC(C)(C)c1cc(Nc2nc(NCc3cc(-c4ccccn4)no3)ncc2Br)n[nH]1]

or a pharmaceutically acceptable salt thereof.

In some embodiments, the compound of Formula III has a structure of Formula III-15:

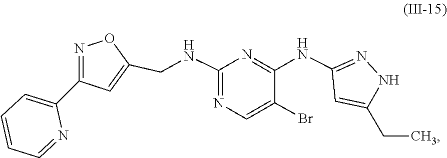 [SMILES: CCc1cc(Nc2nc(NCc3cc(-c4ccccn4)no3)ncc2Br)n[nH]1]

or a pharmaceutically acceptable salt thereof.

In some embodiments, the compound of Formula III has a structure of Formula III-16:

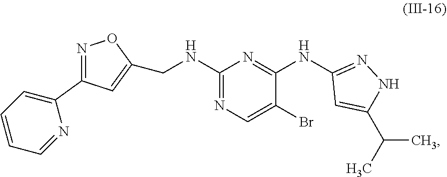 [SMILES: CC(C)c1cc(Nc2nc(NCc3cc(-c4ccccn4)no3)ncc2Br)n[nH]1]

or a pharmaceutically acceptable salt thereof.

In some embodiments, the compound of Formula III has a structure of Formula III-17:

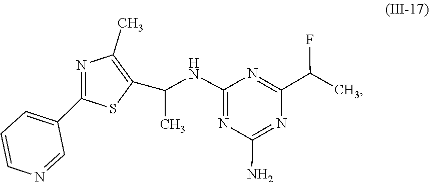 [SMILES: Cc1nc(-c2cccnc2)sc1C(C)Nc1nc(N)nc(C(C)F)n1]

or a pharmaceutically acceptable salt thereof.

In some embodiments, the compound of Formula III has a structure of Formula III-18:

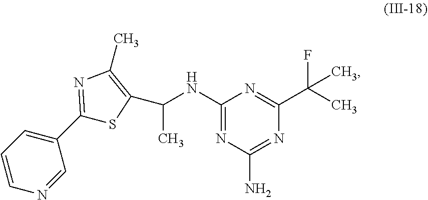 [SMILES: Cc1nc(-c2cccnc2)sc1C(C)Nc1nc(N)nc(C(C)(C)F)n1]

or a pharmaceutically acceptable salt thereof.

In some embodiments, the compound of Formula III has a structure of Formula III-19:

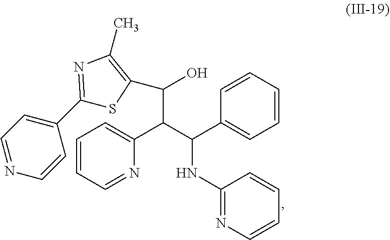 [SMILES: Cc1nc(-c2ccncc2)sc1C(O)C(c1ccccn1)C(Nc1ccccn1)c1ccccc1]

or a pharmaceutically acceptable salt thereof.

In some embodiments, the compound of Formula III has a structure of Formula III-20:

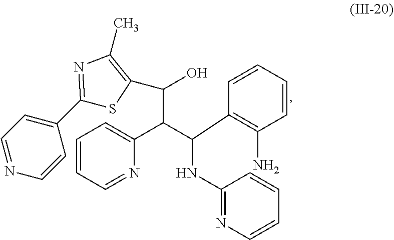 [SMILES: Cc1nc(-c2ccncc2)sc1C(O)C(c1ccccn1)C(Nc1ccccn1)c1ccccc1N]

or a pharmaceutically acceptable salt thereof.

In some embodiments, the compound of Formula III has a structure of Formula III-21:

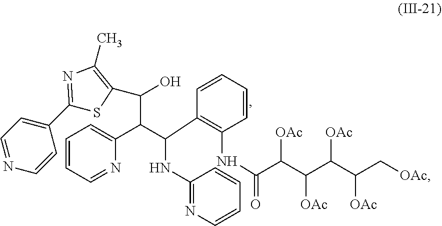 [SMILES: CC(=O)OCC(OC(C)=O)C(OC(C)=O)C(OC(C)=O)C(OC(C)=O)C(=O)Nc1ccccc1C(Nc1ccccn1)C(c1ccccn1)C(O)c1sc(-c2ccncc2)nc1C]

or a pharmaceutically acceptable salt, or a tautomer thereof.

In some embodiments, the compound of Formula III has a structure of Formula III-22:

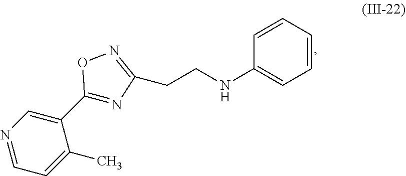 [SMILES: Cc1ccncc1-c1nc(CCNc2ccccc2)no1]

or a pharmaceutically acceptable salt, or a tautomer thereof.

In some embodiments, the compound of Formula III has a structure of Formula III-23:

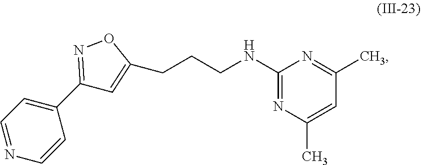 [SMILES: Cc1cc(C)nc(NCCCc2cc(-c3ccncc3)no2)n1]

or a pharmaceutically acceptable salt, or a tautomer thereof.

In some embodiments, the compound of Formula III has a structure of Formula III-24:

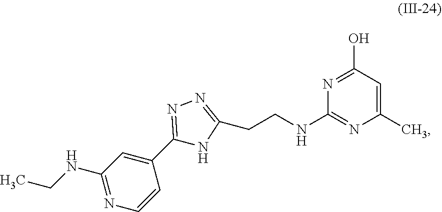 [SMILES: CCNc1cc(-c2nnc(CCNc3nc(C)cc(O)n3)[nH]2)ccn1]

or a pharmaceutically acceptable salt thereof.

In some embodiments, the compound of Formula III has a structure of Formula III-25:

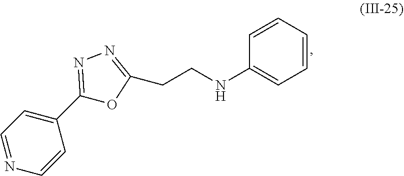 [SMILES: c1ccc(NCCc2nnc(-c3ccncc3)o2)cc1]

or a pharmaceutically acceptable salt thereof.

In some embodiments, the compound of Formula III has a structure of Formula III-26:

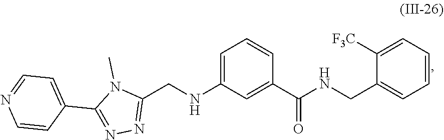 [SMILES: Cn1c(CNc2cccc(C(=O)NCc3ccccc3C(F)(F)F)c2)nnc1-c1ccncc1]

or a pharmaceutically acceptable salt thereof.

In some embodiments, the compound of Formula III has a structure of Formula III-27:

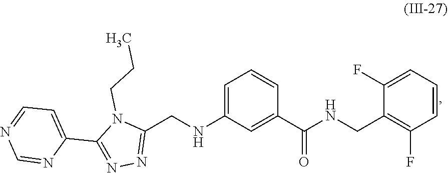 [SMILES: CCCn1c(CNc2cccc(C(=O)NCc3c(F)cccc3F)c2)nnc1-c1ccncn1]

or a pharmaceutically acceptable salt thereof.

In some embodiments, the compound of Formula III has a structure of Formula III-28:

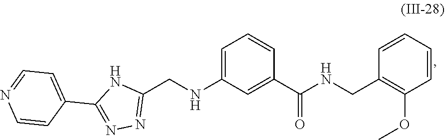 [SMILES: COc1ccccc1CNC(=O)c1cccc(NCc2nnc(-c3ccncc3)[nH]2)c1]

or a pharmaceutically acceptable salt thereof.

In some embodiments, the compound of Formula III has a structure of Formula III-29:

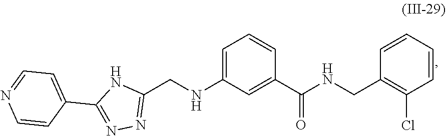 [SMILES: O=C(NCc1ccccc1Cl)c1cccc(NCc2nnc(-c3ccncc3)[nH]2)c1]

or a pharmaceutically acceptable salt thereof.

In some embodiments, the compound of Formula III has a structure of Formula III-30:

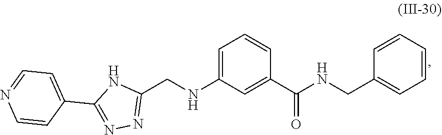 [SMILES: O=C(NCc1ccccc1)c1cccc(NCc2nnc(-c3ccncc3)[nH]2)c1]

or a pharmaceutically acceptable salt thereof.

In some embodiments, the compound of Formula III has a structure of Formula III-31:

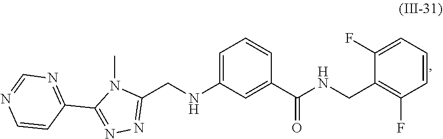 [SMILES: Cn1c(CNc2cccc(C(=O)NCc3c(F)cccc3F)c2)nnc1-c1ccncn1]

or a pharmaceutically acceptable salt thereof.

In some embodiments, the compound of Formula III has a structure of Formula III-32:

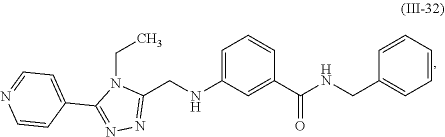 [SMILES: CCn1c(CNc2cccc(C(=O)NCc3ccccc3)c2)nnc1-c1ccncc1]

or a pharmaceutically acceptable salt thereof.

In some embodiments, the compound of Formula III has a structure of Formula III-33:

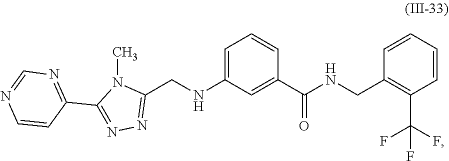 [SMILES: Cn1c(CNc2cccc(C(=O)NCc3ccccc3C(F)(F)F)c2)nnc1-c1ccncn1]

or a pharmaceutically acceptable salt thereof.

In some embodiments, the compound of Formula III has a structure of Formula III-34:

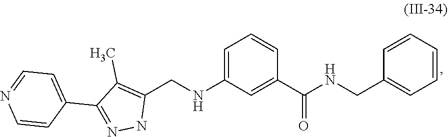 [SMILES: Cc1c(-c2ccncc2)n[nH]c1CNc1cccc(C(=O)NCc2ccccc2)c1]

or a pharmaceutically acceptable salt thereof.

In some embodiments, the compound of Formula III has a structure of Formula III-35:

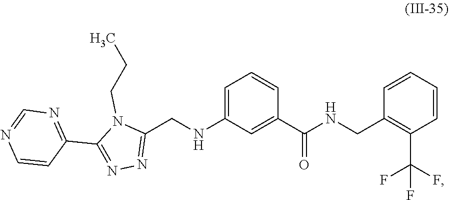 [SMILES: CCCn1c(CNc2cccc(C(=O)NCc3ccccc3C(F)(F)F)c2)nnc1-c1ccncn1]

or a pharmaceutically acceptable salt thereof.

In some embodiments, the compound of Formula III has a structure of Formula III-36:

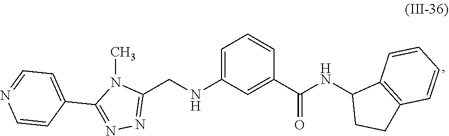 [SMILES: Cn1c(CNc2cccc(C(=O)NC3CCc4ccccc43)c2)nnc1-c1ccncc1]

or a pharmaceutically acceptable salt thereof.

In some embodiments, the compound of Formula III has a structure of Formula III-37:

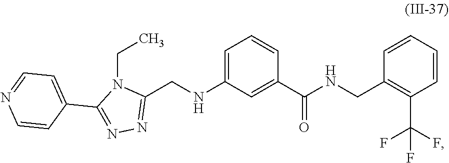 [SMILES: CCn1c(CNc2cccc(C(=O)NCc3ccccc3C(F)(F)F)c2)nnc1-c1ccncc1]

or a pharmaceutically acceptable salt thereof.

In some embodiments, the compound of Formula III has a structure of Formula III-38:

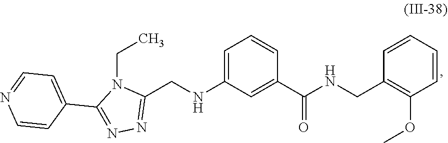 [SMILES: CCn1c(CNc2cccc(C(=O)NCc3ccccc3OC)c2)nnc1-c1ccncc1]

or a pharmaceutically acceptable salt thereof.

In some embodiments, the compound of Formula III has a structure of Formula III-39:

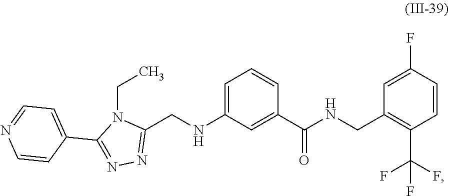 [SMILES: CCn1c(CNc2cccc(C(=O)NCc3cc(F)ccc3C(F)(F)F)c2)nnc1-c1ccncc1]

or a pharmaceutically acceptable salt thereof.

In some embodiments, the compound of Formula III has a structure of Formula III-40:

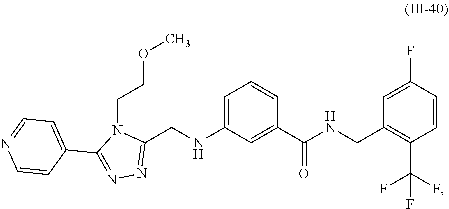 [SMILES: COCCn1c(CNc2cccc(C(=O)NCc3cc(F)ccc3C(F)(F)F)c2)nnc1-c1ccncc1]

or a pharmaceutically acceptable salt thereof.

In some embodiments, the compound of Formula III has a structure of Formula III-41:

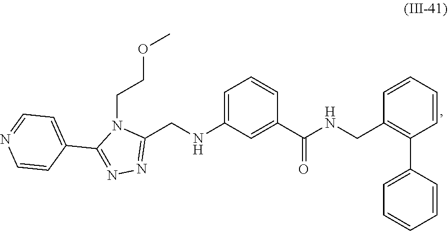 [SMILES: COCCn1c(CNc2cccc(C(=O)NCc3ccccc3-c3ccccc3)c2)nnc1-c1ccncc1]

or a pharmaceutically acceptable salt thereof.

In some embodiments, the compound of Formula III has a structure of Formula III-42:

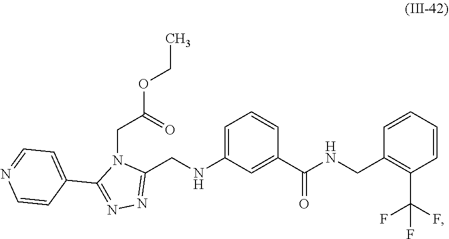 [SMILES: CCOC(=O)Cn1c(CNc2cccc(C(=O)NCc3ccccc3C(F)(F)F)c2)nnc1-c1ccncc1]

or a pharmaceutically acceptable salt thereof.

In some embodiments, the compound of Formula III has a structure of Formula III-43:

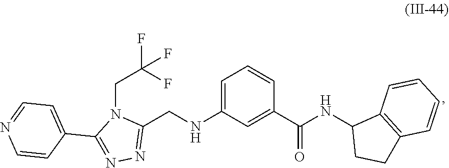 [SMILES: O=C(NC1CCc2ccccc21)c1cccc(NCc2nnc(-c3ccncc3)n2CC(F)(F)F)c1]

or a pharmaceutically acceptable salt thereof.

In some embodiments, the compound of Formula III has a structure of Formula III-44:

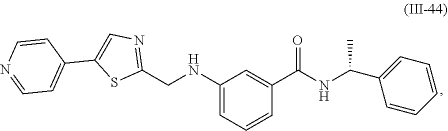 [SMILES: C[C@@H](NC(=O)c1cccc(NCc2ncc(-c3ccncc3)s2)c1)c1ccccc1]

or a pharmaceutically acceptable salt thereof.

In some embodiments, the compound of Formula III has a structure of Formula III-45:

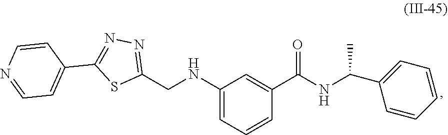 [SMILES: C[C@@H](NC(=O)c1cccc(NCc2nnc(-c3ccncc3)s2)c1)c1ccccc1]

or a pharmaceutically acceptable salt thereof.

In some embodiments, the compound of Formula III has a structure of Formula III-46:

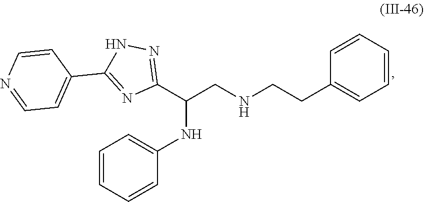 [SMILES: c1ccc(CCNCC(Nc2ccccc2)c2n[nH]c(-c3ccncc3)n2)cc1]

or a pharmaceutically acceptable salt thereof.

In some embodiments, the compound of Formula III has a structure of Formula III-47:

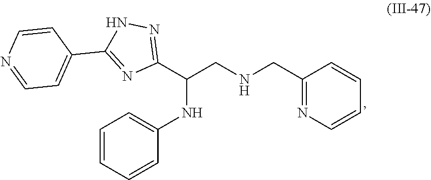 [SMILES: c1ccc(NC(CNCc2ccccn2)c2n[nH]c(-c3ccncc3)n2)cc1]

or a pharmaceutically acceptable salt thereof.

In some embodiments, the compound of Formula III has a structure of Formula III-48:

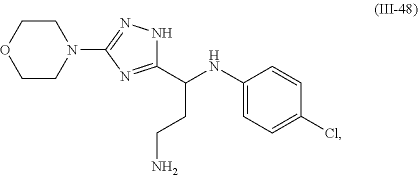 [SMILES: NCCC(Nc1ccc(Cl)cc1)c1nc(N2CCOCC2)n[nH]1]

or a pharmaceutically acceptable salt thereof.

In some embodiments, the compound of Formula III has a structure of Formula III-49:

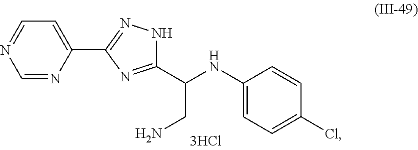 [SMILES: NCC(Nc1ccc(Cl)cc1)c1nc(-c2ccncn2)n[nH]1]

or a pharmaceutically acceptable salt thereof.

In some embodiments, the compound of Formula III has a structure of Formula III-50:

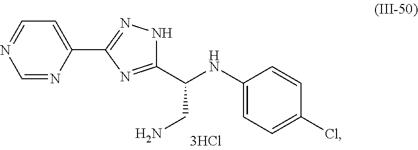 [SMILES: NC[C@@H](Nc1ccc(Cl)cc1)c1nc(-c2ccncn2)n[nH]1]

or a pharmaceutically acceptable salt thereof.

In some embodiments, the compound of Formula III has a structure of Formula III-51:

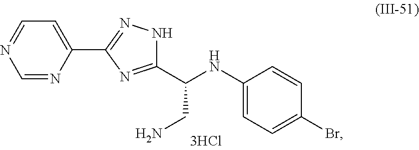 [SMILES: NC[C@@H](Nc1ccc(Br)cc1)c1nc(-c2ccncn2)n[nH]1]

or a pharmaceutically acceptable salt thereof.

In some embodiments, the compound of Formula III has a structure of Formula III-52:

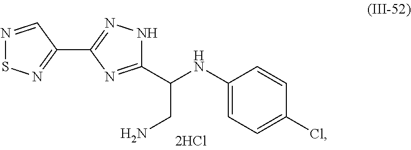 [SMILES: NCC(Nc1ccc(Cl)cc1)c1nc(-c2cnsn2)n[nH]1]

or a pharmaceutically acceptable salt thereof.

In some embodiments, the compound of Formula III has a structure of Formula III-53:

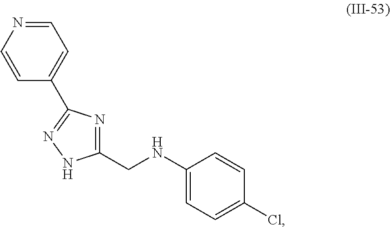 [SMILES: Clc1ccc(NCc2nc(-c3ccncc3)n[nH]2)cc1]

or a pharmaceutically acceptable salt thereof.

In some embodiments, the compound of Formula III has a structure of Formula III-54:

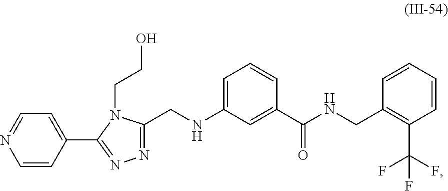 [SMILES: O=C(NCc1ccccc1C(F)(F)F)c1cccc(NCc2nnc(-c3ccncc3)n2CCO)c1]

or a pharmaceutically acceptable salt thereof.

In some embodiments, the compound of Formula III has a structure of Formula III-55:

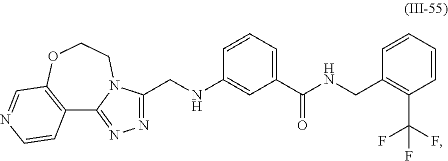 [SMILES: O=C(NCc1ccccc1C(F)(F)F)c1cccc(NCc2nnc3n2CCOc2cnccc2-3)c1]

or a pharmaceutically acceptable salt thereof.

In some embodiments, the compound of Formula III has a structure of Formula III-56:

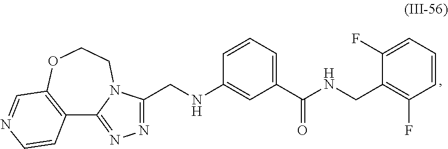 [SMILES: O=C(NCc1c(F)cccc1F)c1cccc(NCc2nnc3n2CCOc2cnccc2-3)c1]

or a pharmaceutically acceptable salt thereof.

Compounds of Formula III may be synthesized by methods known in the art, e.g., those described in International application publications: WO/2005/090328 and WO2007/034846, which are incorporated herein by reference.

In some embodiments of any of the above aspects, the GRK2 small molecule antagonist is a compound of Formula IV:

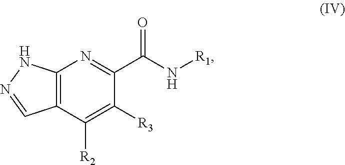

or a physiologically acceptable salt thereof, where;

R₁ is H or (C₁-C₆)alkyl, wherein the (C₁-C₆)alkyl group is unsubstituted or substituted by one or more OH, halogen, or NH₂ groups;

R₂ is phenyl, (C₃-C₁₀)cycloalkyl group, or (C₄-C₁₀)heterocyclyl group, each of which are unsubstituted or substituted independently by 1, 2 or 3 residues selected from:

1) (C₁-C₆)alkyl,
2) (C₂-C₆)alkenyl,
3) (C₂-C₆)alkynyl,
4) (C₁-C₆)alkylene-COOH,
5) (C₁-C₆)alkylene-C(O)O—(C₁-C₆)alkyl,
6) (C₁-C₆)alkylene-C(O)NH₂,
7) (C₁-C₆)alkylene-C(O)NH—(C₁-C₆)alkyl,
8) (C₁-C₆)alkylene-O—(C₁-C₆)alkyl,
9) (C₁-C₆)alkylene-OH,
10) (C₁-C₆)alkylene-NH₂,
11) (C₁-C₆)alkylene-NH—(C₁-C₆)alkyl,
12) (C₁-C₆)alkylene-N[(C₁-C₆)alkyl]₂,

### 13) CN,

### 14) COOH,

15) C(O)O—(C₁-C₆)alkyl,

### 16) C(O)NH₂,

17) C(O)NH—(C₁-C₆)alkyl,
18) C(O)N[(C₁-C₆)alkyl]₂,
19) C(O)—(C₁-C₆)alkyl,
20) halogen,

### 21) NH₂,

22) NH(C₁-C₆)alkyl,
23) N[(C₁-C₆)alkyl]₂,
24) NH—C(O)—(C₁-C₆)alkyl,

### 25) OH,

26) O—(C₁-C₆)alkyl,
27) O—(C₂-C₆)alkenyl,
28) O—(C₂-C₆)alkynyl,
29) O—(C₁-C₆)alkylene-C(O)OH,
30) O—(C₁-C₆)alkylene-C(O)O—(C₁-C₆)alkyl,
31) O—(C₁-C₆)alkylene-C(O)NH₂,
32) O—(C₁-C₆)alkylene-C(O)NH—(C₁-C₆)alkyl,
33) O—(C₁-C₆)alkylene-OH,
34) O—(C₁-C₆)alkylene-O—(C₁-C₆)alkyl,
35) O—(C₁-C₆)alkylene-NH₂,
36) O—(C₁-C₆)alkylene-NH—(C₁-C₆)alkyl,
37) O—C(O)—(C₁-C₆)alkyl,
38) S—(C₁-C₆)alkyl,
39) S(O)₂—(C₁-C₄)alkyl,
40) (C₅-C₁₀)aryl,
41) (C₄-C₁₀)heterocyclyl,
42) (C₁-C₅)alkylene-(C₅-C₁₀)aryl,
43) (C₁-C₆)alkylene-(C₄-C₁₀)heterocyclyl,
44) (C₁-C₅)alkylene-O—(C₅-C₁₀)aryl,
45) (C₁-C₆)alkylene-O—(C₄-C₁₀)heterocyclyl,
46) O—(C₅-C₁₀)aryl,
47) O—(C₄-C₁₀)heterocyclyl,
48) O—(C₁-C₅)alkylene-(C₅-C₁₀)aryl, and
49) O—(C₁-C₆)alkylene-(C₄-C₁₀)heterocyclyl,

where the (C₅-C₁₀)aryl and (C₄-C₁₀)heterocyclyl in residues 40) to 49) are each unsubstituted or substituted independently by 1, 2 or 3 residues selected from OH, halogen, NH₂, O—(C₁-C₆)alkyl, (C₁-C₆)alkyl, S(O)₂—(C₁-C₄)alkyl or (C₃-C₁₀)cycloalkyl; and/or is vicinally substituted by a residue of the formula —O—(CH₂)ₙ—O—, where n is 1, 2 or 3 and where one or more hydrogen atoms may be replaced by halogen atoms; and

R₃ is H, (C₁-C₆)alkyl, or O—(C₁-C₆)alkyl, where the (C₁-C₆)alkyl is unsubstituted or substituted by OH, halogen, NH₂, NH(C₁-C₆)alkyl or N[(C₁-C₆)alkyl]₂, or (C₃-C₁₀)cycloalkyl, which is unsubstituted or substituted by one or more fluoro atoms.

In some embodiments, the compound of Formula IV has a structure of Formula IV-1:

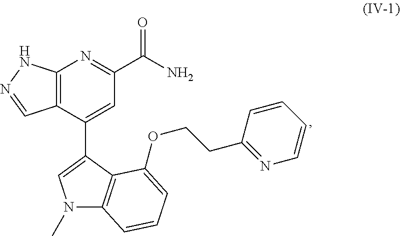 [SMILES: Cn1cc(-c2cc(C(N)=O)nc3[nH]ncc23)c2c(OCCc3ccccn3)cccc21]

or a physiologically acceptable salt thereof.

In some embodiments, the compound of Formula IV has a structure of Formula IV-2:

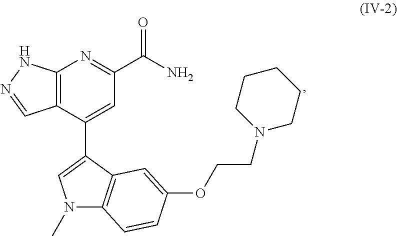 [SMILES: Cn1cc(-c2cc(C(N)=O)nc3[nH]ncc23)c2cc(OCCN3CCCCC3)ccc21]

or a physiologically acceptable salt thereof.

In some embodiments, the compound of Formula IV has a structure of Formula IV-3:

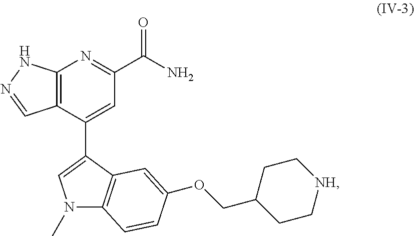 [SMILES: Cn1cc(-c2cc(C(N)=O)nc3[nH]ncc23)c2cc(OCC3CCNCC3)ccc21]

or a physiologically acceptable salt thereof.

In some embodiments, the compound of Formula IV has a structure of Formula IV-4:

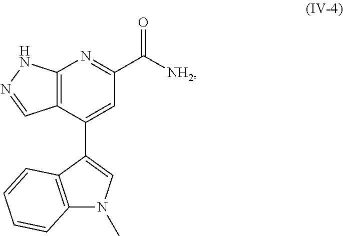 [SMILES: Cn1cc(-c2cc(C(N)=O)nc3[nH]ncc23)c2ccccc21]

or a physiologically acceptable salt thereof.

In some embodiments, the compound of Formula IV has a structure of Formula IV-5:

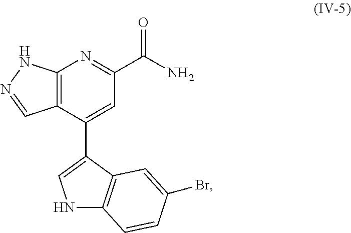 [SMILES: NC(=O)c1cc(-c2c[nH]c3ccc(Br)cc23)c2cn[nH]c2n1]

or a physiologically acceptable salt thereof.

In some embodiments, the compound of Formula IV has a structure of Formula IV-6:

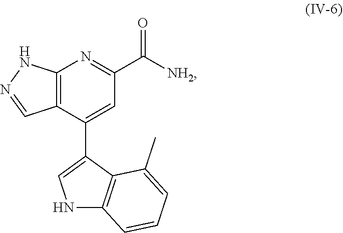 [SMILES: Cc1cccc2[nH]cc(-c3cc(C(N)=O)nc4[nH]ncc34)c12]

or a physiologically acceptable salt thereof.

In some embodiments, the compound of Formula IV has a structure of Formula IV-7:

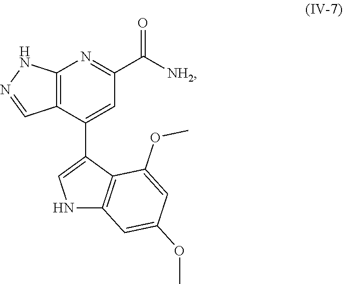 [SMILES: COc1cc(OC)c2c(-c3cc(C(N)=O)nc4[nH]ncc34)c[nH]c2c1]

or a physiologically acceptable salt thereof.

In some embodiments, the compound of Formula IV has a structure of Formula IV-8:

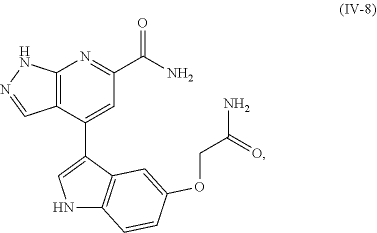 [SMILES: NC(=O)COc1ccc2[nH]cc(-c3cc(C(N)=O)nc4[nH]ncc34)c2c1]

or a physiologically acceptable salt thereof.

In some embodiments, the compound of Formula IV has a structure of Formula IV-9:

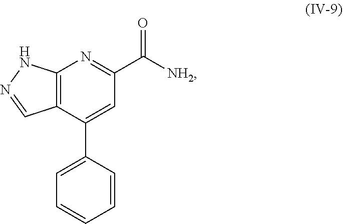 [SMILES: NC(=O)c1cc(-c2ccccc2)c2cn[nH]c2n1]

or a physiologically acceptable salt thereof.

In some embodiments, the compound of Formula IV has a structure of Formula IV-10:

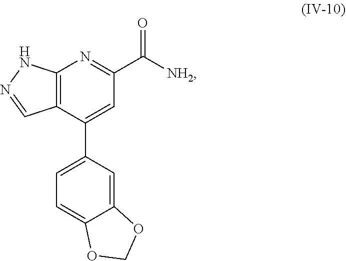 [SMILES: NC(=O)c1cc(-c2ccc3c(c2)OCO3)c2cn[nH]c2n1]

or a physiologically acceptable salt thereof.

In some embodiments, the compound of Formula IV has a structure of Formula IV-11:

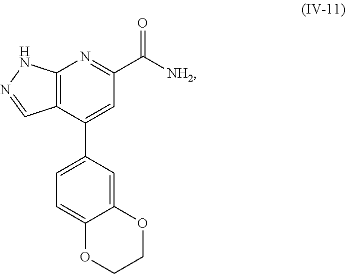 [SMILES: NC(=O)c1cc(-c2ccc3c(c2)OCCO3)c2cn[nH]c2n1]

or a physiologically acceptable salt thereof.

In some embodiments, the compound of Formula IV has a structure of Formula IV-12:

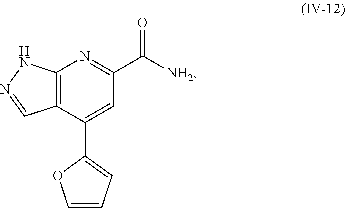 [SMILES: NC(=O)c1cc(-c2ccco2)c2cn[nH]c2n1]

or a physiologically acceptable salt thereof.

In some embodiments, the compound of Formula IV has a structure of Formula IV-13:

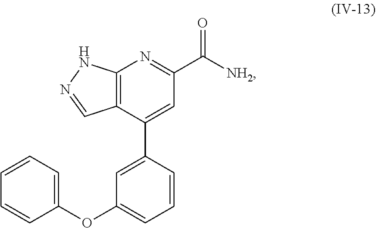 [SMILES: NC(=O)c1cc(-c2cccc(Oc3ccccc3)c2)c2cn[nH]c2n1]

or a physiologically acceptable salt thereof.

In some embodiments, the compound of Formula IV has a structure of Formula IV-14:

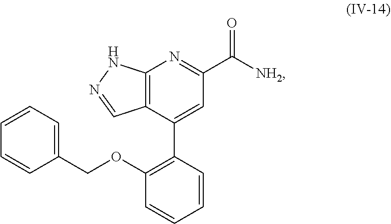 [SMILES: NC(=O)c1cc(-c2ccccc2OCc2ccccc2)c2cn[nH]c2n1]

or a physiologically acceptable salt thereof.

In some embodiments, the compound of Formula IV has a structure of Formula IV-15:

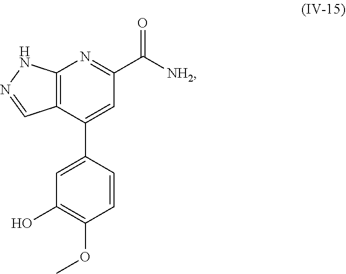 [SMILES: COc1ccc(-c2cc(C(N)=O)nc3[nH]ncc23)cc1O]

or a physiologically acceptable salt thereof.

In some embodiments, the compound of Formula IV has a structure of Formula IV-16:

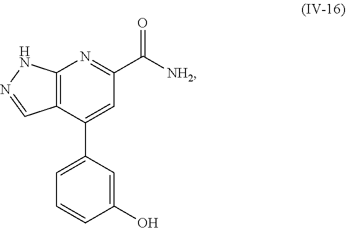 [SMILES: NC(=O)c1cc(-c2cccc(O)c2)c2cn[nH]c2n1]

or a physiologically acceptable salt thereof.

In some embodiments, the compound of Formula IV has a structure of Formula IV-17:

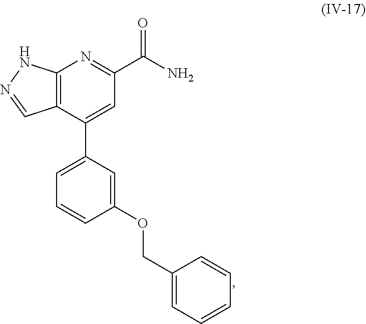 [SMILES: NC(=O)c1cc(-c2cccc(OCc3ccccc3)c2)c2cn[nH]c2n1]

or a physiologically acceptable salt thereof.

In some embodiments, the compound of Formula IV has a structure of Formula IV-18:

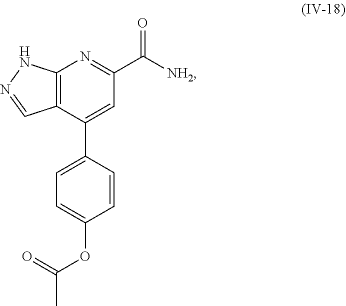 [SMILES: CC(=O)Oc1ccc(-c2cc(C(N)=O)nc3[nH]ncc23)cc1]

or a physiologically acceptable salt thereof.

In some embodiments, the compound of Formula IV has a structure of Formula IV-19:

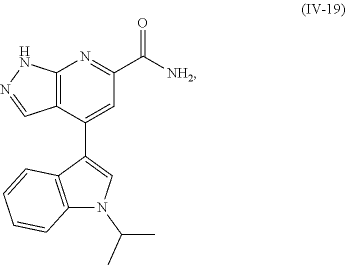 [SMILES: CC(C)n1cc(-c2cc(C(N)=O)nc3[nH]ncc23)c2ccccc21]

or a physiologically acceptable salt thereof.

In some embodiments, the compound of Formula IV has a structure of Formula IV-20:

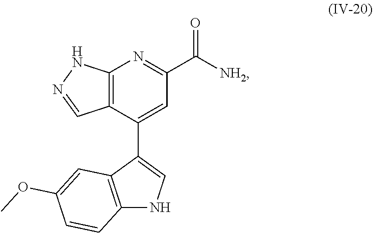 [SMILES: COc1ccc2[nH]cc(-c3cc(C(N)=O)nc4[nH]ncc34)c2c1]

or a physiologically acceptable salt thereof.

In some embodiments, the compound of Formula IV has a structure of Formula IV-21:

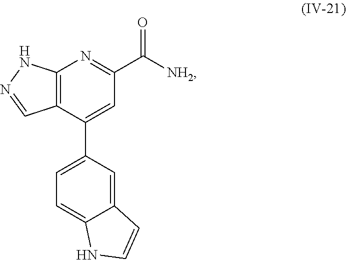 [SMILES: NC(=O)c1cc(-c2ccc3[nH]ccc3c2)c2cn[nH]c2n1]

or a physiologically acceptable salt thereof.

In some embodiments, the compound of Formula IV has a structure of Formula IV-22:

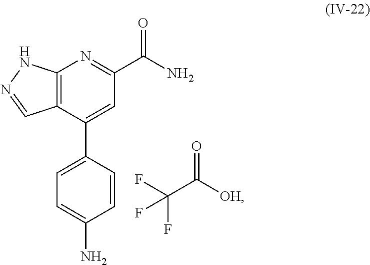

or a physiologically acceptable salt thereof.

In some embodiments, the compound of Formula IV has a structure of Formula IV-23:

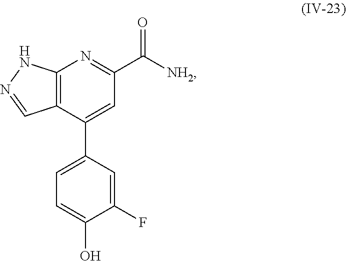 [SMILES: NC(=O)c1cc(-c2ccc(O)c(F)c2)c2cn[nH]c2n1]

or a physiologically acceptable salt thereof.

In some embodiments, the compound of Formula IV has a structure of Formula IV-24:

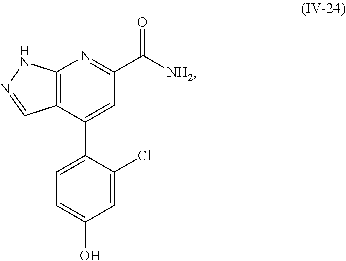 [SMILES: NC(=O)c1cc(-c2ccc(O)cc2Cl)c2cn[nH]c2n1]

or a physiologically acceptable salt thereof.

In some embodiments, the compound of Formula IV has a structure of Formula IV-25:

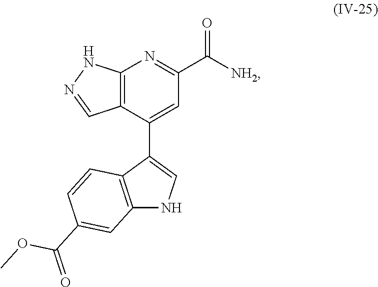 [SMILES: COC(=O)c1ccc2c(-c3cc(C(N)=O)nc4[nH]ncc34)c[nH]c2c1]

or a physiologically acceptable salt thereof.

In some embodiments, the compound of Formula IV has a structure of Formula IV-26:

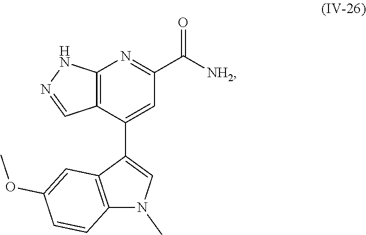 [SMILES: COc1ccc2c(c1)c(-c1cc(C(N)=O)nc3[nH]ncc13)cn2C]

or a physiologically acceptable salt thereof.

In some embodiments, the compound of Formula IV has a structure of Formula IV-27:

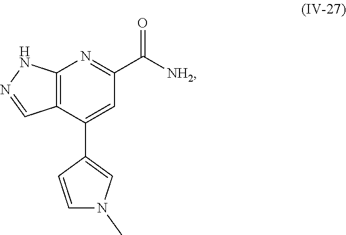 [SMILES: Cn1ccc(-c2cc(C(N)=O)nc3[nH]ncc23)c1]

or a physiologically acceptable salt thereof.

In some embodiments, the compound of Formula IV has a structure of Formula IV-28:

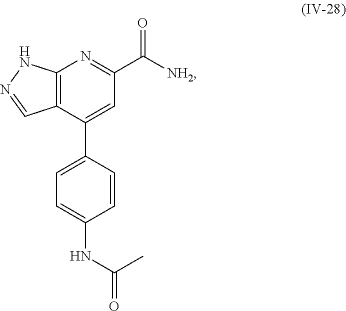 [SMILES: CC(=O)Nc1ccc(-c2cc(C(N)=O)nc3[nH]ncc23)cc1]

or a physiologically acceptable salt thereof.

In some embodiments, the compound of Formula IV has a structure of Formula IV-29:

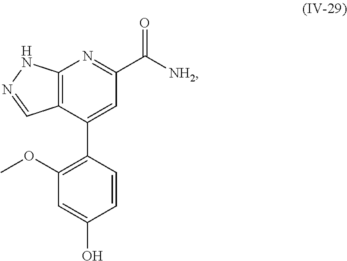 [SMILES: COc1cc(O)ccc1-c1cc(C(N)=O)nc2[nH]ncc12]

or a physiologically acceptable salt thereof.

In some embodiments, the compound of Formula IV has a structure of Formula IV-30:

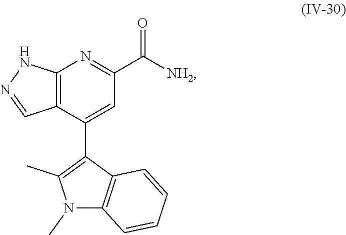 [SMILES: Cc1c(-c2cc(C(N)=O)nc3[nH]ncc23)c2ccccc2n1C]

or a physiologically acceptable salt thereof.

In some embodiments, the compound of Formula IV has a structure of Formula IV-31:

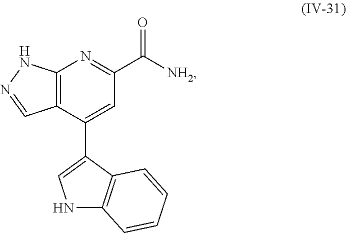 [SMILES: NC(=O)c1cc(-c2c[nH]c3ccccc23)c2cn[nH]c2n1]

or a physiologically acceptable salt thereof.

In some embodiments, the compound of Formula IV has a structure of Formula IV-32:

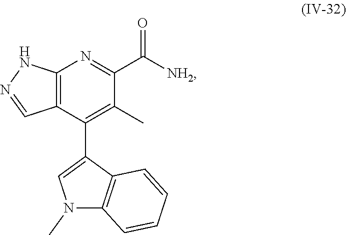 [SMILES: Cc1c(C(N)=O)nc2[nH]ncc2c1-c1cn(C)c2ccccc12]

or a physiologically acceptable salt thereof.

In some embodiments, the compound of Formula IV has a structure of Formula IV-33:

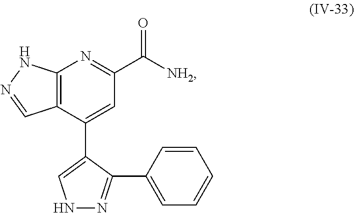 [SMILES: NC(=O)c1cc(-c2c[nH]nc2-c2ccccc2)c2cn[nH]c2n1]

or a physiologically acceptable salt thereof.

In some embodiments, the compound of Formula IV has a structure of Formula IV-34:

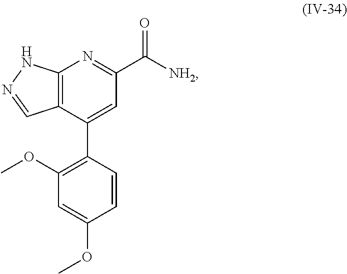 [SMILES: COc1ccc(-c2cc(C(N)=O)nc3[nH]ncc23)c(OC)c1]

or a physiologically acceptable salt thereof.

In some embodiments, the compound of Formula IV has a structure of Formula IV-35:

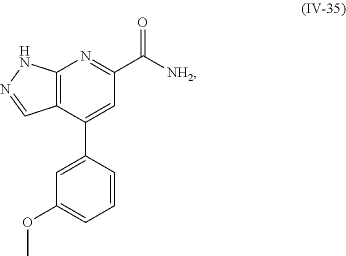 [SMILES: COc1cccc(-c2cc(C(N)=O)nc3[nH]ncc23)c1]

or a physiologically acceptable salt thereof.

In some embodiments, the compound of Formula IV has a structure of Formula IV-36:

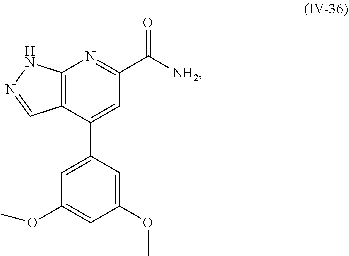 [SMILES: COc1cc(OC)cc(-c2cc(C(N)=O)nc3[nH]ncc23)c1]

or a physiologically acceptable salt thereof.

In some embodiments, the compound of Formula IV has a structure of Formula IV-37:

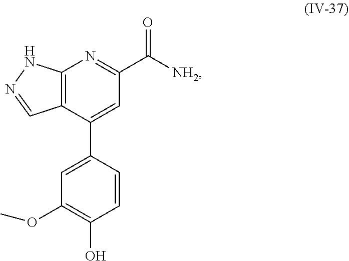 [SMILES: COc1cc(-c2cc(C(N)=O)nc3[nH]ncc23)ccc1O]

or a physiologically acceptable salt thereof.

In some embodiments, the compound of Formula IV has a structure of Formula IV-38:

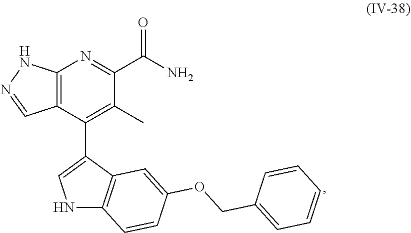 [SMILES: Cc1c(C(N)=O)nc2[nH]ncc2c1-c1c[nH]c2ccc(OCc3ccccc3)cc12]

or a physiologically acceptable salt thereof.

In some embodiments, the compound of Formula IV has a structure of Formula IV-39:

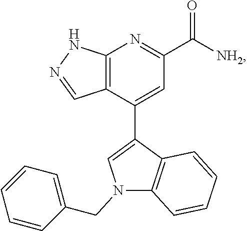 [SMILES: NC(=O)c1cc(-c2cn(Cc3ccccc3)c3ccccc23)c2cn[nH]c2n1]

or a physiologically acceptable salt thereof.

In some embodiments, the compound of Formula IV has a structure of Formula IV-40:

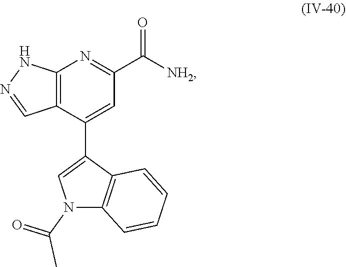 [SMILES: CC(=O)n1cc(-c2cc(C(N)=O)nc3[nH]ncc23)c2ccccc21]

or a physiologically acceptable salt thereof.

In some embodiments, the compound of Formula IV has a structure of Formula IV-41:

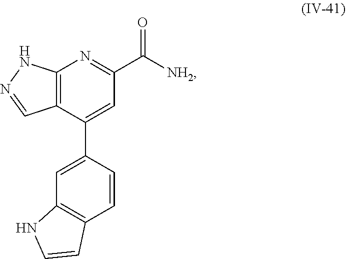 [SMILES: NC(=O)c1cc(-c2ccc3cc[nH]c3c2)c2cn[nH]c2n1]

or a physiologically acceptable salt thereof.

In some embodiments, the compound of Formula IV has a structure of Formula IV-42:

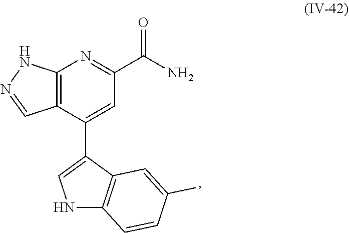

or a physiologically acceptable salt thereof.

In some embodiments, the compound of Formula IV has a structure of Formula IV-43:

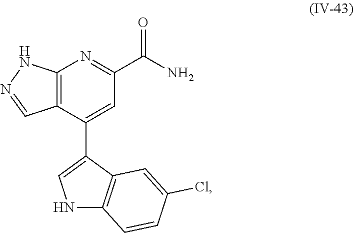 [SMILES: NC(=O)c1cc(-c2c[nH]c3ccc(Cl)cc23)c2cn[nH]c2n1]

or a physiologically acceptable salt thereof.

In some embodiments, the compound of Formula IV has a structure of Formula IV-44:

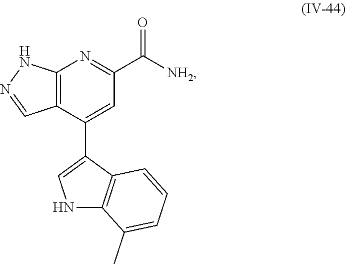 [SMILES: Cc1cccc2c(-c3cc(C(N)=O)nc4[nH]ncc34)c[nH]c12]

or a physiologically acceptable salt thereof.

In some embodiments, the compound of Formula IV has a structure of Formula IV-45:

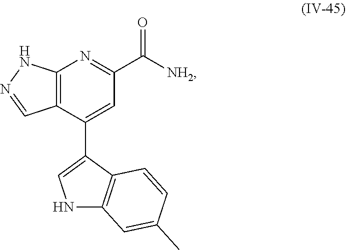 [SMILES: Cc1ccc2c(-c3cc(C(N)=O)nc4[nH]ncc34)c[nH]c2c1]

or a physiologically acceptable salt thereof.

In some embodiments, the compound of Formula IV has a structure of Formula IV-46:

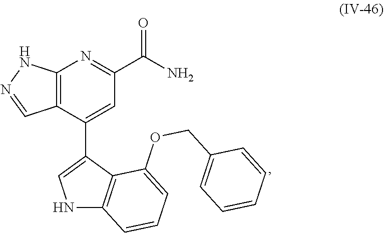 [SMILES: NC(=O)c1cc(-c2c[nH]c3cccc(OCc4ccccc4)c23)c2cn[nH]c2n1]

or a physiologically acceptable salt thereof.

In some embodiments, the compound of Formula IV has a structure of Formula IV-47:

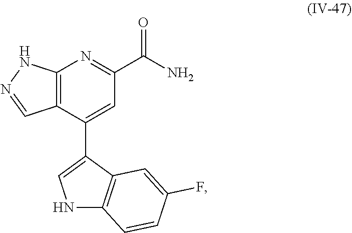 [SMILES: NC(=O)c1cc(-c2c[nH]c3ccc(F)cc23)c2cn[nH]c2n1]

or a physiologically acceptable salt thereof.

In some embodiments, the compound of Formula IV has a structure of Formula IV-48:

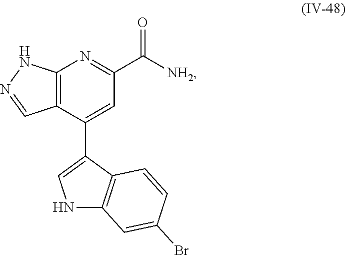 [SMILES: NC(=O)c1cc(-c2c[nH]c3cc(Br)ccc23)c2cn[nH]c2n1]

or a physiologically acceptable salt thereof.

In some embodiments, the compound of Formula IV has a structure of Formula IV-49:

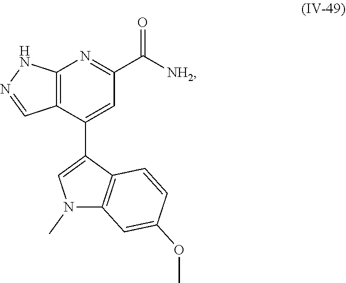 [SMILES: COc1ccc2c(-c3cc(C(N)=O)nc4[nH]ncc34)cn(C)c2c1]

or a physiologically acceptable salt thereof.

In some embodiments, the compound of Formula IV has a structure of Formula IV-50:

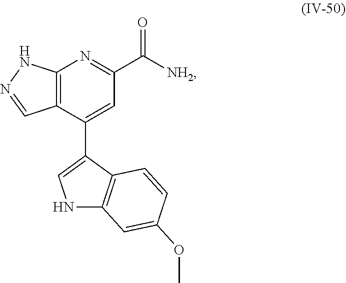 [SMILES: COc1ccc2c(-c3cc(C(N)=O)nc4[nH]ncc34)c[nH]c2c1]

or a physiologically acceptable salt thereof.

In some embodiments, the compound of Formula IV has a structure of Formula IV-51:

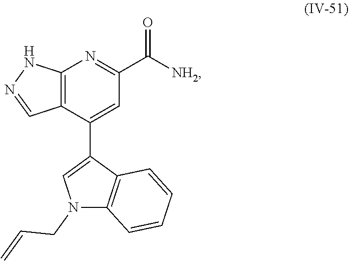 [SMILES: C=CCn1cc(-c2cc(C(N)=O)nc3[nH]ncc23)c2ccccc21]

or a physiologically acceptable salt thereof.

In some embodiments, the compound of Formula IV has a structure of Formula IV-52:

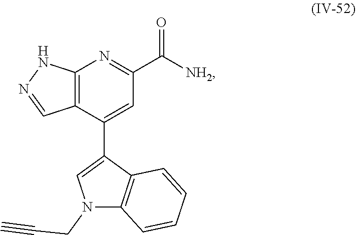 [SMILES: C#CCn1cc(-c2cc(C(N)=O)nc3[nH]ncc23)c2ccccc21]

or a physiologically acceptable salt thereof.

In some embodiments, the compound of Formula IV has a structure of Formula IV-53:

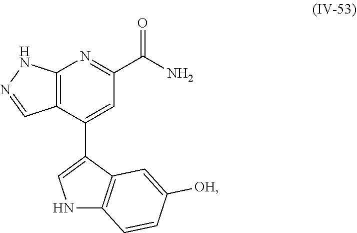 [SMILES: NC(=O)c1cc(-c2c[nH]c3ccc(O)cc23)c2cn[nH]c2n1]

or a physiologically acceptable salt thereof.

In some embodiments, the compound of Formula IV has a structure of Formula IV-54:

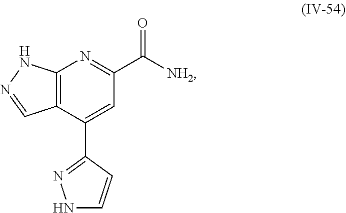 [SMILES: NC(=O)c1cc(-c2cc[nH]n2)c2cn[nH]c2n1]

or a physiologically acceptable salt thereof.

In some embodiments, the compound of Formula IV has a structure of Formula IV-55:

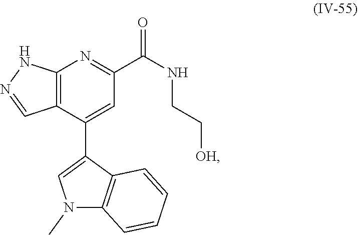 [SMILES: Cn1cc(-c2cc(C(=O)NCCO)nc3[nH]ncc23)c2ccccc21]

or a physiologically acceptable salt thereof.

In some embodiments, the compound of Formula IV has a structure of Formula IV-56:

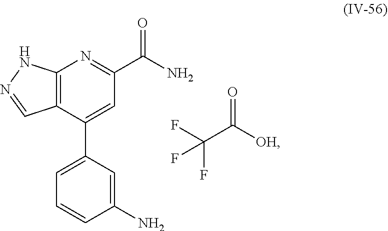 [SMILES: NC(=O)c1cc(-c2cccc(N)c2)c2cn[nH]c2n1 ; O=C(O)C(F)(F)F]

or a physiologically acceptable salt thereof.

In some embodiments, the compound of Formula IV has a structure of Formula IV-57:

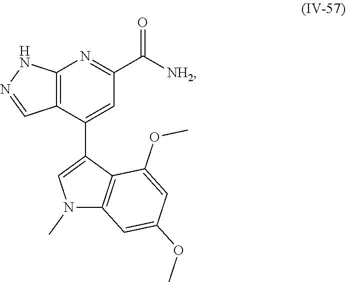 [SMILES: COc1cc(OC)c2c(-c3cc(C(N)=O)nc4[nH]ncc34)cn(C)c2c1]

or a physiologically acceptable salt thereof.

In some embodiments, the compound of Formula IV has a structure of Formula IV-58:

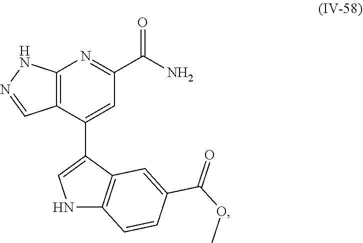 [SMILES: COC(=O)c1ccc2[nH]cc(-c3cc(C(N)=O)nc4[nH]ncc34)c2c1]

or a physiologically acceptable salt thereof.

In some embodiments, the compound of Formula IV has a structure of Formula IV-59:

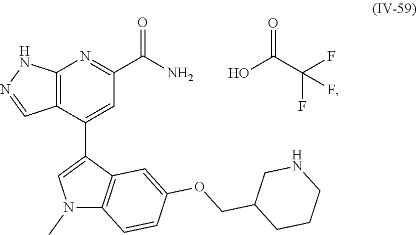 [SMILES: Cn1cc(-c2cc(C(N)=O)nc3[nH]ncc23)c2cc(OCC3CCCNC3)ccc21 ; O=C(O)C(F)(F)F]

or a physiologically acceptable salt thereof.

In some embodiments, the compound of Formula IV has a structure of Formula IV-60:

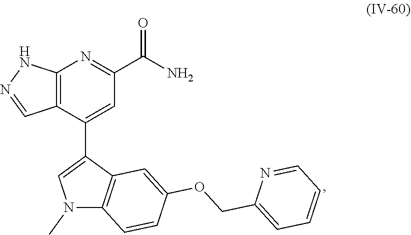 [SMILES: Cn1cc(-c2cc(C(N)=O)nc3[nH]ncc23)c2cc(OCc3ccccn3)ccc21]

or a physiologically acceptable salt thereof.

In some embodiments, the compound of Formula IV has a structure of Formula IV-61:

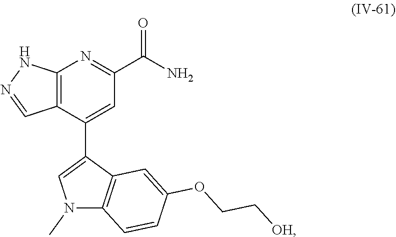 [SMILES: Cn1cc(-c2cc(C(N)=O)nc3[nH]ncc23)c2cc(OCCO)ccc21]

or a physiologically acceptable salt thereof.

In some embodiments, the compound of Formula IV has a structure of Formula IV-62:

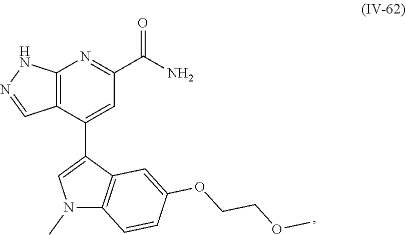 [SMILES: COCCOc1ccc2c(c1)c(-c1cc(C(N)=O)nc3[nH]ncc13)cn2C]

or a physiologically acceptable salt thereof.

In some embodiments, the compound of Formula IV has a structure of Formula IV-63:

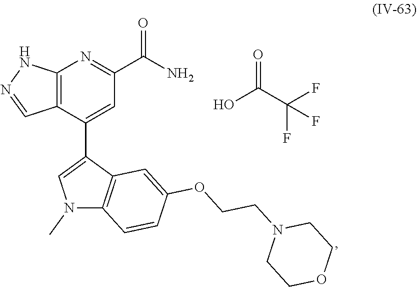 [SMILES: Cn1cc(-c2cc(C(N)=O)nc3[nH]ncc23)c2cc(OCCN3CCOCC3)ccc21 ; O=C(O)C(F)(F)F]

or a physiologically acceptable salt thereof.

In some embodiments, the compound of Formula IV has a structure of Formula IV-64:

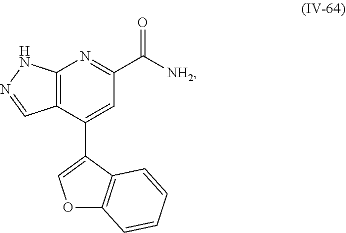 [SMILES: NC(=O)c1cc(-c2coc3ccccc23)c2cn[nH]c2n1]

or a physiologically acceptable salt thereof.

In some embodiments, the compound of Formula IV has a structure of Formula IV-65:

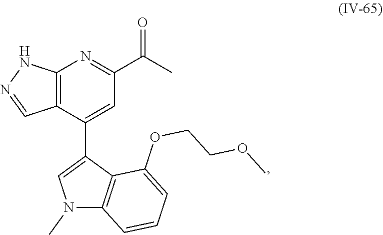 [SMILES: COCCOc1cccc2c1c(-c1cc(C(C)=O)nc3[nH]ncc13)cn2C]

or a physiologically acceptable salt thereof.

In some embodiments, the compound of Formula IV has a structure of Formula IV-66:

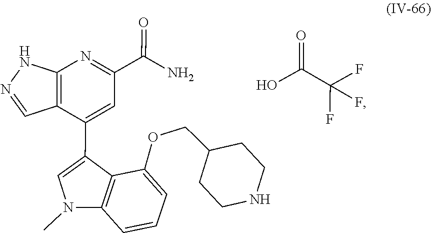 [SMILES: Cn1cc(-c2cc(C(N)=O)nc3[nH]ncc23)c2c(OCC3CCNCC3)cccc21 ; O=C(O)C(F)(F)F]

or a physiologically acceptable salt thereof.

In some embodiments, the compound of Formula IV has a structure of Formula IV-67:

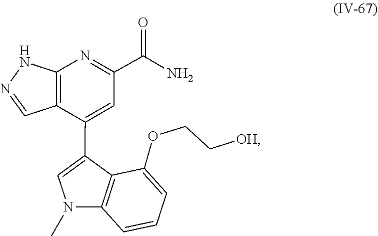 [SMILES: Cn1cc(-c2cc(C(N)=O)nc3[nH]ncc23)c2c(OCCO)cccc21]

or a physiologically acceptable salt thereof.

In some embodiments, the compound of Formula IV has a structure of Formula IV-68:

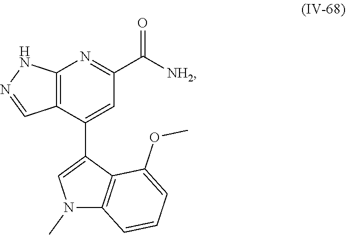 [SMILES: COc1cccc2c1c(-c1cc(C(N)=O)nc3[nH]ncc13)cn2C]

or a physiologically acceptable salt thereof.

In some embodiments, the compound of Formula IV has a structure of Formula IV-69:

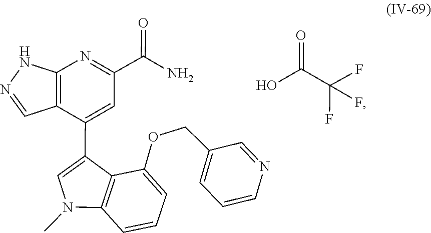 [SMILES: Cn1cc(-c2cc(C(N)=O)nc3[nH]ncc23)c2c(OCc3cccnc3)cccc21 ; O=C(O)C(F)(F)F]

or a physiologically acceptable salt thereof.

In some embodiments, the compound of Formula IV has a structure of Formula IV-70:

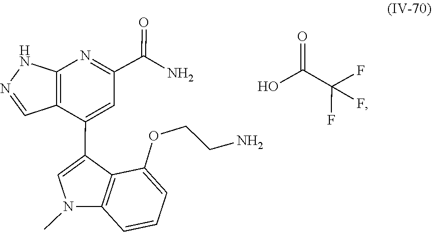 [SMILES: Cn1cc(-c2cc(C(N)=O)nc3[nH]ncc23)c2c(OCCN)cccc21 ; O=C(O)C(F)(F)F]

or a physiologically acceptable salt thereof.

In some embodiments, the compound of Formula IV has a structure of Formula IV-71:

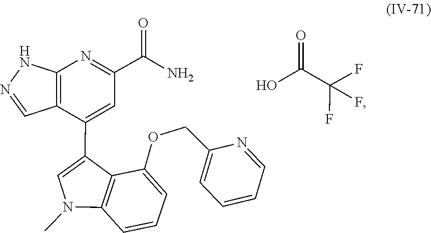 [SMILES: Cn1cc(-c2cc(C(N)=O)nc3[nH]ncc23)c2c(OCc3ccccn3)cccc21 ; O=C(O)C(F)(F)F]

or a physiologically acceptable salt thereof.

In some embodiments, the compound of Formula IV has a structure of Formula IV-72:

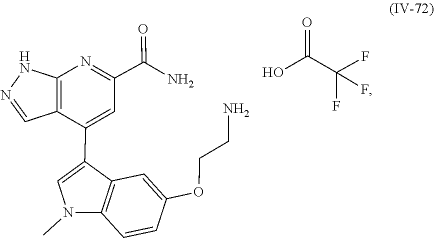 [SMILES: Cn1cc(-c2cc(C(N)=O)nc3[nH]ncc23)c2cc(OCCN)ccc21 ; O=C(O)C(F)(F)F]

or a physiologically acceptable salt thereof.

In some embodiments, the compound of Formula IV has a structure of Formula IV-73:

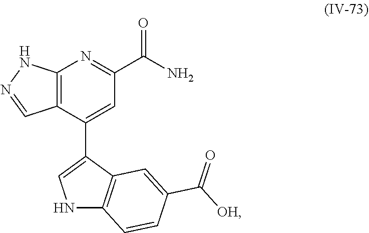 [SMILES: NC(=O)c1cc(-c2c[nH]c3ccc(C(=O)O)cc23)c2cn[nH]c2n1]

or a physiologically acceptable salt thereof.

In some embodiments, the compound of Formula IV has a structure of Formula IV-74:

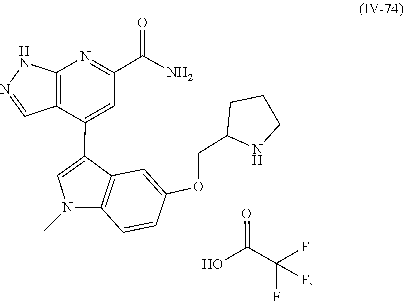 [SMILES: Cn1cc(-c2cc(C(N)=O)nc3[nH]ncc23)c2cc(OCC3CCCN3)ccc21 ; O=C(O)C(F)(F)F]

or a physiologically acceptable salt thereof.

In some embodiments, the compound of Formula IV has a structure of Formula IV-75:

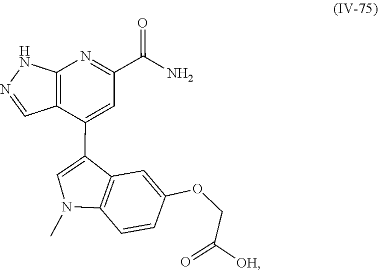 [SMILES: Cn1cc(-c2cc(C(N)=O)nc3[nH]ncc23)c2cc(OCC(=O)O)ccc21]

or a physiologically acceptable salt thereof.

In some embodiments, the compound of Formula IV has a structure of Formula IV-76:

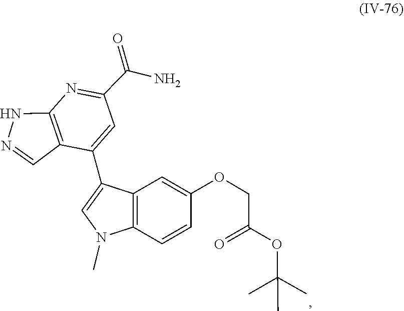 [SMILES: Cn1cc(-c2cc(C(N)=O)nc3[nH]ncc23)c2cc(OCC(=O)OC(C)(C)C)ccc21]

or a physiologically acceptable salt thereof.

In some embodiments, the compound of Formula IV has a structure of Formula IV-77:

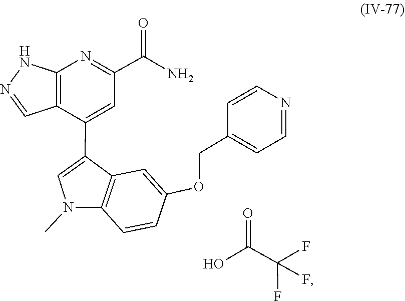 [SMILES: Cn1cc(-c2cc(C(N)=O)nc3[nH]ncc23)c2cc(OCc3ccncc3)ccc21 ; O=C(O)C(F)(F)F]

or a physiologically acceptable salt thereof.

In some embodiments, the compound of Formula IV has a structure of Formula IV-78:

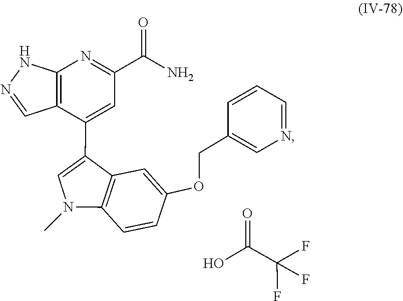 [SMILES: Cn1cc(-c2cc(C(N)=O)nc3[nH]ncc23)c2cc(OCc3cccnc3)ccc21 ; O=C(O)C(F)(F)F]

or a physiologically acceptable salt thereof.

In some embodiments, the compound of Formula IV has a structure of Formula IV-79:

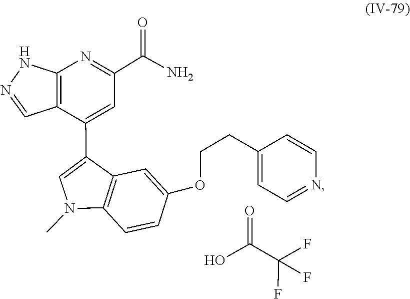

or a physiologically acceptable salt thereof.

In some embodiments, the compound of Formula IV has a structure of Formula IV-80:

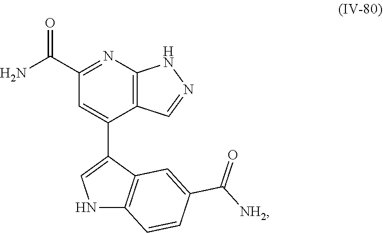 [SMILES: NC(=O)c1ccc2[nH]cc(-c3cc(C(N)=O)nc4[nH]ncc34)c2c1]

or a physiologically acceptable salt thereof.

Compounds of Formula IV may be synthesized by methods known in the art, e.g., those described in U.S. Pat. No. 7,910,602, which is incorporated herein by reference.

In some embodiments of any of the above aspects, the GRK2 small molecule antagonist is selected from Table 1.

TABLE 1
GRK2 SMALL MOLECULE ANTAGONISTS

| Molecule | Structure |
|---|---|
| Compound 1 | 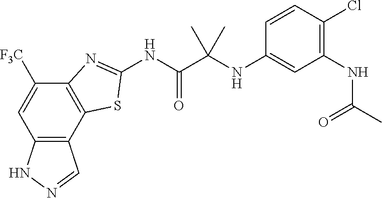 [SMILES: CC(=O)Nc1cc(NC(C)(C)C(=O)Nc2nc3c(C(F)(F)F)cc4[nH]ncc4c3s2)ccc1Cl] |
| Compound 2 | 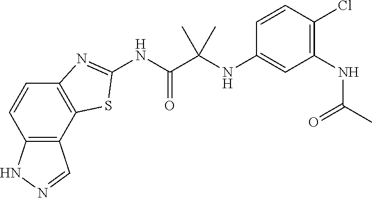 [SMILES: CC(=O)Nc1cc(NC(C)(C)C(=O)Nc2nc3ccc4[nH]ncc4c3s2)ccc1Cl] |
| Compound 3 | 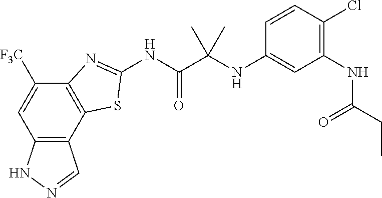 [SMILES: CCC(=O)Nc1cc(NC(C)(C)C(=O)Nc2nc3c(C(F)(F)F)cc4[nH]ncc4c3s2)ccc1Cl] |
| Compound 4 | 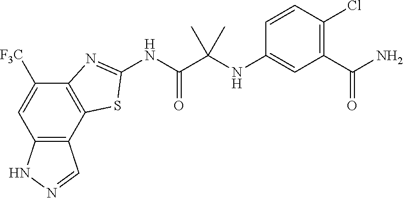 [SMILES: CC(C)(Nc1ccc(Cl)c(C(N)=O)c1)C(=O)Nc1nc2c(C(F)(F)F)cc3[nH]ncc3c2s1] |
| Compound 5 | 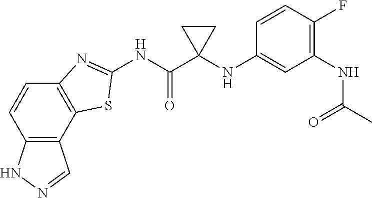 [SMILES: CC(=O)Nc1cc(NC2(C(=O)Nc3nc4ccc5[nH]ncc5c4s3)CC2)ccc1F] |
| Compound 6 | 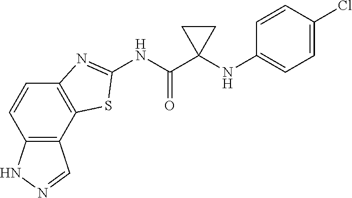 [SMILES: O=C(Nc1nc2ccc3[nH]ncc3c2s1)C1(Nc2ccc(Cl)cc2)CC1] |
| Compound 7 | 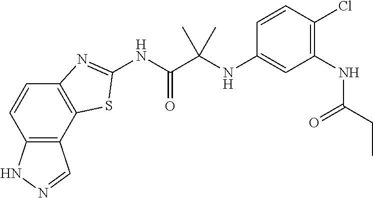 [SMILES: CCC(=O)Nc1cc(NC(C)(C)C(=O)Nc2nc3ccc4[nH]ncc4c3s2)ccc1Cl] |
| Compound 8 | 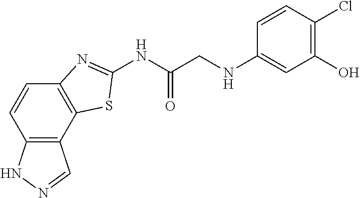 [SMILES: O=C(CNc1ccc(Cl)c(O)c1)Nc1nc2ccc3[nH]ncc3c2s1] |
| Compound 9 | 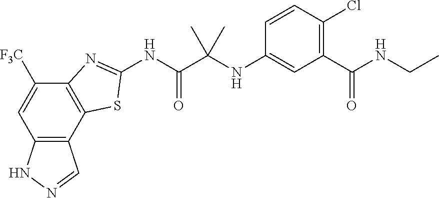 [SMILES: CCNC(=O)c1cc(NC(C)(C)C(=O)Nc2nc3c(C(F)(F)F)cc4[nH]ncc4c3s2)ccc1Cl] |
| Compound 10 | 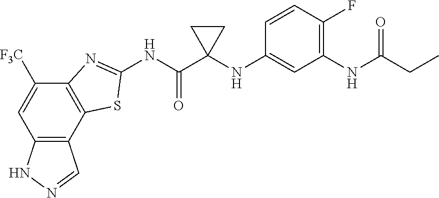 [SMILES: CCC(=O)Nc1cc(NC2(C(=O)Nc3nc4c(C(F)(F)F)cc5[nH]ncc5c4s3)CC2)ccc1F] |
| Compound 11 | 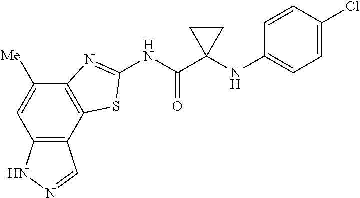 [SMILES: Cc1cc2[nH]ncc2c2sc(NC(=O)C3(Nc4ccc(Cl)cc4)CC3)nc12] |
| Compound 12 | 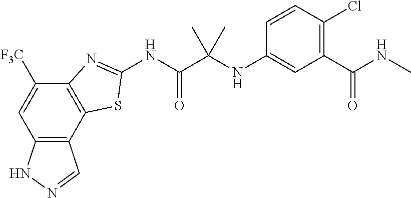 [SMILES: CNC(=O)c1cc(NC(C)(C)C(=O)Nc2nc3c(C(F)(F)F)cc4[nH]ncc4c3s2)ccc1Cl] |
| Compound 13 | 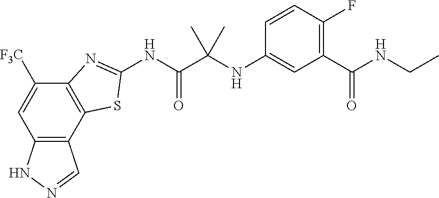 [SMILES: CCNC(=O)c1cc(NC(C)(C)C(=O)Nc2nc3c(C(F)(F)F)cc4[nH]ncc4c3s2)ccc1F] |
| Compound 14 | 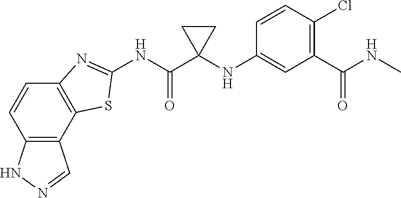 [SMILES: CNC(=O)c1cc(NC2(C(=O)Nc3nc4ccc5[nH]ncc5c4s3)CC2)ccc1Cl] |
| Compound 15 | 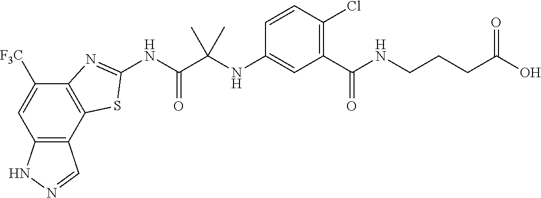 [SMILES: CC(C)(Nc1ccc(Cl)c(C(=O)NCCCC(=O)O)c1)C(=O)Nc1nc2c(C(F)(F)F)cc3[nH]ncc3c2s1] |
| Compound 16 | 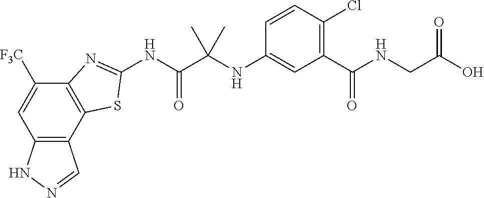 [SMILES: CC(C)(Nc1ccc(Cl)c(C(=O)NCC(=O)O)c1)C(=O)Nc1nc2c(C(F)(F)F)cc3[nH]ncc3c2s1] |
| Compound 17 | 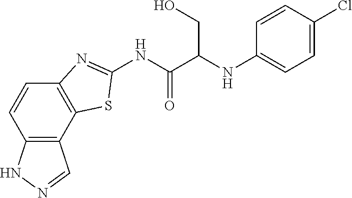 [SMILES: O=C(Nc1nc2ccc3[nH]ncc3c2s1)C(CO)Nc1ccc(Cl)cc1] |
| Compound 18 | 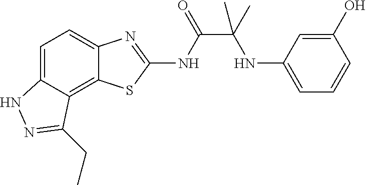 [SMILES: CCc1n[nH]c2ccc3nc(NC(=O)C(C)(C)Nc4cccc(O)c4)sc3c12] |
| Compound 19 | 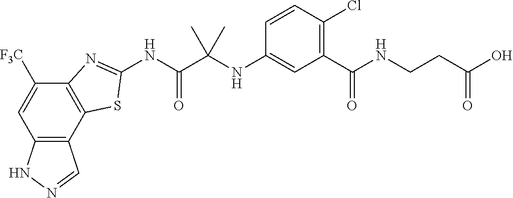 [SMILES: CC(C)(Nc1ccc(Cl)c(C(=O)NCCC(=O)O)c1)C(=O)Nc1nc2c(C(F)(F)F)cc3[nH]ncc3c2s1] |
| Compound 20 | 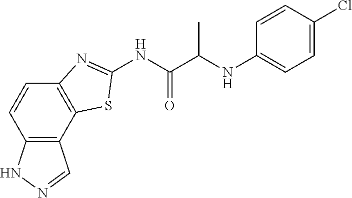 [SMILES: CC(Nc1ccc(Cl)cc1)C(=O)Nc1nc2ccc3[nH]ncc3c2s1] |
| Compound 21 | 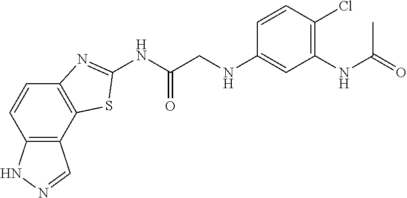 [SMILES: CC(=O)Nc1cc(NCC(=O)Nc2nc3ccc4[nH]ncc4c3s2)ccc1Cl] |
| Compound 22 | 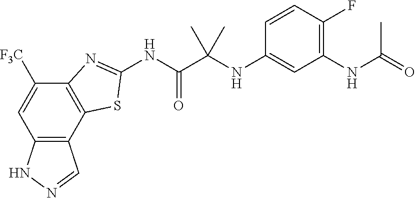 [SMILES: CC(=O)Nc1cc(NC(C)(C)C(=O)Nc2nc3c(C(F)(F)F)cc4[nH]ncc4c3s2)ccc1F] |
| Compound 23 | 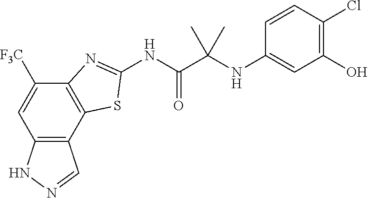 [SMILES: CC(C)(Nc1ccc(Cl)c(O)c1)C(=O)Nc1nc2c(C(F)(F)F)cc3[nH]ncc3c2s1] |
| Compound 24 | 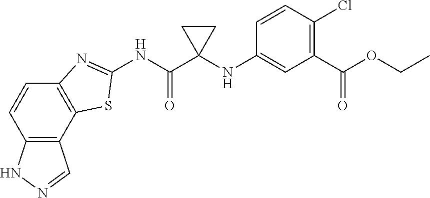 [SMILES: CCOC(=O)c1cc(NC2(C(=O)Nc3nc4ccc5[nH]ncc5c4s3)CC2)ccc1Cl] |
| Compound 25 | 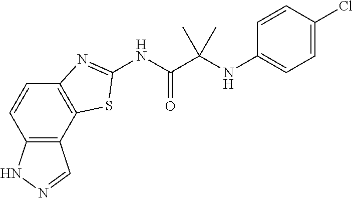 [SMILES: CC(C)(Nc1ccc(Cl)cc1)C(=O)Nc1nc2ccc3[nH]ncc3c2s1] |
| Compound 26 | 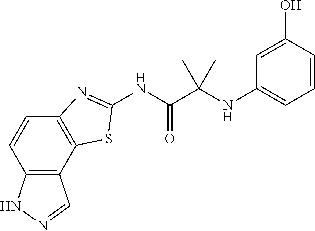 [SMILES: CC(C)(Nc1cccc(O)c1)C(=O)Nc1nc2ccc3[nH]ncc3c2s1] |
| Compound 27 | 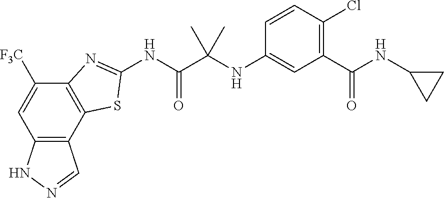 [SMILES: CC(C)(Nc1ccc(Cl)c(C(=O)NC2CC2)c1)C(=O)Nc1nc2c(C(F)(F)F)cc3[nH]ncc3c2s1] |
| Compound 28 | 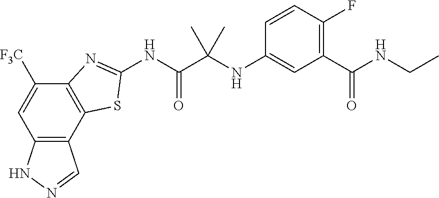 [SMILES: CCNC(=O)c1cc(NC(C)(C)C(=O)Nc2nc3c(C(F)(F)F)cc4[nH]ncc4c3s2)ccc1F] |
| Compound 29 | 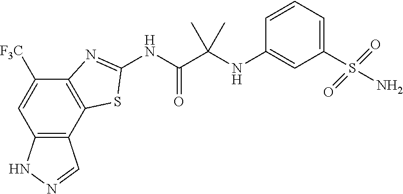 [SMILES: CC(C)(Nc1cccc(S(N)(=O)=O)c1)C(=O)Nc1nc2c(C(F)(F)F)cc3[nH]ncc3c2s1] |
| Compound 30 | 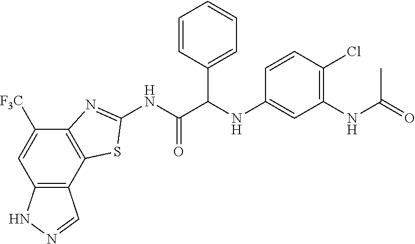 [SMILES: CC(=O)Nc1cc(NC(C(=O)Nc2nc3c(C(F)(F)F)cc4[nH]ncc4c3s2)c2ccccc2)ccc1Cl] |
| Compound 31 | 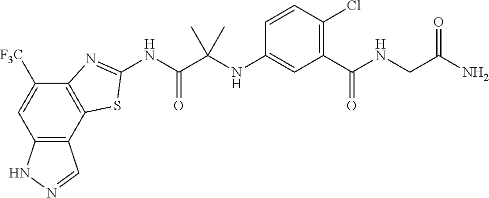 [SMILES: CC(C)(Nc1ccc(Cl)c(C(=O)NCC(N)=O)c1)C(=O)Nc1nc2c(C(F)(F)F)cc3[nH]ncc3c2s1] |
| Compound 32 | 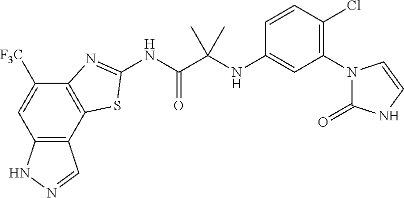 [SMILES: CC(C)(Nc1ccc(Cl)c(-n2cc[nH]c2=O)c1)C(=O)Nc1nc2c(C(F)(F)F)cc3[nH]ncc3c2s1] |
| Compound 33 | 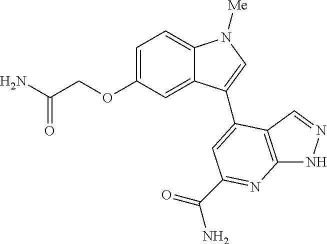 [SMILES: Cn1cc(-c2cc(C(N)=O)nc3[nH]ncc23)c2cc(OCC(N)=O)ccc21] |
| Compound 34 | 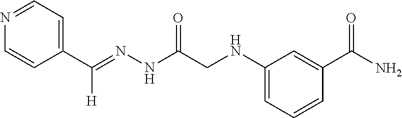 [SMILES: NC(=O)c1cccc(NCC(=O)N/N=C/c2ccncc2)c1] |
| Compound 35 | 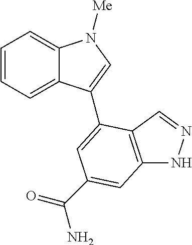 [SMILES: Cn1cc(-c2cc(C(N)=O)cc3[nH]ncc23)c2ccccc21] |
| Compound 36 | 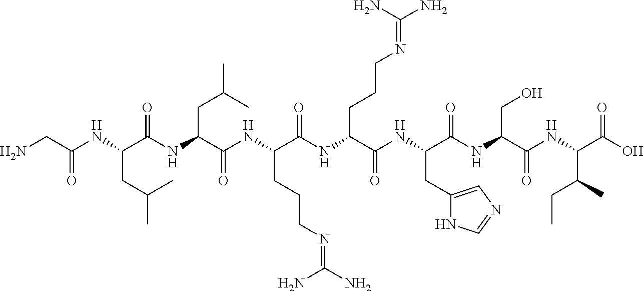 [SMILES: CC[C@H](C)[C@H](NC(=O)[C@H](CO)NC(=O)[C@H](Cc1cnc[nH]1)NC(=O)[C@@H](CCCN=C(N)N)NC(=O)[C@H](CCCN=C(N)N)NC(=O)[C@H](CC(C)C)NC(=O)[C@H](CC(C)C)NC(=O)CN)C(=O)O] |
| Compound 37 | 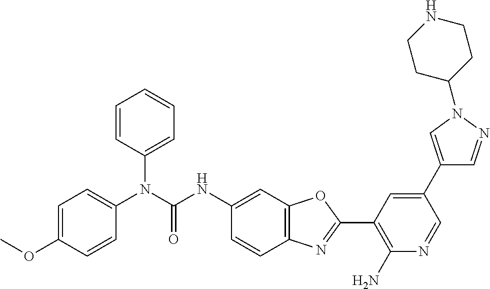 [SMILES: COc1ccc(N(C(=O)Nc2ccc3nc(-c4cc(-c5cnn(C6CCNCC6)c5)cnc4N)oc3c2)c2ccccc2)cc1] |
| Compound 38 | 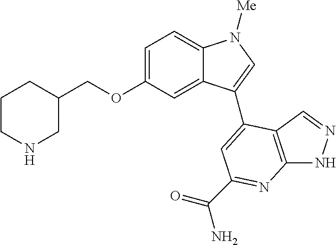 [SMILES: Cn1cc(-c2cc(C(N)=O)nc3[nH]ncc23)c2cc(OCC3CCCNC3)ccc21] |
| Compound 39 | 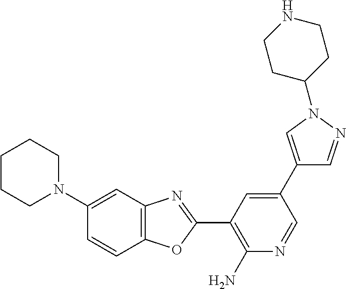 [SMILES: Nc1ncc(-c2cnn(C3CCNCC3)c2)cc1-c1nc2cc(N3CCCCC3)ccc2o1] |
| Compound 40 | 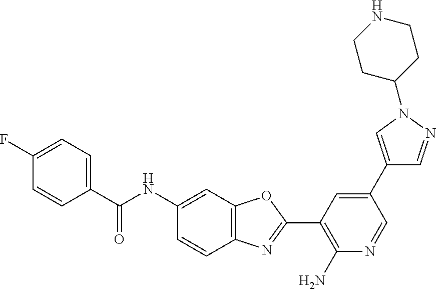 [SMILES: Nc1ncc(-c2cnn(C3CCNCC3)c2)cc1-c1nc2ccc(NC(=O)c3ccc(F)cc3)cc2o1] |
| Compound 41 | 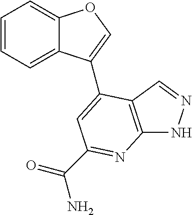 [SMILES: NC(=O)c1cc(-c2coc3ccccc23)c2cn[nH]c2n1] |
| Compound 42 | 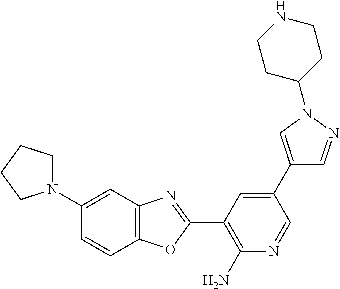 [SMILES: Nc1ncc(-c2cnn(C3CCNCC3)c2)cc1-c1nc2cc(N3CCCC3)ccc2o1] |
| Compound 43 | 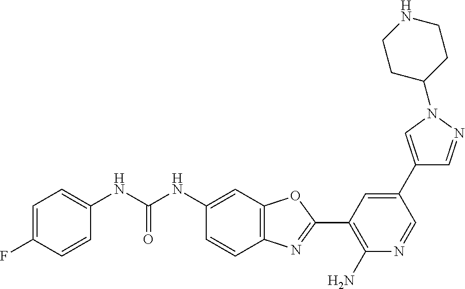 [SMILES: Nc1ncc(-c2cnn(C3CCNCC3)c2)cc1-c1nc2ccc(NC(=O)Nc3ccc(F)cc3)cc2o1] |
| Compound 44 | 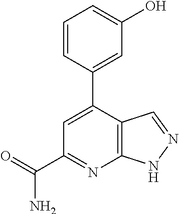 [SMILES: NC(=O)c1cc(-c2cccc(O)c2)c2cn[nH]c2n1] |
| Compound 45 | 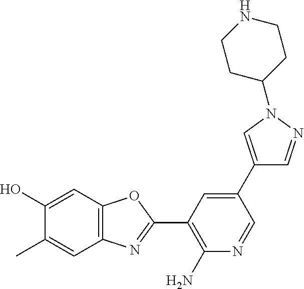 [SMILES: Cc1cc2nc(-c3cc(-c4cnn(C5CCNCC5)c4)cnc3N)oc2cc1O] |
| Compound 46 | 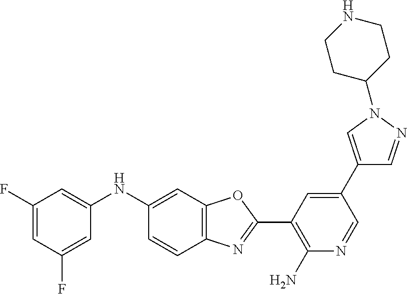 [SMILES: Nc1ncc(-c2cnn(C3CCNCC3)c2)cc1-c1nc2ccc(Nc3cc(F)cc(F)c3)cc2o1] |
| Compound 47 | 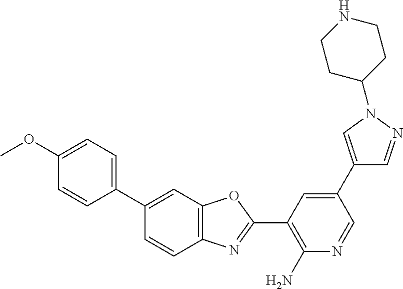 [SMILES: COc1ccc(-c2ccc3nc(-c4cc(-c5cnn(C6CCNCC6)c5)cnc4N)oc3c2)cc1] |
| Compound 48 | 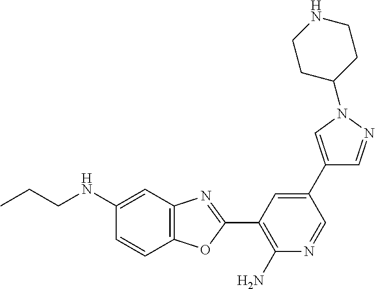 [SMILES: CCCNc1ccc2oc(-c3cc(-c4cnn(C5CCNCC5)c4)cnc3N)nc2c1] |
| Compound 49 | 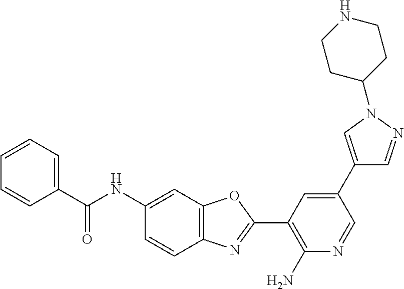 [SMILES: Nc1ncc(-c2cnn(C3CCNCC3)c2)cc1-c1nc2ccc(NC(=O)c3ccccc3)cc2o1] |
| Compound 50 | 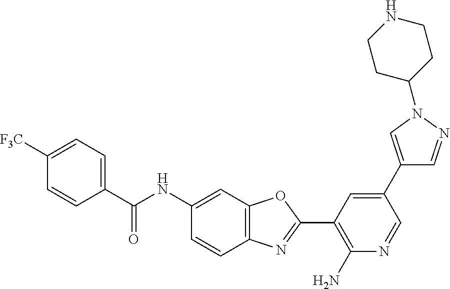 [SMILES: Nc1ncc(-c2cnn(C3CCNCC3)c2)cc1-c1nc2ccc(NC(=O)c3ccc(C(F)(F)F)cc3)cc2o1] |
| Compound 51 | 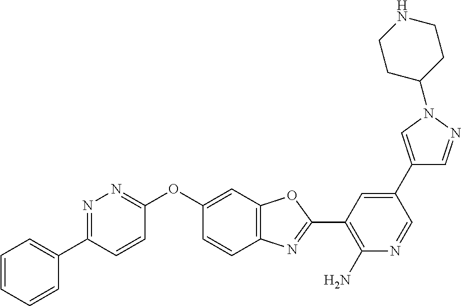 [SMILES: Nc1ncc(-c2cnn(C3CCNCC3)c2)cc1-c1nc2ccc(Oc3ccc(-c4ccccc4)nn3)cc2o1] |
| Compound 52 | 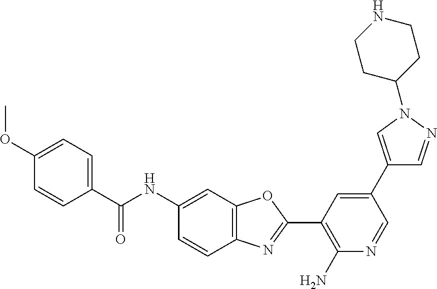 [SMILES: COc1ccc(C(=O)Nc2ccc3nc(-c4cc(-c5cnn(C6CCNCC6)c5)cnc4N)oc3c2)cc1] |
| Compound 53 | 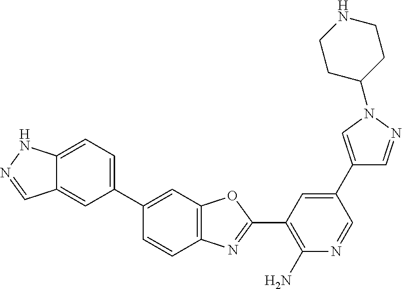 [SMILES: Nc1ncc(-c2cnn(C3CCNCC3)c2)cc1-c1nc2ccc(-c3ccc4[nH]ncc4c3)cc2o1] |
| Compound 54 | 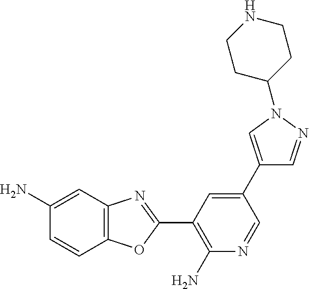 [SMILES: Nc1ccc2oc(-c3cc(-c4cnn(C5CCNCC5)c4)cnc3N)nc2c1] |
| Compound 55 | 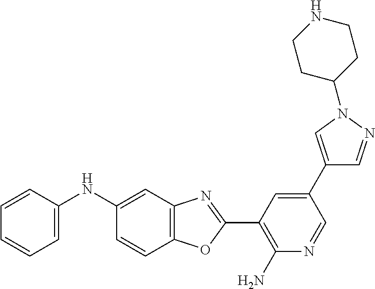 [SMILES: Nc1ncc(-c2cnn(C3CCNCC3)c2)cc1-c1nc2cc(Nc3ccccc3)ccc2o1] |
| Compound 56 | 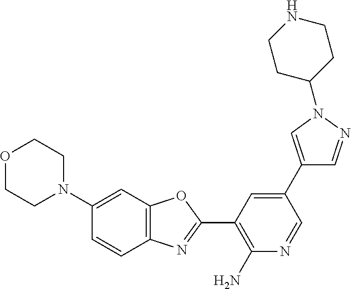 [SMILES: Nc1ncc(-c2cnn(C3CCNCC3)c2)cc1-c1nc2ccc(N3CCOCC3)cc2o1] |
| Compound 57 | 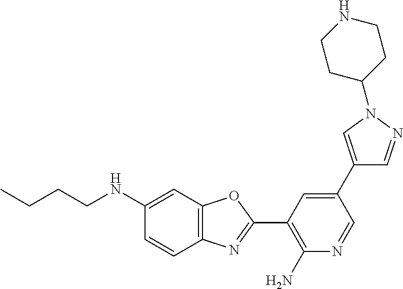 [SMILES: CCCCNc1ccc2nc(-c3cc(-c4cnn(C5CCNCC5)c4)cnc3N)oc2c1] |
| Compound 58 | 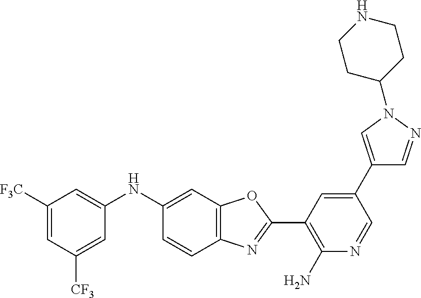 [SMILES: Nc1ncc(-c2cnn(C3CCNCC3)c2)cc1-c1nc2ccc(Nc3cc(C(F)(F)F)cc(C(F)(F)F)c3)cc2o1] |
| Compound 59 | 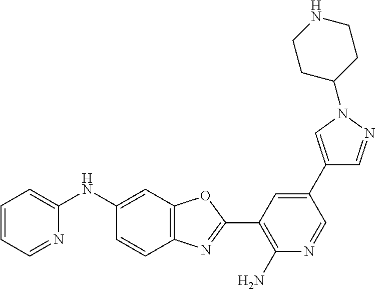 [SMILES: Nc1ncc(-c2cnn(C3CCNCC3)c2)cc1-c1nc2ccc(Nc3ccccn3)cc2o1] |
| Compound 60 | 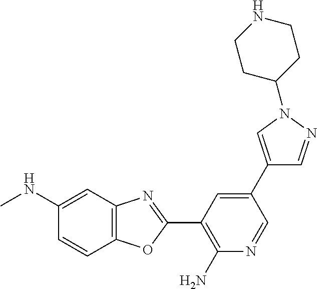 [SMILES: CNc1ccc2oc(-c3cc(-c4cnn(C5CCNCC5)c4)cnc3N)nc2c1] |
| Compound 61 | 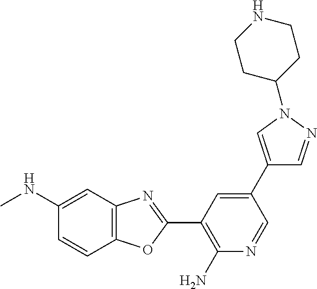 [SMILES: CNc1ccc2oc(-c3cc(-c4cnn(C5CCNCC5)c4)cnc3N)nc2c1] |
| Compound 62 | 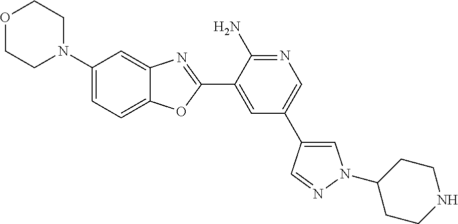 [SMILES: Nc1ncc(-c2cnn(C3CCNCC3)c2)cc1-c1nc2cc(N3CCOCC3)ccc2o1] |
| Compound 63 | 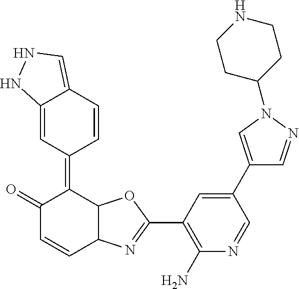 [SMILES: Nc1ncc(-c2cnn(C3CCNCC3)c2)cc1C1=NC2C=CC(=O)/C(=c3/ccc4c(c3)NNC=4)C2O1] |
| Compound 64 | 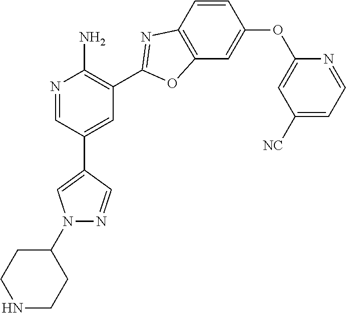 [SMILES: N#Cc1ccnc(Oc2ccc3nc(-c4cc(-c5cnn(C6CCNCC6)c5)cnc4N)oc3c2)c1] |
| Compound 65 | 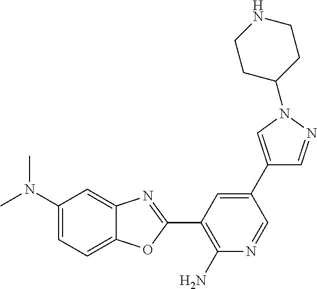 [SMILES: CN(C)c1ccc2oc(-c3cc(-c4cnn(C5CCNCC5)c4)cnc3N)nc2c1] |
| Compound 66 | 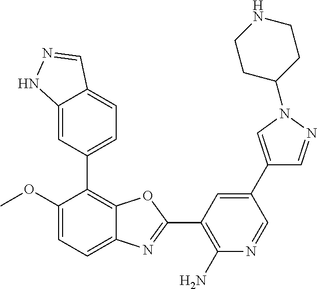 [SMILES: COc1ccc2nc(-c3cc(-c4cnn(C5CCNCC5)c4)cnc3N)oc2c1-c1ccc2cn[nH]c2c1] |
| Compound 67 | 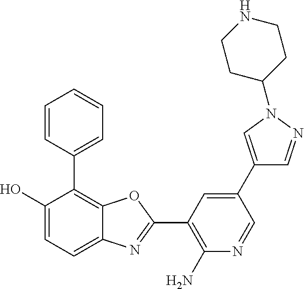 [SMILES: Nc1ncc(-c2cnn(C3CCNCC3)c2)cc1-c1nc2ccc(O)c(-c3ccccc3)c2o1] |
| Compound 68 | 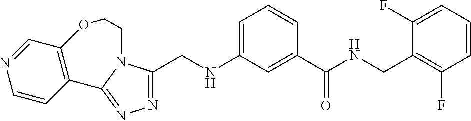 [SMILES: O=C(NCc1c(F)cccc1F)c1cccc(NCc2nnc3n2CCOc2cnccc2-3)c1] |
| Compound 69 | 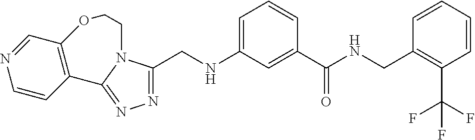 [SMILES: O=C(NCc1ccccc1C(F)(F)F)c1cccc(NCc2nnc3n2CCOc2cnccc2-3)c1] |
| Compound 70 | 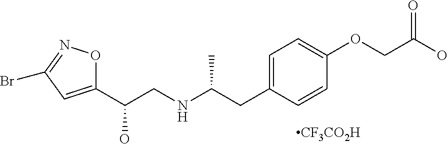 [SMILES: C[C@H](Cc1ccc(OCC(=O)O)cc1)NC[C@H](O)c1cc(Br)no1 ; "·CF3CO2H"] |

In some embodiments of any of the above aspects, the GRK2 inhibitor or GRK2-specific inhibitor is a GRK2 function blocker.

In some embodiments of any of the above aspects, the GRK2 inhibitor is a GRK2 signaling inhibitor.

In some embodiments of any of the above aspects, the GRK2 inhibitor or GRK2-specific inhibitor reduces GRK2 expression or activity.

In some embodiments of any of the above aspects, the GRK2 inhibitor or GRK2-specific inhibitor reduces GRK2 binding to a binding partner.

In some embodiments of any of the above aspects, the GRK2 small molecule antagonist reduces or inhibits GRK2 kinase activity.

In some embodiments of any of the above aspects, the cancer is pancreatic cancer, melanoma, small cell lung cancer, non-small cell lung cancer, gastric cancer, colorectal cancer, head and neck cancer, ovarian cancer, testicular cancer, thymoma, uterine cancer, kidney cancer, acute myeloid leukemia, diffuse large B-cell lymphoma, prostate cancer, breast cancer, or hepatocellular carcinoma.

In some embodiments of any of the above aspects, the cancer is pancreatic cancer.

In some embodiments of any of the above aspects, the cancer is small cell lung cancer. In some embodiments of any of the above aspects, the cancer is non-small cell lung cancer. In some embodiments of any of the above aspects, the cancer is gastric cancer. In some embodiments of any of the above aspects, the cancer is colorectal cancer. In some embodiments of any of the above aspects, the cancer is head and neck cancer. In some embodiments of any of the above aspects, the cancer is ovarian cancer. In some embodiments of any of the above aspects, the cancer is testicular cancer. In some embodiments of any of the above aspects, the cancer is thymoma. In some embodiments of any of the above aspects, the cancer is uterine cancer. In some embodiments of any of the above aspects, the cancer is kidney cancer. In some embodiments of any of the above aspects, the cancer is acute myeloid leukemia. In some embodiments of any of the above aspects, the cancer is diffuse large B-cell lymphoma. In some embodiments of any of the above aspects, the cancer is prostate cancer. In some embodiments of any of the above aspects, the cancer is hepatocellular carcinoma.

In some embodiments of any of the above aspects, the cancer is GRK2-associated cancer.

In some embodiments of any of the above aspects, the GRK2 inhibitor or GRK2-specific inhibitor (e.g., the GRK2 small molecule antagonist) is administered locally. In some embodiments, the GRK2 inhibitor or GRK2-specific inhibitor (e.g., the GRK2 small molecule antagonist) is administered intratumorally. In some embodiments, the GRK2 inhibitor or GRK2-specific inhibitor (e.g., the GRK2 small molecule antagonist) is administered to or near a site of metastasis. In some embodiments, the GRK2 inhibitor or GRK2-specific inhibitor (e.g., the GRK2 small molecule antagonist) is administered to or near a tumor microenvironment.

In some embodiments of any of the above aspects, the method further includes administering a second therapeutic agent.

In some embodiments of any of the above aspects, the GRK2 inhibitor or GRK2-specific inhibitor (e.g., the GRK2 small molecule antagonist) decreases tumor volume, decreases tumor growth, decreases tumor innervation (e.g., the number of nerves or the growth of nerves into a tumor), decreases nerve activity in a tumor (e.g., neurotransmission), decreases cancer cell proliferation, decreases cancer cell invasion, decreases cancer cell migration, decreases cancer cell metastasis, increases cancer cell autophagy, increases cancer cell death, decreases tumor GRK2 expression, treats the cancer or tumor, causes remission, increases time to recurrence, or improves survival. In some embodiments of any of the above aspects, the GRK2 inhibitor or GRK2-specific inhibitor (e.g., the GRK2 small molecule antagonist) decreases tumor growth. In some embodiments of any of the above aspects, the GRK2 inhibitor or GRK2-specific inhibitor (e.g., the GRK2 small molecule antagonist) decreases cancer cell proliferation.

In some embodiments of any of the above aspects, the method further includes measuring one or more of tumor volume, tumor growth, tumor innervation (e.g., the number of nerves or the growth of nerves into a tumor), nerve activity in a tumor (e.g., neurotransmission), cancer cell proliferation, cancer cell invasion, cancer cell migration, cancer cell metastasis, cancer cell autophagy, cancer cell death, or GRK2 expression before administration of the GRK2 inhibitor or GRK2-specific inhibitor (e.g., the GRK2 small molecule antagonist). In some embodiments of any of the above aspects, the method further includes measuring tumor growth before administration of the GRK2 inhibitor or GRK2-specific inhibitor (e.g., the GRK2 small molecule antagonist). In some embodiments of any of the above aspects, the method further includes measuring cancer cell proliferation before administration of the GRK2 inhibitor or GRK2-specific inhibitor (e.g., the GRK2 small molecule antagonist).

In some embodiments of any of the above aspects, the method further includes measuring one or more of tumor volume, tumor growth, tumor innervation (e.g., the number of nerves or the growth of nerves into a tumor), nerve activity in a tumor (e.g., neurotransmission), cancer cell proliferation, cancer cell invasion, cancer cell migration, cancer cell metastasis, cancer cell autophagy, cancer cell death, or GRK2 expression after administration of the GRK2 inhibitor or GRK2-specific inhibitor (e.g., the GRK2 small molecule antagonist). In some embodiments of any of the above aspects, the method further includes measuring tumor growth after administration of the GRK2 inhibitor or GRK2-specific inhibitor (e.g., the GRK2 small molecule antagonist). In some embodiments of any of the above aspects, the method further includes measuring cancer cell proliferation after administration of the GRK2 inhibitor or GRK2-specific inhibitor (e.g., the GRK2 small molecule antagonist).

In some embodiments of any of the above aspects, the GRK2 inhibitor or GRK2-specific inhibitor (e.g., the GRK2 small molecule antagonist) is administered in an amount sufficient to decrease tumor volume, decrease tumor growth, decrease tumor innervation (e.g., the number of nerves or the growth of nerves into a tumor), decrease nerve activity in a tumor (e.g., neurotransmission), decrease cancer cell proliferation, decrease cancer cell invasion, decrease cancer cell migration, decrease cancer cell metastasis, increase cancer cell autophagy, increase cancer cell death, decrease tumor GRK2 expression, treat the cancer or tumor, cause remission, increase time to recurrence, or improve survival. In some embodiments of any of the above aspects, the GRK2 inhibitor or GRK2-specific inhibitor (e.g., the GRK2 small molecule antagonist) is administered in an amount sufficient to decrease tumor growth. In some embodiments of any of the above aspects, the GRK2 inhibitor or GRK2-specific inhibitor (e.g., the GRK2 small molecule antagonist) is administered in an amount sufficient to decrease cancer cell proliferation.

In some embodiments of any of the above aspects, the GRK2 inhibitor is a GRK2 function blocker.

In some embodiments of any of the above aspects, the GRK2 inhibitor is a GRK2 signaling inhibitor.

In some embodiments of any of the above aspects, the GRK2 inhibitor is a GRK2 small molecule antagonist (e.g., an antagonist described herein, such as a compound having the structure of any one of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), or any one of Compounds 1-70).

In another aspect, the invention provides a pharmaceutical composition containing a GRK2 inhibitor.

In another aspect, the invention provides a pharmaceutical composition containing a nuclease directed to GRK2.

In another aspect, the invention provides a pharmaceutical composition containing an inhibitory RNA (e.g., shRNA, siRNA, or miRNA) directed to a GRK2 binding partner listed in Table 2.

In another aspect, the invention provides a pharmaceutical composition containing a nuclease directed to a GRK2 binding partner listed in Table 2.

In some embodiments of any of the above aspects, the GRK2 inhibitor is a GRK2-specific inhibitor. In some embodiments, the GRK2-specific inhibitor is an inhibitory RNA (e.g., shRNA, siRNA, or miRNA) directed to GRK2 or a nuclease directed to GRK2.

In some embodiments of any of the above aspects, the GRK2 inhibitor is a small molecule. In some embodiments, the small molecule is a GRK2 small molecule antagonist (e.g., an antagonist described herein, such as a compound having the structure of any one of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), or any one of Compounds 1-70), or a GRK2 signaling inhibitor listed in Table 3.

In some embodiments of any of the above aspects, the GRK2 inhibitor is an inhibitory RNA (e.g., shRNA, siRNA, or miRNA). In some embodiments of any of the above aspects, the GRK2 inhibitor is an inhibitory RNA directed to GRK2. In some embodiments of any of the above aspects, the GRK2 inhibitor is an inhibitory RNA directed to a GRK2 binding partner listed in Table 2.

In some embodiments of any of the above aspects, the GRK2 inhibitor is a nuclease. In some embodiments of any of the above aspects, the GRK2 inhibitor is a nuclease directed to GRK2. In some embodiments of any of the above aspects, the GRK2 inhibitor is a nuclease directed to a GRK2 binding partner listed in Table 2.

In some embodiments of any of the above aspects, the nuclease is a transcription activator-like effector nuclease (TALEN), zinc-finger nuclease (ZFN), guide RNA (gRNA), or a CRISPR associated protein (Cas) (e.g., Cas9).

In some embodiments of the above aspects, the composition further includes a second therapeutic agent.

In some embodiments of any of the above aspects, the composition further includes a pharmaceutically acceptable excipient.

In some embodiments of any of the above aspects, the second therapeutic agent is an anti-cancer agent, a GRK2 signaling inhibitor, a GRK2 function blocker, a neurotransmission blocker, or a neuronal growth factor blocker. In some embodiments of any of the above aspects, the second therapeutic agent is an anti-cancer agent.

In some embodiments of any of the above aspects, the anti-cancer agent is a checkpoint inhibitor, a chemotherapeutic agent, a biologic cancer agent (e.g., an antibody listed in Table 4), a cancer-specific agent (e.g., an agent listed in Table 5), a cytokine therapy (e.g., an interferon or interleukin, such as IL-2 or IL-12), CAR-T therapy, an oncolytic virus (e.g., tamilogene laherparepvec (T-VEC), canerpaturev, enadenotucirev, pelareorep, pexastimogene devacirepvec (JX-594), or tasadenoturev), an anti-angiogenic drug, a drug that targets cancer metabolism, an antibody that marks a cancer cell surface for destruction, an antibody-drug conjugate, a cell therapy, a commonly used anti-neoplastic agent, or a non-drug therapy. In some embodiments of any of the above aspects, the anti-cancer agent is a checkpoint inhibitor. In some embodiments of any of the above aspects, the anti-cancer agent is a chemotherapeutic agent.

In some embodiments of any of the above aspects, the chemotherapeutic agent is paclitaxel, everolimus, erlotinib hydrochloride, fluorouracil, gemcitabine hydrochloride, irinotecan hydrochloride, mitomycin C, sunitinib malate, lanreotide acetate, lutetium Lu 177-dotatate, FOLFIRINOX, GEMCITABINE-CISPLATIN, GEMCITABINE-OXALIPLATIN, or OFF.

In some embodiments of any of the above aspects, the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

In some embodiments of any of the above aspects, the biologic cancer agent is an antibody listed in Table 4.

In some embodiments of any of the above aspects, the cancer is a cancer listed in column 1 of Table 5 and the second agent is a corresponding anti-cancer agent listed in column 2 of Table 5.

In some embodiments of any of the above aspects, the neurotransmission blocker is neurotoxin listed in Table 10, an antagonist of a neurotransmitter receptor listed in Table 6 or a neurotransmitter listed in Table 7, or a GABA re-uptake inhibitor, GABA analog, or GABA prodrug listed in Table 9. In some embodiments, the antagonist of a neurotransmitter receptor listed in Table 6 or a neurotransmitter listed in Table 7 is an antagonist listed in Tables 8A-8K.

In some embodiments of any of the above aspects, the neuronal growth factor blocker is an antagonist of a neuronal growth factor listed in Table 11. In some embodiments, the antagonist of a neuronal growth factor listed in Table 11 is an antibody listed in Table 12 or an antagonist listed in Table 13. In some embodiments, the antagonist of a neuronal growth factor listed in Table 11 is selected from the group consisting of etanercept, thalidomide, lenalidomide, pomalidomide, pentoxifylline, bupropion, DOI, disitertide, and trabedersen.

In some embodiments of any of the above aspects, the GRK2-specific inhibitor is an inhibitory RNA (e.g., shRNA, siRNA, or miRNA) directed to GRK2. In some embodiments of any of the above aspects, the GRK2-specific inhibitor is a nuclease (e.g., Cas, TALEN, gRNA, or ZFN) directed to GRK2.

In some embodiments of any of the above aspects, the GRK2 function blocker is a GRK2-specific inhibitor. In some embodiments of any of the above aspects, the GRK2 function blocker is a GRK2 small molecule antagonist. In some embodiments of any of the above aspects, the GRK2 function blocker is an inhibitory RNA (e.g., shRNA, siRNA, or miRNA) directed against GRK2. In some embodiments of any of the above aspects, the GRK2 function blocker is an inhibitory RNA (e.g., shRNA, siRNA, or miRNA) directed against a GRK2 binding partner listed in Table 2. In some embodiments of any of the above aspects, the GRK2 function blocker is a nuclease (e.g., Cas, TALEN, gRNA, or ZFN) directed against GRK2. In some embodiments of any of the above aspects, the GRK2 function blocker is a nuclease (e.g., Cas, TALEN, gRNA, or ZFN) directed against a GRK2 binding partner listed in Table 2.

In some embodiments of any of the above aspects, the GRK2 signaling inhibitor is a small molecule inhibitor listed in Table 3.

In some embodiments of any of the above aspects, the GRK2 inhibitor or GRK2-specific inhibitor is selected from the group consisting of a small molecule, an RNA molecule, a gRNA, a nuclease, and a viral vector. In some embodiments, the small molecule is a GRK2 small molecule antagonist (e.g., an antagonist described herein, such as a compound having the structure of any one of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), or any one of Compounds 1-70). In some embodiments, the RNA molecule is an inhibitory RNA (e.g., shRNA, siRNA, or miRNA). In some embodiments, the inhibitory RNA is directed to GRK2. In some embodiments, the inhibitory RNA is directed to a GRK2 binding partner listed in Table 2. In some embodiments, the nuclease is a Cas, TALEN, or ZFN. In some embodiments, the nuclease is directed to GRK2. In some embodiments, the nuclease is directed to a GRK2 binding partner listed in Table 2.

In some embodiments of any of the above aspects, the nuclease is directed to GRK2 by a guide RNA (gRNA) molecule. In some embodiments, the gRNA molecule is encoded by a DNA molecule having a nucleic acid sequence with at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of any one of SEQ ID NOs: 1-6. In some embodiments, the gRNA molecule is encoded by a DNA molecule having the nucleic acid sequence of any one of SEQ ID NOs: 1-6.

In some embodiments of any of the above aspects, the GRK2 inhibitor or GRK2-specific inhibitor does not cross the blood brain barrier (BBB). In some embodiments, the GRK2 inhibitor or GRK2-specific inhibitor has been modified to prevent BBB crossing by conjugation to a targeting moiety, formulation in a particulate delivery system, addition of a molecular adduct, or through modulation of its size, polarity, flexibility, or lipophilicity.

In some embodiments of any of the above aspects, the GRK2 inhibitor or GRK2-specific inhibitor does not have a direct effect on the central nervous system or gut.

In some embodiments of any of the above aspects, the GRK2 inhibitor or GRK2-specific inhibitor decreases tumor volume, decreases tumor growth, decreases tumor innervation (e.g., the number of nerves or the growth of nerves into a tumor), decreases nerve activity in a tumor (e.g., neurotransmission), decreases cancer cell proliferation, decreases cancer cell invasion, decreases cancer cell migration, decreases cancer cell metastasis, increases cancer cell autophagy, increases cancer cell death, or decreases tumor GRK2 expression, increases time to recurrence, or improves survival.

In some embodiments of any of the above aspects, the subject is not diagnosed as having high blood pressure or a cardiac condition (e.g., heart failure or cardiac hypertrophy).

In some embodiments of any of the above aspects, the subject is human.

### Definitions

As used herein, “administration” refers to providing or giving a subject a therapeutic agent (e.g., a GRK2 inhibitor), by any effective route. Exemplary routes of administration are described herein below.

As used herein, the term “agonist” refers to an agent (e.g., a small molecule or antibody) that increases receptor activity. An agonist may activate a receptor by directly binding to the receptor, by acting as a cofactor, by modulating receptor conformation (e.g., maintaining a receptor in an open or active state). An agonist may increase receptor activity by 10%, 20%, 30%, 40%, 50%, 60%, 70%, 80%, 90%, 95%, 98% or more. An agonist may induce maximal receptor activation or partial activation depending on the concentration of the agonist and its mechanism of action.

As used herein, the term “analog” refers to a protein of similar nucleotide or amino acid composition or sequence to any of the proteins or peptides of the invention, allowing for variations that do not have an adverse effect on the ability of the protein or peptide to carry out its normal function (e.g., bind to a receptor or promote synapse formation). Analogs may be the same length, shorter, or longer than their corresponding protein or polypeptide. Analogs may have about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of the naturally occurring protein or peptide. An analog can be a naturally occurring protein or polypeptide sequence that is modified by deletion, addition, mutation, or substitution of one or more amino acid residues.

As used herein, the term “binding partner” refers to a polypeptide or fragment thereof that binds to a protein of interest (e.g., GRK2). Binding partners include receptors and other molecules that selectively bind to the ligand of interest. Exemplary GRK2 binding partners (listed in Table 2) are ADRB1 (Entrez Gene ID: 153), ADRB2 (Entrez Gene ID: 154), RALA (Entrez Gene ID: 5898), LPAR1 (Entrez Gene ID: 1902), LPAR2 (Entrez Gene ID: 9170), CCRS (Entrez Gene ID: 1234), ADRA2A (Entrez Gene ID: 150), ARRB1 (Entrez Gene ID: 408), and GIT1 (Entrez Gene ID: 28964).

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and a “sufficient amount” of a composition, antibody, vector construct, viral vector or cell described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. For example, in the context of treating cancer it is an amount of the composition, nucleotide, small molecule, vector construct, viral vector or cell sufficient to achieve a treatment response as compared to the response obtained without administration of the composition, nucleotide, small molecule, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, nucleotide, small molecule, vector construct, viral vector or cell of the present disclosure is an amount that results in a beneficial or desired result in a subject as compared to a control. As defined herein, a therapeutically effective amount of a composition, nucleotide, small molecule, vector construct, viral vector or cell of the present disclosure may be readily determined by one of ordinary skill by routine methods known in the art. Dosage regimen may be adjusted to provide the optimum therapeutic response.

As used herein, the terms “increasing” and “decreasing” refer to modulating resulting in, respectively, greater or lesser amounts, of function, expression, or activity of a metric relative to a reference. For example, subsequent to administration of a GRK2 inhibitor in a method described herein, the amount of a marker of a metric (e.g., cancer cell death) as described herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “innervated” refers to a tissue (e.g., a tumor) that contains nerves. “Innervation” refers to the process of nerves entering a tissue.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “proliferation” refers to an increase in cell numbers through growth and division of cells.

As used herein, the term “reference” refers to a level, expression level, sample or standard that is used for comparison purposes. For example, a reference sample can be obtained from a healthy individual (e.g., an individual who does not have cancer). A reference level can be the level of expression of one or more reference samples. For example, an average expression (e.g., a mean expression or median expression) among a plurality of individuals (e.g., healthy individuals, or individuals who do not have cancer). In other instances, a reference level can be a predetermined threshold level, e.g., based on functional expression as otherwise determined, e.g., by empirical assays.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “overexpressed” refers to a nucleic acid or polypeptide that is expressed or caused to be expressed or produced in a cell at a greater level than is normally expressed in the corresponding wild-type cell. For example, GRK2 is “overexpressed” in a cancer cell when GRK2 is present at a higher level in the cancer cell compared to the level in a non-cancerous cell of the same tissue or cell type from the same species or individual. GRK2 is overexpressed when GRK2 expression is increased by 1.1-fold or more (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more) compared to a reference (e.g., a non-cancerous cell of the same type).

As used herein, the term “cardiac condition” refers to a medical condition directly affecting the heart or circulatory system. Cardiac conditions include abdominal aortic aneurysm, arrhythmia (e.g., supraventricular tachycardia, inappropriate sinus tachycardia, atrial flutter, atrial fibrillation, ventricular tachycardia, and ventricular fibrillation), angina, atherosclerosis, brugada syndrome, cardiac arrest, cardiac hypertrophy, cardiomyopathy, cardiovascular disease, congenital heart disease, coronary heart disease, catecholaminergic polymorphic ventricular tachycardia (CVPT), familial hypercholesterolaemia, heart attack, heart failure, heart block, heart valve disease (e.g., heart murmur, valve stenosis, mitral valve prolapse, and heart valve regurgitation), inherited heart conditions, long QT syndrome, progressive cardiac conduction deficit (PCCD), pericarditis, venous thromboembolism, peripheral artery disease, and stroke.

As used herein, the terms “high blood pressure” and “hypertension” refer to a chronic medical condition in which the systemic arterial blood pressure is elevated. It is classified as blood pressure above 140/90 mmHg.

As used herein, the term “cancer” refers to a condition characterized by unregulated or abnormal cell growth. The terms “cancer cell,” “tumor cell,” and “tumor” refer to an abnormal cell, mass, or population of cells that result from excessive division that may be malignant or benign and all pre-cancerous and cancerous cells and tissues.

As used herein, the term “GRK2-associated cancer” refers to a cancer in which GRK2 is expressed (e.g., expressed or overexpressed compared to a reference (e.g., a non-cancerous cell of the same type)). GRK2-associated cancers can be identified by assessing a cancer cell or tumor sample for GRK2 expression and comparing it to GRK2 expression in a reference cell.

As used herein, the term “GRK2 inhibitor” refers to an agent that inhibits or reduces GRK2 function, expression, or signaling. GRK2 inhibitors include small molecule antagonists of GRK2 (e.g., GRK2 antagonists having the structure of Formula I, Formula II, or Formula III, Formula IV, or the structure of any one of compounds 1-70 described herein), inhibitory RNAs directed to GRK2 or a GRK2 binding partner listed in Table 2, and nucleases directed to GRK2 or a GRK2 binding partner listed in Table 2 that reduce or inhibit GRK2 expression, GRK2 binding to a binding partner, GRK2 function (e.g., GRK2 kinase activity), or signal transduction downstream of GRK2. GRK2 inhibitors reduce GRK2 function, expression, or signaling by 10% or more (e.g., 10%, 20%, 30%, 40%, 50%, 60%, 70%, 80%, 90%, 95%, 98% or more).

As used herein, the term “GRK2-specific inhibitor” refers to a GRK2 inhibitor that selectively targets, binds to, or knocks down GRK2 without substantially binding to, targeting, or knocking down another protein. GRK2-specific inhibitors include inhibitory RNAs and nucleases directed to GRK2. GRK2-specific inhibitors can reduce GRK2 function or expression by 10% or more (e.g., 10%, 20%, 30%, 40%, 50%, 60%, 70%, 80%, 90%, 95%, 98% or more).

As used herein, the term “GRK2 function blocker” refers to a type of GRK2 inhibitor that reduces or inhibits GRK2 function. GRK2 function blockers include agents that act directly on GRK2, such as GRK2-specific inhibitors, and agents that prevent or reduce the interaction between GRK2 and one or more of its binding partners (e.g., ADRB1 or other binding partners listed in Table 2). Exemplary GRK2 function blockers include small molecule antagonists of GRK2 (e.g., a small molecule antagonist having a structure of Formula I (e.g., having the structure of any one of Formula I-1 to I-39), Formula II (e.g., having the structure of any one of Formula II-1 to II-35), Formula III (e.g., having the structure of any one of Formula III-1 to III-56), Formula IV (e.g., having the structure of any one of Formula IV-1 to IV-80), or any one of Compounds 1-70), inhibitory RNAs directed to GRK2 or a GRK2 binding partner listed in Table 2, and nucleases directed to GRK2 or a GRK2 binding partner listed in Table 2. GRK2 function blockers can reduce GRK2 function (e.g., kinase activity) by 10% or more (e.g., 10%, 20%, 30%, 40%, 50%, 60%, 70%, 80%, 90%, 95%, 98% or more).

As used herein, the term “GRK2 signaling inhibitor” refers to a type of GRK2 inhibitor that reduces or inhibits the intracellular signaling that is downstream of GRK2 activation or interaction with a binding partner. GRK2 signaling inhibitors include small molecules and inhibitory RNAs directed to molecules in the smoothened (SMO)-dependent Hedgehog (Hh) signaling pathway. GRK2 signaling inhibitors reduce downstream signaling by 10% or more (e.g., 10%, 20%, 30%, 40%, 50%, 60%, 70%, 80%, 90%, 95%, 98% or more).

As used herein, the term “GRK2 small molecule antagonist” refers to a small molecule that reduces or inhibits the kinase activity of GRK2 and has an 1050 of 10 μM or lower. GRK2 small molecule antagonists for use in the methods and compositions described herein include compounds having a structure of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), and Compounds 1-70.

As used herein, an agent that “does not cross the blood brain barrier” is an agent that does not significantly cross the barrier between the peripheral circulation and the brain and spinal cord. This can also be referred to as a “blood brain barrier impermeable” agent. Agents will have a limited ability to cross the blood brain barrier (BBB) if they are not lipid soluble or have a molecular weight of over 600

Daltons. Agents that typically cross the BBB can be modified to become BBB impermeable based on chemical modifications that increase the size or alter the hydrophobicity of the agent, packaging modifications that reduce diffusion (e.g., packaging an agent within a microparticle or nanoparticle), and conjugation to biologics that direct the agent away from the BBB (e.g., conjugation to a pancreas-specific antibody). An agent that does not cross the blood brain barrier is an agent for which 30% or less (e.g., 30%, 25%, 20%, 15%, 10%, 5%, 2% or less) of the administered agent crosses the BBB.

As used herein, an agent that “does not have a direct effect on the central nervous system (CNS) or gut” is an agent that does not directly alter neurotransmission, neuronal numbers, or neuronal morphology in the CNS or gut when administered according to the methods described herein. This may be assessed by administering the agents to animal models and performing electrophysiological recordings or immunohistochemical analysis. An agent will be considered not to have a direct effect on the CNS or gut if administration according to the methods described herein has an effect on neurotransmission, neuronal numbers, or neuronal morphology in the CNS or gut that is 50% or less (e.g., 50%, 45%, 40%, 35%, 30%, 25%, 20%, 15%, 10%, 5%, or less) of the effect observed if the same agent is administered directly to the CNS or gut.

As used herein, the term “neuronal growth factor blocker” refers to an agent that decreases or inhibits neuronal growth, development, or survival. Neuronal growth factors include proteins that promote neurogenesis, neuronal growth, and neuronal differentiation (e.g., neurotrophic factors NGF, NT3, BDNF, CNTF, and GDNF), proteins that promote neurite outgrowth (e.g., axon or dendrite outgrowth or stabilization), or proteins that promote synapse formation (e.g., synaptogenesis, synapse assembly, synaptic adhesion, synaptic maturation, synaptic refinement, or synaptic stabilization). These processes lead to innervation of tissue, including neural tissue, muscle, and tumors, and the formation of synaptic connections between two or more neurons and between neurons and non-neural cells (e.g., tumor cells). A neuronal growth factor blocker reduces or inhibits one or more of these processes (e.g., through the use of antibodies that block neuronal growth factors or their receptors). Exemplary neuronal growth factors are listed in Table 11. Neuronal growth factor blockers decrease or inhibit neurite outgrowth, innervation, synapse formation, or any of the aforementioned processes by 10% or more (e.g., 10%, 20%, 30%, 40%, 50%, 60%, 70%, 80%, 90%, 95%, 98% or more).

As used herein, the term “neurotransmission blocker” refers to an agent that decreases or blocks neurotransmission. Neurotransmission blockers can decrease neurotransmission by 10%, 20%, 30%, 40%, 50%, 60%, 70%, 80%, 90%, 95%, 98% or more. Exemplary neurotransmitters and neurotransmitter receptors are listed in Tables 6 and 7. Neurotransmission blockers may decrease neurotransmission by decreasing neurotransmitter synthesis or release, increasing neurotransmitter reuptake or degradation, decreasing neurotransmitter receptor activity, decreasing neurotransmitter receptor synthesis or membrane insertion, increasing neurotransmitter degradation, regulating neurotransmitter receptor conformation, and disrupting the pre- or postsynaptic machinery. Neurotransmission blockers include antibodies that bind to or block the function of neurotransmitters, neurotransmitter receptor antagonists, inhibitory RNAs directed to neurotransmitter receptors, and toxins that disrupt synaptic release.

As used herein, the term “alkoxy” or “alkoxyl” as used herein refers to a “—O-alkyl” group. The alkoxy or alkyoxl group can be unsubstituted or substituted.

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, Ca alkyl refers to an alkyl group that has 4 carbon atoms. C₁-C₇ alkyl refers to an alkyl group having a number of carbon atoms encompassing the entire range (i.e., 1 to 7 carbon atoms), as well as all subgroups (e.g., 1-6, 2-7, 1-5, 3-6, 1, 2, 3, 4, 5, 6, and 7 carbon atoms). Nonlimiting examples of alkyl groups include, methyl, ethyl, n-propyl, isopropyl, n-butyl, sec-butyl (2-methylpropyl), t-butyl (1,1-dimethylethyl), 3,3-dimethylpentyl, and 2-ethylhexyl. Unless otherwise indicated, an alkyl group can be an unsubstituted alkyl group or a substituted alkyl group. When the alkyl group is substituted, the substituent can occur on any carbon of the alkyl chain. Alkyl groups may—if not otherwise stated—be halogenated once or more, i.e. alkyl groups may be fluorinated, i.e. perfluorinated. Examples of halogenated alkyl groups are CF₃ and CH₂CF₃, OCF₃, S—CF₃, —O—(CF₂)₂—O—.

As used herein, the term “alkenyl” is defined identically as “alkyl” except for containing at least one carbon-carbon double bond, and having two to thirty carbon atoms, for example, two to twenty carbon atoms, or two to ten carbon atoms. The term Cₙ means the alkenyl group has “n” carbon atoms. For example, C₄ alkenyl refers to an alkenyl group that has 4 carbon atoms. C₂-C₇ alkenyl refers to an alkenyl group having a number of carbon atoms encompassing the entire range (i.e., 2 to 7 carbon atoms), as well as all subgroups (e.g., 2-6, 2-5, 3-6, 2, 3, 4, 5, 6, and 7 carbon atoms). Specifically contemplated alkenyl groups include ethenyl, 1-propenyl, 2-propenyl, and butenyl. Unless otherwise indicated, an alkenyl group can be an unsubstituted alkenyl group or a substituted alkenyl group.

As used herein, the term “alkylene” refers to an alkyl group having a substituent. For example, the term “alkylene-aryl” refers to an alkyl group substituted with an aryl group. The term Cₙ means the alkylene group has “n” carbon atoms. For example, C₁₋₆ alkylene refers to an alkylene group having a number of carbon atoms encompassing the entire range, as well as all subgroups, as previously described for “alkyl” groups.

As used herein, the term “cycloalkyl” refers to an aliphatic cyclic hydrocarbon group containing three to eight carbon atoms (e.g., 3, 4, 5, 6, 7, 8, 9, or 10 carbon atoms). The term Cₙ means the cycloalkyl group has “n” carbon atoms. For example, C₅cycloalkyl refers to a cycloalkyl group that has 5 carbon atoms in the ring. C₅-C₈cycloalkyl refers to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group. The cycloalkyl groups described herein can be isolated, bridged, or fused to another cycloalkyl group, a heterocycloalkyl group, an aryl group and/or a heteroaryl group.

As used herein, the term “heterocycloalkyl” is defined similarly as cycloalkyl, except the ring contains one to three heteroatoms independently selected from oxygen, nitrogen, or sulfur. Nonlimiting examples of heterocycloalkyl groups include piperdine, tetrahydrofuran, tetrahydropyran, dihydrofuran, morpholine, thiophene, and the like. Cycloalkyl and heterocycloalkyl groups can be saturated or partially unsaturated ring systems optionally substituted with, for example, one to three groups, independently selected alkyl, alkyleneOH, C(O)NH₂, NH₂, oxo (═O), aryl, haloalkyl, halo, and OH. Heterocycloalkyl groups optionally can be further N-substituted with alkyl, hydroxyalkyl, alkylene-aryl, and alkylene-heteroaryl. The heterocycloalkyl groups described herein can be isolated, bridged, or fused to another heterocycloalkyl group, a cycloalkyl group, an aryl group and/or a heteroaryl group.

As used herein, the term “cycloalkenyl” is defined similarly to “cycloalkyl” except for containing at least one carbon-carbon double bond. The term Cₙ means the cycloalkenyl group has “n” carbon atoms. For example, C₅ cycloalkenyl refers to a cycloalkenyl group that has 5 carbon atoms in the ring. C₅-C₈ cycloalkenyl refers to cycloalkenyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkenyl groups include cyclopropenyl, cyclobutenyl, cyclopentenyl, cyclohexenyl, cycloheptenyl, and cyclooctenyl. Unless otherwise indicated, a cycloalkenyl group can be an unsubstituted cycloalkenyl group or a substituted cycloalkenyl group.

As used herein, the term “aryl” refers to monocyclic or polycyclic (e.g., fused bicyclic and fused tricyclic) carbocyclic aromatic ring systems. Examples of aryl groups include, but are not limited to, phenyl, naphthyl, tetrahydronaphthyl, phenanthrenyl, biphenylenyl, indanyl, indenyl, anthracenyl, and fluorenyl. Unless otherwise indicated, an aryl group can be an unsubstituted aryl group or a substituted aryl group.

As used herein, the term “heteroaryl” refers to monocyclic or polycyclic (e.g., fused bicyclic and fused tricyclic) aromatic ring systems, wherein one to four-ring atoms are selected from oxygen, nitrogen, or sulfur, and the remaining ring atoms are carbon, said ring system being joined to the remainder of the molecule by any of the ring atoms. Nonlimiting examples of heteroaryl groups include, but are not limited to, pyridyl, pyrazinyl, pyrimidinyl, pyrrolyl, pyrazolyl, imidazolyl, thiazolyl, tetrazolyl, oxazolyl, isooxazolyl, thiadiazolyl, oxadiazolyl, furanyl, quinolinyl, isoquinolinyl, benzoxazolyl, benzimidazolyl, and benzothiazolyl. Unless otherwise indicated, a heteroaryl group can be an unsubstituted heteroaryl group or a substituted heteroaryl group.

As used herein, the term (C₄-C₁₀) heterocyclyl group means a 4-10 membered mono- or bicyclic ring system which comprises, apart from carbon, one or more heteroatoms such as, for example, e.g. 1, 2, 3 or 4 nitrogen atoms, 1 or 2 oxygen atoms, 1 or 2 sulfur atoms or combinations of different hetero atoms. For example, a C₆-heterocyclyl may contain 5 carbon atoms and 1 nitrogen atom as is the case in pyridyl or piperidinyl. The heterocyclyl residues can be bound at any positions, for example on the 1-position, 2-position, 3-position, 4-position, 5-position, 6-position, 7-position or 8-position. Heterocyclyl comprises (1) aromatic (C₅-C₁₀)heterocyclyl groups [(C₅-C₁₀)heteroaryl groups] or (2) saturated (C₄-C₁₀)heterocyclyl groups or (3) mixed aromatic/saturated fused (C₈-C₁₀) heterocyclyl groups. (C₅-C₁₀) Heteroaryl groups are preferred as (C₄-C₁₀) heterocyclyl group. Suitable (C₄-C₁₀) heterocyclyl group include acridinyl, azetidine, benzimidazolyl, benzofuryl, benzomorpholinyl, benzothienyl, benzothiophenyl, benzoxazolyl, benzthiazolyl, benztriazolyl, benztetrazolyl, benzisoxazolyl, benzisothiazolyl, carbazolyl, 4aH-carbazolyl, carbolinyl, furanyl, quinazolinyl, quinolinyl, 4H-quinolizinyl, quinoxalinyl, quinuclidinyl, chromanyl, chromenyl, cinnolinyl, decahydroquinolinyl, 2H,6H-1,5,2-dithiazinyl, dihydrofuro[2,3-b]-tetrahydrofuran, furyl, furazanyl, homomorpholinyl, homopiperazinyl, imidazolidinyl, imidazolinyl, imidazolyl, 1H-indazolyl, indolinyl, indolizinyl, indolyl, 3H-indolyl, isobenzofuranyl, isochromanyl, isoindazolyl, isoindolinyl, isoindolyl, isoquinolinyl(benzimidazolyl), isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, prolinyl, pteridinyl, purynyl, pyranyl, pyrazinyl, pyroazolidinyl, pyrazolinyl, pyrazolyl, pyridazinyl, pyridonyl, pyridooxazoles, pyridoimidazoles, pyridothiazoles, pyridinyl, pyridyl, pyrimidinyl, pyrrolidinyl, pyrrolinyl, 2H-pyrrolyl, pyrrolyl, tetrahydrofuranyl, tetrahydroisoquinolinyl, tetrahydroquinolinyl, 6H-1,2,5-thiadazinyl, thiazolyl, 1,2,3-thiadiazolyl, 1,2,4-thiadiazolyl, 1,2,5-thiadiazolyl, 1,3,4-thiadiazolyl, thienyl, triazolyl, tetrazolyl and xanthenyl. Pyridyl stands both for 2-, 3- and 4-pyridyl. Thienyl stands both for 2- and 3-thienyl. Furyl stands both for 2- and 3-furyl. Also included are the corresponding N-oxides of these compounds, for example, 1-oxy-2-, 3- or 4-pyridyl. Substitutions in (C₄-C₁₀) heterocyclyl residues can occur on free carbon atoms or on nitrogen atoms. Preferred examples of (C₄-C₁₀) heterocyclyl residues are 2- or 3-thienyl, 2 or 3-furyl, 1-, 2- or 3-pyrrolyl, 1-, 2-, 4- or 5-imidazolyl, 1-, 3-, 4- or 5-pyrazolyl, 1,2,3-triazol-1-, -4 or -5-yl, 1,2,4-triazol-1-, -3 or -5-yl, 1- or 5-tetrazolyl, 2-, 4- or 5-oxazolyl, 3-, 4- or 5-isoxazolyl, 1,2,3-oxadiazol-4 or -5-yl, 1,2,4-oxadiazol-3 or -5-yl, 1,3,4-oxadiazol-2-yl or -5-yl, 2-, 4- or 5-thiazolyl, 3-, 4- or 5-isothiazolyl, 1,3,4-thiadiazol-2 or -5-yl, 1,2,4-thiadiazol-3 or -5-yl, 1,2,3-thiadiazol-4 or -5-yl, 2-, 3- or 4-pyridyl, 2-, 4-, 5- or 6-pyrimidinyl, 3- or 4-pyridazinyl, pyrazinyl, 1-, 2-, 3-, 4-, 5-, 6- or 7-indolyl, 1-, 2-, 4- or 5-benzimidazolyl, 1-, 3-, 4-, 5-, 6- or 7-indazolyl, 2-, 3-, 4-, 5-, 6-, 7- or 8-chinolyl, 1-, 3-, 4-, 5-, 6-, 7- or 8-isochinolyl, 2-, 4-, 5-, 6-, 7- or 8-chinazolinyl, 3-, 4-, 5-, 6-, 7- or 8-cinnolinyl, 2-, 3-, 5-, 6-, 7- or 8-chinoxalinyl, 1-, 4-, 5-, 6-, 7- or 8-phthalazinyl. Enclosed are also the respective n-oxides, for example 1-oxy-2-, -3 or -4-pyridyl. Particularly preferred (C₄-C₁₀) heterocyclyl residues are 2- or 3-furyl, 2- or 3-pyrrolyl, 3-, 4- or 5-pyrazolyl, and 2-, 3- or 4-pyridyl.

As used herein, the term “halogen” means fluoro, chloro, bromo or iodo.

As used herein, pyrazolopyridine substitution patterns are numbered according to IUPAC rules:

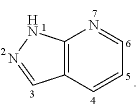

A used herein, the term “substituted,” when used to modify a chemical functional group, refers to the replacement of at least one hydrogen radical on the functional group with a substituent. Substituents can include, but are not limited to, alkyl, cycloalkyl, alkenyl, cycloalkenyl, alkynyl, heterocycloalkyl, ether, polyether, thioether, polythioether, aryl, heteroaryl, hydroxyl, oxy, alkoxy, heteroalkoxy, aryloxy, heteroaryloxy, ester, thioester, carboxy, cyano, nitro, amino, amido, acetamide, and halo (e.g., fluoro, chloro, bromo, or iodo). When a chemical functional group includes more than one substituent, the substituents can be bound to the same carbon atom or to two or more different carbon atoms. A substituted chemical functional group can itself include one or more substituents.

### DETAILED DESCRIPTION

Described herein are compositions and methods for the treatment of cancer in a subject (e.g., a mammalian subject, such as a human) by administering GRK2 inhibitors. GRK2 inhibitors include inhibitors specific to GRK2 (e.g., GRK2-specific inhibitory RNAs), inhibitors that reduce GRK2 function (e.g., GRK2 small molecule antagonists), and inhibitors that target GKR2 binding partners or downstream signaling pathways. These methods and compositions provide new mechanistic approaches for treating cancer.

### GRK2

G-protein-coupled receptor kinase 2 (GRK2, Entrez Gene ID 156, also known as adrenergic beta kinase 1, ADRBK1, and BARK1) is a member of the G protein-coupled receptor kinase family of proteins. GRK2 is a positive effector of certain G protein-coupled receptors (GPCRs) and receptor-tyrosine kinases (RTK) transduction cascades. GRK2 phosphorylates the beta-adrenergic receptor as well as a wide range of other substrates including non-GPCR cell surface receptors, and cytoskeletal, mitochondrial, and transcription factor proteins. GRK2 specifically phosphorylates the agonist-occupied form of the beta-adrenergic and closely related receptors, likely inducing their desensitization. GRK2 is a key regulator of LPAR1 signaling that competes with RALA for binding to LPAR1, thus affecting the signaling properties of the receptor. GRK2 desensitizes LPAR1 and LPAR2 in a phosphorylation-independent manner. GRK2 positively regulates the ciliary smoothened (SMO)-dependent Hedgehog (Hh) signaling pathway by facilitating the trafficking of SMO into the cilium and the stimulation of SMO activity. The reaction catalyzed by GRK2 is as follows:

ATP+[beta-adrenergic receptor]=ADP+[beta-adrenergic receptor] phosphate.

The present invention relates to the discovery that loss of GRK2 in pancreatic cancer cell lines prevented tumor growth in vitro and when the GRK2 knockout cancer cells were implanted in mice. Cancer cell proliferation could not be restored by the introduction of kinase-dead GRK2 into knockout cancer cells, suggesting that loss of GRK2 kinase activity mediates the anti-proliferative effect. Treatment of pancreatic cancer cell lines with a small molecule GRK2 antagonist also reduced cancer cell proliferation. These findings indicate that inhibition of GRK2, particularly using small molecule GRK2 antagonists, can be used as a therapeutic strategy for treating pancreatic cancer and other cancers. These data also suggest that patients with overexpression of GRK2 are at increased risk of developing cancer and would benefit from specific treatments, such as treatment with the compositions and methods described herein.

GRK2 inhibitors

GRK2 inhibitors described herein can reduce or inhibit GRK2 function, expression, or signaling in order to treat cancer. GRK2 inhibitors can be grouped into categories based on their mechanism of action and their effect on GRK2: (i) GRK2-specific inhibitors (e.g., inhibitors that directly bind to, target, or knock down GRK2, such as GRK2-specific inhibitory RNAs and nucleases); (ii) GRK2 function blockers (e.g., inhibitors that prevent GRK2 from binding to a binding partner, or carrying out other processes necessary for normal GRK2 activity, e.g., small molecule antagonists of GRK2 and inhibitory RNAs and nucleases directed to GRK2 binding partners); and (iii) GRK2 signaling inhibitors (e.g., inhibitors that disrupt downstream signaling pathways or intracellular or extracellular events that occur after activation of GRK2).

GRK2-Specific Inhibitors

In some embodiments, the GRK2 inhibitor is a GRK2-specific inhibitor. GRK2-specific inhibitors selectively bind to, target, or knock down GRK2, without directly binding to or targeting other proteins. GRK2-specific inhibitors include inhibitory RNAs (e.g., shRNA, siRNA, or miRNA) and nucleases (e.g., TALENs, ZFNs, gRNAs, or Cas, e.g., Cas9) directed to GRK2. GRK2-specific inhibitors can reduce GRK2 function, expression, or signaling by 5% or more (e.g., 5%, 10%, 15%, 20%, 25%, 30%, 40%, 50%, 60%, 70%, 80%, 90%, 95%, 99%, or more).

In some embodiments, the GRK2-specific inhibitor is an inhibitory RNA (e.g., shRNA, siRNA, or miRNA) directed to GRK2.

In some embodiments, the GRK2-specific inhibitor is a nuclease (e.g., a TALEN, ZFN, gRNA, or Cas, e.g., Cas9) directed to GRK2. In some embodiments, the nuclease is directed to GRK2 by a guide RNA (gRNA) molecule. The gRNA may be introduced into a cell (e.g., a cancer cell) using a DNA construct (e.g., via an integrating virus). Exemplary gRNA molecules that can be used to target a nuclease to GRK2, for use in the methods and compositions described herein, include the gRNA molecules encoded by the DNA molecules used to knockout GRK2 in pancreatic cancer cells (e.g., SEQ ID NOs: 1-6). In some embodiments, the gRNA molecule directed to GRK2 is encoded by a DNA molecule having a nucleic acid sequence with at least 85% (e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%) sequence identity to the nucleic acid sequence of any one of SEQ ID NOs: 1-6. In some embodiments, the gRNA molecule directed to GRK2 is encoded by a DNA molecule having the nucleic acid sequence of any one of SEQ ID NOs: 1-6.

GRK2 Function Blockers

In some embodiments, the GRK2 inhibitor is a GRK2 function blocker. GRK2 function blockers can reduce or inhibit GRK2 function by reducing GRK2 expression, preventing or reducing the interaction of GRK2 with its binding partners (e.g., ADRB1 or other binding partners listed in Table 2), or preventing or reducing GRK2 activity (e.g., kinase activity). GRK2 function blockers include GRK2-specific inhibitors that directly target or bind to GRK2 (e.g., inhibitory RNAs and nucleases directed to GRK2), GRK2 small molecule antagonists, and GRK2 inhibitors directed to GRK2 binding partners (e.g., small molecule inhibitors, inhibitory RNAs, and nucleases directed to binding partners of GRK2, e.g., ADRB1 or other binding partners listed in Table 2).

GRK2 binding partners include ADRB1 (Entrez Gene ID: 153), ADRB2 (Entrez Gene ID: 154), RALA (Entrez Gene ID: 5898), LPAR1 (Entrez Gene ID: 1902), LPAR2 (Entrez Gene ID: 9170), CCRS (Entrez Gene ID: 1234), ADRA2A (Entrez Gene ID: 150), ARRB1 (Entrez Gene ID: 408), and GIT1 (Entrez Gene ID: 28964). In some embodiments, the GRK2 binding partner is a molecule listed in Table 2.

In some embodiments, the GRK2 function blocker is a small molecule antagonist that reduces or inhibits GRK2 activity. GRK2 small molecule antagonists for use in the methods and compositions described herein include compounds having the structure of any one of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), and Compounds 1-70. In some embodiments, the GRK2 small molecule antagonist reduces or inhibits GRK2 function. For example, the GRK2 small molecule inhibitor can reduce or inhibit GRK2 kinase activity. In some embodiments, the GRK2 small molecule inhibitor is a small molecule listed in U.S. Patent Application Publication No. US20170240538, International Application Publication No. WO/2005/090328, International Application Publication No. WO2007/034846, International Application Publication No. WO/2016/210403, or U.S. Pat. No. 7,910,602, the disclosures of which are incorporated herein by reference. In some embodiments, the GRK2 small molecule antagonist has the structure of Formula I-10 (described in Waldschmidt et al., J. Med. Chem. 59:3793-3807, 2016, the disclosure of which is incorporated herein by reference), Formula II-26 (described in Waldschmidt et al., J. Med. Chem. 60:3052-3069, 2017, the disclosure of which is incorporated herein by reference), Formula III-26 (described in Okawa et al., J. Med. Chem. 60:6942-6690, 2017, the disclosure of which is incorporated herein by reference), Formula III-27 (CAS-865609-72-9), Formula IV-8, or Compound 70.

In some embodiments, the GRK2 function blocker is an inhibitory RNA (e.g., shRNA, siRNA, or miRNA) directed to a GRK2 binding partner listed in Table 2. In some embodiments, the GRK2 function blocker is a nuclease (e.g., a TALEN, ZFN, gRNA, or Cas, e.g., Cas9) directed to a GRK2 binding partner listed in Table 2.

GRK2 Signaling Inhibitors

In some embodiments, the GRK2 inhibitor is a GRK2 signaling inhibitor. GRK2 signaling inhibitors include agents that reduce or inhibit signaling that occurs downstream of GRK2 activation or binding to a binding partner, such as small molecule inhibitors of intracellular signaling cascades (e.g., small molecule inhibitors that target the smoothened (SMO)-dependent Hedgehog (Hh) signaling pathway) and inhibitory RNAs directed to components of downstream signaling cascades (e.g., inhibitory RNAs directed to molecules in the SMO-dependent Hh signaling pathway). Small molecule signaling inhibitors for use in the methods and compositions described herein are listed in Table 3.

TABLE 2
GRK2 BINDING PARTNERS

| Symbol | HGNC Accession No. | Entrez Gene ID |
|---|---|---|
| ADRB2 | 286 | 154 |
| ADRB1 | 285 | 153 |
| ADRA2A | 281 | 150 |
| ARRB1 | 711 | 408 |
| GIT1 | 4272 | 28964 |
| CCR5 | 1606 | 1234 |
| LPAR1 | 3166 | 1902 |
| LPAR2 | 3168 | 9170 |
| RALA | 9839 | 5898 |

TABLE 3
GRK2 SIGNALING INHIBITORS

| Pathway | Inhibitors | Structure |
|---|---|---|
| Smoothened (SMO)- dependent Hedgehog (Hh) signaling | LDE225/Sonidegib | 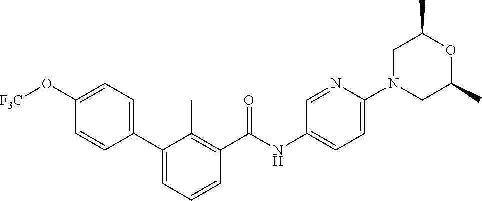 [SMILES: Cc1c(C(=O)Nc2ccc(N3C[C@@H](C)O[C@@H](C)C3)nc2)cccc1-c1ccc(OC(F)(F)F)cc1] |
| | GDC-0449/Vismodegib | 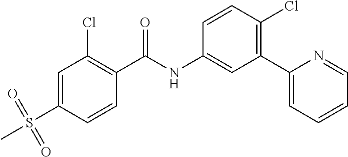 [SMILES: CS(=O)(=O)c1ccc(C(=O)Nc2ccc(Cl)c(-c3ccccn3)c2)c(Cl)c1] |

Agent Modalities

A GRK2 inhibitor can be selected from a number of different modalities. A GRK2 inhibitor can be a nucleic acid molecule (e.g., DNA molecule or RNA molecule, e.g., mRNA or inhibitory RNA molecule (e.g., siRNA, shRNA, or miRNA), or a hybrid DNA-RNA molecule), a small molecule (e.g., a GRK2 small molecule inhibitor, an inhibitor of a signaling cascade, or an epigenetic modifier), or a nuclease (e.g., Cas9, TALEN, or ZFN). Any of these modalities can be a GRK2 inhibitor directed to target (e.g., to reduce or inhibit) GRK2 function, GRK2 expression, GRK2 binding, or GRK2 signaling.

The nucleic acid molecule, small molecule, or nuclease can be modified. For example, the modification can be a chemical modification, e.g., conjugation to a marker, e.g., fluorescent marker or a radioactive marker. In other examples, the modification can include conjugation to a molecule that enhances the stability or half-life of the GRK2 inhibitor (e.g., the Fc domain of an antibody or serum albumin, e.g., human serum albumin). The modification can also include conjugation to an antibody to target the agent to a particular cell or tissue. Additionally, the modification can be a chemical modification, packaging modification (e.g., packaging within a nanoparticle or microparticle), or targeting modification to prevent the agent from crossing the blood brain barrier.

Small Molecules

Numerous small molecule GRK2 inhibitors (e.g., antagonists) useful in the methods of the invention are described herein and additional small molecule GRK2 inhibitors useful as therapies for cancer can also be identified through screening based on their ability to reduce or inhibit GRK2 function or signaling (e.g., GRK2 kinase activity). Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

In some embodiments, the GRK2 inhibitor is a GRK2 small molecule antagonist described herein (e.g., a compound having the structure of Formula I (e.g., having the structure of any one of Formula I-1 to I-39), Formula II (e.g., having the structure of any one of Formula II-1 toll-35), Formula III (e.g., having the structure of any one of Formula III-1 to III-56), or Formula IV (e.g., having the structure of any one of Formula IV-1 to IV-80), or any one of Compounds 1-70). In some embodiments, the GRK2 small molecule antagonist is a small molecule listed in U.S. Patent Application Publication No. US20170240538, International Application Publication No. WO/2005/090328, International Application Publication No. WO2007/034846, International Application Publication No. WO/2016/210403, or U.S. Pat. No. 7,910,602, the disclosures of which are incorporated herein by reference. In some embodiments, the GRK2 small molecule antagonist has the structure of Formula I-10 (described in Waldschmidt et al., J. Med. Chem. 59:3793-3807, 2016), Formula II-26 (described in Waldschmidt et al., J. Med. Chem. 60:3052-3069, 2017), Formula III-26 (described in Okawa et al., J. Med. Chem. 60:6942-6690, 2017), Formula III-27 (CAS-865609-72-9), Formula IV-8, or Compound 70. GRK2 inhibitors can be used to treat a disorder or condition described herein. A pharmaceutical composition containing the GRK2 small molecule antagonist can be formulated for treatment of a cancer described herein. In some embodiments, a pharmaceutical composition that includes the GRK2 small molecule antagonist is formulated for local administration, e.g., to the affected site in a subject.

Nucleic Acids

Inhibitory RNA

In some embodiments, the GRK2 inhibitor is an inhibitory RNA molecule, e.g., that acts by way of the RNA interference (RNAi) pathway. An inhibitory RNA molecule can decrease the expression level (e.g., protein level or mRNA level) of GRK2, a GRK2 binding partner (e.g., a binding partner listed in Table 2), or a molecule required for GRK2 signaling or function (e.g., a component of the SMO-dependent Hh signaling pathway). For example, an inhibitory RNA molecule includes a small interfering RNA (siRNA), short hairpin RNA (shRNA), and/or a microRNA (miRNA) that targets full-length GRK2, a GRK2 binding partner (e.g., a binding partner listed in Table 2), or a molecule required for GRK2 signaling or function (e.g., a component of the SMO-dependent Hh signaling pathway). A siRNA is a double-stranded RNA molecule that typically has a length of about 19-25 base pairs. A shRNA is a RNA molecule containing a hairpin turn that decreases expression of target genes via RNAi. shRNAs can be delivered to cells in the form of plasmids (e.g., viral or bacterial vectors), by transfection, electroporation, or transduction. A microRNA is a non-coding RNA molecule that typically has a length of about 22 nucleotides. MiRNAs bind to target sites on mRNA molecules and silence the mRNA, e.g., by causing cleavage of the mRNA, destabilization of the mRNA, or inhibition of translation of the mRNA. In some embodiments, the inhibitory RNA molecule decreases the level and/or activity of a positive regulator of function. In other embodiments, the inhibitory RNA molecule decreases the level and/or activity of an inhibitor of a positive regulator of function.

An inhibitory RNA molecule can be modified, e.g., to contain modified nucleotides, e.g., 2′-fluoro, 2′-o-methyl, 2′-deoxy, unlocked nucleic acid, 2′-hydroxy, phosphorothioate, 2′-thiouridine, 4′-thiouridine, 2′-deoxyuridine. Without being bound by theory, it is believed that certain modification can increase nuclease resistance and/or serum stability, or decrease immunogenicity.

In some embodiments, the inhibitory RNA molecule decreases the level and/or activity or function of GRK2, a GRK2 binding partner (e.g., a binding partner listed in Table 2), or a molecule required for GRK2 signaling or function (e.g., a component of the SMO-dependent Hh signaling pathway). In some embodiments, the inhibitory RNA molecule inhibits expression of GRK2, a GRK2 binding partner (e.g., a binding partner listed in Table 2), or a molecule required for GRK2 signaling or function (e.g., a component of the SMO-dependent Hh signaling pathway) (e.g., inhibits translation to protein). In other embodiments, the inhibitory RNA molecule increases degradation of GRK2, a GRK2 binding partner (e.g., a binding partner listed in Table 2), or a molecule required for GRK2 signaling or function (e.g., a component of the SMO-dependent Hh signaling pathway), and/or decreases the stability (i.e., half-life) of GRK2, a GRK2 binding partner (e.g., a binding partner listed in Table 2), or a molecule required for GRK2 signaling or function (e.g., a component of the SMO-dependent Hh signaling pathway). The inhibitory RNA molecule can be chemically synthesized or transcribed in vitro.

The making and use of inhibitory therapeutic agents based on non-coding RNA such as ribozymes, RNAse P, siRNAs, and miRNAs are also known in the art, for example, as described in Sioud, RNA Therapeutics: Function, Design, and Delivery (Methods in Molecular Biology). Humana Press 2010.

Gene Editing

In some embodiments, the GRK2 inhibitor is a component of a gene editing system. For example, the GRK2 inhibitor introduces an alteration (e.g., insertion, deletion (e.g., knockout), translocation, inversion, single point mutation, or other mutation) in GRK2 or a GRK2 binding partner (e.g., a binding partner listed in Table 2). Exemplary gene editing systems include the zinc finger nucleases (ZFNs), Transcription Activator-Like Effector-based Nucleases (TALEN), and the clustered regulatory interspaced short palindromic repeat (CRISPR) system. ZFNs, TALENs, and CRISPR-based methods are described, e.g., in Gaj et al. Trends Biotechnol. 31.7(2013):397-405.

CRISPR refers to a set of (or system containing a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archaeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically-designed CRISPR and one or more appropriate Cas proteins. The CRISPR locus is transcribed into RNA and processed by Cas proteins (e.g., Cas9) into small RNAs that contain a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341: 833, 2013. In some examples, the CRISPR system includes the Cas9 protein, a nuclease that cuts on both strands of the DNA. See, e.g., Id.

In some embodiments, in a CRISPR system for use described herein, e.g., in accordance with one or more methods described herein, the spacers of the CRISPR are derived from a target gene sequence, e.g., from a GRK2 sequence or sequence of a GRK2 binding partner (e.g., a binding partner listed in Table 2).

In some embodiments, the GRK2 inhibitor includes a guide RNA (gRNA) for use in a CRISPR system for gene editing. In embodiments, the GRK2 inhibitor contains a ZFN, or an mRNA encoding a ZFN, that targets (e.g., cleaves) a nucleic acid sequence (e.g., DNA sequence) of GRK2 or a GRK2 binding partner (e.g., a binding partner listed in Table 2). In some embodiments, the GRK2 inhibitor contains a TALEN, or an mRNA encoding a TALEN, that targets (e.g., cleaves) a nucleic acid sequence (e.g., DNA sequence) of GRK2 or a GRK2 binding partner (e.g., a binding partner listed in Table 2). In some embodiments, the GRK2 inhibitor contains a Cas (e.g., Cas9), or an mRNA encoding a Cas (e.g., Cas9), that targets (e.g., cleaves) a nucleic acid sequence (e.g., DNA sequence) of GRK2 or a GRK2 binding partner (e.g., a binding partner listed in Table 2).

In some examples, the gRNA or Cas (e.g., Cas9) can be used in a CRISPR system to engineer an alteration in a gene (e.g., GRK2). In other examples, the ZFN and/or TALEN can be used to engineer an alteration in a gene (e.g., GRK2). Exemplary alterations include insertions, deletions (e.g., knockouts), translocations, inversions, single point mutations, or other mutations. The alteration can be introduced in the gene in a cell, e.g., in vitro, ex vivo, or in vivo. In some embodiments, the alteration decreases the level and/or activity of (e.g., knocks down or knocks out) GRK2 or a GRK2 binding partner (e.g., a binding partner listed in Table 2), e.g., the alteration is a negative regulator of function. In yet another example, the alteration corrects a defect (e.g., a mutation causing a defect), in GRK2.

In certain embodiments, the CRISPR system is used to edit (e.g., to add or delete a base pair) a target gene, e.g., GRK2 or a GRK2 binding partner (e.g., a binding partner listed in Table 2). In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In some embodiments, the CRISPR system is used to direct Cas (e.g., Cas9) to a promoter of a target gene, e.g., GRK2 or a GRK2 binding partner (e.g., a binding partner listed in Table 2), thereby blocking an RNA polymerase sterically.

In some embodiments, a CRISPR system can be generated to edit GRK2 or a GRK2 binding partner (e.g., a binding partner listed in Table 2) using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., the gene encoding GRK2 or a GRK2 binding partner (e.g., a binding partner listed in Table 2). In CRISPRi, an engineered Cas9 protein (e.g., nuclease-null dCas9, or dCas9 fusion protein, e.g., dCas9—KRAB or dCas9—SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes described herein, e.g., a gene that inhibits GRK2 or a GRK2 binding partner (e.g., a binding partner listed in Table 2). In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1). The CRISPRi and CRISPRa techniques are described in greater detail, e.g., in Dominguez et al., Nat. Rev. Mol. Cell Biol. 17:5, 2016, incorporated herein by reference.

Viral Vectors

The GRK2 inhibitor can be delivered by a viral vector (e.g., a viral vector expressing a GRK2 inhibitor). Viral vectors can be used to express a transgene encoding an inhibitory nucleotide. A viral vector may be administered to a cell or to a subject (e.g., a human subject or animal model) to increase expression of an inhibitory nucleotide. Viral vectors can also be used to express a neurotoxin from Table 10 for combination therapy with a GRK2 inhibitor. A viral vector expressing a neurotoxin from Table 10 can be administered to a cell or to a subject (e.g., a human subject or animal model) to decrease or block neurotransmission. Viral vectors can be directly administered (e.g., injected) to a tumor to treat cancer.

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., AdS, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

### Blood Brain Barrier Permeability

In some embodiments, the GRK2 inhibitors for use in the present invention are agents that are not capable of crossing, or that do not cross, the blood brain barrier (BBB) of a mammal, e.g., an experimental rodent (e.g., mouse or rat), dog, pig, non-human primate, or a human. The BBB is a highly selective semipermeable membrane barrier that separates the circulating blood from the brain extracellular fluid (e.g., cerebrospinal fluid) in the central nervous system (CNS). The BBB is made up of high-density endothelial cells, which are connected by tight junctions. These cells prevent most molecular compounds in the bloodstream (e.g., large molecules and hydrophilic molecules) from entering the brain. Water, some gases (e.g., oxygen and carbon dioxide), and lipid-soluble molecules (e.g., hydrophobic molecules, such as steroid hormones) can cross the BBB by passive diffusion. Molecules that are needed for neural function, such as glucose and amino acids, are actively transported across the BBB.

A number of approaches can be used to render an agent BBB impermeable. These methods include modifications to increase an agent's size, polarity, or flexibility or reduce its lipophilicity, targeting approaches to direct an agent to another part of the body and away from the brain, and packaging approaches to deliver an agent in a form that does not freely diffuse across the BBB. These approaches can be used to render a BBB permeable GRK2 inhibitor BBB impermeable, and they can also be used to improve the properties (e.g., cell-specific targeting) of a GRK2 inhibitor that does not cross the BBB. The methods that can be used to render an agent BBB impermeable are discussed in greater detail herein below.

Formulation of BBB-Impermeable Agents for Enhanced Cell Targeting

One approach that can be used to render a GRK2 inhibitor BBB impermeable is to conjugate the agent to a targeting moiety that directs it somewhere other than the brain. The targeting moiety can be an antibody for a receptor expressed by the target cell (e.g., N-Acetylgalactosamine for liver transport; DGCR2, GBF1, GPR44 or SerpinB10 for pancreas transport; Secretoglobin, family 1A, member 1 for lung transport). The targeting moiety can also be a ligand of any receptor or other molecular identifier expressed on the target cell in the periphery. These targeting moieties can direct the GRK2 inhibitor of interest to its corresponding target cell, and can also prevent BBB crossing by directing the agent away from the BBB and increasing the size of the GRK2 inhibitor via conjugation of the targeting moiety.

GRK2 inhibitors can also be rendered BBB impermeable through formulation in a particulate delivery system (e.g., a nanoparticle, liposome, or microparticle), such that the agent is not freely diffusible in blood and cannot cross the BBB. The particulate formulation used can be chosen based on the desired localization of the GRK2 inhibitor (e.g., a tumor, tumor microenvironment, or site of metastasis), as particles of different sizes accumulate in different locations. For example, nanoparticles with a diameter of 45 nm or less enter the lymph node, while 100 nm nanoparticles exhibit poor lymph node trafficking. Some examples of the link between particle size and localization in vivo are described in Reddy et al., J Controlled Release 112:26 2006, and Reddy et al., Nature Biotechnology 25:1159 2007.

GRK2 inhibitors can be tested after the addition of a targeting moiety or after formulation in a particulate delivery system to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A GRK2 inhibitor that exhibits BBB impermeability can be used in the methods described herein.

Modification of Existing Compounds to Render them BBB Impermeable

There are multiple parameters that have been empirically derived in the field of medicinal chemistry to predict whether a compound will cross the BBB. The most common numeric value for describing permeability across the BBB is the log BB, defined as the logarithmic ratio of the concentration of a compound in the brain and in the blood. Empirical rules of thumb have been developed to predict BBB permeability, including rules regarding molecular size, polar surface area, sum of oxygen and nitrogen atoms, lipophilicity (e.g., partition coefficient between apolar solvent and water), “lipoaffinity”, molecular flexibility, and number of rotatable bonds (summarized in Muehlbacher et al., J Comput Aided Mol Des. 25: 1095 2011; and Geldenhuys et al., Ther Deliv. 6: 961 2015). Some preferred limits on various parameters for BBB permeability are listed in Table 1 of Ghose et al., ACS Chem Neurosci. 3: 50 2012, which is incorporated herein by reference. Based on the parameters shown in the table, one of skill in the art could modify an existing GRK2 inhibitor to render it BBB impermeable.

One method of modifying a GRK2 inhibitor to prevent BBB crossing is to add a molecular adduct that does not affect the target binding specificity, kinetics, or thermodynamics of the agent. Molecular adducts that can be used to render an agent BBB impermeable include polyethylene glycol (PEG), a carbohydrate monomer or polymer, a dendrimer, a polypeptide, a charged ion, a hydrophilic group, deuterium, and fluorine. GRK2 inhibitors can be tested after the addition of one or more molecular adducts or after any other properties are altered to determine whether or not they cross the BBB. Models for assessing BBB permeability include in vitro models (e.g., monolayer models, co-culture models, dynamic models, multi-fluidic models, isolated brain microvessels), in vivo models, and computational models as described in He et al., Stroke 45:2514 2014; Bickel, NeuroRx 2:15 2005; and Wang et al., Int J Pharm 288:349 2005. A GRK2 inhibitor that exhibits BBB impermeability can be used in the methods described herein.

Screening for or Development of BBB Impermeable Agents

Another option for developing BBB impermeable agents is to find or develop new agents that do not cross the BBB. One method for finding new BBB impermeable agents is to screen for compounds that are BBB impermeable. Compound screening can be performed using in vitro models (e.g., monolayer models, co-culture models, dynamic models, multi-fluidic models, isolated brain microvessels), in vivo models, and computational models, as described in He et al., Stroke 45:2514 2014; Bickel, NeuroRx 2:15 2005; Wang et al., Int J Pharm 288:349 2005, and Czupalla et al., Methods Mol Biol 1135:415 2014. For example, the ability of a molecule to cross the blood brain barrier can be determined in vitro using a transwell BBB assay in which microvascular endothelial cells and pericytes are co-cultured separated by a thin macroporous membrane, see e.g., Naik et al., J Pharm Sci 101:1337 2012 and Hanada et al., Int J Mol Sci 15:1812 2014; or in vivo by tracking the brain uptake of the target molecule by histology or radio-detection. Compounds would be deemed appropriate for use as GRK2 inhibitors in the methods described herein if they do not display BBB permeability in the aforementioned models.

### Cancer

The methods described herein can be used to treat cancer in a subject by administering to the subject an effective amount of a GRK2 inhibitor, e.g., a GRK2 inhibitor described herein. The method may include administering locally (e.g., intratumorally) to the subject a GRK2 inhibitor described herein in a dose (e.g., effective amount) and for a time sufficient to treat the cancer. For example, the stroma associated with the tumor, e.g., fibroblasts, is disrupted such that an essential function, e.g., the production of matrix metalloproteases, is altered to inhibit tumor survival or promote tumor control.

In some embodiments, the GRK2 inhibitor inhibits proliferation or disrupts the function of non-neural cells associated with the cancer, e.g., the method includes administering to the subject an effective amount of a GRK2 inhibitor for a time sufficient to inhibit proliferation or disrupt the function of non-neural cells associated with the cancer. Non-neural cells associated with the cancer include malignant cancer cells, malignant cancer cells in necrotic and hypoxic areas, adipocytes, pericytes, endothelial cells, cancer associated fibroblasts, fibroblasts, mesenchymal stem cells, red blood cells, or extracellular matrix. The proliferation of non-neural cells associated with the cancer may be decreased in the subject at least 1%, 2%, 5%, 10%, 15%, 20%, 25%, 30%, 35%, 40%, 50%, 60%, 70%, 80%, 90%, 95% or more, compared to before the administration. The proliferation of non-neural cells associated with the cancer can be decreased in the subject between 5-20%, between 5-50%, between 10-50%, between 20-80%, between 20-70%.

The GRK2 inhibitor can treat cancer by increasing cancer cell death or cancer cell autophagy in a subject (e.g., a human subject or animal model) or in a cancer cell culture (e.g., a culture generated from a patient tumor sample, a cancer cell line, or a repository of patient samples). A GRK2 inhibitor can increase cancer cell death or cancer cell autophagy by at least 1%, 2%, 5%, 10%, 15%, 20%, 25%, 30%, 35%, 40%, 50%, 60%, 70%, 80%, 90%, 95% or more compared to before administration to a subject or cancer cell culture. A GRK2 inhibitor can increase cancer cell death or cancer cell autophagy in a subject or cancer cell culture between 5-20%, between 5-50%, between 10-50%, between 20-80%, between 20-70%.

The GRK2 inhibitor can also act to inhibit cancer cell growth, proliferation, metastasis, migration, or invasion, e.g., the method includes administering to the subject (e.g., a human subject or animal model) or a cancer cell culture (e.g., a culture generated from a patient tumor sample, a cancer cell line, or a repository of patient samples) a GRK2 inhibitor in an amount (e.g., an effective amount) and for a time sufficient to inhibit cancer cell growth, proliferation, metastasis, migration, or invasion. Cancer cell growth, proliferation, metastasis, migration, or invasion can be decreased in the subject or cancer cell culture at least 1%, 2%, 5%, 10%, 15%, 20%, 25%, 30%, 35%, 40%, 50%, 60%, 70%, 80%, 90%, 95% or more, compared to before the administration. Cancer cell growth, proliferation, metastasis, migration, or invasion can be decreased in the subject or cancer cell culture between 5-20%, between 5-50%, between 10-50%, between 20-80%, between 20-70%.

The GRK2 inhibitor can inhibit cancer cell invasion or metastasis along a nerve, e.g., the method includes administering to the subject (e.g., a human subject or animal model) a GRK2 inhibitor in an amount (e.g., an effective amount) and for a time sufficient to inhibit cancer cell invasion or metastasis along a nerve. The GRK2 inhibitor can decrease cancer cell invasion or metastasis along a nerve in the subject at least 1%, 2%, 5%, 10%, 15%, 20%, 25%, 30%, 35%, 40%, 50%, 60%, 70%, 80%, 90%, 95% or more, compared to before the administration. The GRK2 inhibitor can decrease cancer cell invasion or metastasis along a nerve in the subject between 5-20%, between 5-50%, between 10-50%, between 20-80%, between 20-70%.

The GRK2 inhibitor can also reduce the number of nerve fibers in the affected tissue, reduce the activity of peripheral nerve fibers in the affected tissue (e.g., reduce neurotransmission), or reduce axonal growth or sprouting of nerve fibers in the affected tissue. For example, the method includes administering to the subject (e.g., a human subject or animal model) a GRK2 inhibitor in an amount (e.g., an effective amount) and for a time sufficient to reduce the number of nerve fibers in the affected tissue, reduce the activity of peripheral nerve fibers in the affected tissue (e.g., reduce neurotransmission), or reduce axonal growth or sprouting of nerve fibers in the affected tissue. The affected tissue can be a tumor, a tumor micro-environment, or site of metastasis. The number of nerve fibers in the affected tissue, the activity of peripheral nerve fibers in the affected tissue, or the axonal growth or sprouting of nerve fibers in the affected tissue can be decreased in the subject at least 1%, 2%, 5%, 10%, 15%, 20%, 25%, 30%, 35%, 40%, 50%, 60%, 70%, 80%, 90%, 95% or more, compared to before the administration. The number of nerve fibers in the affected tissue, the activity of peripheral nerve fibers in the affected tissue, or the axonal growth or sprouting of nerve fibers in the affected tissue can be decreased in the subject between 5-20%, between 5-50%, between 10-50%, between 20-80%, between 20-70%.

The nerve fibers that are modulated can be part of the peripheral nervous system, e.g., a somatic nerve, an autonomic nerve, a sensory nerve, a cranial nerve, an optic nerve, an olfactory nerve, a sympathetic nerve, a parasympathetic nerve, a chemoreceptor, a photoreceptor, a mechanoreceptor, a thermoreceptor, a nociceptor, an efferent nerve fiber, or an afferent nerve fiber.

Cancer Types

In the methods described herein, the cancer or neoplasm may be any solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Additional cancers that can be treated according to the methods described herein include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other cancers that can be treated according to the methods described herein include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leyomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastmic carcinoma, adrenocortical carcinoma, cholangiocarcinoma, Merkel cell carcinoma, DCIS, and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigno maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

In some embodiments, the cancer is a paraneoplastic cancer (e.g., a cancer that causes a paraneoplastic syndrome). Paraneoplastic syndromes are rare disorders that are triggered by an altered immune system response to a neoplasm, and are mediated by humoral factors such as hormones, cytokines, or auto-antibodies produced by the tumor. Symptoms of paraneoplastic syndrome may be endocrine, neuromuscular, or musculoskeletal, cardiovascular, cutaneous, hematologic, gastrointestinal, renal, or neurological. Paraneoplastic syndromes commonly present with lung, breast, and ovarian cancer and cancer of the lymphatic system (e.g., lymphoma). Paraneoplastic neurological disorders are disorders that affect the central or peripheral nervous system, and can include symptoms such as ataxia (difficulty with walking and balance), dizziness, nystagmus (rapid uncontrolled eye movements), difficulty swallowing, loss of muscle tone, loss of fine motor coordination, slurred speech memory loss, vision problems, sleep disturbances, dementia, seizures, or sensory loss in the limbs. Breast, ovarian, and lung cancers are most commonly associated with paraneoplastic neurological disorders. Other common types of paraneoplastic syndromes include paraneoplastic cerebellar degeneration, paraneoplastic pemphigus, paraneoplastic autonomic neuropathy, paraneoplastic encephalomyelitis, and cancer-associated autoimmune retinopathy.

Endocrine paraneoplastic syndromes include Cushing syndrome (caused by ectopic ACTH), which is most commonly caused by small cell lung cancer, pancreatic carcinoma, neural tumors, or thymoma; SIADH (caused by antidiuretic hormone), which is most commonly caused by small cell lung cancer and CNS malignancies; hypercalcemia (caused by PTHrp, TGFα, TNF, or IL-1), which is most commonly caused by lung cancer, breast carcinoma, renal and bladder carcinoma, multiple myeloma, adult T cell leukemia/lymphoma, ovarian carcinoma, and squamous cell carcinoma (e.g., lung, head, neck, or esophagus carcinoma); hyperglycemia (caused by insulin insulin-like substance, or “big” IGF-II), which is most commonly caused by fibrosarcoma, mesenchymal sarcomas, insulinoma, and hepatocellular carcinoma; carcinoid syndrome (caused by serotonin or bradykinin), which is most commonly caused by bronchial adenoma, pancreatic carcinoma, and gastric carcinoma; and hyperaldosteronism (caused by aldosterone), which is most commonly caused by adrenal adenoma/Conn's syndrome, non-Hodgkin's lymphoma, ovarian carcinoma, and pulmonary cancer.

Neurological paraneoplastic syndromes include Lambert-Eaton myasthenic syndrome (LEMS), which is most commonly caused by small cell lung cancer; paraneoplastic cerebellar degeneration, which is most commonly caused by lung cancer, ovarian cancer, breast carcinoma, and Hodgkin's lymphoma; encephalomyelitis; limbic encephalitis, which is most commonly caused by small cell lung carcinoma; myasthenia gravis, which is most commonly caused by thymoma; brainstem encephalitis; opsoclonus myoclonus ataxia (caused by autoimmune reaction against Nova-1), which is most commonly caused by breast carcinoma, ovarian carcinoma, small cell lung carcinoma, and neuroblastoma; anti-NMDA receptor encephalitis (caused by autoimmune reaction against NMDAR subunits), which is most commonly caused by teratoma; and polymyositis, which is most commonly caused by lung cancer, bladder cancer, and non-Hodgkin's lymphoma. Mucotaneous paraneoplastic syndromes include acanthosis nigricans, which is most commonly caused by gastric carcinoma, lung carcinoma, and uterine carcinoma; dermatomyositis, which is most commonly caused by bronchogenic carcinoma, breast carcinoma, ovarian cancer, pancreatic cancer, stomach cancer, colorectal cancer, and Non-Hodgkin's lymphoma; Leser-Trelat sign; necrolytic migratory erythema, which is most commonly caused by glucoganoma; Sweet's syndrome; florid cutaneous papillomatosis; pyoderma gangrenosum; and acquired generalized hypertrichosis.

Hematological syndromes include granulocytosis (caused by G-CSF); polycythemia (caused by erythropoietin), which is commonly caused by renal carcinoma, cerebellar hemangioma, and heptatocellular carcinoma; Trousseau sign (caused by mucins), which is commonly caused by pancreatic carcinoma and bronchogenic carcinoma; nonbacterial thrombotic endocarditis, which is caused by advanced cancers; and anemia, which is most commonly caused by thymic neoplasms. Other paraneoplastic syndromes include membranous glomerular nephritis; neoplastic fever; Staffer syndrome, which is caused by renal cell carcinoma; and tumor-induced osteomalacia (caused by FGF23), which is caused by hemangiopericytoma and phosphaturic mesenchymal tumor.

In some embodiments, a subject is identified as having cancer after presenting with symptoms of a paraneoplastic syndrome. A common symptom of paraneoplastic syndrome is fever. Auto-antibodies directed against nervous system proteins are also frequently observed in patients with paraneoplastic syndromes, including anti-Hu, anti-Yo, anti-Ri, anti-amphiphysin, anti-CV2, anti-Ma2, anti-recoverin, anti-transducin, anti-carbonic anhydrase II, anti-arrestin, anti-GCAP1, anti-GCAP2, anti-HSP27, anti-Rab6A, and anti-PNR. Other symptoms that can be used to identify a patient with paraneoplastic cancer include ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs. In some embodiments, a patient presents with symptoms of paraneoplastic syndrome and is then identified as having cancer based on imaging tests (e.g., CT, MRI, or PET scans).

The cancer may be highly innervated, metastatic, non-metastatic cancer, or benign (e.g., a benign tumor). The cancer may be a primary tumor or a metastasized tumor.

In some embodiments, the cancer is a GRK2-associated cancer (e.g., a cancer in which GRK2 is overexpressed).

Subjects who can be treated with the methods disclosed herein include subjects who have had one or more tumors resected, received chemotherapy or other pharmacological treatment for the cancer, received radiation therapy, and/or received other therapy for the cancer. Subjects who have not previously been treated for cancer can also be treated with the methods disclosed herein.

Combination Therapies

A GRK2 inhibitor described herein can be administered in combination with a second therapeutic agent for treatment of cancer. In some embodiments, the second therapeutic agent is selected based on tumor type, tumor tissue of origin, tumor stage, or mutations in genes expressed by the tumor.

Checkpoint Inhibitors

One type of agent that can be administered in combination with a GRK2 inhibitor described herein is a checkpoint inhibitor. Checkpoint inhibitors can be broken down into at least 4 major categories: i) agents such as antibodies that block an inhibitory pathway directly on T cells or natural killer (NK) cells (e.g., PD-1 targeting antibodies such as nivolumab and pembrolizumab, antibodies targeting TIM-3, and antibodies targeting LAG-3, 2B4, CD160, A2aR, BTLA, CGEN-15049, or KIR), ii) agents such as antibodies that activate stimulatory pathways directly on T cells or NK cells (e.g., antibodies targeting OX40, GITR, or 4-1BB), iii) agents such as antibodies that block a suppressive pathway on immune cells or rely on antibody-dependent cellular cytotoxicity to deplete suppressive populations of immune cells (e.g., CTLA-4 targeting antibodies such as ipilimumab, antibodies targeting VISTA, and antibodies targeting PD-L2, Gr1, or Ly6G), and iv) agents such as antibodies that block a suppressive pathway directly on cancer cells or that rely on antibody-dependent cellular cytotoxicity to enhance cytotoxicity to cancer cells (e.g., rituximab, antibodies targeting PD-L1, and antibodies targeting B7-H3, B7-H4, Gal-9, or MUC1). Such agents described herein can be designed and produced, e.g., by conventional methods known in the art (e.g., Templeton, Gene and Cell Therapy, 2015; Green and Sambrook, Molecular Cloning, 2012).

Chemotherapy

A second type of therapeutic agent that can be administered in combination with a GRK2 inhibitor described herein is a chemotherapeutic agent (e.g., a cytotoxic agent or other chemical compound useful in the treatment of cancer). These include alkylating agents, antimetabolites, folic acid analogs, pyrimidine analogs, purine analogs and related inhibitors, vinca alkaloids, epipodopyyllotoxins, antibiotics, L-asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art.

Biologic Cancer Agents

Another type of therapeutic agent that can be administered in combination with a GRK2 inhibitor described herein is a therapeutic agent that is a biologic such a cytokine (e.g., interferon or an interleukin (e.g., IL-2 or IL-12)) used in cancer treatment. In other embodiments the biologic is an anti-angiogenic agent, such as an anti-VEGF agent, e.g., bevacizumab. In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include Rituximab; Daclizumab; Basiliximab; Palivizumab; Infliximab; Trastuzumab; Gemtuzumab ozogamicin; Alemtuzumab; Ibritumomab tiuxetan; Adalimumab; Omalizumab; Tositumomab-I-131; Efalizumab; Cetuximab; Bevacizumab; Natalizumab; Tocilizumab; Panitumumab; Ranibizumab; Eculizumab; Certolizumab pegol; Golimumab; Canakinumab; Ustekinumab; Ofatumumab; Denosumab; Motavizumab; Raxibacumab; Belimumab; Ipilimumab; Brentuximab Vedotin; Pertuzumab; Ado-trastuzumab emtansine; and Obinutuzumab. Also included are antibody-drug conjugates. Examples of biologic cancer agents that can be used in combination with GRK2 inhibitors described herein are shown in Table 4 below.

TABLE 4
APPROVED CANCER ANTIBODIES

| Antibody | Company | Antigen | Indication |
|---|---|---|---|
| ado-trastuzumab | Genentech | HER2 | Metastatic breast cancer |
| emtansine | | | |
| alemtuzumab | Genzyme | CD52 | B-cell chronic lymphocytic leukemia |
| atezolizumab | Genentech | PD-L1 | Urothelial carcinoma |
| | | | Metastatic non-small cell lung cancer |
| avelumab | EMD Serono | PD-L1 | Metastatic Merkel cell carcinoma |
| bevacizumab | Genentech | VEGF | Metastatic colorectal cancer |
| blinatumomab | Amgen | CD19 | Precursor B-cell acute lymphoblastic |
| | | | leukemia |
| brentuximab | Seattle Genetics | CD30 | Hodgkin lymphoma |
| vedotin | | | Anaplastic large-cell lymphoma |
| cetuximab | ImClone Systems | EGFR | Metastatic colorectal carcinoma |
| daratumumab | Janssen Biotech | CD38 | Multiple myeloma |
| dinutuximab | United Therapeutics | GD2 | Pediatric high-risk neuroblastoma |
| durvalumab | AstraZeneca | PD-L1 | Urothelial carcinoma |
| elotuzumab | Bristol-Myers | SLAMF7 | Multiple myeloma |
| | Squibb | | |
| ibritumomab | Spectrum | CD20 | Relapsed or refractory low-grade, |
| tiuxetan | Pharmaceuticals | | follicular, or transformed B-cell non- |
| | | | Hodgkin's lymphoma |
| ipilimumab | Bristol-Myers | CTLA-4 | Metastatic melanoma |
| | Squibb | | |
| necitumumab | Eli Lilly | EGFR | Metastatic squamous non-small cell lung |
| | | | carcinoma |
| nivolumab | Bristol-Myers | PD-1 | Metastatic melanoma |
| | Squibb | | Metastatic squamous non-small cell lung |
| | | | carcinoma |
| obinutuzumab | Genentech | CD20 | Chronic lymphocytic leukemia |
| ofatumumab | Glaxo Grp | CD20 | Chronic lymphocytic leukemia |
| olaratumab | Eli Lilly | PDGFRA | Soft tissue sarcoma |
| panitumumab | Amgen | EGFR | Metastatic colorectal cancer |
| pembrolizumab | Merck | PD-1 | Metastatic melanoma |
| pertuzumab | Genentech | HER2 | Metastatic breast cancer |
| ramucirumab | Eli Lilly | VEGFR2 | Gastric cancer |
| rituximab | Genentech | CD20 | B-cell non-Hodgkin's lymphoma |
| trastuzumab | Genentech | HER2 | Metastatic breast cancer |

Cancer-Specific Agents

In some embodiments, the therapeutic agents administered with the GRK2 inhibitors described herein are cancer-specific. Cancer-specific agents are agents that have been shown to be particularly effective against certain types of cancer. Cancer-specific agents that can be administered with the GRK2 inhibitors described herein are listed in Table 5 below.

TABLE 5
CANCER-SPECIFIC AGENTS

| Cancer type | Agents |
|---|---|
| Pancreatic | Chemotherapeutics (Paclitaxel Albumin-stabilized Nanoparticle Formulation, |
| cancer | Erlotinib Hydrochloride, Everolimus, Fluorouracil Injection, Gemcitabine |
| | Hydrochloride, Irinotecan Hydrochloride Liposome, Mitomycin C, Sunitinib |
| | Malate, FOLFIRINOX, GEMCITABINE-CISPLATIN, GEMCITABINE- |
| | OXALIPLATIN, OFF, Lanreotide Acetate, Abraxane, Gemcitabine, Irinotecan, |
| | 5-FU, Oxaliplatin) lutetium Lu 177-dotatate |
| Melanoma | Checkpoint inhibitors (pembro, ipi, nivolumab, durvalumab), BRaf inhibitors |
| | (vemurafenib, debrafenib), MEK inhibitors, CDK4 inhibitors (ribociclib) |
| Renal cell | Checkpoint inhibitors (pembro, ipi, nivolumab, durvalumab), mTOR inhibitors |
| carcinoma | (everolimus), bevacizumab |
| Lung cancer | Checkpoint inhibitors (pembro, ipi, nivolumab, durvalumab), EGFR inhibitors |
| | (erlotinib, gefitinib, cetuximab) |
| Esophageal cancer | Chemotherapeutic agents (5FU, docetaxel), trastuzumab |
| Ovarian cancer | Chemotherapeutics (taxanes, cisplatin) |
| Uterine cancer | Chemotherapeutics (taxanes, cisplatin) |
| Head and Neck | Checkpoint inhibitors (pembro, ipi, nivolumab, durvalumab), EGFR inhibitors |
| cancer | (erlotinib, gefitinib, cetuximab) |
| Mesothelioma | Chemotherapeutics (pemetrexed, cisplatin) |

Non-Drug Therapies

Another type of agent that can be administered in combination with a GRK2 inhibitor is a therapeutic agent that is a non-drug treatment. For example, the second therapeutic agent is radiation therapy, cryotherapy, hyperthermia and/or surgical excision of tumor tissue.

CAR-T Therapy

Another therapy that can be employed in combination with the methods and compositions described herein is chimeric antigen receptor (CAR)-T therapy, or therapy with lymphocytes, such as autologous or allogeneic T cells, that have been modified to express a CAR that recognizes specific cancer antigens. Commonly, CARs contain a single chain fragment variable (scFv) region of an antibody or a binding domain specific for a tumor associated antigen (TAA) coupled via hinge and transmembrane regions to cytoplasmic domains of T cell signaling molecules. The most common lymphocyte activation moieties include a T cell costimulatory domain (e.g., CD28 and/or CD137) in tandem with a T cell effector function triggering (e.g. CD3) moiety. CARs have the ability to redirect T cell reactivity and specificity toward a selected target in a non-MHC restricted manner, exploiting the antigen-binding properties of monoclonal antibodies. The non-MHC restricted antigen recognition gives CAR-T cells the ability to bypass a major mechanism of tumor escape.

Oncolytic Viruses

Another type of therapeutic agent that can be administered in combination with a GRK2 inhibitor described herein is an oncolytic virus. Oncolytic viruses are naturally occurring and genetically engineered viruses that can selectively infect, replicate in, and kill cancer cells without harming normal cells. These viruses may be considered a type of immunotherapy, a treatment that harnesses the immune system to fight cancer, as a growing body of research suggests that oncolytic viruses may work by triggering an immune response against cancer. In some embodiments, the oncolytic virus administered in combination with a GRK2 inhibitor described herein is tamilogene laherparepvec (T-VEC), maraba virus, PVS-RIPO, canerpaturev, enadenotucirev, pelareorep, pexastimogene devacirepvec (JX-594), or tasadenoturev.

Neurotransmission Blockers

In some embodiments, the GRK2 inhibitor is administered in combination with a neurotransmission blocker (e.g., an agent that decreases neurotransmission). A neurotransmission blocker can be used to reduce or inhibit neural activity in a cancer or tumor that is innervated by nerves or to decrease the number of nerves in the tumor. For example, in some embodiments, the neurotransmission blocker is an antagonist of a neurotransmitter receptor listed in Table 6. Exemplary antagonists are listed in Tables 8A-8K. Neurotransmission blockers also include agents that decrease neurotransmitter synthesis or release (e.g., agents that decrease the activity of a biosynthetic protein encoded by a gene in Table 6 via inhibition or downregulation, or agents that decrease the activity of a synaptic or vesicular protein via blocking, disrupting, downregulating, or antagonizing the protein), increase neurotransmitter reuptake or degradation (e.g., agents that agonize, open, or stabilize transporters that remove neurotransmitter from the synaptic cleft), decrease neurotransmitter receptor activity (e.g., agents that decrease the activity of a signaling protein encoded by a gene in Table 6 via blocking or antagonizing the protein, or agents that block, antagonize, or downregulate a neurotransmitter receptor listed in Table 6), decrease neurotransmitter receptor synthesis or membrane insertion, increase neurotransmitter degradation, regulate neurotransmitter receptor conformation (e.g., agents that bind to a receptor and keep it in a “closed” or “inactive” conformation), and disrupt the pre- or postsynaptic machinery (e.g., agents that block or disrupt a structural protein, or agents that block, disrupt, downregulate, or antagonize a synaptic or vesicular protein). In some embodiments, the neurotransmitter receptor is a channel (e.g., a ligand or voltage gated ion channel), the activity of which can be decreased by blockade, antagonism, or inverse agonism of the channel. Neurotransmission blockers further include agents that sequester, block, antagonize, or degrade a neurotransmitter listed in Tables 6 or 7. Neurotransmission blockers include antibodies that bind to or block the function of neurotransmitters, neurotransmitter receptor antagonists, and toxins that disrupt synaptic release. Neurotransmission modulators can decrease neurotransmission by 10%, 20%, 30%, 40%, 50%, 60%, 70%, 80%, 90%, 95%, 98% or more. Neurotransmission blockers can be administered in any of the modalities described herein (e.g., antibody, small molecule, nucleic acid, polypeptide, or viral vector).

TABLE 6
NEUROTRANSMITTER GENES & PATHWAYS

| | | | Accession | Entrez |
|---|---|---|---|---|
| Gene | Pathway | Type | Number | Gene ID |
| ABAT | Neurotransmitter | Biosynthesis | P80404 | 18 |
| ACHE | Neurotransmitter | Biosynthesis | P22303 | 43 |
| ADORA2A | Neurotransmitter | Receptor | P29274 | 135 |
| ADORA2B | Neurotransmitter | Receptor | P29275 | 136 |
| Adra1a | Adrenergic/ | Receptor | P35348 | 148 |
| | Neurotransmitter | | | |
| Adra1b | Adrenergic/ | Receptor | P35368 | 147 |
| | Neurotransmitter | | | |
| Adra1d | Adrenergic/ | Receptor | P25100 | 146 |
| | Neurotransmitter | | | |
| Adra2a | Adrenergic/ | Receptor | P08913 | 150 |
| | Neurotransmitter | | | |
| Adra2b | Adrenergic/ | Receptor | P18089 | 151 |
| | Neurotransmitter | | | |
| Adra2c | Adrenergic/ | Receptor | P18825 | 152 |
| | Neurotransmitter | | | |
| Adrb1 | Adrenergic/ | Receptor | P08588 | 153 |
| | Neurotransmitter | | | |
| Adrb2 | Adrenergic/ | Receptor | P07550 | 154 |
| | Neurotransmitter | | | |
| Adrb3 | Adrenergic/ | Receptor | P13945 | 155 |
| | Neurotransmitter | | | |
| Adrbk1 | Adrenergic | Kinase | P25098 | 156 |
| Adrbk2 | Adrenergic | Kinase | P35626 | 157 |
| BACE1 | Neurotransmitter | Biosynthesis | P56817 | 23621 |
| BCHE | Neurotransmitter | Biosynthesis | P06276 | 590 |
| BRS3 | Neuromodulator | Receptor | P32247 | P32247 |
| C6orf89 | Neuromodulator | Receptor | Q6UWU4 | 221477 |
| CHAT | Neurotransmitter | Biosynthesis | P28329 | 1103 |
| CHRFAM7A | Neurotransmitter | Receptor | Q494W8 | 89832 |
| Chrm1 | Cholinergic/ | Receptor | P11229 | 1128 |
| | Neurotransmitter | | | |
| Chrm2 | Cholinergic/ | Receptor | P08172 | 1129 |
| | Neurotransmitter | | | |
| Chrm3 | Cholinergic/ | Receptor | P20309 | 1131 |
| | Neurotransmitter | | | |
| Chrm4 | Cholinergic/ | Receptor | P08173 | 1132 |
| | Neurotransmitter | | | |
| Chrm5 | Cholinergic/ | Receptor | P08912 | 1133 |
| | Neurotransmitter | | | |
| Chrna1 | Cholinergic/ | Receptor | P02708 | 1134 |
| | Neurotransmitter | | | |
| Chrna10 | Cholinergic/ | Receptor | Q9GZZ6 | 57053 |
| | Neurotransmitter | | | |
| Chrna2 | Cholinergic/ | Receptor | Q15822 | 1135 |
| | Neurotransmitter | | | |
| Chrna3 | Cholinergic/ | Receptor | P32297 | 1136 |
| | Neurotransmitter | | | |
| Chrna4 | Cholinergic/ | Receptor | P43681 | 1137 |
| | Neurotransmitter | | | |
| Chrna5 | Cholinergic/ | Receptor | P30532 | 1138 |
| | Neurotransmitter | | | |
| Chrna6 | Cholinergic/ | Receptor | Q15825 | 8973 |
| | Neurotransmitter | | | |
| Chrna7 | Cholinergic/ | Receptor | P36544 | 1139 |
| | Neurotransmitter | | | |
| Chrna9 | Cholinergic/ | Receptor | Q9UGM1 | 55584 |
| | Neurotransmitter | | | |
| Chrnb1 | Cholinergic/ | Receptor | P11230 | 1140 |
| | Neurotransmitter | | | |
| Chrnb2 | Cholinergic/ | Receptor | P17787 | 1141 |
| | Neurotransmitter | | | |
| Chrnb3 | Cholinergic/ | Receptor | Q05901 | 1142 |
| | Neurotransmitter | | | |
| Chrnb4 | Cholinergic/ | Receptor | P30926 | 1143 |
| | Neurotransmitter | | | |
| Chrnd | Cholinergic/ | Receptor | Q07001 | 1144 |
| | Neurotransmitter | | | |
| Chrne | Cholinergic/ | Receptor | Q04844 | 1145 |
| | Neurotransmitter | | | |
| Chrng | Cholinergic/ | Receptor | P07510 | 1146 |
| | Neurotransmitter | | | |
| CNR1 | Cannabinoid/ | Receptor | P21554 | 1268 |
| | Neurotransmitter | | | |
| CNR2 | Cannabinoid/ | Receptor | P34972 | 1269 |
| | Neurotransmitter | | | |
| CNRIP1 | Neurotransmitter | Receptor | Q96F85 | 25927 |
| COMT | Neurotransmitter | Biosynthesis | P21964 | 1312 |
| CPA4 | Neurotransmitter | Biosynthesis | Q9UI42 | 51200 |
| CPE | Neuropeptide/ | Biosynthesis | P16870 | 1363 |
| | Neurotransmitter | | | |
| CREM | Neurotransmitter | Signaling | Q03060 | 1390 |
| DAGLA | Neurotransmitter | Biosynthesis | Q9Y4D2 | 747 |
| | (Cannabinoid) | | | |
| DAGLB | Neurotransmitter | Biosynthesis | Q8NCG7 | 221955 |
| | (Cannabinoid) | | | |
| DBH | Neurotransmitter | Biosynthesis | P09172 | 1621 |
| DDC | Neurotransmitter | Biosynthesis | P20711 | 1644 |
| DGKI | Neurotransmitter | Biosynthesis | O75912 | 9162 |
| DOPO | Dopaminergic | Receptor | P09172 | 1621 |
| DPP4 | Neurotransmitter | Biosynthesis | P27487 | 1803 |
| Drd1 | Dopaminergic/ | Receptor | P21728 | 1812 |
| | Neurotransmitter | | | |
| Drd2 | Dopaminergic/ | Receptor | P14416 | 1813 |
| | Neurotransmitter | | | |
| Drd3 | Dopaminergic/ | Receptor | P35462 | 1814 |
| | Neurotransmitter | | | |
| Drd4 | Dopaminergic/ | Receptor | P21917 | 1815 |
| | Neurotransmitter | | | |
| Drd5 | Dopaminergic/ | Receptor | P21918 | 1816 |
| | Neurotransmitter | | | |
| ECEL1 | Neurotransmitter | Biosynthesis | O95672 | 9427 |
| FAAH | Neurotransmitter | Biosynthesis | O00519 | 2166 |
| FNTA | Neurotransmitter | Signaling | P49354 | 2339 |
| GABARAP | Neurotransmitter | Receptor | O95166 | 11337 |
| GABARAPL1 | Amine | Receptor | Q9H0R8 | 23710 |
| | Neuromodulator | | | |
| GABARAPL2 | Amine | Receptor | P60520 | 11345 |
| | Neuromodulator | | | |
| GABBR1 | Neurotransmitter | Receptor | Q9UBS5 | 2550 |
| GABBR2 | Amine | Receptor | O75899 | 9568 |
| | Neuromodulator | | | |
| GABRA1 | Neurotransmitter | Receptor | P14867 | 2554 |
| GABRA2 | Neurotransmitter | Receptor | P47869 | 2555 |
| GABRA3 | Neurotransmitter | Receptor | P34903 | 2556 |
| GABRA4 | Neurotransmitter | Receptor | P48169 | 2557 |
| GABRA5 | Neurotransmitter | Receptor | P31644 | 2558 |
| GABRA6 | Neurotransmitter | Receptor | Q16445 | 2559 |
| GABRB1 | Neurotransmitter | Receptor | P18505 | 2560 |
| GABRB2 | Neurotransmitter | Receptor | P47870 | 2561 |
| GABRB3 | Neurotransmitter | Receptor | P28472 | 2562 |
| GABRD | Neurotransmitter | Receptor | O14764 | 2563 |
| GABRE | Neurotransmitter | Receptor | P78334 | 2564 |
| GABRG1 | Neurotransmitter | Receptor | Q8N1C3 | 2565 |
| GABRG2 | Neurotransmitter | Receptor | P18507 | 2566 |
| GABRG3 | Neurotransmitter | Receptor | Q99928 | 2567 |
| GABRP | Neurotransmitter | Receptor | O00591 | 2568 |
| GABRQ | Neurotransmitter | Receptor | Q9UN88 | 55879 |
| GABRR1 | Neurotransmitter | Receptor | P24046 | 2569 |
| GABRR2 | Neurotransmitter | Receptor | P28476 | 2570 |
| GABRR3 | Neurotransmitter | Receptor | A8MPY1 | 200959 |
| GAD1 | Neurotransmitter | Biosynthesis | Q99259 | 2571 |
| GAD2 | Neurotransmitter | Biosynthesis | Q05329 | 2572 |
| GCHFR | Neurotransmitter | Biosynthesis | P30047 | 2644 |
| GLRA1 | Neurotransmitter | Receptor | P23415 | 2741 |
| GLRA2 | Neurotransmitter | Receptor | P23416 | 2742 |
| GLRA3 | Neurotransmitter | Receptor | O75311 | 8001 |
| GLRA4 | Neurotransmitter | Receptor | Q5JXX5 | 441509 |
| GLRB | Neurotransmitter | Receptor | P48167 | 2743 |
| GLS | Neurotransmitter | Biosynthesis | O94925 | 2744 |
| GLS2 | Neurotransmitter | Biosynthesis | Q9UI32 | 27165 |
| GluA1 (GluR1) | Amine | Receptor | P42261 | 2890 |
| | Neuromodulator | | | |
| GluK1 (GluR5) | Amine | Receptor | P39086 | 2897 |
| | Neuromodulator | | | |
| GLUL | Neurotransmitter | Biosynthesis | P15104 | 2752 |
| GluN1(NR1) | Amine | Receptor | Q05586 | 2902 |
| | Neuromodulator | | | |
| GNMT | Neurotransmitter | Biosynthesis | Q14749 | 27232 |
| GPER1 | Neurotransmitter | Receptor | Q99527 | 2852 |
| GPR1 | Neurotransmitter | Receptor | P46091 | 2825 |
| GPR139 | Neurotransmitter | Receptor | Q6DWJ6 | 124274 |
| GPR143 | Neurotransmitter | Receptor | P51810 | 4935 |
| GPR149 | Neurotransmitter | Receptor | Q86SP6 | 344758 |
| GPR18 | Neurotransmitter | Receptor | Q14330 | 2841 |
| GPR21 | Neurotransmitter | Receptor | Q99679 | 2844 |
| GPR26 | Neurotransmitter | Receptor | Q8NDV2 | 2849 |
| GPR3 | Neurotransmitter | Receptor | P46089 | 2827 |
| GPR35 | Neurotransmitter | Receptor | Q9HC97 | 2859 |
| GPR52 | Neurotransmitter | Receptor | Q9Y2T5 | 9293 |
| GPR55 | Neurotransmitter | Receptor | Q9Y2T6 | 9290 |
| GPR78 | Neurotransmitter | Receptor | Q96P69 | 27201 |
| GPR83 | Neurotransmitter | Receptor | Q9NYM4 | 10888 |
| GPR84 | Neurotransmitter | Receptor | Q9NQS5 | 53831 |
| GPRASP1 | Neurotransmitter | Receptor | Q5JY77 | 9737 |
| GPR50 | Amine | Receptor | Q13585 | 9248 |
| | Neuromodulator | | | |
| GRIA1 | Neurotransmitter | Receptor | P42261 | 2890 |
| GRIA2 | Neurotransmitter | Receptor | P42262 | 2891 |
| GRIA3 | Neurotransmitter | Receptor | P42263 | 2892 |
| GRIA4 | Neurotransmitter | Receptor | P48058 | 2893 |
| GRID1 | Neurotransmitter | Receptor | Q9ULK0 | 2894 |
| GRID2 | Neurotransmitter | Receptor | O43424 | 2895 |
| GRIK1 | Neurotransmitter | Receptor | P39086 | 2897 |
| GRIK2 | Neurotransmitter | Receptor | Q13002 | 2898 |
| GRIK3 | Neurotransmitter | Receptor | Q13003 | 2899 |
| GRIK4 | Neurotransmitter | Receptor | Q16099 | 2900 |
| GRIK5 | Neurotransmitter | Receptor | Q16478 | 2901 |
| GRIN1 | Neurotransmitter | Receptor | Q05586 | 2902 |
| GRIN2A | Neurotransmitter | Receptor | Q12879 | 2903 |
| GRIN2B | Neurotransmitter | Receptor | Q13224 | 2904 |
| GRIN2C | Neurotransmitter | Receptor | Q14957 | 2905 |
| GRIN2D | Neurotransmitter | Receptor | O15399 | 2906 |
| GRIN3A | Neurotransmitter | Receptor | Q8TCU5 | 116443 |
| GRIN3B | Neurotransmitter | Receptor | O60391 | 116444 |
| GRK2 | Neurotransmitter | Receptor | P25098 | 156 |
| GRK3 | Neurotransmitter | Receptor | P35626 | 157 |
| GRM1 | Neurotransmitter | Receptor | Q13255 | 2911 |
| GRM2 | Neurotransmitter | Receptor | Q14416 | 2912 |
| GRM3 | Neurotransmitter | Receptor | Q14832 | 2913 |
| GRM4 | Neurotransmitter | Receptor | Q14833 | 2914 |
| GRM5 | Neurotransmitter | Receptor | P41594 | 2915 |
| GRM6 | Neurotransmitter | Receptor | O15303 | 2916 |
| GRM7 | Neurotransmitter | Receptor | Q14831 | 2917 |
| GRM8 | Neurotransmitter | Receptor | O00222 | 2918 |
| HNMT | Neurotransmitter | Biosynthesis | P50135 | 3176 |
| HOMER1 | Neurotransmitter | Receptor | Q86YM7 | 9456 |
| HRH1 | Neurotransmitter | Receptor | P35367 | 3269 |
| HRH2 | Neurotransmitter | Receptor | P25021 | 3274 |
| HRH3 | Neurotransmitter | Receptor | Q9Y5N1 | 11255 |
| HRH4 | Neurotransmitter | Receptor | Q9H3N8 | 59340 |
| Htr1a | Neurotransmitter | Receptor | P08908 | 3350 |
| Htr1b | Neurotransmitter | Receptor | P28222 | 3351 |
| Htr1c | Neurotransmitter | Receptor | P28335 | |
| Htr1d | Neurotransmitter | Receptor | P28221 | 3352 |
| Htr1e | Neurotransmitter | Receptor | P28566 | 3354 |
| Htr1f | Neurotransmitter | Receptor | P30939 | 3355 |
| Htr2a | Neurotransmitter | Receptor | P28223 | 3356 |
| Htr2b | Neurotransmitter | Receptor | P41595 | 3357 |
| Htr2c | Neurotransmitter | Receptor | P28335 | 3358 |
| Htr3a | Neurotransmitter | Receptor | P46098 | 3359 |
| Htr3b | Neurotransmitter | Receptor | O95264 | 9177 |
| Htr3c | Neurotransmitter | Receptor | Q8WXA8 | 170572 |
| Htr3d | Neurotransmitter | Receptor | Q70Z44 | 200909 |
| HTR3E | Neurotransmitter | Receptor | A5X5Y0 | 285242 |
| Htr4 | Neurotransmitter | Receptor | Q13639 | 3360 |
| Htr5a | Neurotransmitter | Receptor | P47898 | 3361 |
| Htr5b | Neurotransmitter | Receptor | P35365 | 79247 |
| HTR5BP | Neurotransmitter | Receptor | | 645694 |
| Htr6 | Neurotransmitter | Receptor | P50406 | 3362 |
| Htr7 | Neurotransmitter | Receptor | P32305 | 3363 |
| ITPR1 | Neurotransmitter | Signaling | Q14643 | 3708 |
| ITPR2 | Neurotransmitter | Signaling | Q14571 | 3709 |
| ITPR3 | Neurotransmitter | Signaling | Q14573 | 3710 |
| LYNX1 | Neurotransmitter | Receptor | Q9BZG9 | 66004 |
| MAOA | Neurotransmitter | Biosynthesis | P21397 | 4128 |
| MAOB | Neurotransmitter | Biosynthesis | P27338 | 4129 |
| NAMPT | Neurotransmitter | Biosynthesis | P43490 | 10135 |
| NISCH | Neurotransmitter | Receptor | Q9Y2I1 | 11188 |
| NOS1 | Neurotransmitter | Biosynthesis | P29475 | 4842 |
| NPTN | Neurotransmitter | Receptor | Q9Y639 | 27020 |
| P2RX1 | Neurotransmitter | Receptor | P51575 | 5023 |
| P2RX2 | Neurotransmitter | Receptor | Q9UBL9 | 22953 |
| P2RX3 | Neurotransmitter | Receptor | P56373 | 5024 |
| P2RX4 | Neurotransmitter | Receptor | Q99571 | 5025 |
| P2RX5 | Neurotransmitter | Receptor | Q93086 | 5026 |
| P2RX6 | Neurotransmitter | Receptor | O15547 | 9127 |
| P2RX7 | Neurotransmitter | Receptor | Q99572 | 5027 |
| P2RY11 | Neurotransmitter | Receptor | Q96G91 | 5032 |
| PAH | Neurotransmitter | Biosynthesis | P00439 | 5053 |
| PC | Neurotransmitter | Biosynthesis | P11498 | 5091 |
| PDE1B | Neurotransmitter | Signaling | Q01064 | 5153 |
| PDE4A | Neurotransmitter | Signaling | P27815 | 5141 |
| PDE4D | Neurotransmitter | Signaling | Q08499 | 5144 |
| PHOX2A | Neurotransmitter | Biosynthesis | O14813 | 401 |
| PHOX2B | Neurotransmitter | Biosynthesis | Q99453 | 8929 |
| PIK3CA | Neurotransmitter | Signaling | P42336 | 5290 |
| PIK3CB | Neurotransmitter | Signaling | P42338 | 5291 |
| PIK3CG | Neurotransmitter | Signaling | P48736 | 5294 |
| PLCB1 | Neurotransmitter | Signaling | Q9NQ66 | 23236 |
| PLCB2 | Neurotransmitter | Signaling | Q00722 | 5330 |
| PLCB3 | Neurotransmitter | Signaling | Q01970 | 5331 |
| PLCB4 | Neurotransmitter | Signaling | Q15147 | 5332 |
| PLCD1 | Neurotransmitter | Signaling | P51178 | 5333 |
| PLCE1 | Neurotransmitter | Signaling | Q9P212 | 51196 |
| PLCG1 | Neurotransmitter | Signaling | P19174 | 5335 |
| PLCL1 | Neurotransmitter | Signaling | Q15111 | 5334 |
| PLCL2 | Neurotransmitter | Signaling | Q9UPR0 | 23228 |
| PPP1CB | Neurotransmitter | Signaling | P62140 | 5500 |
| PPP1CC | Neurotransmitter | Signaling | P36873 | 5501 |
| PRIMA1 | Neurotransmitter | Biosynthesis | Q86XR5 | 145270 |
| PRKACG | Neurotransmitter | Signaling | P22612 | 5568 |
| PRKAR2B | Neurotransmitter | Signaling | P31323 | 5577 |
| PRKCG | Neurotransmitter | Signaling | P05129 | 5582 |
| PRKX | Neurotransmitter | Signaling | P51817 | 5613 |
| RIC3 | Neurotransmitter | Receptor | Q7Z5B4 | 79608 |
| SHANK3 | Neurotransmitter | Signaling | Q9BYB0 | 85358 |
| SLC6A1 | Amine | Transferase | P30531 | 6529 |
| | Neuromodulator | | | |
| SLC6A13 | Amine | Transferase | Q9NSD5 | 6540 |
| | Neuromodulator | | | |
| Slc6a4 | Serotonin | Transporter | P31645 | 6532 |
| SNX13 | Neurotransmitter | Signaling | Q9Y5W8 | 23161 |
| TAAR1 | Amine | Receptor | Q96RJ0 | 134864 |
| | Neuromodulator | | | |
| TAAR2 | Amine | Receptor | Q9P1P5 | 9287 |
| | Neuromodulator | | | |
| TAAR5 | Neurotransmitter | Receptor | O14804 | 9038 |
| TH | Neurotransmitter | Biosynthesis | P07101 | 7054 |
| TPH1 | Neurotransmitter | Biosynthesis | P17752 | 7166 |
| TPH2 | Neurotransmitter | Biosynthesis | Q8IWU9 | 121278 |
| TRHDE | Neurotransmitter | Biosynthesis | Q9UKU6 | 29953 |

TABLE 7
NEUROTRANSMITTERS

| Ligand | Pathway | Type |
|---|---|---|
| 2-Arachidonoylglycerol | Endocannabinoid | Ligand |
| 2-Arachidonyl glyceryl ether | Endocannabinoid | Ligand |
| 3-methoxytyramine | Amines | Ligand |
| Acetylcholine | Amino Acids | Ligand |
| Adenosine | Purine | Ligand |
| Adenosine triphosphate | Purine | Ligand |
| Agmatine | Amino Acids | Ligand |
| Anandamide | Endocannabinoid | Ligand |
| Aspartate | Amino Acids | Ligand |
| Carbon monoxide | Gas | Ligand |
| D-serine | Amino Acids | Ligand |
| Dopamine | Monoamines | Ligand |
| Dynorphin | Opioids | Ligand |
| Endorphin | Opioids | Ligand |
| Enkephalin | Opioids | Ligand |
| Epinephrine | Monoamines | Ligand |
| Gamma-aminobutyric acid | Amino Acids | Ligand |
| Glutamate | Amino Acids | Ligand |
| Glycine | Amino Acids | Ligand |
| Histamine | Monoamines | Ligand |
| N-Acetylaspartylglutamate | Neuropeptides | Ligand |
| N-Arachidonoyl dopamine | Endocannabinoid | Ligand |
| N-methylphenethylamine | Amines | Ligand |
| N-methyltryptamine | Amines | Ligand |
| Nitric oxide | Gas | Ligand |
| Norepinephrine | Monoamines | Ligand |
| Octopamine | Amines | Ligand |
| Phenethylamine | Amines | Ligand |
| Serotonin | Monoamines | Ligand |
| Synephrine | Amines | Ligand |
| Tryptamine | Amines | Ligand |
| Tyramine | Amines | Ligand |
| Virodhamine | Endocannabinoid | Ligand |

TABLE 8A
AGONISTS AND ANTAGONIST AGENTS

| Gene | Agonist | Antagonist |
|---|---|---|
| Adrb2 | NCX 950 | Alprenolol |
| Accession | Bitolterol | Carvedilol |
| Number: | Isoetarine | Desipramine |
| P07550 | Norepinephrine | Nadolol |
| | Phenylpropanolamine | Levobunolol |
| | Dipivefrin | Metipranolol |
| | Epinephrine | Bevantolol |
| | Orciprenaline | Oxprenolol |
| | Dobutamine | Nebivolol |
| | Ritodrine | Asenapine |
| | Terbutaline | Bupranolol |
| | Salmeterol | Penbutolol |
| | Formoterol | Celiprolol |
| | Salbutamol | Pindolol |
| | Isoprenaline | Acebutolol |
| | Arbutamine | Bopindolol |
| | Arformoterol | |
| | Fenoterol | |
| | Pirbuterol | |
| | Ephedra | |
| | Procaterol | |
| | Clenbuterol | |
| | Bambuterol | |
| | Indacaterol | |
| | Droxidopa | |
| | Olodaterol | |
| | Vilanterol | |
| | Pseudoephedrine | |
| | Cabergoline | |
| | Mirtazepine | |
| Adra1d | Midodrine | Dapiprazole |
| Accession | Norepinephrine | Amitriptyline |
| Number: | Clonidine | Alfuzosin |
| P25100 | Oxymetazoline | Promazine |
| | Pergolide | Prazosin |
| | Bromocriptine | Imipramine |
| | Droxidopa | Nortriptyline |
| | Xylometazoline | Doxazosin |
| | Ergotamine | Nicardipine |
| | Cirazoline | Dronedarone |
| | Cabergoline | Tamsulosin |
| | Methoxamine | Propiomazine |
| | Epinephrine | Phenoxybenzamine |
| | | Carvedilol |
| | | Doxepin |
| | | Terazosin |
| | | Quetiapine |
| | | Methotrimeprazine |
| | | Silodosin |
| Adrb1 | Isoetarine | Esmolol |
| Accession | Norepinephrine | Betaxolol |
| Number: | Phenylpropanolamine | Metoprolol |
| P08588 | Epinephrine | Atenolol |
| | Dobutamine | Timolol |
| | Salbutamol | Sotalol |
| | Isoprenaline | Propranolol |
| | Arbutamine | Labetalol |
| | Fenoterol | Bisoprolol |
| | Pirbuterol | Alprenolol |
| | Ephedra | Amiodarone |
| | Clenbuterol | Carvedilol |
| | Droxidopa | Nadolol |
| | Pseudoephedrine | Levobunolol |
| | Carteolol | Metipranolol |
| | Cabergoline | Bevantolol |
| | Mirtazapine | Practolol |
| | Loxapine | Oxprenolol |
| | Vortioxetine | Celiprolol |
| | Desipramine | Nebivolol |
| | | Asenapine |
| | | Bupranolol |
| | | Penbutolol |
| | | Pindolol |
| | | Acebutolol |
| | | Bopindolol |
| | | Cartelol |
| Adrb3 | SR 58611 | Bopindolol |
| Accession | Norepinephrine | Propranolol |
| Number: | Epinephrine | Bupranolol |
| P13945 | Isoprenaline | |
| | Arbutamine | |
| | Fenoterol | |
| | Ephedra | |
| | Clenbuterol | |
| | Droxidopa | |
| | Mirabegron | |
| Adrbk1 | ATP | Alprenolol |
| Accession | Carbachol | Heparin |
| Number: | Dopamine | |
| P25098 | Isoproterenol | |
| | Morphine | |
| | DAMGO | |
| | histamine | |
| | Acetylcholine | |
| | Etorphine | |
| | NMDA | |
| | Dopamine | |
| Adrbk2 | Isoproterenol | Propranolol |
| Accession | DAMGO | |
| Number: | ATP | |
| P26819 | | |
| Chrm3 | cgmp | MT3 |
| Accession | ATP | Hexocyclium |
| Number: | Cevimeline | Himbacine |
| P20309 | arecoline | Biperiden |
| | oxotremorine-M | lithocholylcholine |
| | NNC 11-1314 | AFDX384 |
| | xanomeline | 4-DAMP |
| | oxotremorine | hexahydrodifenidol |
| | pentylthio-TZTP | VU0255035 |
| | arecaidine propargyl ester | N-methyl scopolamine |
| | NNC 11-1607 | Darifenacin |
| | furmethide | Thiethylperazine |
| | NNC 11-1585 | methoctramine |
| | Acetylcholine | silahexocyclium |
| | methylfurmethide | Strychnine |
| | Bethanechol | MT7 |
| | Carbachol | Heparin |
| | Succinylcholine | Olanzapine |
| | ALKS 27 | Pirenzepine |
| | itopride | Clidinium |
| | methacholine | Ipratropium |
| | Meperidine | Propantheline |
| | Cinnarizine | Dicyclomine |
| | Trimipramine | Darifenacin |
| | | Tiotropium |
| | | Atropine |
| | | Scopolamine |
| | | Amitriptyline |
| | | Doxepin |
| | | Lidocaine |
| | | Nortriptyline |
| | | Tropicamide |
| | | Metixene |
| | | Homatropine Methylbromide |
| | | Solifenacin |
| | | Glycopyrrolate |
| | | Propiomazine |
| | | Diphemanil Methylsulfate |
| | | Promethazine |
| | | Diphenidol |
| | | Pancuronium |
| | | Ziprasidone |
| | | Quetiapine |
| | | Imipramine |
| | | Clozapine |
| | | Cyproheptadine |
| | | Aripiprazole |
| | | Nicardipine |
| | | Amoxapine |
| | | Loxapine |
| | | Promazine |
| | | Oxyphencyclimine |
| | | Anisotropine Methylbromide |
| | | Tridihexethyl |
| | | Chlorpromazine |
| | | Ketamine |
| | | Cyclosporin A |
| | | Paroxetine |
| | | Benzquinamide |
| | | Tolterodine |
| | | Oxybutynin |
| | | Alcuronium |
| | | WIN 62,577 |
| | | Tramadol |
| | | Chlorprothixene |
| | | Aclidinium |
| | | Methotrimeprazine |
| | | Umeclidinium |
| | | Cryptenamine |
| | | Mepenzolate |
| | | Maprotiline |
| | | Brompheniramine |
| | | Isopropamide |
| | | Trihexyphenidyl |
| | | Ipratropium bromide |
| | | Hyoscyamine |
| | | Procyclidine |
| | | Pipecuronium |
| | | Fesoterodine |
| | | Disopyramide |
| | | Desipramine |
| | | Mivacurium |
| Chrna3 | Nicotine | A-867744 |
| Accession | Varenicline | NS1738 |
| Number: | Acetylcholine | Hexamethonium |
| P32297 | Ethanol | Mecamylamine |
| | Cytisine | Dextromethorphan |
| | Levamisole | Pentolinium |
| | Galantamine | Levomethadyl Acetate |
| | | Bupropion |
| Chrna6 | Nicotine | Hexamethonium |
| Accession | Cytisine | Mecamylamine |
| Number: | Varenicline | |
| Q15825 | Galantamine | |
| Chrna9 | Nicotine | Hexamethonium |
| Accession | Galantamine | Mecamylamine |
| Number: | Ethanol | Tetraethylammonium |
| Q9UGM1 | | Muscarine |
| | ATG003 | Strychnine |
| | Lobeline | |
| | RPI-78M | |
| Chrnb1 | Galantamine | |
| Accession | | |
| Number: | | |
| P11230 | | |
| Chrnb4 | Nicotine | Atropine |
| Accession | Varenicline | Oxybutynin |
| Number: | PNU-120596 | Pentolinium |
| P30926 | Ethanol | Dextromethorphan |
| | Galantamine | |
| Chrng | Galantamine | |
| Accession | | |
| Number: | | |
| P07510 | | |
| Adcyap1 | Nicotine | Atropine |
| Accession | CGMP | PPADS |
| Number: | Apomorphine | Onapristone |
| P18509 | Suramin | Muscarine |
| | Nifedipine | Haloperidol |
| | ATP | Astressin |
| | Dihydrotestosterone | Melatonin |
| | Maxadilan | Scopolamine |
| | Dexamethasone | Tetrodotoxin |
| | Acetylcholine | Apamin |
| | Histamine | Hexamethonium |
| | Carbachol | Indomethacin |
| | NMDA | Propranolol |
| | Dopamine | Bumetanide |
| | Isoproterenol | Progesterone |
| | Salbutamol | Charybdotoxin |
| | Morphine | Prazosin |
| | Clonidine | |
| | Nimodipine | |
| | 2,6-Diamino-Hexanoic Acid Amide | |
| CYSLTR1 | Salbutamol | Montelukast |
| Accession | Dexamethasone | Zafirlukast |
| Number: | Arachidonic acid | Cinalukast |
| Q9Y271 | Histamine | Pranlukast |
| | | Nedocromil |
| | | Theophylline |
| | | Indomethacin |
| | | Zileuton |
| | | Iralukast |
| | | Pobilukast |
| | | Sulukast |
| | | Verlukast |
| LTB4R | LTB | U75302 |
| Accession | ATP | CP105696 |
| Number: | Dexamethasone | CP-195543 |
| Q15722 | cholesterol | Etalocib |
| | 20-hydroxy-LTB< | SC-41930 |
| | 12R-HETE | LY255283 |
| | arachidonic acid | Zafirlukast |
| | | ONO-4057 |
| | | RO5101576 |
| | | BILL 260 |
| PENK | Dopamine | Naltrexone |
| Accession | kainate | Naloxone |
| Number: | NMDA | Progesterone |
| P01210 | DAMGO | |
| | Morphine | |
| Htr2c | Apomorphine | Melatonin |
| Accession | Bifeprunox | SB 224289 |
| Number: | Tramadol | LY334362 |
| P28335 | AL-37350A | FR260010 |
| | 5-MeO-DMT | Sulpiride |
| | BW723C86 | Thiethylperazine |
| | CGS-12066 | cyamemazine |
| | DOI | Mesulergine |
| | 5-CT | SB 221284 |
| | YM348 | Zotepine |
| | LSD | Metergoline |
| | xanomeline | methiothepin |
| | WAY-163909 | Spiperone |
| | Dopamine | SB 215505 |
| | LY344864 | Tiospirone |
| | VER-3323 | SB 228357 |
| | TFMPP | Pizotifen |
| | 8-OH-DPAT | SB 206553 |
| | MK-212 | SB 204741 |
| | NMDA | SDZ SER-082 |
| | org 12962 | Ritanserin |
| | 5-MeOT | SB 242084 |
| | RU 24969 | S33084 |
| | Acetylcholine | Roxindole |
| | QUINPIROLE | RS-127445 |
| | quipazine | Terguride |
| | tryptamine | EGIS-7625 |
| | Ro 60-0175 | SB 243213 |
| | Oxymetazoline | RS-102221 |
| | Ergotamine | Olanzapine |
| | Cabergoline | Aripiprazole |
| | Lorcaserin | Agomelatine |
| | Pergolide | Ziprasidone |
| | Methylergonovine | Quetiapine |
| | Renzapride | Sarpogrelate |
| | Pramipexole | Perphenazine |
| | GR-127935 | Thioridazine |
| | BRL-15572 | Sertindole |
| | ipsapirone | Loxapine |
| | SB 216641 | Methysergide |
| | SL65.0155 | Risperidone |
| | S 16924 | Asenapine |
| | Bromocriptine | Mianserin |
| | Lisuride | Clozapine |
| | Tegaserod | Trifluoperazine |
| | Epicept NP-1 | Trazodone |
| | dapoxetine | Doxepin |
| | Dexfenfluramine | Nortriptyline |
| | 3,4- | Chlorprothixene |
| | Methylenedioxymethamphetamine | |
| | Ropinirole | Minaprine |
| | Maprotiline | Propiomazine |
| | Desipramine | Mirtazapine |
| | | Amoxapine |
| | | Yohimbine |
| | | Cyproheptadine |
| | | Imipramine |
| | | Amitriptyline |
| | | Promazine |
| | | Chlorpromazine |
| | | Ketamine |
| | | Propranolol |
| | | Fluoxetine |
| | | Ketanserin |
| | | Mesulergine |
| | | AC-90179 |
| | | Ergoloid mesylate 2 |
| | | Methotrimeprazine |
| | | Paliperidone |
| | | Clomipramine |
| | | Trimipramine |
| | | Captodiame |
| | | Nefazodone |
| GABA Receptor | Bamaluzole | bicuculline |
| Accession | GABA | Metrazol |
| Numbers | Gabamide | Flumazenil |
| (Q9UBS5, O95166, | GABOB | Thiothixine |
| O75899, P28472, | Gaboxadol | Bupropion |
| P18507, P47870, | Ibotenic acid | Caffeine |
| P47869, O14764) | Isoguvacine | |
| | Isonipecotic acid | |
| | Muscimol | |
| | Phenibut | |
| | Picamilon | |
| | Progabide | |
| | Quisqualamine | |
| | SL 75102 | |
| | Thiomuscimol | |
| | Alcohols (e.g., ethanol, isopropanol) | |
| | Avermectins (e.g., ivermectin) | |
| | Barbiturates (e.g., phenobarbital) | |
| | Benzodiazepines | |
| | Bromides (e.g., potassium bromide | |
| | Carbamates (e.g., meprobamate, | |
| | carisoprodol) | |
| | Chloralose | |
| | Chlormezanone | |
| | Clomethiazole | |
| | Dihydroergolines (e.g., ergoloid | |
| | (dihydroergotoxine)) | |
| | Etazepine | |
| | Etifoxine | |
| | Imidazoles (e.g., etomidate) | |
| | Kavalactones (found in kava) | |
| | Loreclezole | |
| | Neuroactive steroids (e.g., | |
| | allopregnanolone, ganaxolone) | |
| | Nonbenzodiazepines (e.g., | |
| | zaleplon, zolpidem, zopiclone, | |
| | eszopiclone) | |
| | Petrichloral | |
| | Phenols (e.g., propofol) | |
| | Piperidinediones (e.g., glutethimide, | |
| | methyprylon) | |
| | Propanidid | |
| | Pyrazolopyridines (e.g., etazolate) | |
| | Quinazolinones (e.g., | |
| | methaqualone) | |
| | Skullcap constituents | |
| | Stiripentol | |
| | Sulfonylalkanes (e.g., | |
| | sulfonmethane, tetronal, trional) | |
| | Valerian constituents (e.g., valeric | |
| | acid, valerenic acid) | |
| | Volatiles/gases (e.g., chloral | |
| | hydrate, chloroform, diethyl ether, | |
| | sevoflurane) | |
| Glutamate | 3,5-dihydroxyphenylglycine | APICA |
| Receptor | eglumegad | EGLU |
| Accession | Biphenylindanone A | LY-341,495 |
| Number: | DCG-IV | |
| (P42261, P39086, | L-AP4 | |
| P39086, Q13585, | | |
| P42261, P42262, | | |
| P42263, P48058, | | |
| P39086, Q13002, | | |
| Q13003, Q13003, | | |
| Q16478, Q12879, | | |
| Q14957, Q13224, | | |
| Q14957, O15399, | | |
| Q8TCU5, O60391) | | |
| CNR1/CNR2 | N-Arachidonoylethanolamine | SR 141716A |
| Accession | 2-Arachidonoyl-glycerol | LY-320135 |
| Number: | 2-Arachidonoyl-glycerylether | AM251 |
| (P21554, P34972) | N-Arachidonoyl-dopamine | AM281 |
| | O-Arachidonoyl-ethanolamine | SR 144528 |
| | N-Arachidonoylethanolamine | AM630 |
| | 2-Arachidonoyl-glycerol | |
| | 2-Arachidonoyl-glycerylether | |
| | N-Arachidonoyl-dopamine | |
| | O-Arachidonoyl-ethanolamine | |
| | Δ-9-THC | |
| | CP-55,940 | |
| | R(+)-WIN 55,212-2 | |
| | HU-210 | |
| | Levonantradol | |
| | Nabilone | |
| | Methanandamide | |
| | ACEA | |
| | O-1812 | |
| | Δ9-THC | |
| | CP-55,940 | |
| | R(+)-WIN 55,212-2 | |
| | HU-210 | |
| | Levonantradol | |
| | Nabilone | |
| | Methanandamide | |
| | JWH-015 | |
| | JWH-133 | |

TABLE 8B
ADRENERGIC AGONISTS AND ANTAGONISTS

| Receptor | Agonist | Antagonist |
|---|---|---|
| Non-selective | adrenaline (epinephrine), | carvedilol, arotinolol, and labetalol |
| | noradrenaline (norepinephrine), | |
| | isoprenaline (isoproterenol), | |
| | dopamine, caffeine, nicotine, | |
| | tyramine, methylphenidate, | |
| | ephedrine and pseudophedrine. | |
| α1 selective (ADRA1A, | phenylephrine, methoxamine, | acepromazine, alfuzosin, doxazosin, |
| ADRA1B, ADRA1D) | midodrine, cirazoline, | labetalol, phenoxybenzamine, |
| | xylometazoline, metaraminol | KW3902, phentolamine, prazosin, |
| | chloroehtylclonidine, | tamsulosin, terazosin, tolazoline, |
| | oxymetazoline | trazodone, amitriptyline, silodosin, |
| | | clomipramine, doxepin, trimipramine, |
| | | typical and atypical antipsychotics, |
| | | and antihistamines, such as |
| | | hyroxyzine |
| α2 selective (ADRA2A, | α-methyl dopa, clonidine, | phentolamine, phenoxybenzamine, |
| ADRA2B, ADRA2C) | brimonidine, agmatine, | yohimbine, idazoxan, atipamezole, |
| | dexmedetomidine, | mirtazapine, tolazoline, trazodone, |
| | medetomidine, romifidine | and typical and atypical |
| | chloroethylclonidine, | antipsychotics |
| | detomidine, lofexidine, xylazine, | |
| | tizanidine, guanfacine, and amitraz | |
| β1 selective (ADRB1) | Dobutamine | metroprolol, atenolol, acebutolol, |
| | | bisoprolol, betaxolol, levobetaxolol, |
| | | esmolol, celiprolol, carteolol, |
| | | landiolol, oxprenolol, propanolol, |
| | | practolol, penbutolol, timolol, |
| | | labetalol, nebivolol, levobunolol, |
| | | nadolol, pindolol, sotalol, |
| | | metipranolol, tertatolol, vortioxene |
| β2 selective (ADRB2) | salbutamol, albuterol, bitolterol | butaxamine, acebutolol, timolol, |
| | mesylate, levabuterol, ritodrine, | propanolol, levobunolol, carteolol, |
| | metaproterenol, terbutaline, | labetalol, pindolol, oxprenolol, |
| | salmeterol, formoterol, and | nadolol, metipranolol, penbutolol, |
| | pirbuterol | tertatolol, sotalol |
| β3 selective (ADRB3) | L-796568, amibegron, solabegron, | SR 59230A, arotinolol |
| | mirabegron | |

TABLE 8C
DOPAMINE AGONISTS AND ANTAGONISTS

| Receptor | Agonist | Antagonist |
|---|---|---|
| Non-selective | pramipexole, ropinirole, rotigotine, | haloperidol, paliperidone, clozapine, |
| | apomorphine, | risperidone, olanzapine, quetiapine, |
| | propylnorapomorphine, | ziprasidone, metoclopramide, |
| | bromocriptine, cabergoline, ciladopa, | droperidol, domperidone, amoxapine, |
| | dihydrexidine, dinapsoline, | clomipramine, trimipramine, choline, |
| | doxamthrine, epicriptine, lisuride, | melatonin, acepromazine, |
| | pergolide, piribedil, quinagolide, | amisulpride, asenapine, azaperone, |
| | roxindole, dopamine | benperidol, bromopride, butaclamol, |
| | | chlorpromazine, clebopride, |
| | | chlorprothixene, clopenthixol, |
| | | clocapramine, eticlopride, |
| | | flupenthixol, fluphenazine, |
| | | fluspirilene, hydroxyzine, itopride, |
| | | iodobenzamide, levomepromazine, |
| | | levosulpiride, loxapine, mesoridazine, |
| | | metopimazine, mosapramine, |
| | | nafadotride, nemonapride, |
| | | penfluridol, perazine, perphenazine, |
| | | pimozide, prochlorperazine, |
| | | promazine, pipotiazine, raclopride, |
| | | remoxipride, spiperone, spiroxatrine, |
| | | stepholidine, sulpiride, sultopride, |
| | | tetrahydropalmatine, thiethylperazine, |
| | | thioridazine, thiothixene, tiapride, |
| | | trifluoperazine, trifluperidol, |
| | | triflupromazine, thioproperazine, |
| | | taractan, zotepine, zuclopenthixol, |
| | | ziprasidone, ANP-010, NGD-94-4 |
| D1 (DRD1) | Fenoldopam, A-86929, | SCH-23,390, SKF-83,959, Ecopipam, |
| | dihydrexidine, dinapsoline, | Clebopride, Flupenthixol, |
| | dinoxyline, doxanthrine, SKF-81297, | Zuclopenthixol, Taractan, PSYRX- |
| | SKF-82958, SKF-38393, G-BR-APB, | 101, LuAF-35700, GLC-756, |
| | dopexamine | ADX10061, Zicronapine |
| D2 (DRD2) | Cabergoline, pergolide, quinelorane, | Chloroethylnorapomorphine, |
| | sumanirole, talipexole, piribedil, | desmethoxyfallypride, domperidone, |
| | quinpirole, quinelorane, dinoxyline, | eticlopride, fallypride, hydroxyzine, |
| | dopexamine | itopride, L-741,626, SV 293, |
| | | yohimbine, raclopride, sulpiride, |
| | | paliperidone, penfluridol, quetiapine, |
| | | lurasidone, risperidone, olanzapine, |
| | | blonanserin, perphenazine, |
| | | metoclopramide, trifluoperazine, |
| | | clebopride, levosulpiride, flupenthixol, |
| | | haloperidol, thioridazine, alizapride, |
| | | amisulpride, asenapine, bromopride, |
| | | bromperidol, clozapine, fluphenazine, |
| | | perphanazine, loxapine, |
| | | nemonapride, pericyazine, |
| | | pipamperone, prochlorperazine, |
| | | thioproperazine, thiethylperazine, |
| | | tiapride, ziprasidone, zuclopenthixol, |
| | | taractan, fluanisone, melperone, |
| | | molindone, remoxipride, sultopride, |
| | | ALKS 3831, APD-403, ONC201, |
| | | pridopidine, DSP-1200, NG-101, |
| | | TAK-906, ADN-1184, ADN-2013, |
| | | AG-0098, DDD-016, IRL-626, KP303, |
| | | ONC-206, PF-4363467, PGW-5, CG- |
| | | 209, ABT-925, AC90222, ACP-005, |
| | | ADN-2157, CB030006, CLR-136, |
| | | Egis-11150, Iloperidone, JNJ- |
| | | 37822681, DLP-115, AZ-001, S- |
| | | 33138, SLV-314, Y-931, YKP1358, |
| | | YK-P1447, APD405, CP-903397, |
| | | ocaperidone, zicronapine, TPN-902 |
| D3 (DRD3) | Piribedil, quinpirole, captodiame, | Domperidone, FAUC 365, |
| | compound R, R-16, FAUC 54, FAUC | nafadotride, raclopride, PNU-99,194, |
| | 73, PD-128,907, PF-219,061, PF- | SB-277011-A, sulpiride, risperidone, |
| | 592,379, CJ-1037, FAUC 460, FAUC | YQA14, U99194, SR 21502, |
| | 346, cariprazine | levosulpiride, amisulpride, |
| | | nemonapride, ziprasidone, taractan, |
| | | sultopride, APD-403, F17464, |
| | | ONC201, NG-101, TAK-906, ONC- |
| | | 206, PF-4363467, ABT-127, ABT- |
| | | 614, GSK-598809, GSK-618334, |
| | | S-14297, S-33138, YKP1358, YK- |
| | | P1447 |
| D4 (DRD4) | WAY-100635, A-412,997, ABT-724, | A-381393, FAUC 213, L-745,870, L- |
| | ABT-670, FAUC 316, PD-168, 077, | 570,667, ML-398, fananserin, |
| | CP-226,269 | clozapine, PNB-05, SPI-376, SPI- |
| | | 392, Lu-35-138, NGD-94-1 |
| D5 (DRD5) | Dihydrexidine, rotigotine, SKF- | SCH 23390 |
| | 83,959, fenoldopam, | |
| Partial | aplindore, brexpiprazole, | |
| | aripiprazole, CY-208,243, | |
| | pardoprunox, phencyclidine, and | |
| | salvinorin A | |

TABLE 8D
GABA AGONISTS AND ANTAGONISTS

| Receptor | Agonist | Antagonist |
|---|---|---|
| GABA_{A} | barbiturates (e.g., allobarbital, | bicuculline, gabazine, hydrastine, |
| | amobarbital, aprobarbital, alphenal, | pitrazepin, sinomenine, tutin, |
| | barbital, brallobarbital, phenobarbital, | thiocolchicoside, metrazol, |
| | secobarbital, thiopental), bamaluzole, | securinine, gabazine |
| | GABA, GABOB, gaboxadol, ibotenic | |
| | acid, isoguvacine, isonipecotic acid, | |
| | muscimol, phenibut, picamilon, | |
| | progabide, quisqualamine, SL 75102, | |
| | thiomuscimol, positive allosteric | |
| | modulators (PAMs) (e.g., alcohols, | |
| | such as ethanol and isopropanol; | |
| | avermectins, such as ivermectin; | |
| | benzodiazepines, such as diazepam, | |
| | alprazolam, chlordiazepoxide, | |
| | clonazepam, flunitrazepam, | |
| | lorazepam, midazolam, oxazepam, | |
| | prazepam, brotizolam, triazolam, | |
| | estazolam, lormetazepam, | |
| | nitrazepam, temazepam, flurazepam, | |
| | clorazepate halazepam, prazepam, | |
| | nimetazapem, adinazolam, and | |
| | climazolam; bromides, such as | |
| | potassium bromide; carbamates, | |
| | such as meprobamate and | |
| | carisoprodol; chloralose; | |
| | chlormezanone; chlomethiazole; | |
| | dihydroergolines, such as ergoloid; | |
| | etazepine; etifoxine; imidazoles, such | |
| | as etomidate; imidazopyridines, such | |
| | as alpidem and necopdiem; | |
| | kavalactones; loreclezole; | |
| | neuroactive steroids, such as | |
| | allogregnanolone, pregnanolone, | |
| | dihydrodeoxycorticosterone, | |
| | tetrahydrodeoxycortisosterone, | |
| | androstenol, androsterone, | |
| | etiocholanolone, 3α-androstanediol, | |
| | 5α, 5β, or 3α-dihydroprogesterone, | |
| | and ganaxolone; | |
| | nonbenzodiazepines, such as | |
| | zalepon, zolpidem, zopiclone, and | |
| | eszopiclone; petrichloral; phenols, | |
| | such as propofol; piperidinediones, | |
| | such as glutethimide and | |
| | methyprylon; propanidid; | |
| | pyrazolopyridines, such as etazolate; | |
| | pyrazolopyrimidines, such as | |
| | divaplon and fasiplon; | |
| | cyclopyrrolones, sush as pagoclone | |
| | and suproclone; β-cabolines, such as | |
| | abecarnil and geodecarnil; | |
| | quinazolinones, such as | |
| | methaqualone; Scutellaria | |
| | constituents; stiripentol; | |
| | sulfonylalkanes, such as | |
| | sulfonomethane, teronal, and trional; | |
| | Valerian constituents, such as valeric | |
| | acid and valerenic acid; and gases, | |
| | such as chloral hydrate, chloroform, | |
| | homotaurine, diethyl ether, and | |
| | sevoflurane. | |
| GABA_{B} | 1,4-butanediol, baclofen, GABA, | CGP-35348, homotaurine, |
| | Gabamide, GABOB, gamma- | phaclofen, saclofen, and |
| | butyrolactone, gamma- | SCH-50911 |
| | hydroxybutyric acid, gamma- | |
| | hyrdoxyvaleric acid, gamma- | |
| | valerolactone, isovaline, | |
| | lesogaberan, phenibut, picamilon, | |
| | progabide, homotaurine, SL-75102, | |
| | tolgabide | |
| GABA_{A}-ρ | CACA, CAMP, GABA, GABOB, N4- | gabazine, gaboxadol, isonipecotic |
| | chloroacetylcytosine arabinoside, | acid, SKF-97,541, and (1,2,5,6- |
| | picamilon, progabide, tolgabide, and | Tetrahydropyridin-4- |
| | neuroactive steroids, such as | yl)methylphosphinic acid |
| | allopregnanolone, THDOC, and | |
| | alphaxolone | |

TABLE 8E
MUSCARINC AGONISTS AND ANTAGONISTS

| Receptor | Agonist | Antagonist |
|---|---|---|
| Chrm1 | AF102B, AF150(S), AF267B, | atropine, dicycloverine, hyoscyamine, |
| | acetylcholine, carbachol, cevimeline, | ipratropium, mamba toxin muscarinic |
| | muscarine, oxotremorine, | toxin 7 (MT7), olanzapine, oxybutynin, |
| | pilocarpine, vedaclidine, 77-LH-28-1, | pirenzepine, telenzepine, and |
| | CDD-0097, McN-A-343, L689,660, | tolterodine |
| | and xanomeline | |
| Chrm2 | acetylcholine, methacholine, iper-8- | atropine, dicycloverine, |
| | naph, berbine, and (2S,2′R,3′S,5′R)- | hyoscyamine, otenzepad, AQRA-741, |
| | 1-methyl-2-(2-methyl-1,3-oxathiolan- | AFDX-384, thorazine, |
| | 5-yl)pyrrolidine 3-sulfoxide methyl | diphenhydramine, dimenhydrinate, |
| | iodide | ipratropium, oxybutynin, pirenzepine, |
| | | methoctramine, tripitramine, |
| | | gallamine, and tolterodine |
| Chrm3 | acetylcholine, bethanechol, | atropine, dicycloverine, hyoscyamine, |
| | carbachol, L689, 660, oxotremorine, | alcidium bromide, 4-DAMP, |
| | pilocarpine, aceclidine, arecoline, | darifenacin, DAU-5884, HL-031,120, |
| | and cevimeline | ipratropium, J-104,129, oxybutynin, |
| | | tiotropium, zamifenacin, and |
| | | tolterodine |
| Chrm4 | acetylcholine, carbachol, and | AFDX-384, dicycloverine, himbacine, |
| | oxotremorine), and Chrm5 agonists | mamba toxin 3, PD-102,807, PD- |
| | (e.g., acetylcholine, milameline, | 0298029, and tropicamide |
| | sabcomeline | |
| Chrm5 | acetylcholine, milameline, | VU-0488130, xanomeline |
| | sabcomeline | |
| Non- | | scopolamine, hydroxyzine, |
| selective | | doxylamine, dicyclomine, flavoxate, |
| | | cyclopentolate, atropine methonitrate, |
| | | trihexyphenidyl/benzhexol, |
| | | solifenacin, benzatropine, |
| | | mebeverine, and procyclidine |

TABLE 8F
NICOTINIC AGONISTS AND ANTAGONISTS

| Receptor | Agonist | Antagonist |
|---|---|---|
| Chrna | choline, acetylcholine, carbachol, | turbocurarine, bupropion, |
| receptors | methacholine, nicotine, varenicline | mecamylamine, 18- |
| | tartrate, galantamine hydrobromide, | methozycoronaridine, |
| | suxamethonium chloride | hexamethonium, trimethaphan, |
| | (succinylcholine chloride), | atraciurium, doxacurium, mivacurium, |
| | epibatidine, iobeline, | pancuronium, vecuronium, |
| | decamethonium, isopronicline/TC- | succinylcholine, dextromethorphan, |
| | 1734/AZD3480 (TC-1734), AZD1446 | neramexane, dextrophan, and 3- |
| | (TC-6683), TC-5619, TC-5214, MEM | methoxymorphinan |
| | 3454 (RG3487), ABT-894, ABT-560, | |
| | EVP-6124, EVP-4473, PNU-282987, | |
| | AR-R17779, SSR 189711, JN403, | |
| | ABBF, PHA-543613, SEN12333, | |
| | GTS-21/DMXB-A, AZD0328, A- | |
| | 582941, ABT-418, 5-iodo-A-85380, | |
| | SIB-1765F, ABT-089, and ABT-594 | |

TABLE 8G
SEROTONIN AGONISTS AND ANTAGONISTS

| Receptor | Agonist | Antagonist |
|---|---|---|
| 5-HT_{1A} | azapirones, such as alnespirone, | pindolol, tertatolol, alprenolol, AV- |
| | binosperone, buspirone, | 965, BMY-7,378, cyanopindolol, |
| | enilospirone, etapirone, geprione, | dotarizine, flopropione, GR-46,611, |
| | ipsaprione, revospirone, zalospirone, | iodocyanopindolol, isamoltane, |
| | perospirone, tiosperone, | lecozotan, mefway, methiothepin, |
| | umespirone, and tandospirone; 8- | methysergide, MPPF, NAN-190, |
| | OH-DPAT, befiradol, F-15,599, | oxprenolol, pindobind, propanolol, |
| | lesopitron, MKC-242, LY-283,284, | risperidone, robalzotan, SB-649,915, |
| | osemozotan, repinotan, U-92,016-A, | SDZ-216,525, spiperone, spiramide, |
| | RU-24969, 2C-B, 2C-E, 2C-T-2, | spiroxatrine, UH-301, WAY-100,135, |
| | aripiprazole, asenapine, bacoside, | WAY-100,635, and xylamidine |
| | befiradol, brexpiprazole, bufotenin, | |
| | cannabidiol, and fibanserin | |
| 5-HT_{1B} | triptans, such as sumatriptan, | methiothepin, yohimbine, |
| | rizatriptan, eletriptan, donitripatn, | metergoline, aripiprazole, isamoltane, |
| | almotriptan, frovatriptan, avitriptan, | AR-A000002, SB-216,641, SB- |
| | zolmitriptan, and naratriptan; | 224,289, GR-127,935, SB-236,057 |
| | ergotamine, 5- | |
| | carboxamidotryptamine, CGS- | |
| | 12066A, CP-93,129, CP-94,253, CP- | |
| | 122,288, CP-135,807, RU-24969, | |
| | vortioxetine, ziprasidone, and | |
| | asenapine | |
| 5-HT_{1D} | triptans, such as sumatriptan, | ziprasidone, methiothepin, |
| | rizatriptan, and naratriptan; | yohimbine, metergoline, ergotamine, |
| | ergotamine, 5-(nonyloxy)tryptaime, | BRL-15572, vortioxetine, GR- |
| | 5-(t-butyl)-N-methyltryptamine, CP- | 127,935, LY-310,762, LY-367,642, |
| | 286,601, PNU-109,291, PNU- | LY-456,219, and LY-456,220 |
| | 142,633, GR-46611, L-694,247, L- | |
| | 772,405, CP-122,288, and CP- | |
| | 135,807 | |
| 5-HT_{1E} | BRL-54443, eletriptan | |
| 5-HT_{1F} | LY-334,370, 5-n-butyryloxy-DMT, | |
| | BRL-54443, eletriptan, LY-344,864, | |
| | naratriptan, and lasmiditan | |
| 5-HT_{2A} | 25I-NBOH, 25I-NBOMe, (R)-DOI, | cyproheptadine, methysergide, |
| | TCB-2, mexamine, O-4310, PHA- | quetiapine, nefazodone, olanzapine, |
| | 57378, OSU-6162, 25CN-NBOH, | asenapine, pizotifen, LY-367,265, |
| | juncosamine, efavirenz, mefloquine, | AMDA, hydroxyzine, 5-MeO-NBpBrT, |
| | lisuride, and 2C-B | and niaprazine |
| 5-HT_{2B} | fenfluramine, pergolide, cabergoline, | agomelatine, aripiprazole, |
| | mefloquine, BW-723C86, Ro60- | sarpogrelate, lisuride, tegaserod, |
| | 0175, VER-3323, 6-APB, | metadoxine, RS-127,445, SDZ SER- |
| | guanfacine, norfenfluramine, 5-MeO- | 082, EGIS-7625, PRX-08066, SB- |
| | DMT, DMT, mCPP, aminorex, | 200,646, SB-204,741, SB-206,553, |
| | chlorphentermine, MEM, MDA, LSD, | SB-215,505, SB-228,357, LY- |
| | psilocin, MDMA | 266,097, and LY-272,015 |
| 5-HT_{2C} | lorcaserin, lisuride, A-372,159, AL- | agomelatine, CPC, eltoprazine, |
| | 38022A, CP-809,101, fenfluramine, | etoperidone, fluoxetine, FR-260,010, |
| | mesulergine, MK-212, | LU AA24530, methysergide, |
| | naphthyllisopropylamine, | nefazodone, norfluoxetine, O- |
| | norfenfluramine, ORG-12,962, ORG- | desmethyltramadol, RS-102,221, SB- |
| | 37,684, oxaflozane, PNU-22395, | 200,646, SB-221,284, SB-242,084, |
| | PNU-181731, lysergamides, | SDZ SER-082, tramadol, and |
| | phenethylamines, piperazines, | trazodone |
| | tryptamines, Ro60-0175, | |
| | vabicaserin, WAY-629, WAY- | |
| | 161,503, WAY-163,909, and YM-348 | |
| 5-HT_{2A/2C} | | ketanserin, risperidone, trazodone, |
| | | mirtazapine, clozapine |
| 5-HT₃ | 2-methyl-5-HT, alpha- | dolasetron, granisetron, ondansetron, |
| | methyltryptamine, bufotenin, | palonosetron, tropisetron, alosetron, |
| | chlorophenylbiguanide, ethanol, | cilanosetron, mirtazapine, AS-8112, |
| | ibogaine, phenylbiguanide, | bantopride, metroclopramide, |
| | quipazine, RS-56812, SR-57227, | renzapride, zacopride, mianserin, |
| | varenicline, and YM-31636 | vortioxetine, clozapine, olanzapine, |
| | | quetiapine, menthol, thujone, |
| | | lamotigrine, and 3-tropanyl indole-3- |
| | | carboxylate |
| 5-HT₄ | cisapride, tegaserod, prucalopride, | piboserod, GR-113,808, GR- |
| | BIMU-8, CJ-033,466, ML-10302, | 125,487, RS-39604, SB-203,186, |
| | mosapride, renzapride, RS-67506, | SB-204,070, and chamomile |
| | RS-67333, SL65.1055, zacopride, | |
| | metoclopramide, and sulpride | |
| 5-HT_{5A} | valeronic acid | ASP-5736, AS-2030680, AS- |
| | | 2674723, latrepiridine, risperidone, |
| | | and SB-699,551 |
| 5-HT₆ | EMDT, WAY-181,187, WAY- | ALX-1161, AVN-211, BVT-5182, |
| | 208,466, N-(inden-5- | BVT-74316, cerlapiridine, EGIS- |
| | yl)imidazothiazole-5-sulfonamide, E- | 12233, idalopiridine, interpridine, |
| | 6837, E-6801, and EMD-386,088 | latrepiridine, MS-245, PRX-07034, |
| | | SB-258,585, SB-271,046, SB- |
| | | 357,134, SB-339,885, Ro 04-6790, |
| | | Ro-4368554, sertindole, olanzapine, |
| | | asenapine, clozapine, rosa rugosa |
| | | extract, and WAY-255315 |
| 5-HT₇ | AS-19, 5-CT, 5-MeOT, 8-OH-DAPT, | amisulpride, amitriptyline, |
| | aripiprazole, E-55888, E-57431, LP- | amoxapine, clomipramine, clozapine, |
| | 12, LP-44, MSD-5a, RA-7, and N,N- | DR-4485, fluphenazine, fluperlapine, |
| | Dimethyltryptamine | ICI 169,369, imipramine, ketanserine, |
| | | JNJ-18038683, loxapine, lurasidone, |
| | | LY-215,840, maprotiline, |
| | | methysergide, mesulergine, |
| | | mianserin, olanzepine, pimozide, |
| | | ritanserin, SB-258,719, SB-258,741, |
| | | SB-269,970, SB-656,104-A, SB- |
| | | 691,673, sertindole, spiperone, |
| | | tenilapine, TFMPP, vortioxetine, |
| | | trifluoperazine, ziprasidone, and |
| | | zotepine |
| Non- | | chlorpromazine, cyproheptadine, |
| selective | | pizotifen, oxetorone, spiperone, |
| 5-HT | | ritanserin, parachlorophenylalanine, |
| antagonists | | metergoline, propranolol, mianserin, |
| | | carbinoxamine, methdilazine, |
| | | promethazine, pizotifen, oxatomide, |
| | | feverfew, fenclonin, and reserpine |

TABLE 8H
GLUATAMATE RECEPTOR AGONISTS AND ANTAGONISTS

| Receptor | Agonist | Antagonist |
|---|---|---|
| lonotropic | AMPA, glutamic | AP5, AP7, CPPene, selfotel, |
| (GRIA-14, | acid, ibotenic | HU-211, Huperzine A, gabapentin, |
| GRIK1-5, | acid, kainic acid, | remacemide, amantadine, |
| and GRIN1- | NMDA, | atomoxetine, AZD6765, agmatine, |
| 3B) | quisqualic acid | chloroform, dextrallorphan, |
| | | dextromethorphan, dextrorphan, |
| | | diphenidine, dizocilpine (MK-801), |
| | | ethanol, eticyclidine, gacyclidine, |
| | | ibogaine, ifenprodil, ketamine, |
| | | kynurenic acid, memantine, |
| | | magnesium, methoxetamine, |
| | | nitromemantine, nitrous oxide, PD- |
| | | 137889, perampanel, phencyclidine, |
| | | rolicyclidine, tenocyclidine, |
| | | methoxydine, tiletamine, |
| | | neramexane, eliprodil, etoxadrol, |
| | | dexoxadrol, WMS-2539, NEFA, |
| | | delucemine, 8A-PDHQ, aptiganel, |
| | | rhynchophylline |
| Metabotropic | L-AP4, ACPD, | AIDA, fenobam, MPEP, |
| (GRM1-8) | L-QA, CHPG, | LY-367,385, EGLU, CPPG, |
| | LY-379,268, | MAP4, MSOP, LY-341,495 |
| | LY-354,740, | |
| | ACPT, | |
| | VU 0155041 | |
| Glycine | | rapastinel, NRX-1074, 7- |
| antagonists | | chlorokynurenic acid, 4- |
| | | chlorokynurenine, 5,7- |
| | | dichlorokynurenic acid, |
| | | kynurenic acid, TK-40, 1- |
| | | aminocyclopropanecarboxylic |
| | | acid (ACPC), L-phenylalanine, |
| | | and xenon |

TABLE 8I
HISTAMINE AGONISTS AND ANTAGONISTS

| Receptor | Agonist | Antagonist |
|---|---|---|
| Non- | histamine dihydrochloride, HTMT | |
| selective | dimaleate, 2-pyridylethlyamine | |
| | dihydrochloride | |
| H₁ | | acrivastine, azelastine, astemizole, |
| | | bilastine, bromodiphenhydramine, |
| | | brompheniramine, buclizine, |
| | | carbinoxamine, cetirizine, cetirizine |
| | | dihydrochloride, clemastine fumarate, |
| | | clemizole hydrochloride, |
| | | chlorodiphenhydramine, |
| | | chlorphenamine, chlorpromazine, |
| | | clemastine, cyclizine, |
| | | cyproheptadine, |
| | | dexbrompheniramine, |
| | | dexchlorpheniramine, |
| | | dimenhydrinate, dimethindene |
| | | maleate, dimetindene, |
| | | diphenhydramine, diphenhydramine |
| | | hydrochloride, doxepin hydrochloride, |
| | | doxylamine, ebastine, embramine, |
| | | fexofenadine, fexofenadine |
| | | hydrochloride, hydroxyzine, ketotifen |
| | | fumarate, loratadine, meclizine, |
| | | meclizine dihydrochloride, |
| | | mepyramine maleate, mirtazapine, |
| | | olopatadine, olopatadine |
| | | hydrochloride, orphenadrine, |
| | | phenindamine, pheniramine, |
| | | phenyltoloxamine, promethazine, |
| | | quetiapine, rupatadine, terfenadine, |
| | | tripelennamine, zotepine, trans- |
| | | triprolidine hydrochloride, and |
| | | triprolidine |
| H₁ inverse | | cetirizine, levocetirizine, |
| agonists | | desloratadine, and pyrilamine |
| H₂ | betazole, impromidine, dimaprit | aminopotentidine, cimetidine, |
| | dihydrochloride, and amthamine | famotidine, ICI 162,846, lafutidine, |
| | dihyrdobromide | nizatidine, ranitidine, ranitidine |
| | | hyrdochloride, roxatidine, zolantadine |
| | | dimaleate, and toitidine |
| H₃ | imetit dihydropbromide, immepip | clobenpropit, clobenpropit |
| | dihyrdrobromide, immethridine | dihydrobromide, A 3314440 |
| | dihydrobromide, α-Methylhistamine | dihyrdochloride, BF 2649 |
| | dihydrobromide, N-methylhistamine | hydrochloride, carcinine |
| | dihydrochloride, proxyfan oxalate, | ditrifluoroacetate, ABT-239, |
| | and betahistine | ciprofaxin, conessine, GT 2016, A- |
| | | 349,821, impentamine |
| | | dihydrobromide, iodophenpropit |
| | | dihydrobromide, JNJ 10181457 |
| | | dihydrochloride, JNJ 5207852 |
| | | dihydrochloride, ROS 234 dioxalate, |
| | | SEN 12333, VUF 5681 |
| | | dihydrobromide, and thioperamide |
| H₄ | imetit dihydropbromide, immepip | thioperamide, JNJ 7777120, A |
| | dihyrdrobromide, 4-methylhistamine | 943931 dihydrochloride, A 987306, |
| | dihydrochloride, clobenpropit | JNJ 10191584 maleate, and VUF- |
| | dihydrobromide, VUF 10460, and | 6002 |
| | VUF 8430 dihydrobromide | |

TABLE 8J
CANNABINOID AGONISTS AND ANTAGONISTS

| Receptor | Agonist | Antagonist |
|---|---|---|
| Cannabinoid receptor | Anandamide, N-Arachidonoyl | |
| (non-selective) | dopamine, 2-Arachidonoylglycerol | |
| | (2-AG), 2-Arachidonyl glyceryl ether, | |
| | Δ-9-Tetrahydrocannabinol, EGCG, | |
| | Yangonin, AM-1221, AM-1235, AM- | |
| | 2232, UR-144, JWH-007, JWH-015, | |
| | JWH-018, ACEA, ACPA, arvanil, CP | |
| | 47497, DEA, leelamine, | |
| | methanandamide, NADA, noladin | |
| | ether, oleamide, CB 65, GP-1a, GP- | |
| | 2a, GW 405833, HU 308, JWH-133, | |
| | L-759,633, L-759,656, LEI 101, MDA | |
| | 19, and SER 601 | |
| CB₁ receptor | ACEA, ACPA, RVD-Hpα, (R)-(+)- | rimonabant, cannabidiol, Δ⁹- |
| | methanandamide | tetrahydrocannabivarin (THCV), |
| | | taranabant, otenabant, surinabant, |
| | | rosonabant, SLV-319, AVE1625, |
| | | V24343, AM 251, AM 281, AM 6545, |
| | | hemopressin, LY 320135, MJ 15, CP |
| | | 945598, NIDA 41020, PF 514273, |
| | | SLV 319, SR 1141716A, and TC-C |
| | | 14G |
| CB₂ receptor | CB 65, GP 1a, GP 2a, GW 405833, | cannabidiol, Δ⁹- |
| | HU 308, JWH 133, L-759,656, L- | tetrahydrocannabivarin (THCV), AM |
| | 759,633, SER 601, LEI 101 | 630, COR 170, JTE 907, and SR |
| | | 144528 |

TABLE 8K
PURINERGIC RECEPTOR AGONISTS AND ANTAGONISTS

| Receptor | Agonist | Antagonist |
|---|---|---|
| ADORA1 (P1 | Adenosine, N6- | Caffeine, theophylline, 8- |
| adenosine receptor) | Cyclopentyladenosine, N6-3- | Cyclopenty1-1,3-dimethylxanthine |
| | methoxyl-4-hydroxybenzyl adenine | (CPX), 8-Cyclopenty1-1,3- |
| | riboside (B2), CCPA, tecadenoson, | dipropylxanthine (DPCPX), 8- |
| | selodenoson, Certain | Phenyl-1,3-dipropylxanthine, |
| | Benzodiazepines and Barbiturates, | bamifylline, BG-9719, BG09928, FK- |
| | 2′-MeCCPA, GR 79236, and SDZ | 453, FK838, rolofylline, N-0861, and |
| | WAG 994 | PSB 36 |
| ADORA2A (P1 | Adenosine, N6-3-methoxyl-4- | Caffeine, theophylline, istradefylline, |
| adenosine receptor) | hydroxybenzyl adenine riboside (B2), | SCH-58261, SCH-442,416, ATL- |
| | YT-146, DPMA, UK-423,097, | 444, MSX-3, preladenant, SCH- |
| | limonene, NECA, CV-3146, | 412,348, VER-6623, VER-6947, |
| | binodenoson, ATL-146e, CGS- | VER-7835, vipadenant, and ZM- |
| | 21680, and Regadenoson | 241,385 |
| ADORA2B (P1 | Adenosine, 5′-N- | Caffeine, theophylline, CVT-6883, |
| adenosine receptor) | ethylcarboxamidoadenosine, BAY | ATL-801, compound 38, MRS-1706, |
| | 60-6583, LUF-5835, NECA, (S)- | MRS-1754, OSIP-339,391, PSB- |
| | PHPNECA, and LUF-5845 | 603, PSB-0788, and PSB-1115 |
| ADORA3 (P1 | Adenosine, 2-(1-Hexynyl)-N- | Caffeine, theophylline, MRS-1191, |
| adenosine receptor) | methyladenosine, CF-101 (IB- | MRS-1220, MRS-1334, MRS-1523, |
| | MECA), CF-102, 2-C1-IB-MECA, | MRS-3777, MRE3008F20, |
| | CP-532,903, inosine, LUF-6000, | MRE3005F20, OT-7999, |
| | and MRS-3558 | SSR161421, KF-26777, PSB-10, |
| | | PSB-11, and VUF-5574 |
| P2Y receptor | ATP, ADP, UTP, UDP, UDP-glucose, | clopidogrel, elinogrel, prasugrel, |
| | 2-methylthioladenosine 5′ | ticlopidine, ticagrelor, AR-C |
| | diphosphate (2-MeSADP), | 118925XX, AR-C 66096, AR-C |
| | lysophosphatidic acid, PSB 1114, | 69931, AZD 1283, MRS 2179, MRS |
| | PSB 0474, NF 546, MRS 2365, MRS | 2211, MRS 2279, MRS 2500, MRS |
| | 2690, MRS 2693, MRS 2768, MRS | 2578, NF 157, NF 340, PPADS, |
| | 2905, MRS 2957, MRS 4062, and | PPTN hydrochloride, PSD 0739, |
| | denufosol (P2Y₂agonist) | SAR 216471, and suramin |
| P2X receptor | ATP | A 438079, A 740003, A 804598, A |
| | | 839977, AZ 10606120, AZ |
| | | 11645373, 5-BDBD, BX 430, Evans |
| | | Blue, JNJ 47965567, KN-62, NF |
| | | 023, NF 110, NF 157, NF 279, NF |
| | | 449, PPADS, iso-PPADS, PPNDS, |
| | | Ro 0437626, Ro 51, RO-3, TC-P |
| | | 262, suramin, TNP-ATP, and P2X₇ |
| | | antagonists NF279, calmidazolium, |
| | | and KN-62 |

TABLE 9
NEUROTRANSMISSION MODULATORS

| Type | Modulators |
|---|---|
| Norepinephrine reuptake inhibitors | amedalin, atomoxetine, CP-39,332, daledalin, |
| (increase adrenergic neurotransmission) | edivoxetine, esreboxetine, lortalamine, nisoxetine, |
| | reboxetine, talopram, talsupram, tandamine, |
| | viloxazine, bupropion, ciclazindol, manifaxine, |
| | maprotiline, radafaxine, tapentadol, teniloxazine, |
| | protriptyline, nortriptyline, and desipramine |
| Norepineprhine-dopamine reuptake inhibitors | amineptine, bupropion, desoxypipradrol, |
| (increase adrenergic and dopamine | dexmethylphenidate, difemetorex, diphenylprolinol, |
| neurotransmission) | ethylphenidate, fencamfamine, fencamine, |
| | lefetamine, methylenedioxypyrovalerone, |
| | methylphenidate, nomifensine, O-2172, oxolinic |
| | acid, pipradrol, prolintane, pyrovalerone, |
| | tametraline, and WY-46824 |
| Serotonin-norepinephrine-dopamine reuptake | mazindol, nefazodone, sibutramine, venlafaxine, |
| inhibitors (SNDRIs) and serotonin-norepinephrine | esketamine, duloxetine, ketamine, phencyclidine, |
| reuptake inhibitors (SNRIs) | tripelennamine, mepiprazole, amitifadine, AN788, |
| (increase adrengergic, dopamine, and serotonin | ansofaxine, centanafadine, atomoxetine, |
| neurotransmission) | desvenlafaxine, milnacipran, levomilnacipran, |
| | dasotraline, Lu AA34893, Lu AA37096, NS-2360, |
| | tedatioxetine, tesofensine, bicifadine, BMS- |
| | 866,949, brasofensine, diclofensine, DOV-216,303, |
| | EXP-561, liafensine, NS-2359, RG-7166, SEP- |
| | 227,162, SEP-228,425, SEP-228,432, naphyrone, |
| | 3,3-Diphenylcyclobutanamine, 3,4- |
| | Dichlorotametraline, D-161, desmethylsertraline, |
| | DMNPC, DOV-102,677, fezolamine, |
| | GSK1360707F, indatraline, JNJ-7925476, JZ-IV- |
| | 10, JZAD-IV-22, LR-5182, methylnaphthidate, MI-4, |
| | PRC200-SS, PRC050, PRC025, SKF-83,959, TP1, |
| | phenyltropanes (e.g., WF-23, dichloropane, and |
| | RTI-55), Ginkgo biloba extract, St John's Wort, |
| | hyperforin, adhyperforin, and uliginosin B |
| Dopamine reuptake inhibitors | Dopamine reuptake inhbiitors (e.g., altropane, |
| (increase dopamine neurotransmission) | amfonelic acid, amineptine, BTCP, 3C-PEP, DBL- |
| | 583, difluoropine, GBR-12783, GBR-12935, GBR- |
| | 13069, GBR-13098, GYKI-52895, lometopane, |
| | methylphenidate, ethylphenidate, modafinil, |
| | armodafinil, RTI-229, vanoxerine, adrafinil, |
| | benztropine, bupropion, fluorenol, medifoxamine, |
| | metaphit, rimcazole, venlafaxine, Chaenomeles |
| | speciosa, and oroxylin A), dopamine releasing |
| | agents (e.g., p-Tyramine), dextroamphetamine, |
| | lisdexamfetamine, dexmethylphenidate, and |
| | cathinone |
| Dopamine prodrugs | Levopoda, docarpamine |
| (increase dopamine neurotransmission) | |
| GABA reuptake inhibitors | CL-996, deramciclane, gabaculine, guvacine, |
| (increase GABA neurotransmission) | nipecotic acid, NNC-711, NNC 05-2090, SKF- |
| | 89976A, SNAP-5114, tiagabine, and hyperforin |
| GABA analogs | gabapentin, butyric acid, valproic acid, valpromide, |
| (increase GABA neurotransmission) | valnoctamide, 3-hydroxybutanal, GHB, sodium, |
| | oxybate, aceburic acid, GBL, GHBAL, GHV, GVL, |
| | GHC, GCL, HOCPCA, UMB68, pregabalin, tolibut, |
| | phaclofen, sacolfen, arecaidine, gaboxadol, |
| | isonipecotic acid, 3-Methyl-GABA, AABA, BABA, |
| | DAVA, GAVA, Glutamic acid, hopantenic acid, |
| | piracetam, and vigabatrin |
| GABA prodrugs | L-Glutamine, N-lsonicotinoyl-GABA, picamilon, |
| (increase GABA neurotransmission) | progabide, tolgabide |
| Acetylcholinesterase inhibitors | carbamates, physostigmine, neostigmine, |
| (increase nicotinic and muscarinic | pyridostigmine, ambenonium, demecarium, |
| neurotransmission) | rivastigmine, phenanthrene derivatives, |
| | galantamine, caffeine, rosmarinic acid, alpha- |
| | pinene, piperidines, donepezil, tacrine, |
| | edrophonium, Huperzine A, ladostigil, ungeremine, |
| | lactucopicrin, dyflos, echothiophate, parathion, and |
| | quasi-irreversible acetylcholinesterase inhibitors |
| Serotonin reuptake inhibitors | alaproclate, cericlamine, citalopram, dapoxetine, |
| (increase serotonin neurotransmission) | escitalopram, femoxetine, fluoxetine, fluvoxamine, |
| | ifoxetine, indalpine, omiloxetine, panuramine, |
| | paroxetine, pirandamine, RTI-353, sertraline, |
| | zimelidine, desmethylcitalopram, |
| | didesmethylcitalopram, seproxetine ((S)- |
| | norfluoxetine), desvenlafaxine, cianopramine, |
| | litoxetine, lubazodone, SB-649,915, trazodone, |
| | vilazodone, vortioxetine, dextromethorphan, |
| | dextropropoxyphene, dimenhydrinate, |
| | diphenhydramine, mepyramine (pyrilamine), |
| | mifepristone, delucemine, mesembrenone, |
| | mesembrine, roxindole, duloxetine, |
| | levomilnacipran, milnacipran, dapoxetine, |
| | sibutramine, chlorpheniramine, |
| | dextropmethorphan, and methadone |
| Serotonin releasing agents | chlorphentermine, cloforex, dexfenfluramine, |
| (increase serotonin neurotransmission) | etolorex, fenfluramine, flucetorex, indeloxazine, |
| | levofenfluramine, tramadol, carbamazepine, |
| | amiflamine (FLA-336), viqualine (PK-5078), 2- |
| | Methyl-3,4-methylenedioxyamphetamine (2-Methyl- |
| | MDA), 3-Methoxy-4-methylamphetamine (MMA), 3- |
| | Methyl-4,5-methylenedioxyamphetamine (5-Methyl- |
| | MDA), 3,4-Ethylenedioxy-N-methylamphetamine |
| | (EDMA), 4-Methoxyamphetamine (PMA), 4- |
| | Methoxy-N-ethylamphetamine (PMEA), 4-Methoxy- |
| | N-methylamphetamine (PMMA), 4- |
| | Methylthioamphetamine (4-MTA), 5-(2- |
| | Aminopropyl)-2,3-dihydrobenzofuran (5-APDB), 5- |
| | Indanyl-2-aminopropane (IAP), 5-Methoxy-6- |
| | methylaminoindane (MMAI), 5-Trifluoromethyl-2- |
| | aminoindane (TAI), 5,6-Methylenedioxy-2- |
| | aminoindane (MDAI), 5,6-Methylenedioxy-N- |
| | methyl-2-aminoindane (MDMAI), 6-Chloro-2- |
| | aminotetralin (6-CAT), 6-Tetralinyl-2-aminopropane |
| | (TAP), 6,7-Methylenedioxy-2-aminotetralin (MDAT), |
| | 6,7-Methylenedioxy-N-methyl-2-aminotetralin |
| | (MDMAT), N-Ethyl-5-trifluoromethyl-2-aminoindane |
| | (ETAI), N-Methyl-5-indanyl-2-aminopropane, |
| | aminorex, MDMA, MDEA, MDA, MBDB, and |
| | tryptamines, such as DMT, αMT, 5MeO-NMT, |
| | NMT, NETP, Dimethyl-Serotonin, 5MeO-NET, αET |
| | and αMT |
| Excitatory amino acid reuptake inhibitors | didydrokanic acid, WAY-213,613, L-trans-2,4-PDC, |
| (increase Glutamate receptor neurotransmission) | amphetamine, and L-Theanine |
| Glycine reuptake inhibitors | bitopertin, Org 24598, Org 25935, ALX-5407, |
| (increase Glutamate receptor neurotransmission) | sacrosine, Org 25543, and N-arachidonylglycerine |
| Histidine decarboxylase inhibitors | Tritoqualine, catechin |
| (decrease histamine neurotransmission) | |
| Endocannabinoid enhancers | AM404, fatty acid amide hydrolase inhibitors (e.g., |
| (increase cannabinoid neurotransmission) | AM374, ARN2508, BIA 10-2472, BMS-469908, |
| | CAY-10402, JNJ-245, JNJ-1661010, JNJ- |
| | 28833155, JNJ-40413269, JNJ-42119779, JNJ- |
| | 42165279, MK-3168, MK-4409, MM-433593, OL- |
| | 92, OL-135, PF-622, PF-750, PF-3845, PF- |
| | 04457845, PF-04862853, RN-450, SA-47, SA-73, |
| | SSR-411298, ST-4068, TK-25, URB524, URB597, |
| | URB694, URB937, VER-156084, and V-158866 |
| Monoacylglycerol lipase inhibitors | N-arachidonoyl maleimide, JZL184 |
| (increase cannabinoid neurotransmission) | |
| Endocannabinoid transporter inhibitors | SB-FI-26 |
| (increase cannabinoid neurotransmission) | |
| Endocannabinoid reuptake inhibitors | AM404, AM1172, LY-2183240, O-2093, OMDM-2, |
| (increase cannabinoid neurotransmission) | UCM-707, VDM-11, guineensine, ETI-T-24_B_I, |
| | WOBE437, and RX-055 |
| Adenosine uptake inhibitors | cilostazol, dilazep, and dipyramidole |
| (increase purinergic neurotransmission) | |
| Nucleoside transporter inhibitors | 8MDP, Decynium 22, 5-iodotubercidin, NBMPR, |
| (increase purinergic neurotransmission) | and TC-T 6000 |

In some embodiments, the neurotransmission blocker is a neurotoxin listed in Table 10, or a functional fragment or variant thereof. Neurotoxins include, without limitation, convulsants, nerve agents, parasympathomimetics, and uranyl compounds. Neurotoxins may be bacterial in origin, or fungal in origin, or plant in origin, or derived from a venom or other natural product. Neurotoxins may be synthetic or engineered molecules, derived de novo or from a natural product. Suitable neurotoxins include but are not limited to botulinum toxin and conotoxin. Exemplary neurotoxins are listed in Table 10.

TABLE 10
NEUROTOXINS
NEUROTOXINS

2,4,5-Trihydroxyamphetamine
2,4,5-Trihydroxymethamphetamine
3,4-Dichloroamphetamine
5,7-Dihydroxytryptamine
5-Iodowillardiine
Ablomin
Aconitine
Aconitum
Aconitum anthora
AETX
Agelenin
Agitoxin
Aldrin
Alpha-Methyldopamine
Alpha-neurotoxin
Altitoxin
Anatoxin-a
Androctonus australis hector insect toxin
Anisatin
Anthopleurin
Antillatoxin
Anuroctoxin
Apamin
Arum italicum
Arum maculatum
Babycurus toxin 1
Batrachotoxin
BDS-1
Bestoxin
Beta-Methylamino-L-alanine
BgK
Birtoxin
BmKAEP
BmTx3
BotlT2
BotlT6
Botulinum toxin
Brevetoxin
Bukatoxin
Butantoxin
Calcicludine
Calciseptine
Calitoxin
Caramboxin
Carbon disulfide
CgNa toxin
Charybdotoxin
Cicutoxin
Ciguatoxin
Cll1
Clostridium botulinum
Conantokins
Conhydrine
Coniine
Conotoxin
Contryphan
CssII
CSTX
Curare
Cyanide poisoning
Cylindrospermopsin
Cypermethrin
Delta atracotoxin
Dendrotoxin
Dieldrin
Diisopropyl fluorophosphates
Dimethylmercury
Discrepin
Domoic acid
Dortoxin
DSP-4
Ergtoxin
Falcarinol
Fenpropathrin
Gabaculine
Ginkgotoxin
Grammotoxin
Grayanotoxin
Hainantoxin
Halcurin
Hefutoxin
Helothermine
Heteroscodratoxin-1
Histrionicotoxin
Homoquinolinic acid
Hongotoxin
Huwentoxin
Ibotenic acid
Ikitoxin
inhibitor cystine knot
Jingzhaotoxin
Kainic acid
Kaliseptine
Kappa-bungarotoxin
Kodaikanal mercury poisoning
Kurtoxin
Latrotoxin
Lq2
Maitotoxin
Margatoxin
Maurotoxin
Mercury (element)
Methanol
Methiocarb
MPP+
MPTP
Nemertelline
Neosaxitoxin
Nicotine
N-Methylconiine
Oenanthotoxin
Oxalyldiaminopropionic acid
Oxidopamine
Oxotoxin
Pahutoxin
Palytoxin
Pandinotoxin
Para-Bromoamphetamine
Para-Chloroamphetamine
Para-Chloromethamphetamine
Para-Iodoamphetamine
Penitrem A
Phaiodotoxin
Phenol
Phoneutria nigriventer toxin-3
Phrixotoxin
Polyacrylamide
Poneratoxin
Psalmotoxin
Pumiliotoxin
Quinolinic acid
Raventoxin
Resiniferatoxin
Samandarin
Saxitoxin
Scyllatoxin
Sea anemone neurotoxin
Slotoxin
SNX-482
Stichodactyla toxin
Taicatoxin
Taipoxin
Tamapin
Tertiapin
Tetanospasmin
Tetraethylammonium
Tetramethylenedisulfotetramine
Tetrodotoxin
Tityustoxin
Tricresyl phosphate
TsIV
Vanillotoxin
Veratridine

Antibodies

Neurotransmission modulators also include antibodies that bind to neurotransmitters or neurotransmitter receptors listed in Tables 6 and 7 and decrease neurotransmission. These antibodies include blocking and neutralizing antibodies. Antibodies to neurotransmitters or neurotransmitter receptors listed in Tables 6 and 7 can be generated by those of skill in the art using well established and routine methods.

Neuronal Growth Factor Blockers

In some embodiments, the GRK2 inhibitor is administered with a neuronal growth factor blocker (e.g., an agent that decreases neurogenic/axonogenic signals, e.g., an antagonist of a neurotrophic factor, neuronal growth factor, or neuronal growth factor receptor). For example, the neuronal growth factor blocker is an antagonist of a neuronal growth factor or neuronal growth factor receptor listed in Table 11. A neuronal growth factor blocker may decrease neurogenesis, neuronal growth, neuronal differentiation, neurite outgrowth, synapse formation, synaptic maturation, synaptic refinement, or synaptic stabilization. Neuronal growth factor blockers decrease tissue innervation (e.g., innervation of a tumor) and the formation of synaptic connections between two or more neurons and between neurons and non-neural cells. A neuronal growth factor blocker may block one or more of these processes (e.g., through the use of antibodies that block neuronal growth factors or their receptors or inhibitory RNAs directed to neuronal growth factors or their receptors). Neuronal growth factor blockers can decrease one of the above-mentioned processes by 10%, 20%, 30%, 40%, 50%, 60%, 70%, 80%, 90%, 95%, 98%, 200%, 500% or more.

In some embodiments, the neuronal growth factor blocker decreases neurogenic/axonogenic signals, e.g., the method includes administering to the subject or contacting a cell with a neuronal growth factor blocker in an amount and for a time sufficient to decrease neurogenesis, axonogenesis, or innervation. For example, the neuronal growth factor blocker that leads to a decrease in neurogenesis or axonogenesis is a blocking or neutralizing antibody against a neurotrophic factor. Relevant neurotrophic factors include NGF, BDNF, ProNGF, Sortilin, TGFβ and TGFβ family ligands and receptors (e.g., TGFβR₁, TGFβR₂, TGFβ1, TGFβ2 TGFβ4), GFRα family ligands and receptors (e.g., GFRα1, GFRα2, GFRα3, GFRα4, GDNF), CNTF, LIF, neurturin, artemin, persephin, neurotrophin, chemokines, cytokines, and others listed in Table 11. Receptors for these factors can also be targeted, as well as downstream signaling pathways including Jak-Stat inducers, and cell cycle and MAPK signaling pathways. In some embodiments, the neuronal growth factor blocker decreases neurogenesis, axonogenesis or any of the processes mentioned above by sequestering, blocking, antagonizing, degrading, or downregulating a neuronal growth factor or a neuronal growth factor receptor listed in Table 11. In some embodiments, the neuronal growth factor blocker decreases neurogenesis, axonogenesis or any of the processes mentioned above by blocking or antagonizing a signaling protein that is downstream of a neuronal growth factor. In some embodiments, the neuronal growth factor blocker decreases neurogenesis, axonogenesis or any of the processes mentioned above by blocking, disrupting, or antagonizing a synaptic or structural protein. Neurogenesis, axonogenesis, neuronal growth, neuronal differentiation, neurite outgrowth, synapse formation, synaptic maturation, synaptic refinement, synaptic stabilization, or tissue innervation can be decreased in the subject at least 1%, 2%, 5%, 10%, 15%, 20%, 25%, 30%, 35%, 40%, 50%, 60%, 70%, 80% or more, compared to before the administration. Neurogenesis, axonogenesis, neuronal growth, neuronal differentiation, neurite outgrowth, synapse formation, synaptic maturation, synaptic refinement, synaptic stabilization, or tissue innervation can be decreased in the subject between 5-20%, between 5-50%, between 10-50%, between 20-80%, between 20-70%. Neuronal growth factor blockers can be administered in any of the modalities described herein (e.g., antibody, small molecule, nucleic acid, polypeptide, or viral vector).

In some embodiments, the neuronal growth factor blocker decreases the number of nerves in an affected tissue. For example, the subject has cancer (e.g., the subject has a highly innervated tumor). For example, the neuronal growth factor blocker is administered in an amount and for a time sufficient to decrease neurogenesis/axonogenesis.

Neuronal growth factor blockers include antibodies that bind to neuronal growth factors or neuronal growth factor receptors and decrease their signaling (e.g., blocking antibodies). Exemplary neuronal growth factor blocking antibodies are listed below in Table 12. Antibodies to neuronal growth factors listed in Table 11 can also be generated by those of skill in the art using well established and routine methods.

TABLE 11
NEURONAL GROWTH FACTORS

| Gene | Type | Accession Number | Entrez Gene ID |
|---|---|---|---|
| ARTN | Ligand | Q5T4W7 | 9048 |
| BDNF | Ligand | P23560 | 627 |
| BDNF-AS | Ligand | | 497258 |
| BEX1 | Signaling | Q9HBH7 | 55859 |
| BEX3 | Signaling | Q00994 | 27018 |
| CD34 | Receptor | P28906 | 947 |
| CDNF | Ligand | Q49AH0 | 441549 |
| CNTF | Ligand | P26441 | 1270 |
| CNTFR | Receptor | P26992 | 1271 |
| CRLF1 | Receptor | O75462 | 9244 |
| CSPG5 | Ligand | O95196 | 10675 |
| DCLK1 | Signaling | O15075 | 9201 |
| DISC1 | Signaling | Q9NRI5 | 27185 |
| DNAJC5 | Signaling | Q9H3Z4 | 80331 |
| DPYSL2 | Signaling | Q16555 | 1808 |
| DVL1 | Signaling | O14640 | 1855 |
| EFNA5 | Ligand | P52803 | 1946 |
| EGR3 | Signaling | Q06889 | 1960 |
| ENO2 | Signaling | P09104 | 2026 |
| EphA1 | Receptor | P21709 | 2041 |
| EphA10 | Receptor | Q5JZY3 | 284656 |
| EphA2 | Receptor | P29317 | 1969 |
| EphA3 | Receptor | P29320 | 2042 |
| EphA4 | Receptor | P29317 | 2043 |
| EphA5 | Receptor | P54756 | 2044 |
| EphA6 | Receptor | Q9UF33 | 285220 |
| EphA7 | Receptor | Q15375 | 2045 |
| EphA8 | Receptor | P29322 | 2046 |
| EphB1 | Receptor | P54762 | 2047 |
| EphB2 | Receptor | P29323 | 2048 |
| EphB3 | Receptor | P54753 | 2049 |
| EphB4 | Receptor | P54760 | 2050 |
| EphB6 | Receptor | O15197 | 2051 |
| ETBR2 | Receptor | O60883 | 9283 |
| FSTL4 | Receptor | Q6MZW2 | 23105 |
| GDNF | Ligand | P39905 | 2668 |
| GFRA1 | Receptor | P56159 | 2674 |
| GFRA2 | Receptor | O00451 | 2675 |
| GFRA3 | Receptor | O60609 | 2676 |
| GFRA4 | Receptor | Q9GZZ7 | 64096 |
| GPR37 | Receptor | O15354 | 2861 |
| GPRIN1 | Signaling | Q7Z2K8 | 114787 |
| GPRIN2 | Signaling | O60269 | 9721 |
| GPRIN3 | Signaling | Q6ZVF9 | 285513 |
| GRB2 | Signaling | P62993 | 2885 |
| GZF1 | Signaling | Q9H116 | 64412 |
| IFNA1 | Ligand | P01562 | 3439 |
| IGF1 | Ligand | P05019 | 3479 |
| IGF2 | Ligand | P01344 | 3481 |
| IL11RA | Receptor | Q14626 | 3590 |
| IL1B | Ligand | P01584 | 3553 |
| IL3 | Ligand | P08700 | 3562 |
| IL4 | Ligand | P05112 | 3565 |
| IL6 | Ligand | P05231 | 3569 |
| IL6R | Receptor | P08887 | 3570 |
| IL6ST | Signaling | P40189 | 3572 |
| INS | Ligand | P01308 | 3630 |
| L1CAM | Signaling | P32004 | 3897 |
| LIF | Ligand | P15018 | 3976 |
| LIFR | Receptor | P42702 | 3977 |
| MAGED1 | Signaling | Q9Y5V3 | 9500 |
| MANF | Ligand | P55145 | 7873 |
| NDNF | Ligand | Q8TB73 | 79625 |
| NENF | Ligand | Q9UMX5 | 29937 |
| NENFP1 | Ligand | | 106480294 |
| NENFP2 | Ligand | | 100129880 |
| NENFP3 | Ligand | | 106481703 |
| NGF | Ligand | P01138 | 4803 |
| NGFR | Receptor | P08138 | 4804 |
| NRG1 | Ligand | Q02297 | 3084 |
| NRP1 | Receptor | O14786 | 8829 |
| NRTN | Ligand | Q99748 | 902 |
| NTF3 | Ligand | P20783 | 4908 |
| NTF4 | Ligand | P34130 | 4909 |
| NTRK1 | Receptor | P04629 | 4914 |
| NTRK2 | Receptor | Q16620 | 4915 |
| NTRK3 | Receptor | Q16288 | 4916 |
| PDPK1 | Signaling | O15530 | 5170 |
| PEDF | Ligand | P36955 | 5176 |
| PLEKHH3 | Signaling | Q7Z736 | 79990 |
| PSAP | Ligand | P07602 | 5660 |
| PSEN1 | Signaling | P49768 | 5663 |
| PSPN | Ligand | O70300 | 5623 |
| PTN | Ligand | P21246 | 5764 |
| RELN | Ligand | P78509 | 5649 |
| RET | Signaling | P07949 | 5979 |
| ROR1 | Receptor | Q01973 | 4919 |
| ROR2 | Receptor | Q01974 | 4920 |
| RPS6KA3 | Signaling | P51812 | 6197 |
| SDC3 | Receptor | O75056 | 9672 |
| SEMA3E | Ligand | O15041 | 9723 |
| SERPINE2 | Ligand | P07093 | 5270 |
| SERPINF1 | Ligand | P36955 | 5176 |
| SHC1 | Signaling | P51812 | 6464 |
| SNTG1 | Biosynthesis | P07602 | 54212 |
| SORCS1 | Receptor | O75056 | 114815 |
| SORCS2 | Receptor | O15041 | 57537 |
| SORCS3 | Receptor | P07093 | 22986 |
| SORT1 | Receptor | Q99523 | 6272 |
| SULF1 | Signaling | Q8IWU6 | 23213 |
| SULF2 | Signaling | Q8IWU5 | 55959 |
| TGFB1 | Ligand | P01137 | 7040 |
| TGFB2 | Ligand | P61812 | 7042 |
| TGFB3 | Ligand | P10600 | 7043 |
| TMEM158 | Receptor | Q8WZ71 | 25907 |
| TNF | Ligand | P01375 | 7124 |
| TPM3 | Receptor | P06753 | 7170 |
| VEGFA | Ligand | P15692 | 7422 |
| VEGFB | Ligand | P49765 | 7423 |
| VGF | Ligand | O15240 | 7425 |
| XCR1 | Receptor | P46094 | 2829 |
| ZN274 | Signaling | Q96GC6 | 10782 |

TABLE 12
NEURONAL GROWTH FACTOR ANTIBODIES

| Neuronal | | |
|---|---|---|
| Growth | | |
| Factor | Antibody | Company |
| BDNF | 38B8 (agonist antibody) | Pfizer |
| BDNF | 29D7 (agonist antibody) | Pfizer |
| EphA3 | KB004 | KaloBios Pharmaceuticals, Inc. |
| IFNA1 | Faralimomab | Creative Biolabs |
| IFNA1 | Sifalimumab (MEDI-545) | MedImmune |
| IFNA1 | Rontalizumab | Genentech |
| IGF | Figitumumab (CP-751,871) - an | Pfizer |
| | IGR-1R MAb | |
| IGF | SCH717454 (Robatumamab, | Merck |
| | inhibits IGF initiated | |
| | phosphorylation) | |
| IGF | Cixutumumab (IGF-1R antibody) | Eli Lilly |
| IGF | Teprotumumab (IGF-1R | Genmab/Roche |
| | blocking antibody) | |
| IGF-2 | Dusigitumab | MedImmune/AstraZeneca |
| IGF-2 | DX-2647 | Dyax/Shire |
| IGF | Xentuzumab | Boehringer Ingelheim/Eli Lilly |
| IGF | Dalotuzumab (IGFR1 blocking | Merck & Co. |
| | antibody) | |
| IGF | Figitumumab (IGFR1 blocking | Pfizer |
| | antibody) | |
| IGF | Ganitumab (IGFR1 blocking | Amgen |
| | antibody) | |
| IGF | Robatumumab (IGFR1 blocking | Roche/Schering-Plough |
| | antibody) | |
| IL1B | Canakinumab | Novartis |
| IL1B | APX002 | Apexigen |
| IL1B | Gevokizumab | XOMA |
| IL4 | Pascolizumab | GlaxoSmithKline |
| IL4 | Dupilumab | Regeneraon/Sanofi |
| IL6 | Siltuximab | Janssen Biotech, Inc. |
| IL6 | Olokizumab | UCB/R-Pharm |
| IL6 | Elsilimomab | Orphan Pharma International |
| IL6 | Sirukumab | Centocor |
| IL6 | Clazakizumab | Bristol Myers Squib/Alder |
| | | Biopharmaceuticals |
| IL6 | Gerilimzumab (ARGX-109) | arGEN-X/RuiYi |
| IL6 | FE301 | Ferring Pharmaceuticals |
| IL6 | FM101 | Femta Pharmaceuticals |
| IL-6R | Sarilumab (directed against | Regeneron/Sanofi |
| | IL6R) | |
| IL-6R | Tocilizumab | Hoffmann-La Roche/Chugai |
| IL-6R | Sapelizumab | Chugai |
| IL-6R | Vobarilizumab | Ablynx |
| L1CAM | AB417 | Creative biolabs |
| L1CAM | L1-9.3 | Creative biolabs |
| L1CAM | L1-14.10 | Biolegend |
| NGF | Tanezumab | Pfizer |
| NGF | Fulranumab (JNJ-42160443), | Amgen |
| NGF | MNAC13 (anti-TrkA, the NGF | Creative Biolabs |
| | receptor) | |
| NGF | mAb 911 | Rinat/Pfizer |
| NGF | Fasinumab | Regeneron/Teva |
| NRG1 | 538.24 | Hoffman-La Roche |
| NRP1 | Vesencumab | Genentech/Roche |
| ROR1 | Cirmtuzumab | Oncternal Therapeutics |
| SAP | GSK2398852 | GlaxoSmithKline |
| TGFβ | Fresolimumab (pan-TGFβ | Genzyme/Aventis |
| | antibody) | |
| TGFβ | IMC-TR1 (LY3022859) (MAb | Eli Lilly |
| | against TGFβRII) | |
| TGFβ | TβM1 (anti-TGFβ1 MAb) | Eli Lilly |
| TGFβ2 | Lerdelimumab (CAT-152) | Genzyme |
| TGFβ1 | Metelimumab | Genzyme |
| TGFβ1 | LY2382770 | Eli Lilly |
| TGFβ | PF-03446962 (MAb against | Pfizer |
| | TGFβRI) | |
| TNF | Infliximab | Janssen Biotech, Inc. |
| TNF | Adalimumab | AbbVie Inc. |
| TNF | Certolizumab pegol | UCB |
| TNF | Golimumab | Janssen Biotech, Inc. |
| TNF | Afelimomab | |
| TNF | Placulumab | Teva Pharmaceutical Industries, |
| | | Inc. |
| TNF | Nerelimomab | Chiron/Celltech |
| TNF | Ozoralizumab | Pfizer/Ablynx |
| VEGFA | Bevacizumab | Genentech |
| VEGFA | Ranibizumab | Genentech |
| VEGF | Alacizumab pegol (anti- | UCB |
| | VEGFR2) | |
| VEGFA | Brolucizumab | Novartis |
| VEGF | Icrucumab (anti-VEGFR1) | Eli Lilly |
| VEGF | Ramucirumab (anti-VEGFR2) | Eli Lilly |

Neuronal growth factor blockers also include agents that antagonize neuronal growth factors and neuronal growth factor receptors. For example, neuronal growth factor blockers include TNF inhibitors (e.g., etanercept, thalidomide, lenalidomide, pomalidomide, pentoxifylline, bupropion, and DOI), TGFβ1 inhibitors, (e.g., disitertide (P144)), and TGFβ2 inhibitors (e.g., trabedersen (AP12009)). Exemplary neuronal growth factor antagonists are listed in Table 13.

TABLE 13
NEURONAL GROWTH FACTOR AGONISTS
AND ANTAGONISTS

| | Agonist | Antagonist |
|---|---|---|
| TrkA | NGF, amitriptyline, and | ALE-0540 |
| | gambogic amide, gambogic | |
| | acid | |
| TrkB | BDNF, NT3, NT4, 3,7- | ANA-12, cyclotraxin B, |
| | Dihydroxyflavone, 3,7,8,2′- | and gossypetin |
| | Tetrahydroxyflavone, 4′- | |
| | Dimethylamino-7,8- | |
| | dihydroxyflavone, 7,3′- | |
| | Dihydroxyflavone, 7,8- | |
| | Dihydroxyflavone, 7,8,2′- | |
| | Trihydroxyflavone, 7,8,3′- | |
| | Trihydroxyflavone, | |
| | Amitriptyline^{,} Deoxygedunin, | |
| | Diosmetin, HIOC, LM22A-4, | |
| | N-Acetylserotonin, | |
| | Norwogonin (5,7,8-THF), | |
| | R7, LM22A4, and TDP6 | |
| Pan-Trk | | entrectinib (RXDX-101), |
| receptor | | AG 879, GNF 5837, GW |
| | | 441756, and PF 06273340 |
| GFRα1R | GDNF and XIB4035 | |
| VEGF | | AEE 788, AG 879, AP 24534, |
| receptor | | axitinib, DMH4, GSK |
| | | 1363089, Ki 8751, RAF 265, |
| | | SU 4312, SU 5402, SU 5416, |
| | | SU 6668, sunitinib, |
| | | toceranib, vatalanib, XL 184, |
| | | ZM 306416, and ZM 323881 |
| TGFβRI | | galunisertib (LY2157299), |
| | | TEW-7197, SB-431542, A |
| | | 83-01, D 4476, GW 788388, |
| | | LY 364947, R 268712, |
| | | RepSox, SB 505124, SB |
| | | 525334, and SD 208 |

In any of the combination therapy approaches described herein, the first and second therapeutic agent (e.g., a GRK2 inhibitor described herein and the additional therapeutic agent) are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

Diagnosis and Prognosis of GRK2-Associated Cancer

The methods described herein include methods of diagnosing or identifying patients with GRK2-associated cancer. Subjects who can be diagnosed or identified as having GRK2-associated cancer are subjects who have cancer (e.g., subjects identified as having cancer), or subjects suspected of having cancer. Subjects can be diagnosed or identified as having GRK2-associated cancer based on screening of patient cancer samples (e.g., tumor biopsies). GRK2 expression can be assessed in a cancer sample isolated from a subject using standard techniques known in the art, such as immunohistochemistry, western blot analysis, quantitative RT-PCR, RNA sequencing, fluorescent in situ hybridization, cDNA microarray, and droplet digital PCR. GRK2 expression can be assessed by comparing measurements obtained from subject cancer samples to measurements of GRK2 expression obtained from a reference sample (e.g., a non-cancerous cell of the same type or a cell that does not express GRK2, e.g., a HEK cell). Reference samples can be obtained from healthy subjects (e.g., subjects without cancer), or they can be obtained from databases in which average measurements of GRK2 expression are cataloged for a variety of types of healthy (e.g., non-cancerous) cells from many subjects.

Subjects are diagnosed or identified as having GRK2-associated cancer if GRK2 expression is elevated or increased in the cancer sample compared to the reference sample. An increase of GRK2 expression of 1.1-fold or more (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more) in the cancer sample compared to the reference indicates that the subject has GRK2-associated cancer. Subjects diagnosed or identified as having GRK2-associated cancer can be treated with the methods and compositions described herein (e.g., GRK2 inhibitors). Subjects can also be selected for treatment with the methods and compositions described herein if the cancer sample from the subject is found to express GRK2.

The methods described herein also include methods of predicting patient response (e.g., the response of cancer in a subject) to GRK2 inhibitors in order to determine whether GRK2 inhibitors can be used for cancer treatment. In some embodiments, a cancer sample (e.g., a tumor biopsy or cancer cell) is isolated from a subject and contacted with one or more GRK2 inhibitors or GRK2-specific inhibitors (e.g., cancer samples are cultured and contacted with one or more inhibitors in vitro). The response of the cancer sample to the one or more GRK2 inhibitors or GRK2-specific inhibitors is evaluated to predict response to treatment. Responses that are evaluated include cancer cell or tumor growth, cancer cell or tumor proliferation, cancer cell or tumor migration, cancer cell or tumor metastasis, cancer cell or tumor invasion, cancer cell or tumor death, cancer or cell or tumor autophagy, cancer cell or tumor innervation, or cancer cell or tumor GRK2 expression. A decrease of at least 5% or more (e.g., 5%, 10%, 15%, 20%, 25%, 30%, 40%, 50%, 60%, 70%, 80%, 90%, 99%, or more) in cancer cell or tumor growth, cancer cell or tumor proliferation, cancer cell or tumor migration, cancer cell or tumor metastasis, cancer cell or tumor invasion, cancer cell or tumor innervation, or cancer cell or tumor GRK2 expression in treated cells compared to untreated or control-treated cells, or an increase of at least 5% or more (e.g., 5%, 10%, 15%, 20%, 25%, 30%, 40%, 50%, 60%, 70%, 80%, 90%, 99%, or more) in cancer cell or tumor death or cancer cell or tumor autophagy in treated cells compared to untreated or control-treated cells indicates that the cancer would respond to treatment with a GRK2 inhibitor.

The methods used above to diagnose or identify a subject with GRK2-associated cancer can also be used to predict patient response (e.g., the response of cancer in a subject) to treatment with a GRK2 inhibitor. If the expression of GRK2 is elevated or increased in a cancer sample compared to a reference (e.g., 1.1, 1.2, 1.3, 1.4, 1.5, 2.0, 2.5, 3.0, 3.5, 4.0, 4.5, 5.0, 6.0, 7.0, 8.0, 9.0, 10.0-fold or more higher in the cancer sample compared to the reference), the subject can be predicted to respond to treatment with a GRK2 inhibitor. Subjects predicted to respond to treatment with a GRK2 inhibitor or GRK2-specific inhibitor can be treated using the methods and compositions described herein (e.g., GRK2 inhibitors).

Methods of Treatment

Administration

An effective amount of a GRK2 inhibitor described herein for treatment of cancer can be administered to a subject by standard methods. For example, the agent can be administered by any of a number of different routes including, e.g., intravenous, intradermal, subcutaneous, percutaneous injection, oral, transdermal (topical), or transmucosal. The GRK2 inhibitor can be administered orally or administered by injection, e.g., intramuscularly, or intravenously. The most suitable route for administration in any given case will depend on the particular agent administered, the patient, the particular disease or condition being treated, pharmaceutical formulation methods, administration methods (e.g., administration time and administration route), the patient's age, body weight, sex, severity of the diseases being treated, the patient's diet, and the patient's excretion rate. The agent can be encapsulated or injected, e.g., in a viscous form, for delivery to a chosen site, e.g., a tumor site. The agent can be provided in a matrix capable of delivering the agent to the chosen site. Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, and cosmetic appearance and interface properties. One example is a collagen matrix.

The agent (e.g., GRK2 inhibitor, e.g., polypeptide, small molecule, nucleic acid, or antibody) can be incorporated into pharmaceutical compositions suitable for administration to a subject, e.g., a human. Such compositions typically include the agent and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GRK2 inhibitor described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art.

Nucleic acid molecule agents described herein can be administered directly (e.g., therapeutic mRNAs) or inserted into vectors used as gene therapy vectors. Gene therapy vectors can be delivered to a subject by, for example, intravenous injection, local administration (see U.S. Pat. No. 5,328,470) or by stereotactic injection (see, e.g., Chen et al., PNAS 91:3054 1994). The pharmaceutical preparation of the gene therapy vector can include the gene therapy vector in an acceptable diluent, or can include a slow release matrix in which the gene delivery vehicle is embedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of formulating pharmaceutical agents are known in the art, e.g., Niazi, Handbook of Pharmaceutical Manufacturing Formulations (Second Edition), CRC Press 2009, describes formulation development for liquid, sterile, compressed, semi-compressed and OTC forms. Transdermal and mucosal delivery, lymphatic system delivery, nanoparticles, controlled drug release systems, theranostics, protein and peptide drugs, and biologics delivery are described in Wang et al., Drug Delivery: Principles and Applications (Second Edition), Wiley 2016; formulation and delivery of peptide and protein agent is described, e.g., in Banga, Therapeutic Peptides and Proteins: Formulation, Processing, and Delivery Systems (Third Edition), CRC Press 2015.

In some embodiments, the GRK2 inhibitors described herein are formulated for delivery in exosomes, small membrane vesicles (30 nm to 100 nm in diameter) of endocytic origin that are released into the extracellular environment following fusion of multivesicular bodies with the plasma membrane. Exosomes can be loaded with varied therapeutics such as small molecule drugs and nucleic acid molecules (e.g., inhibitory RNA molecules, such as siRNA, shRNA, or miRNA, or nucleases, such as gRNAs, ZFNs, or TALENs) to form therapeutic exosomes, which constitute an attractive carrier and delivery system for therapeutics. Unlike liposomes and other synthetic nanoparticle carriers of therapeutics, exosomes contain transmembrane- and membrane-anchored proteins that likely enhance endocytosis, thus promoting more efficient delivery of their internal content. Another advantage of therapeutic exosomes over synthetic nanoparticle carriers (e.g., liposomes) is enhanced stability and reduced clearance from circulation. The use of therapeutic exosomes as carrier and delivery system for therapeutics might also minimize cytotoxic effects observed with the use of synthetic nanoparticles (e.g., liposomes) in vivo. Exosomes can be produced by the methods described in Kamerkar et al. (Nature 546(7659): 498-503, 2017), Melo et al. (Nature 523(7559): 177-182, 2015), and U.S. Pat. Nos. 9,085,778, 9,629,929, and 9,889,210, the disclosures of which are incorporated herein by reference.

Exosomes can be isolated from supernatant of donor cells (e.g., fibroblast, fibroblast-like mesenchymal cells, mast cells, cancer cells, tumor cells, and/or cells from cancer tissue) by differential centrifugation processes. Following isolation, the donor exosomes (i.e., exosomes isolated from donor cells) can be modified so as to remove genetic materials (e.g., miRNA, and mRNA) from the donor exosomes. To avoid interference with undesirable or irrelevant genetic material, it may be preferable to use donor exosomes that lack genetic contents (e.g., miRNA, or mRNA). Empty donor exosomes can be used for direct transfer to recipient cells or for direct transfection or microinjection of a therapeutic agent into the exosomes. Methods of transferring therapeutic agents directly into exosomes include transformation, transfection and microinjection.

Local Administration

The GRK2 inhibitors described herein can be administered locally, e.g., to the site of cancer in the subject. Examples of local administration include epicutaneous, inhalational, intra-articular, intrathecal, intravaginal, intravitreal, intrauterine, intra-lesional administration, lymph node administration, intratumoral administration and administration to a mucous membrane of the subject, wherein the administration is intended to have a local and not a systemic effect. As an example, for the treatment of a cancer described herein, the GRK2 inhibitor may be administered locally (e.g., intratumorally) in a compound-impregnated substrate such as a wafer, microcassette, or resorbable sponge placed in direct contact with the affected tissue. Alternatively, the GRK2 inhibitor is infused into the brain or CSF using standard methods. As yet another example, a pulmonary cancer described herein may be treated, for example, by administering the GRK2 inhibitor locally by inhalation, e.g., in the form of an aerosol spray from a pressured container or dispenser which contains a suitable propellant, e.g., a gas such as carbon dioxide or a nebulizer. A GRK2 inhibitor for use in the methods described herein can be administered at the site of a tumor, e.g., intratumorally. In certain embodiments, the agent is administered to a mucous membrane of the subject.

Combination Therapy

The GRK2 inhibitors described herein may be administered in combination with one or more additional therapies (e.g., 1, 2, 3 or more additional therapeutic agents). The two or more agents can be administered at the same time (e.g., administration of all agents occurs within 15 minutes, 10 minutes, 5 minutes, 2 minutes or less). The agents can also be administered simultaneously via co-formulation. The two or more agents can also be administered sequentially, such that the action of the two or more agents overlaps and their combined effect is such that the reduction in a symptom, or other parameter related to the disorder is greater than what would be observed with one agent or treatment delivered alone or in the absence of the other. The effect of the two or more treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, local routes, and direct absorption through mucous membrane tissues. The therapeutic agents can be administered by the same route or by different routes. For example, a first therapeutic agent of the combination may be administered by intravenous injection while a second therapeutic agent of the combination can be administered locally in a compound-impregnated microcassette. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

For use in treating cancer, the second agent may be a checkpoint inhibitor, a chemotherapeutic drug, a biologic cancer agent (e.g., an antibody listed in Table 4), a cancer specific agent (e.g., an agent listed in Table 5), a non-drug therapy, a cytokine therapy (e.g., an interferon or interleukin, e.g., IL-2 or IL-12), CAR-T therapy, an oncolytic virus (e.g., tamilogene laherparepvec (T-VEC), maraba virus, PVS-RIPO, canerpaturev, enadenotucirev, pelareorep, pexastimogene devacirepvec (JX-594), or tasadenoturev), a neurotransmission blocker, or a neuronal growth factor blocker. In one embodiment, the inhibitor of checkpoint is an inhibitory antibody (e.g., a monospecific antibody such as a monoclonal antibody). The antibody may be, e.g., humanized or fully human. In other embodiments, the inhibitor of checkpoint is a fusion protein, e.g., an Fc-receptor fusion protein. In some embodiments, the inhibitor of checkpoint is an agent, such as an antibody, that interacts with a checkpoint protein. In other embodiments, the inhibitor of checkpoint is an agent, such as an antibody, that interacts with the ligand of a checkpoint protein. In one embodiment, the inhibitor of checkpoint is an inhibitor (e.g., an inhibitory antibody or small molecule inhibitor) of CTLA-4 (e.g., an anti-CTLA4 antibody such as ipilimumab or tremelimumab). In one embodiment, the inhibitor of checkpoint is an inhibitor (e.g., an inhibitory antibody or small molecule inhibitor) of PD-1 (e.g., nivolumab; pembrolizumab; pidilizumab/CT-011). In one embodiment, the inhibitor of checkpoint is an inhibitor (e.g., an inhibitory antibody or small molecule inhibitor) of PDL1 (e.g., MPDL3280A/RG7446; MED14736; MSB0010718C; BMS 936559). In one embodiment, the inhibitor of checkpoint is an inhibitor (e.g., an inhibitory antibody or Fc fusion or small molecule inhibitor) of PDL2 (e.g., a PDL2/Ig fusion protein such as AMP 224). In one embodiment, the inhibitor of checkpoint is an inhibitor (e.g., an inhibitory antibody or small molecule inhibitor) of B7-H3 (e.g., MGA271), B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. The second agent may also be an anti-angiogenic drug, e.g., an anti-VEGF antibody, or the second agent may be an oncolytic agent e.g., a chemotherapy, a drug that targets cancer metabolism, an antibody that marks a cancer cell surface for destruction, e.g., rituximab or trastuzumab, an antibody-drug conjugate, e.g., trastuzumab emtansine, a cell therapy, or other commonly-used anti-neoplastic agent.

Dosing

Subjects that can be treated as described herein are subjects with cancer or at risk of developing cancer. The cancer may be a primary tumor or a metastasized tumor. In some embodiments, the cancer is a GRK2-associated cancer. Subjects who can be treated with the methods disclosed herein include subjects who have had one or more tumors resected, received chemotherapy or other pharmacological treatment for the cancer, received radiation therapy, and/or received other therapy for the cancer. Subjects who have never previously been treated for cancer can also be treated using the methods described herein.

In some embodiments, the agent is administered in an amount and for a time effective to result in one of (or more, e.g., 2 or more, 3 or more, 4 or more of): (a) reduced tumor size, (b) reduced rate of tumor growth, (c) increased tumor cell death (d) reduced tumor progression, (e) reduced number of metastases, (f) reduced rate of metastasis, (g) reduced tumor migration, (h) reduced tumor invasion, (i) reduced tumor volume, (j) decreased tumor recurrence, (k) increased survival of subject, (l) increased progression free survival of subject.

The methods described herein may include a step of selecting a treatment for a patient. The method includes (a) identifying (e.g., diagnosing) a patient who has cancer or is at risk of developing cancer, and (b) selecting a GRK2 inhibitor, e.g., a GRK2 inhibitor described herein, to treat the condition in the patient. In some embodiments, the method includes administering the selected treatment to the subject. In some embodiments, a patient is identified as having cancer based on imaging (e.g., MRI, CT, or PET scan), biopsy, or blood sample (e.g., detection of blood antigen markers, circulating tumor DNA (e.g., by PCR). In some embodiments, a patient is identified as having cancer after presenting with one or more symptoms of a paraneoplastic syndrome (e.g., fever, auto-antibodies directed against nervous system proteins, ataxia, dizziness, nystagmus, difficulty swallowing, loss of muscle tone, loss of fine motor coordination, slurred speech memory loss, vision loss, sleep disturbances, dementia, seizures, dysgeusia, cachexia, anemia, itching, or sensory loss in the limbs). In some embodiments, a patient presents with symptoms of paraneoplastic syndrome and is then identified as having cancer based on imaging (e.g., CT, MRI, or PET scans).

The method may also include (a) identifying (e.g., diagnosing) a patient who has a neoplasm, (b) optionally evaluating the neoplasm for innervation, and (c) selecting a GRK2 inhibitor (e.g., a GRK2 inhibitor described herein) to treat the patient if the neoplasm is highly innervated (e.g., if the level of innervation is at least 10% higher (e.g., at least 20%, 30%, 40%, 50%, 60%, 70%, 80% higher) than the level of innervation in control tissue, e.g., non-cancerous tissue of the same subject). Innervation may be measured by staining tissue sections for neural markers e.g., immuno-histochemical staining for tyrosine hydroxylase, vesicular acetylcholine transporter; NGF-Inducible Large External glycoprotein, choline acetyltransferase, parvalbumin, neurofilament protein, Synapsin, synaptophysin, NeuN, NSE, MAP2, Beta III tubulin, 160 kD Neurofilament medium/200 kD Neurofilament Heavy, NSE, PSD93/PSD95, Doublecortin (DCX), c-fos, PSA-NCAM, NeuroD or Beta2, Tau, Calbindin-D28k, Calretinin, Neurofilament Protein (NFP), Glial fibrillary acidic protein (GFAP), S100β, Vimentin and CNPase; or by staining tissue sections with cell-identifying stains, e.g., H&E stain, Nissl Stain, Cresyl violet, Neutral red, Thionine and Toluidine blue, Luxol Fast blue stain, Weigert's Chromium hematoxylin method, Page's iron-eriochrome cyanine R, Dextran Conjugates (Fluorescein, Tetramethylrhodamine, Texas Red, Rhodamine Green), Hydrazides & Biocytins, Isolectin GS-1B4 conjugates, Golgi silver stain, or myelin stain; or by imaging the nervous system, e.g., by MRI, CT, PET, EEG, EMG, Myelogram, or magnetoencephalography. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, prostate cancer, lung cancer, breast cancer, skin cancer (e.g., melanoma), renal cell carcinoma, or colorectal cancer. In some embodiments, the cancer is a cancer listed in Table 5. In some embodiments, the neoplasm is derived from a secretory tissue, glandular tissue, or endocrine or hormonal tissue.

In one embodiment, the method includes (a) identifying (e.g., diagnosing) a patient who has a neoplasm, (b) optionally evaluating the neoplasm for perineural invasion, and (c) selecting a GRK2 inhibitor to treat the patient if the neoplasm exhibits perineural invasion. In some embodiments, the neoplasm is selected from: head and neck squamous cell carcinoma, adenoid cystic carcinoma, lymphoma, rhabdomyosarcoma, biliary tract cancer, gastric cancer, pancreatic cancer, and prostate cancer.

In one embodiment, the method includes (a) identifying (e.g., diagnosing) a patient who has a neoplasm, (b) optionally evaluating the subject for metastasis to brain or spinal cord, and (c) selecting a GRK2 inhibitor to treat the patient if the neoplasm exhibits metastasis to brain or spinal cord. In some embodiments, the neoplasm is a lung cancer (e.g., small cell lung cancer, non-small cell lung cancer), breast cancer, ovarian cancer, skin cancer (e.g., melanoma), lymphoma, renal cell carcinoma, GI tract cancer (e.g., gastric cancer), head and neck cancer, prostate cancer, pancreatic cancer, or colorectal cancer.

In one embodiment, the method includes (a) identifying (e.g., diagnosing) a patient who has cancer, (b) optionally evaluating the subject for GRK2 overexpression, and (c) selecting a GRK2 inhibitor to treat the patient if the cancer exhibits GRK2 overexpression (e.g., if the patient has GRK2-associated cancer). In some embodiments, the neoplasm is a lung cancer (e.g., small cell lung cancer, non-small cell lung cancer), breast cancer, skin cancer (e.g., melanoma), lymphoma, renal cell carcinoma, GI tract cancer, prostate cancer, ovarian cancer, uterine cancer, head and neck cancer, esophageal cancer, mesothelioma, colorectal cancer, or gastric cancer. GRK2 expression can be measured in a cancer sample collected from a subject using standard techniques known in the art, such as immunohistochemistry, western blot analysis, quantitative RT-PCR, RNA sequencing, fluorescent in situ hybridization, cDNA microarray, and droplet digital PCR. A cancer sample can be evaluated for increased expression of GRK2 by comparison to a reference sample (e.g., a non-cancerous cell of the same type).

In some embodiments, the method includes administering the selected treatment to the subject.

The method may also include a step of assessing the subject for a parameter of cancer progression or remission, e.g., assessing the subject for one or more (e.g., 2 or more, 3 or more, 4 or more) of: primary tumor size (e.g., by imaging), number of metastases (e.g., by imaging or biopsy), cell death in situ (e.g., by biopsy), blood antigen markers (e.g., by ELISA), circulating tumor DNA (e.g., by PCR), or function of the affected organ (e.g., by a test of circulating enzymes for liver, albuminuria for kidney, lung capacity for lung, etc.).

In some embodiments, the tumor is treated with a GRK2 inhibitor and a second therapeutic agent. The second therapeutic agent can be selected based on tumor type, tumor tissue of origin, tumor stage, tumor innervation, or mutations in genes expressed by the tumor.

In certain embodiments, a GRK2 inhibitor administered according to the methods described herein does not have a direct effect on the CNS or gut. Any effect on the CNS or gut is reduced compared to the effect observed if the GRK2 inhibitor is administered directly to the CNS or gut. In some embodiments, direct effects on the CNS or gut are avoided by modifying the GRK2 inhibitor not to cross the BBB, as described herein above, or administering the agent locally to a subject.

Subjects with cancer or at risk of developing cancer are treated with an effective amount of a GRK2 inhibitor. The methods described herein also include contacting a tumor or cancer cell with an effective amount of a GRK2 inhibitor. In some embodiments, an effective amount of a GRK2 inhibitor is an amount sufficient to decrease tumor innervation or nerve activity in a tumor. In some embodiments, an effective amount of a GRK2 inhibitor is an amount sufficient to treat the cancer or tumor, cause remission, reduce tumor growth, reduce tumor volume, reduce tumor metastasis, reduce tumor invasion, reduce tumor proliferation, reduce tumor migration, or reduce tumor number, reduce GRK2 expression, increase cancer cell death, increase cancer cell autophagy, increase time to recurrence, or improve survival.

The GRK2 inhibitors described herein are administered in an amount (e.g., an effective amount) and for a time sufficient to effect one of the outcomes described above. The GRK2 inhibitor may be administered once or more than once. The GRK2 inhibitor may be administered once daily, twice daily, three times daily, once every two days, once weekly, twice weekly, three times weekly, once biweekly, once monthly, once bimonthly, twice a year, or once yearly. Treatment may be discrete (e.g., an injection) or continuous (e.g., treatment via an implant or infusion pump). Subjects may be evaluated for treatment efficacy 1 week, 2 weeks, 1 month, 2 months, 3 months, 4 months, 5 months, 6 months or more following administration of a GRK2 inhibitor depending on the GRK2 inhibitor and route of administration used for treatment. Depending on the outcome of the evaluation, treatment may be continued or ceased, treatment frequency or dosage may change, or the patient may be treated with a different GRK2 inhibitor. Subjects may be treated for a discrete period of time (e.g., 1, 2, 3, 4, 5, 6, 7, 8, 9, 10, 11, or 12 months) or until the disease or condition is alleviated, or treatment may be chronic depending on the severity and nature of the disease or condition being treated.

### Kits

The invention also features a kit containing (a) a pharmaceutical composition including a GRK2 inhibitor described herein, and (b) instructions for administering the pharmaceutical composition to treat cancer.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1. Identification of GRK2 as Essential for Growth and Proliferation of Pancreatic Cancer

To identify neurological genes relevant to the proliferation of pancreatic cancer, a CRISPR library of lentiviral-encoded guide RNAs (gRNA) that individually target neuronal genes was synthesized (Cellecta, Inc.), with a library coverage of six unique gRNAs per gene. Pancreatic cancer cell lines PANC1, MIAPACA2, and BXPC3, along with cell lines derived from several patient-derived xenograft (PDX) models were transduced with a lentiviral-encoded Cas9 nuclease at a high multiplicity of infection (MOI), then transduced with the gRNA lentivirus library at a low MOI of 0.5 to ensure that individual cells received no more than one gRNA. One day post transduction, cells were incubated with puromycin for four days to select for successfully transduced cells (the gRNA library vector also encodes puromycin resistance).

Following transduction and selection, 10×10⁶ cells were harvested to serve as the “baseline” population from which the growth effects of individual genes would be compared. For the in vitro arm, 10×10⁶ cells were plated in manufacturer's recommended medium and split twice weekly for four weeks, always re-plating 10×10⁶ cells. For the in vivo subcutaneous arm, 5×10⁶ cells were implanted subcutaneously into NOD-SCID mice, with three mice per replicate, and allowed to grow for four weeks. For the in vivo orthotopic arm, 5×10⁵ cells were implanted orthotopically into the pancreas of NOD-SCID mice, with 10 mice per replicate, and allowed to grow for four weeks.

Following the four weeks of cell/tumor growth, DNA from in vitro and in vivo samples was isolated by tissue homogenization and lysis using Qiagen DNeasy Blood and Tissue Kit, and concentrated by ethanol precipitation. The DNA samples were amplified by two rounds of PCR using manufacturer recommended primers and analyzed by next-generation sequencing (BGIAmerica).

Sequencing results were analyzed to call hits using a Model-based Analysis of Genome-wide CRISPR-Cas9 Knockout (MAGeCK) algorithm as described by Li W et al., Genome Biology 2014 and Li W et al., Genome Biology 2015. Essentially, sequencing reads were normalized to their medians, the variance of read counts for individual gRNAs were estimated and normalized, and individual gRNA read count differences were ranked against each other. Essential genes were called based on whether multiple of the gRNAs targeting this gene rank near the top of the gRNA ranking list. The gRNAs that were used to target GRK2 were encoded by DNA molecules with the sequences listed in Table 14.

TABLE 14
DNA SEQUENCES ENCODING GRK2-TARGETING gRNA

| Gene | DNA sequence encoding GRK2-targeting gRNA |
|---|---|
| GRK2 | TGTCATGCAGAAGTACCTGG (SEQ ID NO: 1) |
| GRK2 | GATTTGTCAAAACCTCCGAG (SEQ ID NO: 2) |
| GRK2 | GGTCCCGGTAGACCACGAAG (SEQ ID NO: 3) |
| GRK2 | GCACCCCAGAGAGAACCAGT (SEQ ID NO: 4) |
| GRK2 | CTTTCTTGCGAGCCTCCAGC (SEQ ID NO: 5) |
| GRK2 | GCTGCGCGACGCCTACCGCG (SEQ ID NO: 6) |

Significant genes were quantified along three parameters: the Beta score, essentially the magnitude of the effect (log-fold change in gRNA count); the p-value; and the false discovery rate (FDR). Beta scores <0 indicated that the six gRNAs targeting a single gene were absent from the late stage sample compared to the baseline sample, and are a good indication that the gene is “dropping out” in the course of tumor growth. P-value and FDR both reflect the confidence that the result is not spurious, with a lower value indicating higher confidence. Thresholds for calling hits were P-value <0.1 and FDR <0.5.

GRK2 showed up as a significant hit in multiple model systems, as shown in Table 15 below. The data indicate that GRK2 is essential to the growth and proliferation of pancreatic cancer and suggest that inhibiting GRK2 may be useful for the treatment of cancer.

TABLE 15
GRK2 CRISPR RESULTS

| Gene | B-score | P-value | FDR | Condition |
|---|---|---|---|---|
| GRK2 | −0.73 | 0.00025 | 0.0069 | in vitro BXPC3 |
| GRK2 | −0.56 | 0 | 0 | 21 d in vitro PAXF1657 |
| GRK2 | −0.89 | 0 | 0 | 28 d in vitro PAXF1657 |
| GRK2 | −1.4 | 0.002 | 0.18 | Ortho PAXF1657 |
| GRK2 | −0.71 | 0.024 | 0.37 | SubQ PAXF1657 |

To validate the findings from the CRISPR screen, Cas9-expressing pancreatic cell lines were generated that express doxycycline inducible sgRNAs targeting GRK2. Treatment of these cells with 100 ng/ml doxycycline for 3, 7 or 10 days induced a time-dependent depletion of Grk2 protein, confirming the on-target efficacy of the sgRNAs. Grk2 levels were measured using Western Blot analysis, and the results are shown in Table 16.

TABLE 16
GRK2 SGRNAS REDUCED GRK2 PROTEIN LEVELS

| | | | | | GAPDH | GRK2 | GRK2/ | GRK2/GAPDH |
|---|---|---|---|---|---|---|---|---|
| | | | | | Avg. | Avg. | GAPDH | normalized |
| Cell Line | sgRNA | Days | | Dox | Intensity | Intensity | ratio | to −dox |
| Miapaca | sgNTC | D | 3 | − | 850,134.00 | 100,566.00 | 0.12 | |
| Miapaca | sgNTC | D | 3 | + | 720,778.00 | 68,452.00 | 0.09 | 0.80 |
| Miapaca | sgNTC | D | 7 | − | 977,426.00 | 125,362.00 | 0.13 | |
| Miapaca | sgNTC | D | 7 | + | 1,304,839.00 | 184,822.00 | 0.14 | 1.10 |
| Miapaca | sgNTC | D | 10 | − | 1,031,499.00 | 136,972.00 | 0.13 | |
| Miapaca | sgNTC | D | 10 | + | 996,960.00 | 112,064.00 | 0.11 | 0.85 |
| Miapaca | sgGRK2- | D | 3 | − | 730,069.00 | 29,494.00 | 0.04 | |
| | pooled | | | | | | | |
| Miapaca | sgGRK2- | D | 3 | + | 686,300.00 | 7,540.00 | 0.01 | 0.27 |
| | pooled | | | | | | | |
| Miapaca | sgGRK2- | D | 7 | − | 909,144.00 | 52,534.00 | 0.06 | |
| | pooled | | | | | | | |
| Miapaca | sgGRK2- | D | 7 | + | 950,955.00 | −4,392.00 | 0.00 | −0.08 |
| | pooled | | | | | | | |
| Miapaca | sgGRK2- | D | 10 | − | 778,200.00 | 37,758.00 | 0.05 | |
| | pooled | | | | | | | |
| Miapaca | sgGRK2- | D | 10 | + | 953,273.00 | 1,128.00 | 0.00 | 0.02 |
| | pooled | | | | | | | |
| Panc1 | sgNTC | D | 3 | − | 660,115.00 | 113,134.00 | 0.17 | |
| Panc1 | sgNTC | D | 3 | + | 680,708.00 | 114,472.00 | 0.17 | 0.98 |
| Panc1 | sgNTC | D | 7 | − | 1,123,100.00 | 201,864.00 | 0.18 | |
| Panc1 | sgNTC | D | 7 | + | 791,235.00 | 202,452.00 | 0.26 | 1.42 |
| Panc1 | sgNTC | D | 10 | − | 458,807.00 | 83,888.00 | 0.18 | |
| Panc1 | sgNTC | D | 10 | + | 527,138.00 | 88,814.00 | 0.17 | 0.92 |
| Panc1 | sgGRK2- | D | 3 | − | 101,675.00 | 9,250.00 | 0.09 | |
| | pooled | | | | | | | |
| Panc1 | sgGRK2- | D | 3 | + | 286,459.00 | 3,152.00 | 0.01 | 0.12 |
| | pooled | | | | | | | |
| Panc1 | sgGRK2- | D | 7 | − | 663,706.00 | 33,356.00 | 0.05 | |
| | pooled | | | | | | | |
| Panc1 | sgGRK2- | D | 7 | + | 777,941.00 | 1,264.00 | 0.00 | 0.03 |
| | pooled | | | | | | | |
| Panc1 | sgGRK2- | D | 10 | − | 379,379.00 | 17,864.00 | 0.05 | |
| | pooled | | | | | | | |
| Panc1 | sgGRK2- | D | 10 | + | 380,889.00 | −1,010.00 | 0.00 | −0.06 |
| | pooled | | | | | | | |
| BXPC3 | sgNTC | D | 3 | − | 138,204.00 | 3,882.00 | 0.03 | |
| BXPC3 | sgNTC | D | 3 | + | 774,148.00 | 81,590.00 | 0.11 | 3.75 |
| BXPC3 | sgNTC | D | 7 | − | 200,710.00 | 104,800.00 | 0.52 | |
| BXPC3 | sgNTC | D | 7 | + | 1,171,479.00 | 211,288.00 | 0.18 | 0.35 |
| BXPC3 | sgNTC | D | 10 | − | 1,100,387.00 | 151,640.00 | 0.14 | |
| BXPC3 | sgNTC | D | 10 | + | 1,207,918.00 | 173,510.00 | 0.14 | 1.04 |
| BXPC3 | sgGRK2- | D | 3 | − | 716,863.00 | 138,426.00 | 0.19 | |
| | pooled | | | | | | | |
| BXPC3 | sgGRK2- | D | 3 | + | 1,019,754.00 | 120,906.00 | 0.12 | 0.61 |
| | pooled | | | | | | | |
| BXPC3 | sgGRK2- | D | 7 | − | 485,164.00 | 97,116.00 | 0.20 | |
| | pooled | | | | | | | |
| BXPC3 | sgGRK2- | D | 7 | + | 889,095.00 | 115,170.00 | 0.13 | 0.65 |
| | pooled | | | | | | | |
| BXPC3 | sgGRK2- | D | 10 | − | 604,521.00 | 131,636.00 | 0.22 | |
| | pooled | | | | | | | |
| BXPC3 | sgGRK2- | D | 10 | + | 864,103.00 | 85,668.00 | 0.10 | 0.46 |
| | pooled | | | | | | | |
| PAXF1997 | sgNTC | D | 3 | − | 1,495,184.00 | 258,488.00 | 0.17 | |
| PAXF1997 | sgNTC | D | 3 | + | 1,730,788.00 | 332,610.00 | 0.19 | 1.11 |
| PAXF1997 | sgNTC | D | 7 | − | 1,921,289.00 | 260,904.00 | 0.14 | |
| PAXF1997 | sgNTC | D | 7 | + | 2,102,602.00 | 381,128.00 | 0.18 | 1.33 |
| PAXF1997 | sgNTC | D | 10 | − | 1,918,507.00 | 429,332.00 | 0.22 | |
| PAXF1997 | sgNTC | D | 10 | + | 2,588,082.00 | 537,898.00 | 0.21 | 0.93 |
| PAXF1997 | sgGRK2- | D | 3 | − | 1,929,746.00 | 292,512.00 | 0.15 | |
| | pooled | | | | | | | |
| PAXF1997 | sgGRK2- | D | 3 | + | 1,516,350.00 | 61,756.00 | 0.04 | 0.27 |
| | pooled | | | | | | | |
| PAXF1997 | sgGRK2- | D | 7 | − | 2,165,121.00 | 394,554.00 | 0.18 | |
| | pooled | | | | | | | |
| PAXF1997 | sgGRK2- | D | 7 | + | 2,343,604.00 | 133,038.00 | 0.06 | 0.31 |
| | pooled | | | | | | | |
| PAXF1997 | sgGRK2- | D | 10 | − | 2,108,003.00 | 489,080.00 | 0.23 | |
| | pooled | | | | | | | |
| PAXF1997 | sgGRK2- | D | 10 | + | 2,366,810.00 | 81,574.00 | 0.03 | 0.15 |
| | pooled | | | | | | | |
| PAXF1997 | sgGRK2 | D | 3 | − | 657,735.68 | 1,900,268.97 | 0.35 | |
| | pooled | | | | | | | |
| PAXF1997 | sgGRK2 | D | 3 | + | 204,037.28 | 1,881,941.79 | 0.11 | 0.31 |
| | pooled | | | | | | | |
| PAXF1997 | sgGRK2 | D | 7 | − | 676,658.40 | 1,980,927.72 | 0.34 | |
| | pooled | | | | | | | |
| PAXF1997 | sgGRK2 | D | 7 | + | 264,522.88 | 2,296,446.76 | 0.12 | 0.34 |
| | pooled | | | | | | | |
| PAXF1997 | sgGRK2 | D | 10 | − | 787,256.80 | 1,828,088.97 | 0.43 | |
| | pooled | | | | | | | |
| PAXF1997 | sgGRK2 | D | 10 | + | 135,021.12 | 1,464,604.41 | 0.09 | 0.21 |
| | pooled | | | | | | | |
| PAXF1997 | sgGRK2 | D | 14 | − | 749,224.16 | 1,757,432.97 | 0.43 | |
| | pooled | | | | | | | |
| PAXF1997 | sgGRK2 | D | 14 | + | 148,905.28 | 1,468,029.79 | 0.10 | 0.24 |
| | pooled | | | | | | | |
| PAXF1997 | sgGRK2 | D | 18 | − | 348,556.16 | 1,540,794.90 | 0.23 | |
| | pooled | | | | | | | |
| PAXF1997 | sgGRK2 | D | 18 | + | 111,389.76 | 1,759,827.31 | 0.06 | 0.28 |
| | pooled | | | | | | | |

| | | | | | | | |
|---|---|---|---|---|---|---|---|
| PAXF1657 | sgNTC | 3 | − | 1,648,078.11 | 6,387,847.41 | 0.26 | |
| PAXF1657 | sgNTC | 3 | + | 2,330,340.78 | 5,279,594.79 | 0.44 | 1.71 |
| PAXF1657 | sgNTC | 7 | − | 3,709,716.22 | 4,510,160.86 | 0.82 | |
| PAXF1657 | sgNTC | 7 | + | 3,301,525.00 | 4,306,119.99 | 0.77 | 0.93 |
| PAXF1657 | sgGRK2-1 | 7 | − | 619,833.22 | 3,759,964.63 | 0.16 | |
| | (SEQ ID | | | | | | |
| | NO: 1) | | | | | | |
| PAXF1657 | sgGRK2-1 | 7 | + | 190,054.33 | 4,541,178.51 | 0.04 | 0.25 |
| | (SEQ ID | | | | | | |
| | NO: 1) | | | | | | |
| PAXF1657 | sgGRK2-2 | 7 | − | 939,202.78 | 3,309,926.48 | 0.28 | |
| | (SEQ ID | | | | | | |
| | NO: 3) | | | | | | |
| PAXF1657 | sgGRK2-2 | 7 | + | 119,039.00 | 2,133,921.03 | 0.06 | 0.20 |
| | (SEQ ID | | | | | | |
| | NO: 3) | | | | | | |
| PAXF1657 | sgGRK2-3 | 7 | − | 1,090,217.00 | 1,925,734.51 | 0.57 | |
| | (SEQ ID | | | | | | |
| | NO: 4) | | | | | | |
| PAXF1657 | sgGRK2-3 | 7 | + | 318,007.56 | 2,026,964.66 | 0.16 | 0.28 |
| | (SEQ ID | | | | | | |
| | NO: 4) | | | | | | |
| PAXF1657 | sgGRK2- | 7 | − | 2,668,943.00 | 4,579,845.04 | 0.58 | |
| | pooled | | | | | | |
| PAXF1657 | sgGRK2- | 7 | + | 473,372.00 | 5,793,544.31 | 0.08 | 0.14 |
| | pooled | | | | | | |

Next, experiments were performed to evaluate whether GRK2 knockout induced by doxycycline treatment results in a proliferation defect in pancreatic cancer cells. Pancreatic cancer cell lines expressing GRK2 sgRNAs as well as RFP were plated in a ratio of 1:1 with GFP expressing pancreatic cancer cells. Co-plated cells were cultured in the presence or absence of 100 ng/ml doxycycline over the course of 21 days, and the percentage of GFP and RFP-expressing cells was measured over time. In this competition assay, knockout of GRK2 by individual or pooled sgRNAs showed an anti-proliferative effect in all pancreatic cancer cell lines tested, with the strongest phenotypes observed in PAXF1657, BXPC3 and PAXF1997, correlating with the pooled CRISPR screen results. These data are shown in Tables 17A-17E, below, with the RFP/GFP ratio +dox normalized to RFP/GFP ratio −dox.

TABLE 17A
DOXYCYCLINE-INDUCED GRK2 KNOCKOUT- PAXF1657

| | | | | sgGRK2-1 | sgGRK2-2 | sgGRK2-3 |
|---|---|---|---|---|---|---|
| | | | sgGRK2 | (SEQ ID | (SEQ ID | (SEQ ID |
| days | sgNTC | sgPCNA | pooled | NO: 1) | NO: 3) | NO: 4) |
| 3 | 0.96 | 0.95 | 0.87 | 0.98 | 1.00 | 0.92 |
| 7 | 1.05 | 0.57 | 0.85 | 0.91 | 0.89 | 0.83 |
| 10 | 1.12 | 0.29 | 0.65 | 0.85 | 0.76 | 0.64 |
| 14 | 1.04 | 0.17 | 0.49 | 0.78 | 0.69 | 0.44 |
| 17 | 0.90 | 0.11 | 0.40 | 0.68 | 0.57 | 0.27 |
| 21 | 0.95 | 0.11 | 0.40 | 0.53 | 0.44 | 0.24 |

TABLE 17B
DOXYCYCLINE-INDUCED GRK2 KNOCKOUT- MIAPACA2

| days | sgNTC | sgPCNA | sgGRK2 pooled |
|---|---|---|---|
| 3 | 0.95 | 0.77 | 0.91 |
| 7 | 0.92 | 0.22 | 0.82 |
| 14 | 0.97 | 0.09 | 0.66 |
| 18 | 0.95 | 0.07 | 0.60 |
| 21 | 0.94 | 0.05 | 0.56 |

TABLE 17C
DOXYCYCLINE-INDUCED GRK2 KNOCKOUT- PANC1

| days | sgNTC | sgPCNA | sgGRK2 pooled |
|---|---|---|---|
| 3 | 1.01 | 0.62 | 0.83 |
| 7 | 0.93 | 0.14 | 0.65 |
| 14 | 0.86 | 0.02 | 0.45 |
| 18 | 0.98 | 0.02 | 0.48 |
| 21 | 0.94 | 0.01 | 0.49 |

TABLE 17D
DOXYCYCLINE-INDUCED GRK2 KNOCKOUT- BXPC3

| days | sgNTC | sgPCNA | sgGRK2 pooled |
|---|---|---|---|
| 3 | 0.93 | 0.99 | 0.86 |
| 7 | 1.01 | 0.94 | 0.82 |
| 14 | 0.91 | 0.41 | 0.53 |
| 18 | 1.01 | 0.32 | 0.50 |
| 21 | 0.94 | 0.29 | 0.39 |

TABLE 17E
DOXYCYCLINE-INDUCED GRK2 KNOCKOUT- PAXF1997

| days | sgNTC | sgPCNA | sgGRK2 pooled |
|---|---|---|---|
| 3 | 1.07 | 1.00 | 0.90 |
| 7 | 1.09 | 0.81 | 0.78 |
| 14 | 1.12 | 0.49 | 0.55 |
| 18 | 1.16 | 0.39 | 0.46 |
| 21 | 1.28 | 0.30 | 0.32 |

### Example 2. The Anti-Proliferative Effect of GRK2 Inhibition is Due to Loss of Grk2 Kinase Activity

To test whether the Grk2 sgRNAs act through targeting Grk2, and to test whether the anti-proliferative effect results from loss of the Grk2 kinase activity, Grk2 sgRNA expressing PAXF1657 cell lines overexpressing Cas9/sgRNA-cleavage-resistant Grk2 wild-type or kinase-dead cDNA constructs were generated. Re-expression of Cas9/sgRNA-cleavage-resistant Grk2 wild-type or kinase-dead cDNA constructs led to increased Grk2 protein levels, as measured by Western Blot analysis and shown in Table 18. Re-expression of Grk2 wild-type cDNA in sgGrk2-2 (SEQ ID NO: 3) expressing PAXF1657 cells reduced the sgRNA-induced growth inhibition, whereas re-expression of the Grk2 kinase-dead cDNA did not, as shown in Table 19 (values represent the RFP/GFP ratio +dox normalized to RFP/GFP ratio −dox). This suggests that the kinase activity of Grk2 is essential for the role of Grk2 on cell survival. In the context of sgGrk2-3 (SEQ ID NO: 4) expressing PAXF1657 cells, rescue of growth inhibition by re-expression of Grk2 wild-type cDNA was less efficient, potentially due to lower re-expression of Grk2 in these cells or due to some off-target activity of the sgRNA.

TABLE 18
GRK2 LEVELS IN PAXF1657 CELL LINES OVEREXPRESSING CAS9/SGRNA-
CLEAVAGE-RESISTANT GRK2 WT OR KINASE-DEAD CDNA CONSTRUCTS

| | | | | | | GRK2/ |
|---|---|---|---|---|---|---|
| | | | | | | GAPDH |
| | | | GRK2 | GAPDH | GRK2/ | normalized |
| Cell Line | cDNA | Treatment | Adj. Vol. (Int) | Adj. Vol. (Int) | GAPDH | to −dox |
| PAXF1657- | Empty | −dox | 517,219.94 | 12,741,232.67 | 0.04 | |
| sgNT | vector | | | | | |
| PAXF1657- | Empty | +dox | 690,882.92 | 21,016,384.00 | 0.03 | 0.81 |
| sgNT | vector | | | | | |
| PAXF1657- | sgGRK2#2 | −dox | 10,343,005.94 | 23,974,881.67 | 0.43 | |
| sgNT | res | | | | | |
| PAXF1657- | sgGRK2#2 | +dox | 5,659,532.02 | 20,612,415.33 | 0.27 | 0.64 |
| sgNT | res | | | | | |
| PAXF1657- | sgGRK2#2 | −dox | 1,273,895.69 | 9,517,351.33 | 0.13 | |
| sgNT | KD res | | | | | |
| PAXF1657- | sgGRK2#2 | +dox | 1,556,476.86 | 11,498,672.00 | 0.14 | 1.01 |
| sgNT | KD res | | | | | |
| PAXF1657- | sgGRK2#3 | −dox | 6,501,271.10 | 18,889,385.00 | 0.34 | |
| sgNT | res | | | | | |
| PAXF1657- | sgGRK2#3 | +dox | 8,133,567.10 | 20,580,295.67 | 0.40 | 1.15 |
| sgNT | res | | | | | |
| PAXF1657- | sgGRK2#3 | −dox | 3,037,131.76 | 23,103,834.33 | 0.13 | |
| sgNT | KD res | | | | | |
| PAXF1657- | sgGRK2#3 | +dox | 3,610,963.69 | 20,987,549.33 | 0.17 | 1.31 |
| sgNT | KD res | | | | | |
| PAXF1657- | Empty | −dox | 933,193.82 | 18,372,389.33 | 0.05 | |
| sgGRK2-2 | vector | | | | | |
| PAXF1657- | Empty | +dox | −189,385.63 | 15,831,400.67 | −0.01 | −0.24 |
| sgGRK2-2 | vector | | | | | |
| PAXF1657- | sgGRK2#2 | −dox | 10,774,146.31 | 14,680,373.00 | 0.73 | |
| sgGRK2-2 | res | | | | | |
| PAXF1657- | sgGRK2#2 | +dox | 9,575,312.76 | 13,308,717.33 | 0.72 | 0.98 |
| sgGRK2-2 | res | | | | | |
| PAXF1657- | sgGRK2#2 | −dox | 2,591,844.94 | 13,897,219.33 | 0.19 | |
| sgGRK2-2 | KD res | | | | | |
| PAXF1657- | sgGRK2#2 | +dox | 4,784,299.06 | 13,531,826.33 | 0.35 | 1.90 |
| sgGRK2-2 | KD res | | | | | |
| PAXF1657- | Empty | −dox | 293,372.12 | 17,075,476.00 | 0.02 | |
| sgGRK2-3 | vector | | | | | |
| PAXF1657- | Empty | +dox | −514,147.69 | 12,133,157.00 | −0.04 | −2.47 |
| sgGRK2-3 | vector | | | | | |
| PAXF1657- | sgGRK2#3 | −dox | 3,254,562.84 | 24,094,330.00 | 0.14 | |
| sgGRK2-3 | res | | | | | |
| PAXF1657- | sgGRK2#3 | +dox | 4,806,774.61 | 15,187,382.00 | 0.32 | 2.34 |
| sgGRK2-3 | res | | | | | |
| PAXF1657- | sgGRK2#3 | −dox | 6,259,054.49 | 17,030,119.33 | 0.37 | |
| sgGRK2-3 | KD res | | | | | |
| PAXF1657- | sgGRK2#3 | +dox | 9,225,716.59 | 13,838,994.33 | 0.67 | 1.81 |
| sgGRK2-3 | KD res | | | | | |

TABLE 19
GROWTH OF PAXF1657 CELL LINES OVEREXPRESSING CAS9/SGRNA-
CLEAVAGE-RESISTANT GRK2 WT OR KINASE-DEAD CDNA CONSTRUCTS

| | | | sgNTC- | | sgNTC- | | | sgGRK2- | | | |
|---|---|---|---|---|---|---|---|---|---|---|---|
| | | sgNTC- | +GRK2 | | +GRK2 | | sgGRK2- | 2 +GRK2 | | sgGRK2- | sgGRK2- |
| | | +GRK2 | kinase | sgNTC- | kinase | | 2 +GRK2 | kinase | | 3 +GRK2 | 3 +GRK2 |
| | | wild-type | dead | +GRK2 | dead | | wild-type | dead | | wild-type | kinase |
| | | cDNA | cDNA | wt cDNA | cDNA | | cDNA | cDNA | | cDNA | dead cDNA |
| | sgNTC | (res. to | (res. to | (res. to | (res. to | sgGRK2-2 + | (res. to | (res. to | sgGRK2-3 + | (res. to | (res. to |
| | empty | sgRNA2 | sgRNA2 | sgRNA3 | sgRNA3 | empty | sgRNA2 | sgRNA2 | empty | sgRNA3 | sgRNA3 |
| days | vector | cleavage) | cleavage) | cleavage) | cleavage) | vector | cleavage) | cleavage) | vector | cleavage) | cleavage) |
| 3 | 1.09 | 1.02 | 0.83 | 0.97 | 0.83 | 0.92 | 0.85 | 0.85 | 0.79 | 0.97 | 0.90 |
| 7 | 0.85 | 0.93 | 1.07 | 0.81 | 1.18 | 0.66 | 0.99 | 1.06 | 0.78 | 0.78 | 0.82 |
| 10 | 0.99 | 1.05 | 0.91 | 0.91 | 0.99 | 0.40 | 0.75 | 0.68 | 0.61 | 0.73 | 0.49 |
| 14 | 0.76 | 0.85 | 1.00 | 0.85 | 0.88 | 0.28 | 0.91 | 0.45 | 0.38 | 0.57 | 0.39 |
| 17 | 0.76 | 0.89 | 1.59 | 0.81 | 1.08 | 0.17 | 0.53 | 0.23 | 0.27 | 0.54 | 0.26 |
| 21 | 0.84 | 0.88 | 1.23 | 1.13 | 0.85 | 0.10 | 0.69 | 0.15 | 0.24 | 0.34 | 0.23 |

### Example 3. The GRK2 Small Molecule Antagonist of Formula III-26 Reduces Pancreatic Cancer Cell Proliferation

The GRK2 small molecule antagonist of Formula III-26 was used to evaluate the effect of a small molecule GRK2 antagonist on the proliferation of pancreatic cancer cell lines Miapaca2, Panc1, BXPC3, PAXF1657, PAXF1997 and Capan1. Values represent percent viability normalized to DMSO. As shown in Table 20, the compound having the structure of Formula III-26 reduced cell proliferation.

TABLE 20
PROLIFERATION OF CELLS TREATED WITH THE COMPOUND OF FORMULA III-26

| Dose | Miapaca2 | | Panc1 | | BXPC3 | | PAXF1657 | | PAXF1997 | | Capan1 | |
|---|---|---|---|---|---|---|---|---|---|---|---|---|
| (μM) | Avg | STDV | Avg | STDV | Avg | STDV | Avg | STDV | Avg | STDV | Avg | STDV |
| 30.000 | 40.0 | 1.36 | 86.2 | 6.34 | 75.8 | 1.55 | 69.0 | 1.72 | 81.4 | 0.56 | 95.4 | 1.61 |
| 10.000 | 66.1 | 3.16 | 85.2 | 6.10 | 77.0 | 3.20 | 88.6 | 0.31 | 83.7 | 0.91 | 90.0 | 1.52 |
| 3.333 | 86.6 | 5.87 | 100.2 | 7.60 | 91.5 | 4.60 | 101.7 | 2.47 | 104.1 | 3.75 | 100.4 | 2.04 |
| 1.111 | 99.3 | 4.12 | 112.8 | 9.04 | 102.4 | 1.72 | 109.5 | 3.56 | 110.0 | 3.24 | 106.4 | 2.94 |
| 0.370 | 113.7 | 7.43 | 113.3 | 13.07 | 107.6 | 1.22 | 115.4 | 0.40 | 118.2 | 5.74 | 111.9 | 4.53 |
| 0.123 | 110.9 | 4.88 | 112.7 | 8.48 | 104.0 | 0.74 | 117.6 | 2.83 | 113.8 | 5.50 | 114.2 | 3.76 |
| 0.041 | 108.3 | 3.10 | 113.4 | 6.92 | 104.4 | 2.94 | 116.0 | 1.34 | 111.5 | 6.27 | 112.3 | 4.07 |
| 0.014 | 104.8 | 5.68 | 112.4 | 9.69 | 104.6 | 2.49 | 110.8 | 0.98 | 111.3 | 3.65 | 108.4 | 2.57 |
| 0.005 | 102.4 | 6.89 | 103.1 | 5.49 | 105.8 | 1.78 | 111.4 | 1.49 | 111.2 | 3.73 | 107.2 | 2.46 |
| 0.000 | 100.0 | 4.31 | 100.0 | 5.82 | 100.0 | 1.85 | 100.0 | 2.50 | 100.0 | 2.15 | 100.0 | 2.67 |

Different concentrations of the compound of Formula III-26 were then used to evaluate its effect on pancreatic cancer cell line proliferation in a long-term colony formation assay (10-14 days). As shown in Table 21, the GRK2 inhibitor of Formula III-26 also reduced pancreatic cancer cell line proliferation in this assay.

TABLE 21
PROLIFERATION OF CELLS TREATED WITH THE COMPOUND
OF FORMULA III-26 - COLONY FORMATION ASSAY

| | | Concentration | | Mean | Normalized |
|---|---|---|---|---|---|
| Cell Line | Drug | μM | | intensity | to DMSO |
| BXPC3 | Compound of Formula III-26 | 0.1 | μM | 396.24 | 0.99 |
| BXPC3 | Compound of Formula III-26 | 0.3 | μM | 445.015 | 1.11 |
| BXPC3 | Compound of Formula III-26 | 1 | μM | 418.98 | 1.04 |
| BXPC3 | Compound of Formula III-26 | 3 | μM | 189.24 | 0.47 |
| BXPC3 | Compound of Formula III-26 | 10 | μM | 82.2 | 0.20 |
| BXPC3 | Compound of Formula III-26 | 30 | μM | 76.19 | 0.19 |
| BXPC3 | DMSO | | | 401.82 | 1.00 |
| Miapaca | Compound of Formula III-26 | 0.1 | μM | 447.5 | 1.09 |
| Miapaca | Compound of Formula III-26 | 0.3 | μM | 461.465 | 1.13 |
| Miapaca | Compound of Formula III-26 | 1 | μM | 398.675 | 0.97 |
| Miapaca | Compound of Formula III-26 | 3 | μM | 386.35 | 0.94 |
| Miapaca | Compound of Formula III-26 | 10 | μM | 80.16 | 0.20 |
| Miapaca | Compound of Formula III-26 | 30 | μM | 75.66 | 0.18 |
| Miapaca | DMSO | | | 409.79 | 1.00 |
| Panc1 | Compound of Formula III-26 | 0.1 | μM | 477.265 | 1.16 |
| Panc1 | Compound of Formula III-26 | 0.3 | μM | 478.195 | 1.17 |
| Panc1 | Compound of Formula III-26 | 1 | μM | 509.365 | 1.24 |
| Panc1 | Compound of Formula III-26 | 3 | μM | 504.735 | 1.23 |
| Panc1 | Compound of Formula III-26 | 10 | μM | 254.47 | 0.62 |
| Panc1 | Compound of Formula III-26 | 30 | μM | 181.7 | 0.44 |
| Panc1 | DMSO | | | 409.78 | 1.00 |
| PAXF1657 | Compound of Formula III-26 | 0.1 | μM | 617.13 | 0.97 |
| PAXF1657 | Compound of Formula III-26 | 0.3 | μM | 660.9 | 1.04 |
| PAXF1657 | Compound of Formula III-26 | 1 | μM | 574.375 | 0.90 |
| PAXF1657 | Compound of Formula III-26 | 3 | μM | 410.77 | 0.64 |
| PAXF1657 | Compound of Formula III-26 | 10 | μM | 326.68 | 0.51 |
| PAXF1657 | Compound of Formula III-26 | 30 | μM | 271.275 | 0.43 |
| PAXF1657 | DMSO | | | 637.245 | 1.00 |
| PAXF1997 | Compound of Formula III-26 | 0.1 | μM | 461.65 | 1.07 |
| PAXF1997 | Compound of Formula III-26 | 0.3 | μM | 500.34 | 1.16 |
| PAXF1997 | Compound of Formula III-26 | 1 | μM | 455.7 | 1.06 |
| PAXF1997 | Compound of Formula III-26 | 3 | μM | 379.285 | 0.88 |
| PAXF1997 | Compound of Formula III-26 | 10 | μM | 296.635 | 0.69 |
| PAXF1997 | Compound of Formula III-26 | 30 | μM | 288.38 | 0.67 |
| PAXF1997 | DMSO | | | 431.47 | 1.00 |

To assess the target specificity of the GRK2 small molecule antagonist of Formula III-26, sgGRK2-expressing cells were treated with the compound of Formula III-26. If GRK2 knockout is complete and the compound of Formula III-26 inhibits only GRK2, the addition of the compound of Formula III-26 should not induce any additional growth defect compared to sgRNA KO alone. Indeed, in Miapaca2, addition of 1 μM or 3 μM the compound of Formula III-26 to sgGrk2-expressing cells plated in a colony formation assay did not have any additional anti-proliferative effect compared to knockout alone. In contrast, in PAXF1657, addition of the compound of Formula III-26 further decreased the growth of PAXF1657 cells in the presence of GRK2 knockout, as shown in Table 22. This is potentially due to the lower GRK2 knockout efficiency that can be achieved in PAXF1657 compared to Miapaca2, as shown in Table 16.

TABLE 22
PROLIFERATION OF SGGRK2-EXPRESSING CELLS TREATED
WITH THE COMPOUND OF FORMULA III-26

| | | | | | Difference between |
|---|---|---|---|---|---|
| | | | | | rel +dox |
| Cell Line | Treatment | sgRNA | Rel −dox | Rel +dox | and −dox signal |
| PAXF1657 | DMSO | sgNTC | 1.00 | 0.84 | 0.16 |
| PAXF1657 | DMSO | sgGRK2-2 | 1.00 | 0.79 | 0.21 |
| | | (SEQ ID NO: 3) | | | |
| PAXF1657 | DMSO | sgGRK2-3 | 1.00 | 0.64 | 0.36 |
| | | (SEQ ID NO: 4) | | | |
| PAXF1657 | DMSO | sgGRK2-pooled | 1.00 | 0.65 | 0.35 |
| PAXF1657 | 1 μM Compound of | sgNTC | 0.73 | 0.78 | −0.04 |
| | Formula III-26 | | | | |
| PAXF1657 | 1 μM Compound of | sgGRK2-2 | 0.89 | 0.44 | 0.45 |
| | Formula III-26 | (SEQ ID NO: 3) | | | |
| PAXF1657 | 1 μM Compound of | sgGRK2-3 | 0.78 | 0.40 | 0.37 |
| | Formula III-26 | (SEQ ID NO: 4) | | | |
| PAXF1657 | 1 μM Compound of | sgGRK2-pooled | 0.78 | 0.49 | 0.29 |
| | Formula III-26 | | | | |
| PAXF1657 | 3 μM Compound of | sgNTC | 0.57 | 0.56 | 0.01 |
| | Formula III-26 | | | | |
| PAXF1657 | 3 μM Compound of | sgGRK2-2 | 0.67 | 0.41 | 0.26 |
| | Formula III-26 | (SEQ ID NO: 3) | | | |
| PAXF1657 | 3 μM Compound of | sgGRK2-3 | 0.55 | 0.22 | 0.32 |
| | Formula III-26 | (SEQ ID NO: 4) | | | |
| PAXF1657 | 3 μM Compound of | sgGRK2-pooled | 0.59 | 0.48 | 0.11 |
| | Formula III-26 | | | | |
| Miapaca | DMSO | sgNTC | 1.00 | 1.08 | −0.08 |
| Miapaca | DMSO | sgGRK2-pooled | 1.00 | 0.78 | 0.22 |
| Miapaca | 1 μM Compound of | sgNTC | 0.81 | 0.77 | 0.05 |
| | Formula III-26 | | | | |
| Miapaca | 1 μM Compound of | sgGRK2-pooled | 0.75 | 0.76 | 0.00 |
| | Formula III-26 | | | | |
| Miapaca | 3 μM Compound of | sgNTC | 0.65 | 0.53 | 0.12 |
| | Formula III-26 | | | | |
| Miapaca | 3 μM Compound of | sgGRK2-pooled | 0.47 | 0.43 | 0.04 |
| | Formula III-26 | | | | |

### Example 4. Evaluating the Effect of GRK2 Small Molecule Antagonists on Cancer Cell Proliferation

Cellular anti-proliferative activity of GRK2 small molecule antagonists was assessed using pancreatic cancer cell line PAXF1657 stably overexpressing GRK2 cDNA or the control empty vector or a stable clone of GRK2 knockout. Cell lines were seeded into tissue culture treated, white-walled, 96-well plates at a density of 500 cells/well in RPM11640 media supplemented with 10% H.I. FBS and penicillin/streptomycin. Plates were incubated overnight at 37° C., 5% CO₂ to allow cells to adhere to the wells. GRK2 small molecule antagonists were added to the cells using a ten-point or five-point dilution series with a final concentration ranging from 10 mM-0.015 mM in 0.1% DMSO. At the time of compound addition, a set of plates that were not treated with compounds were collected and cell viability was measured using CellTiter-Glo (Promega). CellTiter-Glo reagent was added to the plates and luminescence was measured using a Biotek Synergy plate reader. The compound treated cells were incubated for 3 days at 37° C., 5% CO₂. The media was then aspirated from each well and replaced with fresh media containing GRK2 small molecule antagonists. The compound treated cells were then incubated for another 4 days at 37° C., 5% CO₂ and cell viability was assessed at the end of the 7-day compound treatment by CellTiterGlo. The results of this experiment are shown below in Table 23.

TABLE 23
PROLIFERATION OF PAXF1657 CELLS TREATED
WITH GRK2 SMALL MOLECULE ANTAGONISTS

| | | | | | Maximum % |
|---|---|---|---|---|---|
| | | | | GI50, | Growth inhibition |
| | | | | geometric | relative to DMSO, |
| sgRNA | cDNA | Compound ID | Treatment | mean, μM | geometric mean |
| sgNTC | pReceiver- | Compound of | 5 point dose | 1.49 | 43.3 |
| | empty vector | Formula II-26 | curve of compound | | |
| sgNTC | pReceiver- | Compound of | 10 point dose | 9.49 | 61 |
| | empty vector | Formula III-26 | curve of compound | | |
| sgNTC | pReceiver- | Compound of | 10 point dose | 2.52 | 89 |
| | empty vector | Formula III-54 | curve of compound | | |
| sgNTC | pReceiver- | Compound of | 10 point dose | 0.11 | 82.3 |
| | empty vector | Formula III-55 | curve of compound | | |
| sgNTC | pReceiver- | Compound of | 5 point dose | >10 | 46.5 |
| | empty vector | Formula III-56 | curve of compound | | |
| sgNTC | pReceiver- | Compound of | 10 point dose | 0.68 | 83.6 |
| | empty vector | Formula III-35 | curve of compound | | |
| sgNTC | pReceiver- | Compound of | 5 point dose | >10 | 31.6 |
| | empty vector | Formula III-31 | curve of compound | | |
| sgNTC | pReceiver- | Compound of | 5 point dose | 6.27 | 42.7 |
| | empty vector | Formula II-33 | curve of compound | | |
| sgNTC | pReceiver- | Compound of | 10 point dose | 4.91 | 73.1 |
| | empty vector | Formula III-30 | curve of compound | | |
| sgNTC | pReceiver- | Compound of | 10 point dose | 0.653 | 76.8 |
| | empty vector | Formula III-28 | curve of compound | | |
| sgNTC | pReceiver- | Compound of | 10 point dose | 3.37 | 99.3 |
| | empty vector | Formula II-35 | curve of compound | | |
| sgNTC | pReceiver- | Compound of | 5 point dose | >10 | 25.5 |
| | GRK2 | Formula II-26 | curve of compound | | |
| sgNTC | pReceiver- | Compound of | 10 point dose | >10 | 24.8 |
| | GRK2 | Formula III-26 | curve of compound | | |
| sgNTC | pReceiver- | Compound of | 10 point dose | 2.71 | 87.3 |
| | GRK2 | Formula III-54 | curve of compound | | |
| sgNTC | pReceiver- | Compound of | 10 point dose | 0.17 | 85.9 |
| | GRK2 | Formula III-55 | curve of compound | | |
| sgNTC | pReceiver- | Compound of | 5 point dose | >10 | 28.3 |
| | GRK2 | Formula III-56 | curve of compound | | |
| sgNTC | pReceiver- | Compound of | 10 point dose | 0.614 | 81.4 |
| | GRK2 | Formula III-35 | curve of compound | | |
| sgNTC | pReceiver- | Compound of | 5 point dose | >10 | 29.8 |
| | GRK2 | Formula III-31 | curve of compound | | |
| sgNTC | pReceiver- | Compound of | 5 point dose | >10 | 0 |
| | GRK2 | Formula II-33 | curve of compound | | |
| sgNTC | pReceiver- | Compound of | 10 point dose | 3.4 | 65.5 |
| | GRK2 | Formula III-30 | curve of compound | | |
| sgNTC | pReceiver- | Compound of | 10 point dose | 14.7 | 64.7 |
| | GRK2 | Formula III-28 | curve of compound | | |
| sgNTC | pReceiver- | Compound of | 10 point dose | 5.94 | 99.3 |
| | GRK2 | Formula II-35 | curve of compound | | |
| sgGRK2 #2, | none | Compound of | 5 point dose | not tested | not tested |
| clonal KO | | Formula II-26 | curve of compound | | |
| sgGRK2 #2, | none | Compound of | 10 point dose | not tested | not tested |
| clonal KO | | Formula III-26 | curve of compound | | |
| sgGRK2 #2, | none | Compound of | 10 point dose | not tested | not tested |
| clonal KO | | Formula III-54 | curve of compound | | |
| sgGRK2 #2, | none | Compound of | 10 point dose | not tested | not tested |
| clonal KO | | Formula III-55 | curve of compound | | |
| sgGRK2 #2, | none | Compound of | 5 point dose | not tested | not tested |
| clonal KO | | Formula III-56 | curve of compound | | |
| sgGRK2 #2, | none | Compound of | 10 point dose | not tested | not tested |
| clonal KO | | Formula III-35 | curve of compound | | |
| sgGRK2 #2, | none | Compound of | 5 point dose | not tested | not tested |
| clonal KO | | Formula III-31 | curve of compound | | |
| sgGRK2 #2, | none | Compound of | 5 point dose | not tested | not tested |
| clonal KO | | Formula II-33 | curve of compound | | |
| sgGRK2 #2, | none | Compound of | 10 point dose | >30 | 63.5 |
| clonal KO | | Formula III-30 | curve of compound | | |
| sgGRK2 #2, | none | Compound of | 10 point dose | 17.9 | 78.8 |
| clonal KO | | Formula III-28 | curve of compound | | |
| sgGRK2 #2, | none | Compound of | 10 point dose | 8.8 | 99.3 |
| clonal KO | | Formula II-35 | curve of compound | | |

### Example 5. Generation of a GRK2-Specific Inhibitory Antibody

The protein sequence of GRK2 is recombinantly expressed from a mammalian cell culture system, e.g., HEK or CHO cells. Using routine methods such as phage display, yeast display, or animal immunization, an antibody is raised that is specific to GRK2 as measured by ELISA.

### Example 6. Treatment of a Patient with Cancer with a GRK2 Inhibitor

According to the methods disclosed herein, a physician of skill in the art can treat a patient, such as a human patient with cancer (e.g., pancreatic cancer), so as to inhibit cancer growth, reduce tumor burden, increase cancer cell death, or slow disease progression. The method of treatment can include diagnosing or identifying a patient as a candidate for treatment with a GRK2 inhibitor based on GRK2 expression in a biopsy. For example, a tissue sample can be collected from a patient's cancer and analyzed for RNA expression by qPCR or RNAseq analysis, and the cancer can be found to express high levels of GRK2. To treat the patient, a physician of skill in the art can administer a GRK2 inhibitor that decreases GRK2 expression or function (e.g., an inhibitory RNA or nuclease directed to GRK2 or a GRK2 small molecule antagonist, e.g., a small molecule antagonist described herein, such as an antagonist having a structure of Formula I (e.g., having the structure of any one of Formula I-1 to I-39), Formula II (e.g., having the structure of any one of Formula II-1 to II-35), Formula III (e.g., having the structure of any one of Formula III-1 to III-56), or Formula IV (e.g., having the structure of any one of Formula IV-1 to IV-80), or any one of Compounds 1-70). The GRK2 inhibitor can be administered locally (e.g., injected into the tumor or tumor microenvironment) to decrease tumor growth or volume. The GRK2 inhibitor is administered in a therapeutically effective amount, such as from 10 μg/kg to 500 mg/kg (e.g., 10 μg/kg, 100 μg/kg, 500 μg/kg, 1 mg/kg, 10 mg/kg, 50 mg/kg, 100 mg/kg, 250 mg/kg, or 500 mg/kg). In some embodiments, the GRK2 inhibitor is administered bimonthly, once a month, once every two weeks, or at least once a week or more (e.g., 1, 2, 3, 4, 5, 6, or 7 times a week or more).

The GRK2 inhibitor is administered to the patient in an amount sufficient to decrease tumor growth, decrease tumor burden, increase cancer cell death, or increase progression free survival by 10% or more (e.g., 10%, 20%, 30%, 40%, 50%, 60%, 70%, 80%, 90%, 95% or more). Tumor growth and tumor burden can be assessed using standard imaging methods (e.g., digital radiography, PET scan, CT scan, or MRI scan). Images from before and after administration of the GRK2 inhibitor can be compared to evaluate the efficacy of the treatment, and the rate of disease progression can be assessed by comparison to the patient's medical history prior to administration of the GRK2 inhibitor. A finding of a reduction in the total number of tumors, number of primary tumors, volume of tumors, growth of tumors, or rate of disease progression indicates that the GRK2 inhibitor has successfully treated the cancer.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

1. A method of treating a subject with cancer, the method comprising administering to the subject an effective amount of a G-protein-coupled receptor kinase 2 (GRK2) small molecule antagonist.

2. A method of treating a subject with cancer, the method comprising contacting a tumor, tumor microenvironment, site of metastasis, cancer cell, metastatic cancer cell, or stromal cell in a tumor microenvironment with an effective amount of a GRK2 small molecule antagonist.

3. A method of treating a subject identified as having cancer, the method comprising administering to the subject an effective amount of a GRK2 small molecule antagonist.

4. A method of treating a subject identified as having cancer, the method comprising contacting a tumor, tumor microenvironment, site of metastasis, cancer cell, metastatic cancer cell, or stromal cell in a tumor microenvironment with an effective amount of a GRK2 small molecule antagonist.

5. A method of reducing or inhibiting cancer cell proliferation in a subject in need thereof, the method comprising administering to the subject an effective amount of a GRK2 small molecule antagonist.

6. A method of reducing or inhibiting cancer cell proliferation in a subject in need thereof, the method comprising contacting a cancer cell with an effective amount of a GRK2 small molecule antagonist.

7. A method of reducing or inhibiting cancer cell or tumor growth in a subject in need thereof, the method comprising administering to the subject an effective amount of a GRK2 small molecule antagonist.

8. A method of reducing or inhibiting cancer cell or tumor growth in a subject in need thereof, the method comprising contacting a tumor, tumor microenvironment, site of metastasis, cancer cell, metastatic cancer cell, or stromal cell in a tumor microenvironment with an effective amount of a GRK2 small molecule antagonist.

9. The method of any one of claims 1-8, wherein the cancer is GRK2-associated cancer.

10. The method of any one of claims 1-9, wherein the method comprises identifying the cancer as GRK2-associated cancer prior to administration of the GRK2 small molecule antagonist.

11. A method of treating a subject with cancer, the method comprising: (a) identifying a subject with GRK2-associated cancer; and (b) administering to the subject an effective amount of a GRK2 small molecule antagonist.

12. A method of treating a subject with cancer, the method comprising: (a) identifying a subject with GRK2-associated cancer; and (b) contacting a tumor, tumor microenvironment, site of metastasis, cancer cell, metastatic cancer cell, or stromal cell in a tumor microenvironment with an effective amount of a GRK2 small molecule antagonist.

13. A method of treating a subject with GRK2-associated cancer, the method comprising administering to the subject an effective amount of a GRK2 small molecule antagonist.

14. A method of treating a subject with GRK2-associated cancer, the method comprising contacting a tumor, tumor microenvironment, site of metastasis, cancer cell, metastatic cancer cell, or stromal cell in a tumor microenvironment with an effective amount of a GRK2 small molecule antagonist.

15. The method of claim 2, 4, 8, 12, or 14, wherein the wherein the method comprises contacting a tumor with an effective amount of a GRK2 small molecule antagonist.

16. The method of claim 2, 4, 8, 12, or 14, wherein the wherein the method comprises contacting a tumor microenvironment with an effective amount of a GRK2 small molecule antagonist.

17. The method of claim 2, 4, 8, 12, or 14, wherein the wherein the method comprises contacting a site of metastasis with an effective amount of a GRK2 small molecule antagonist.

18. The method of claim 2, 4, 8, 12, or 14, wherein the wherein the method comprises contacting a cancer cell with an effective amount of a GRK2 small molecule antagonist.

19. The method of claim 2, 4, 8, 12, or 14, wherein the wherein the method comprises contacting a metastatic cancer cell with an effective amount of a GRK2 small molecule antagonist.

20. The method of claim 2, 4, 8, 12, or 14, wherein the wherein the method comprises contacting a stromal cell in a tumor microenvironment with an effective amount of a GRK2 small molecule antagonist.

21. A method of predicting the response of a cancer in a subject to treatment with a GRK2 inhibitor, the method comprising contacting a cancer cell isolated from the subject with a GRK2 small molecule antagonist and evaluating the response of the cancer cell.

22. The method of claim 21, wherein the evaluating comprises assessing cancer cell growth, cancer cell proliferation, cancer cell metastasis, cancer cell invasion, cancer cell migration, cancer cell death, cancer cell autophagy, cancer cell GRK2 expression, or cancer cell innervation.

23. The method of claim 22, wherein the evaluating comprises assessing cancer cell proliferation.

24. A method of predicting the response of a cancer in a subject to treatment with a GRK2 inhibitor, the method comprising: (a) isolating a cancer cell from the subject; (b) measuring the expression of GRK2 in the cancer cell; and (c) comparing GRK2 expression in the cancer cell to a reference, wherein increased expression of GRK2 in the cancer cell as compared to the reference indicates that the subject will respond to treatment with a small molecule antagonist.

25. The method of claim 24, wherein the method further comprises providing a GRK2 small molecule antagonist suitable for administration to the subject.

26. The method of claim 24, wherein the method further comprises administering to the subject an effective amount of a GRK2 small molecule antagonist.

27. The method of any one of claims 1-26, wherein the GRK2 small molecule antagonist is a compound of Formula I, Formula II, or Formula III, Formula IV, or any one of compounds 1-70.

28. The method of claim 27, wherein the GRK2 small molecule antagonist is a compound of Formula I.

29. The method of claim 28, wherein the GRK2 small molecule antagonist of Formula I is a compound of any one of Formula I-1 to I-39.

30. The method of claim 27, wherein the GRK2 small molecule antagonist is a compound of Formula II.

31. The method of claim 30, wherein the GRK2 small molecule antagonist of Formula II is a compound of any one of Formula II-1 to II-35.

32. The method of claim 27, wherein the GRK2 small molecule antagonist is a compound of Formula III.

33. The method of claim 32, wherein the GRK2 small molecule antagonist of Formula III is a compound of any one of Formula III-1 to III-56.

34. The method of claim 27, wherein the GRK2 small molecule antagonist is a compound of Formula IV.

35. The method of claim 34, wherein the GRK2 small molecule antagonist of Formula IV is a compound of any one of Formula IV-1 to IV-80.

36. The method of claim 27, wherein the GRK2 small molecule antagonist is any one of compounds 1-70.

37. The method of any one of claims 1-36, wherein the GRK2 small molecule antagonist reduces GRK2 kinase activity.

38. The method of any one of claims 1-37, wherein the cancer is pancreatic cancer, melanoma, small cell lung cancer, non-small cell lung cancer, gastric cancer, colorectal cancer, head and neck cancer, ovarian cancer, testicular cancer, thymoma, uterine cancer, kidney cancer, acute myeloid leukemia, diffuse large B-cell lymphoma, prostate cancer, breast cancer, or hepatocellular carcinoma.

39. The method of claim 38, wherein the cancer is pancreatic cancer.

40. The method of any one of claims 1-39, wherein the GRK2 small molecule antagonist is administered locally.

41. The method of claim 40, wherein the GRK2 small molecule antagonist is administered intratumorally.

42. The method of any one of claims 1-41, wherein the method further comprises administering a second therapeutic agent.

43. The method of claim 42, wherein the second therapeutic agent is an anti-cancer agent, a GRK2 signaling inhibitor, a GRK2 function blocker, a neurotransmission blocker, or a neuronal growth factor blocker.

44. The method of claim 43, wherein the second therapeutic agent is an anti-cancer agent.

45. The method of claim 44, wherein the anti-cancer agent is a chemotherapeutic agent, a checkpoint inhibitor, a biologic cancer agent, a cancer-specific agent, a cytokine therapy, an anti-angiogenic drug, a drug that targets cancer metabolism, an antibody that marks a cancer cell surface for destruction, an antibody-drug conjugate, a cell therapy, a commonly used anti-neoplastic agent, a CAR-T therapy, an oncolytic virus, or a non-drug therapy.

46. The method of claim 45, wherein the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

47. The method of any one of claims 1-46, wherein the GRK2 small molecule antagonist does not cross the blood brain barrier (BBB).

48. The method of claim 47, wherein the GRK2 small molecule antagonist has been modified to prevent BBB crossing by conjugation to a targeting moiety, formulation in a particulate delivery system, addition of a molecular adduct, or through modulation of its size, polarity, flexibility, or lipophilicity.

49. The method of any one of claims 1-48, wherein the GRK2 small molecule antagonist does not have a direct effect on the central nervous system or gut.

50. The method of any one of claims 1-49, wherein the GRK2 small molecule antagonist decreases tumor volume, decreases tumor growth, decreases tumor innervation, decreases nerve activity in a tumor, decreases cancer cell proliferation, decreases cancer cell invasion, decreases cancer cell migration, decreases cancer cell metastasis, increases cancer cell autophagy, increases cancer cell death, decreases tumor GRK2 expression, causes remission, treats the cancer or tumor, increases time to recurrence, or improves survival.

51. The method of any one of claims 1-50, wherein the method further comprises measuring one or more of tumor volume, tumor growth, tumor innervation, nerve activity in a tumor, cancer cell proliferation, cancer cell invasion, cancer cell migration, cancer cell metastasis, cancer cell autophagy, cancer cell death, or GRK2 expression before administration of the GRK2 small molecule antagonist.

52. The method of any one of claims 1-51, wherein the method further comprises measuring one or more of tumor volume, tumor growth, tumor innervation, nerve activity in a tumor, cancer cell proliferation, cancer cell invasion, cancer cell migration, cancer cell metastasis, cancer cell autophagy, cancer cell death, or GRK2 expression after administration of the GRK2 small molecule antagonist.

53. The method of any one of claims 1-52, wherein the GRK2 small molecule antagonist is administered in an amount sufficient to decrease tumor volume, decrease tumor growth, decrease tumor innervation, decrease nerve activity in a tumor, decrease cancer cell proliferation, decrease cancer cell invasion, decrease cancer cell migration, decrease cancer cell metastasis, increase cancer cell autophagy, increase cancer cell death, decrease tumor GRK2 expression, cause remission, treat the cancer or tumor, increase time to recurrence, or improve survival.

54. The method of any one of claims 1-53, wherein the subject is not diagnosed as having high blood pressure or a cardiac condition.

55. The method of any one of claims 1-54, wherein the subject is human.

56. An anti-cancer therapy comprising a GRK2 small molecule antagonist a second agent selected from the group consisting of chemotherapeutic agents, checkpoint inhibitors, biologic cancer agents, cancer-specific agents, cytokine therapies, anti-angiogenic drugs, drugs that target cancer metabolism, antibodies that mark a cancer cell surface for destruction, antibody-drug conjugates, cell therapies, commonly used anti-neoplastic agents, CAR-T therapies, oncolytic viruses, non-drug therapies, neurotransmission blockers, and neuronal growth factor blockers.

57. The anti-cancer therapy of claim 56, wherein the GRK2 small molecule antagonist is a compound of Formula I, Formula II, Formula III, or Formula IV, or any one of compounds 1-70.

58. The anti-cancer therapy of claim 57, wherein the GRK2 small molecule antagonist is a compound of Formula I.

59. The anti-cancer therapy of claim 58, wherein the GRK2 small molecule antagonist of Formula I is a compound of any one of Formula I-1 to I-39.

60. The anti-cancer therapy of claim 57, wherein the GRK2 small molecule antagonist is a compound of Formula II.

61. The anti-cancer therapy of claim 60, wherein the GRK2 small molecule antagonist of Formula II is a compound of any one of Formula II-1 to II-35.

62. The anti-cancer therapy of claim 57, wherein the GRK2 small molecule antagonist is a compound of Formula III.

63. The anti-cancer therapy of claim 62, wherein the GRK2 small molecule antagonist of Formula III is a compound of any one of Formula III-1 to III-56.

64. The anti-cancer therapy of claim 57, wherein the GRK2 small molecule antagonist is a compound of Formula IV.

65. The anti-cancer therapy of claim 64, wherein the GRK2 small molecule antagonist of Formula IV is a compound of any one of Formula IV-1 to IV-80.

66. The anti-cancer therapy of claim 57, wherein the GRK2 small molecule antagonist is any one of compounds 1-70.

67. The anti-cancer therapy of any one of claims 56-66, wherein the second agent is a chemotherapeutic agent.

68. The anti-cancer therapy of any one of claims 56-66, wherein the second agent is a checkpoint inhibitor.

69. The anti-cancer therapy of claim 68, wherein the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

70. A pharmaceutical composition comprising a GRK2 small molecule antagonist and an anti-cancer therapeutic.

71. The pharmaceutical composition of claim 70, wherein the GRK2 small molecule antagonist is a compound of Formula I, Formula II, Formula III, or Formula IV, or any one of compounds 1-70.

72. The pharmaceutical composition of claim 71, wherein the GRK2 small molecule antagonist is a compound of Formula I.

73. The pharmaceutical composition of claim 72, wherein the GRK2 small molecule antagonist of Formula I is a compound of any one of Formula I-1 to I-39.

74. The pharmaceutical composition of claim 71, wherein the GRK2 small molecule antagonist is a compound of Formula II.

75. The pharmaceutical composition of claim 74, wherein the GRK2 small molecule antagonist of Formula II is a compound of any one of Formula II-1 toll-35.

76. The pharmaceutical composition of claim 71, wherein the GRK2 small molecule antagonist is a compound of Formula III.

77. The pharmaceutical composition of claim 76, wherein the GRK2 small molecule antagonist of Formula III is a compound of any one of Formula III-1 to III-56.

78. The pharmaceutical composition of claim 71, wherein the GRK2 small molecule antagonist is a compound of Formula IV.

79. The pharmaceutical composition of claim 78, wherein the GRK2 small molecule antagonist of Formula IV is a compound of any one of Formula IV-1 to IV-80.

80. The pharmaceutical composition of claim 71, wherein the GRK2 small molecule antagonist is any one of compounds 1-70.

81. The pharmaceutical composition of any one of claims 70-80, wherein the anti-cancer therapeutic is a chemotherapeutic agent, a checkpoint inhibitor, a biologic cancer agent, a cancer-specific agent, a cytokine therapy, an anti-angiogenic drug, a drug that targets cancer metabolism, an antibody that marks a cancer cell surface for destruction, an antibody-drug conjugate, a cell therapy, a commonly used anti-neoplastic agent, a CAR-T therapy, an oncolytic virus, or a non-drug therapy.

82. The pharmaceutical composition of claim 81, wherein the anti-cancer therapeutic is a chemotherapeutic agent.

83. The pharmaceutical composition of claim 81, wherein the anti-cancer agent is a checkpoint inhibitor.

84. The pharmaceutical composition of claim 83, wherein the checkpoint inhibitor is an inhibitory antibody, a fusion protein, an agent that interacts with a checkpoint protein, an agent that interacts with the ligand of a checkpoint protein, an inhibitor of CTLA-4, an inhibitor of PD-1, an inhibitor of PDL1, an inhibitor of PDL2, or an inhibitor of B7-H3, B7-H4, BTLA, HVEM, TIM3, GAL9, LAG3, VISTA, KIR, 2B4, CD160, CGEN-15049, CHK 1, CHK2, A2aR, or B-7 family ligands.

85. The pharmaceutical composition of any one of claims 70-84, wherein the composition further comprises a pharmaceutically acceptable excipient.
